(19) United States
(12) Patent Application Publication
(10) Pub. No.: US 2016/0177284 A1
(43) Pub. Date: Jun. 23, 2016

(54) CELL-TARGETED MOLECULES COMPRISING AMINO-TERMINUS PROXIMAL OR AMINO-TERMINAL SHIGA TOXIN A SUBUNIT EFFECTOR REGIONS

(71) Applicant: MOLECULAR TEMPLATES, INC., Georgetown, TX (US)

(72) Inventors: Eric POMA, New York, NY (US); Erin WILLERT, Round Rock, TX (US); Jack HIGGINS, Georgetown, TX (US); Jason KIM, Austin, TX (US)

(73) Assignee: MOLECULAR TEMPLATES, INC., Georgetown, TX (US)

(21) Appl. No.: 14/965,882

(22) Filed: Dec. 10, 2015

Related U.S. Application Data
(63) Continuation-in-part of application No. PCT/US2015/019708, filed on Mar. 10, 2015.
Application No. PCT/US2015/019708 is a continuation-in-part of application No. PCT/US2015/035179, filed on Jun. 10, 2015.
Application No. PCT/US2015/035179 is a continuation of application No. PCT/US2015/012970, filed on Jan. 26, 2015.
(60) Provisional application No. 61/951,121, filed on Mar. 11, 2014.
(60) Provisional application No. 62/010,918, filed on Jun. 11, 2014.
(60) Provisional application No. 62/049,325, filed on Sep. 11, 2014.
(60) Provisional application No. 61/932,000, filed on Jan. 27, 2014.

(51) Int. Cl. C12N 9/24; C07K 16/28; C07K 16/14; C07K 16/20; C07K 16/10; C07K 16/18; C07K 16/40; C07K 16/08

(52) U.S. Cl. CPC C12N 9/2497; C07K 16/40; C07K 16/2863; C07K 16/2803; C07K 16/2833; C07K 16/085; C07K 16/20; C07K 16/286; C07K 16/2866; C07K 16/1063; C07K 16/088; C07K 16/18; C07K 16/2887; C07K 16/14; C07K 16/1054; C12Y 302/02022; C07K 2317/622; C07K 2319/55; C07K 2319/33; C07K 2317/77; C07K 2317/92; C07K 2317/76; A61K 2039/505

(57) ABSTRACT

The present invention provides cell-targeted molecules comprising binding regions for cell-type specific targeting and Shiga toxin A Subunit effector regions for Shiga toxin effector functions, wherein the Shiga toxin effector regions are at and/or proximal to an amino-terminus of a polypeptide component of the cell-targeted molecule, and optionally comprising a disrupted, furin-cleavage motif between the Shiga toxin effector region and the binding region. The cell-targeted molecules of the invention exhibit a more optimized cytotoxicity and/or improved, in vivo tolerability as compared to related molecules comprising less amino-terminus proximal, Shiga toxin effector regions and/or furin-cleavage sensitive, wild-type, Shiga toxin effector regions. The cell-targeted molecules of the invention have uses, such as, e.g., in methods involving targeted killing of cells, delivering exogenous materials into cells, labeling subcellular compartments of cells, and diagnosing and/or treating a variety of conditions, including cancers, tumors, other growth abnormalities, immune disorders, and microbial infections.

### SEQUENCE LISTING

The instant application contains a Sequence Listing which has been submitted electronically in ASCII format and is hereby incorporated by reference in its entirety. Said ASCII copy, created on Nov. 27, 2015, is named 14-04PCT-CIP_SL.txt and is 139,212 bytes in size.

### FIELD OF THE INVENTION

The present invention relates to proteins comprising immunoglobulin-type binding regions for mediating cell targeting and Shiga toxin effector regions that are linked such that the Shiga toxin effector regions are at and/or proximal to amino-terminals of polypeptide components of the proteins. The molecules of the present invention have uses, e.g., for the selective killing of specific cell types, delivering exogenous materials inside target cells, labeling subcellular compartments of target cells, and as therapeutic molecules for the treatment of a variety of diseases, disorders, and conditions, including, inter alia, cancers, tumors, growth abnormalities, immune disorders, and microbial infections.

### BACKGROUND

The development of therapeutic molecules from natural protein toxins that are effective as therapeutics has challenged scientists for decades (see e.g. Pastan I et al., Annu Rev Med 58: 221-37 (2007)). One hurdle is that the enzymatic, toxin components of the therapeutic molecules must be able to efficiently reach their target substrates to be potently cytotoxic and provide viable therapeutic windows (Dosio F et al., Toxins (Basel) 3: 848-83 (2011); Govindan S, Goldenberg D, Expert Opin Biol Ther 12: 873-90 (2012); Feld J et al., Oncotarget 4: 397-412 (2013)). For therapeutics derived from bacterial or plant toxins with cytosolic substrates, the cytotoxic potency of the therapeutic might depend on its efficiency in intracellular routing (Pirie C et al., J Biol Chem 286: 4165-72 (2011)); however, understanding how protein toxins direct their own intracellular transport from endosomes to the cytosol remains a challenge to scientific inquiry (Antignani A, Fitzgerald D, Toxins 5: 1486-502 (2013)).

Many proteins contain conserved polypeptide sequences called domains, which form self-contained, folding units of protein structure that can function independently of the entire protein or when recombined into an orthologous protein (Kirshner M, Gerhart J, Proc Natl Acad Sci USA 95: 8420-7 (1988)). The use of modular protein domains in the creation of novel proteins offers almost limitless possibilities (Nixon A et. al, Proc Natl Acad Sci USA 94: 1069-73 (1997)). One possibility is the molecular engineering of chimeric molecules composed of targeting domains and protein toxin domains. Naturally occurring toxins or truncated toxin fragments have been linked or fused to immunoglobulin domains or receptor ligands through chemical conjugation or recombinant protein engineering techniques with the hope of creating cell-targeted therapeutic molecules (Moolten F, Cooperband S, Science 169: 68-70 (1970); Thorpe P et al., Nature 271: 752-5 (1978); Krolick K et al., Proc Natl Acad Sci USA 77: 5419-23 (1980); Krolick K et al., Cancer Immunol Immunother 12: 39-41 (1981); Blythman H et al., Nature 290: 145-46 (1981); Chaudhary V et al., Nature 339: 394-7 (1989); Strom T et al., Semin Immunol 2: 467-79 (1990); Pastan I et al., Annu Rev Biochem 61: 331-54 (1992); Foss F et al., Curr Top Microbiol Immunol 234: 63-81 (1998)). One aim of such molecular engineering is to design chimeric molecules, such as immunotoxins and ligand-toxin fusions, with the dual functionality of: 1) delivering toxins to specific cell types or places within an organism after systemic administration; and 2) effectuating a targeted cytotoxicity to specific cells using potent cytotoxicity mechanisms effective in eukaryotic cells.

The Shiga toxin family of related protein toxins, notably toxins isolated from S. dysenteriae and E. coli, is composed of various naturally occurring toxins which are structurally and functionally related (Johannes L, Römer W, Nat Rev Microbiol 8: 105-16 (2010)). For example, the Shiga toxin family encompasses true Shiga toxin (Stx) isolated from S. dysenteriae serotype 1, Shiga-like toxin 1 variants (SLT1 or Stx1 or SLT-1 or Slt-I) isolated from serotypes of enterohemorrhagic E. coli, and Shiga-like toxin 2 variants (SLT2 or Stx2 or SLT-2) isolated from serotypes of enterohemorrhagic E. coli. SLT1 differs by only one residue from Stx, and both have been referred to as Verocytotoxins or Verotoxins (VTs) (O'Brien A et al., Curr Top Microbiol Immunol 180: 65-94 (1992)). Members of the Shiga toxin family share the same overall structure and mechanism of action (Engedal N et al., Microbial Biotech 4: 32-46 (2011)). For example, Stx, SLT-1 and SLT-2 display indistinguishable enzymatic activity in cell free systems (Head S et al., J Biol Chem 266: 3617-21 (1991); Tesh V et al., Infect Immun 61: 3392-402 (1993); Brigotti M et al., Toxicon 35:1431-1437 (1997)). Members of the Shiga toxin family contain targeting domains that preferentially bind a specific glycosphingolipid present on the surface of some host cells and an enzymatic domain capable of permanently inactivating ribosomes once inside a cell (Johannes L, Römer W, Nat Rev Microbiol 8: 105-16 (2010)).

Members of the Shiga toxin family are employed by bacteria as virulence factors during infection of a host (Johannes L, Römer W, Nat Rev Microbiol 8: 105-16 (2010)). In an infected host, Shiga toxins are cytotoxic because of the toxins' potent ability to inhibit protein synthesis and to trigger apoptotic cell death (Johannes L, Römer W, Nat Rev Microbiol 8: 105-16 (2010)). The potent cytotoxic effects of Shiga toxins on host cells can result in hemorrhagic colitis and hemolytic uremic syndrome (Johannes L, Römer W, Nat Rev Microbiol 8: 105-16 (2010)).

Members of the Shiga toxin family share a common, multimeric, protein structure characterized by an A(B)₅ arrangement of Shiga protein subunits (Johannes L, Römer W, Nat Rev Microbiol 8: 105-16 (2010)). Each Shiga toxin is composed of two protein subunits, A and B, that associate in an A(B)₅ arrangement to form a holotoxin protein complex. The Shiga toxin A Subunit is a 32 kilodalton monomer that contains an enzymatic domain, and the Shiga toxin B Subunit is a 7.7 kilodalton subunit that associates with four other Shiga toxin B Subunits to form a pentamer of Shiga toxin B Subunits. The pentamer of B subunits associates with one A subunit to form the Shiga holotoxin, which is about 70 kilodaltons (O'Brien A, Holmes, R, Microbiol Rev 51: 206-20 (1987)).

Members of the Shiga toxin family share a common process for the intoxication of a host cell that can be divided into five main phases: cell surface binding, endocytosis, retrograde subcellular movement to the endoplasmic reticulum, translocation from the endoplasmic reticulum to the cytosol, and the enzymatic inactivation of ribosomes in the cytosol. First, Shiga holotoxins are directed to the cellular surfaces of specific host cells by the B subunit's ability to bind glycosphingolipid globotriaosylceramides, such as Gb3 (also known as CD77), present on the exoplasmic membrane leaflet (Ling, H et al, Biochemistry 37: 1777-88 (1998); Thorpe C et al., Infect Immun 67: 5985-93 (1999); Soltyk A et al., J Biol Chem 277: 5351-59 (2002)). Second, Shiga holotoxins exploit the host cell's endocytotic machinery to enter into the host cell, where the holotoxins are initially contained within the endosomes (Sandvig K et al., J Cell Biol 108: 1331-43 (1989); Sandvig K et al., Histochem Cell Biol 117: 131-141 (2002)). Third, Shiga holotoxins exploit the host cell's intracellular-transport machinery to reach the endoplasmic reticulum and gain access to the cytosol (Nichols B et al., J Cell Biol 153: 529-41 (2001); Lauvrak S et al., J Cell Sci 117: 2321-31 (2004); Saint-Pol A et al., Dev. Cell 6: 525-38 (2004)). Fourth, enzymatically active fragments of the Shiga holotoxins retrotranslocate from the lumen of the endoplasmic reticulum to the cytosol (Yu M, Haslam D, Infect Immun 73: 2524-32 (2005); LaPointe P et al., J Biol Chem 280: 23310-18 (2005); Falguieres T, Johannes L, Biol Cell 98: 125-34 (2006); Tam P, Lingwood C, Microbiology 153: 2700-10 (2007)). Fifth, the cytosolic fraction of Shiga toxin enzymatically active fragments causes cytotoxicity by inactivating host-cell ribosomes (Tam P, Lingwood C, Microbiology 153: 2700-10 (2007)).

During the Shiga toxin intoxication process, the Shiga toxin A Subunit is proteolytically cleaved between a conserved arginine residue and a methionine residue (e.g. Arg251-Met252 in StxA and SLT-1A) by furin, a host cell endoprotease (Garred Ø et al., J Biol Chem 270: 10817-21 (1995)). The amino-terminal fragment of the furin-cleaved, Shiga toxin A Subunit is called the Shiga toxin “A1 fragment” (or Stxn-A1, SLTn-A1, SLT-nA1), and the carboxy-terminal fragment of the A Subunit is called the Shiga toxin “A2 fragment.” The Shiga toxin A1 fragment is an approximately 27.5 kilodalton polypeptide which contains the catalytic domain of the Shiga toxin (Fraser M et al., Nat Struct Biol 1: 59-64 (1994)). The mechanism of Shiga toxin cytotoxicity to host cells is predominantly through the A1 fragment's potent catalytic inactivation of eukaryotic ribosomes and cell-wide inhibition of protein synthesis (Johannes L, Römer W, Nat Rev Microbiol 8: 105-16 (2010)).

The Shiga toxin A1 fragment inhibits protein translation by its potent depurination activity towards a universally conserved adenine nucleobase at position 4,324 in the alpha-sarcin-ricin loop of the 28S ribosomal RNA of the eukaryotic ribosome (Johannes L, Römer W, Nat Rev Microbiol 8: 105-16 (2010)). After a threshold number of ribosomes is inactivated, the host cell is predicted to experience sufficient reduction in protein synthesis to induce cell death via apoptosis (Iordanov M et al., Mol Cell Biol 17: 3373-81 (1997); Smith W et al., Infect Immun 71: 1497-504 (2003); Lee S et al., Cell Microbiol 10: 770-80 (2008); Tesh V, Future Microbiol 5: 431-53 (2010)).

A ribosome-inactivating A-B toxin can permanently cripple one ribosome after another within the same cell at a rate of approximately 1,500 ribosomes per minute (Endo Y, Tsurugi K, Eur J Biochem 171: 45-50 (1988); Endo Y et al., J Biol Chem 263: 8735-9 (1988)). The potency of A-B toxins is reported to be extremely high, such that as little as one toxin molecule can kill a cell (Yamaizumi M et al., Cell 15: 245-50 (1978); Antignani A, Fitzgerald D, Toxins 5: 1486-502 (2013)). It is believed that a single molecule of A-B toxin can irreversibly inactivate 300 ribosomes in 35 minutes and is sufficient to kill a cancer cell (Weldon J, Pastan I, FEBS Journal 278: 4683-700 (2011)). This level of cytotoxic potency is further predicted for Shiga toxin A Subunit, for which it has been suggested that one molecule translocated into the cytosol would be sufficient to kill a cell (Tam P, Lingwood C, Microbiology 153: 2700-10 (2007)).

Holotoxins of the Shiga toxin family are predicted to be too toxic for untargeted use as a therapeutic (Jain, R, Tumor physiology and antibody delivery, Front Radiat Ther Oncol 24: 32-46 (1990)). However, members of the Shiga toxin family have the potential to be synthetically engineered for therapeutic applications by rational alterations to the toxin's structure, characteristics, and biological activities (Johannes L, Römer W, Nat Rev Microbiol 8: 105-16 (2010); Engedal N, Microbial Biotech 4: 32-46 (2011)). Shiga holotoxins have a bipartite structure composed of two non-covalently attached, modular parts: an A-moiety containing the enzymatically active A1 fragment and a B-moiety containing binding sites to target specific, cell-surface glycosphingolipids. Because the Shiga toxin subunits are modular, it has been hypothesized that therapeutic compositions may be created based on the separate structures and functions of the A and B moieties (WO2007033497; US20090156417 A1; Johannes L, Römer W, Nat Rev Microbiol 8: 105-16 (2010); Engedal N, Microbial Biotech 4: 32-46 (2011); US20130196928 A1, each of which is incorporated by reference herein in its entirety).

The A-moiety of members of the Shiga toxin family is stable, enzymatically active, and cytotoxic independent of any B-moiety (Engedal N, Microbial Biotech 4: 32-46 (2011)). The Shiga toxin 1 A Subunit is catalytically active, capable of enzymatically inactivating ribosomes in vitro, and cytotoxic even if truncated or fused to other protein domains (Haddad J et al., J Bacteriol 175: 4970-8 (1993); A1-Jaufy A et al., Infect Immun 62: 956-60 (1994); A1-Jaufy A et al., Infect Immun 63: 3073-8 (1995); LaPointe P et al., J Biol Chem 280: 23310-18 (2005); Di R et al., Toxicon 57: 535-39 (2011)). Shiga-like toxin 1 A Subunit truncations are catalytically active, capable of enzymatically inactivating ribosomes in vitro, and cytotoxic when expressed within a cell (LaPointe P et al., J Biol Chem 280: 23310-18 (2005)).

Efficient cell killing by Shiga toxins requires the intracellular cleavage of the Shiga toxin A Subunit in a conserved, surface-exposed, extended loop, structure by the endoprotease furin (Garred Ø et al., Exp Cell Res 218: 39-49 (1995); Garred Ø et al., J Biol Chem 270: 10817-21 (1995); Lea N et al., Microbiology 145: 999-1004 (1999); Kurmanova A et al., Biochem Biophys Res Commun 357: 144-9 (2007)). Only the Shiga toxin A1 fragment localizes to the cytosol in intoxicated cells as the Shiga toxin A2 fragment and B Subunits remain in the endoplasmic reticulum (Tam P, Lingwood C, Microbiology 153: 2700-10 (2007)). The proteolytic cleavage of Shiga toxin A Subunits at this conserved, extended loop structure contributes to the liberation of the catalytic A1 fragment and the subcellular routing of the A1 fragment to the cytosol (Johannes L, Römer W, Nat Rev Microbiol 8: 105-16 (2010)). The Shiga toxin A2 fragment is an approximately 4.5-4.7 kildalton polypeptide that is superfluous for catalytic activity (Haddad J et al., J Bacteriol 175: 4970-8 (1993); Backer M et al., J Control Release 74: 349-55 (2001); Backer M, Backer J, Bioconjug Chem 12: 1066-73 (2001); LaPointe P et al., J Biol Chem 280: 23310-18 (2005); Di R et al., Toxicon 57: 535-39 (2011)).

Shiga toxin activity is increased by proteolytic cleavage (Brown J et al., FEBS Lett 117: 84-8 (1980); Reisbig R et al., J Biol Chem 256: 8739-44 (1981)). Shiga toxins require the intracellular cleavage of their A Subunits by the endoprotease furin in intoxicated cells for the most efficient killing of intoxicated cells (Garred Ø et al., Exp Cell Res 218: 39-49 (1995); Garred Ø et al., J Biol Chem 270: 10817-21 (1995); Lea N et al., Microbiology 145: 999-1004 (1999); Kurmanova A et al., Biochem Biophys Res Commun 357: 144-9 (2007)). This proteolytic processing must be accounted for in the design of molecules comprising Shiga toxin A Subunit derived components to supply the most efficient toxin activation and/or subcellular routing required for maximal, Shiga toxin cytotoxicity (see Garred Ø et al., Exp Cell Res 218: 39-49 (1995); Lea N et al., Microbiology 145: 999-1004 (1999); Kurmanova A et al., Biochem Biophys Res Commun 357: 144-9 (2007)).

It would be desirable to have cytotoxic, cell-targeted molecules comprising Shiga toxin A Subunit derived components which are as cytotoxic as possible. The cytotoxic potency of a Shiga toxin A Subunit based construct depends on the ability of its Shiga toxin A Subunit, enzymatic component to efficiently access the cytosol (Tam P, Lingwood C, Microbiology 153: 2700-10 (2007)). It would be desirable to have highly cytotoxic, cell-targeted molecules comprising Shiga-toxin-Subunit-A-derived, polypeptide regions that self-direct their own intracellular routing and display potent cytotoxicity for applications involving the targeted killing of specific cell types and for use as therapeutics in the treatment of a variety of diseases, such as e.g., cancers, tumors, immune disorders, and microbial infections. It would also be desirable to have cytotoxic, cell-targeted molecules with reduced nonspecific toxicities, improved stabilities, increased in vivo half-lives, and/or improved, general, toxicity profiles after administration to mammals. Thus, there remains a need in the art for ways of engineering 1) cytotoxic, cell-targeted molecules comprising Shiga toxin A Subunit effector regions that retain potent toxin effector functionalities, such as self-directed intracellular routing and cytotoxicity, after being linked to heterologous, polypeptide binding regions for cell targeting; and 2) more stable, cytotoxic, cell-targeted molecules comprising potent Shiga toxin A Subunit effectors regions but with reduced off-target toxicities.

### SUMMARY OF THE INVENTION

The present invention provides various cell-targeted molecules (e.g., cell-targeted proteins), each comprising 1) a binding region capable of specifically binding an extracellular target biomolecule and 2) a Shiga toxin A Subunit effector polypeptide region; wherein the Shiga toxin effector polypeptide regions are at and/or proximal to an amino-terminus of a polypeptide component of the cell-targeted molecule. Optionally, the cell-targeted molecules of the present invention may have a disrupted furin-cleavage motif at the carboxy-terminus of the Shiga toxin A1 fragment region of the Shiga toxin effector polypeptide, such as, e.g., a disrupted furin-cleavage motif located between the Shiga toxin effector region and the cell-targeting binding region, in order to reduce the likelihood of the premature separation of the Shiga toxin effector polypeptide region from the cell-targeting binding region.

In certain embodiments, the cell-targeted molecule of the present invention comprises 1) a binding region capable of specifically binding at least one extracellular target biomolecule and 2) a Shiga toxin effector polypeptide region having an amino-terminus; wherein the amino-terminus of the Shiga toxin effector polypeptide is at and/or proximal to an amino-terminus of a polypeptide component of the cell-targeted molecule. In certain further embodiments, the binding region comprises one or more peptides and/or polypeptides.

In certain embodiments, the cell-targeted molecule of the present invention has an amino-terminus and comprises 1) a binding region comprising a peptide or polypeptide and capable of specifically binding at least one extracellular target biomolecule; and 2) a Shiga toxin effector polypeptide region; wherein the Shiga toxin effector polypeptide region is positioned at and/or proximal to the amino-terminus of the cell-targeted molecule. In certain further embodiments, the binding region comprises one or more peptides and/or polypeptides.

In certain embodiments, the cell-targeted molecule of the present invention has an amino-terminus and comprises 1) a binding region comprising a peptide or polypeptide and capable of specifically binding at least one extracellular target biomolecule; and 2) a Shiga toxin effector polypeptide region having an amino-terminus; wherein the amino-terminus of the Shiga toxin effector polypeptide is positioned at and/or proximal to the amino-terminus of the cell-targeted molecule. In certain further embodiments, the binding region comprises one or more peptides and/or polypeptides.

In certain embodiments, the cell-targeted molecule of the present invention comprises 1) a heterologous, binding region for cell targeting; and 2) a cytotoxic, Shiga toxin effector polypeptide region. In certain embodiments of the cell-targeted molecule of the present invention, the binding region and Shiga toxin effector polypeptide region are physically arranged or oriented within the cytotoxic protein such that the binding region is not located proximally to the amino-terminus of the Shiga toxin effector polypeptide region. In certain embodiments of the cell-targeted molecule of the present invention, the specific order or orientation of the Shiga toxin effector polypeptide region and binding region is fixed such that the binding region is located within the cell-targeted molecule more proximal to the carboxy-terminus of the Shiga toxin effector polypeptide region than to the amino-terminus of the Shiga toxin effector polypeptide region.

In certain embodiments, the binding region of the cell-targeted molecule of the present invention is linked, either directly or indirectly, to the cell-targeted molecule of the present invention at a position carboxy-terminal to the Shiga toxin effector polypeptide region.

In certain embodiments, the binding region is linked, either directly or indirectly, to the Shiga toxin effector polypeptide region by at least one covalent bond which is not a disulfide bond. In certain further embodiments, the binding region is fused, either directly or indirectly, to the carboxy-terminus of the Shiga toxin effector polypeptide to form a single, continuous polypeptide.

In certain embodiments, the binding region of the cell-targeted molecule of the present invention is capable of binding to an extracellular target biomolecule selected from the group consisting of: CD20, CD22, CD40, CD74, CD79, CD25, CD30, HER2/neu/ErbB2, EGFR, EpCAM, EphB2, prostate-specific membrane antigen, Cripto, CDCP1, endoglin, fibroblast activated protein, Lewis-Y, CD19, CD21, CS1/SLAMF7, CD33, CD52, CD133, CEA, gpA33, mucin, TAG-72, tyrosine-protein kinase transmembrane receptor, carbonic anhydrase IX, folate binding protein, ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANK, FAP, tenascin, CD64, mesothelin, BRCA1, MART-1/MelanA, gp100, tyrosinase, TRP-1, TRP-2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen, prostate specific antigen, prostate stem cell antigen, human aspartyl (asparaginyl) beta-hydroxylase, EphA2, HER3/ErbB-3, MUC1, MART-1/MelanA, gp100, tyrosinase associated antigen, HPV-E7, Epstein-Barr virus antigen, Bcr-Abl, alpha-fetoprotein antigen, 17-A1, bladder tumor antigen, CD38, CD15, CD23, CD53, CD88, CD129, CD183, CD191, CD193, CD244, CD294, CD305, C3AR, FceRIa, galectin-9, mrp-14, PD-L1 (programmed death-ligand 1), Siglec-8, Siglec-10, CD49d, CD13, CD44, CD54, CD63, CD69, CD123, TLR4, FceRIa, IgE, CD107a, CD203c, CD14, CD68, CD80, CD86, CD105, CD115, F4/80, ILT-3, galectin-3, CD11a-c, GITRL, MHC class I molecule, MHC class II molecule, CD284 (TLR4), CD107-Mac3, CD195 (CCR5), HLA-DR, CD16/32, CD282 (TLR2), and any immunogenic fragment of any of the foregoing.

In certain embodiments, the cell-targeted molecule of the present invention is capable when introduced to cells of exhibiting cytotoxicity that is greater than that of a second cell-targeted molecule having an amino-terminus and comprising the binding region and the Shiga toxin effector polypeptide region which is not positioned at or proximal to the amino-terminus of the second cell-targeted molecule.

In certain embodiments, the cell-targeted molecule of the present invention has an amino-terminus and comprises 1) a binding region capable of specifically binding at least one extracellular target biomolecule and 2) a Shiga toxin effector polypeptide region; wherein the Shiga toxin effector polypeptide region is positioned at and/or proximal to the amino-terminus of the cell-targeted molecule; and wherein the cell-targeted molecule is capable when introduced to cells of exhibiting cytotoxicity that is greater than that of a second cell-targeted molecule having an amino-terminus and comprising the binding region and the Shiga toxin effector polypeptide region which is not positioned at or proximal to the amino-terminus of the second cell-targeted molecule. In certain further embodiments, the cell-targeted molecule of the present invention exhibits cytotoxicity that is greater by 3-fold, 4-fold, 5-fold, 6-fold, 7-fold, 8-fold, 9-fold, 10-fold than the cytotoxicity of the second cell-targeted molecule. In certain further embodiments, the cell-targeted molecule of the present invention exhibits cytotoxicity that is greater by 3-fold, 4-fold, 5-fold, 6-fold, 7-fold, 8-fold, 9-fold, 10-fold than the cytotoxicity of the second cell-targeted molecule as assayed by CD50 values, such as, e.g., by using a cell-kill assay described herein. In certain further embodiments, the cytotoxicity of the cell-targeted molecule of the present invention to target positive cells is 3-fold, 4-fold, 5-fold, 6-fold, 7-fold, 8-fold, 9-fold, 10-fold or greater than the cytotoxicity of the second cell-targeted molecule to target positive cells as assayed by CD₅₀ values.

In certain embodiments, the cell-targeted molecule of the present invention has an amino-terminus and comprises 1) a binding region capable of specifically binding at least one extracellular target biomolecule and 2) a Shiga toxin effector polypeptide region having an amino-terminus; wherein the amino-terminus of the Shiga toxin effector polypeptide is positioned at and/or proximal to the amino-terminus of the cell-targeted molecule; and wherein the cell-targeted molecule is capable when introduced to cells of exhibiting cytotoxicity that is greater than that of a second cell-targeted molecule comprising the binding region and the Shiga toxin effector polypeptide region wherein the amino-terminus of the Shiga toxin effector polypeptide region is not positioned at or proximal to the amino-terminus of the cell targeted molecule. In certain further embodiments, the cell-targeted molecule of the present invention exhibits cytotoxicity that is greater by 3-fold, 4-fold, 5-fold, 6-fold, 7-fold, 8-fold, 9-fold, 10-fold than the cytotoxicity of the second cell-targeted molecule. In certain further embodiments, the cell-targeted molecule of the present invention exhibits cytotoxicity that is greater by 3-fold, 4-fold, 5-fold, 6-fold, 7-fold, 8-fold, 9-fold, 10-fold than the cytotoxicity of the second cell-targeted molecule as assayed by CD50 values, such as, e.g., by using a cell-kill assay described herein. In certain further embodiments, the cytotoxicity of the cell-targeted molecule of the present invention to target positive cells is 3-fold, 4-fold, 5-fold, 6-fold, 7-fold, 8-fold, 9-fold, 10-fold or greater than the cytotoxicity of the second cell-targeted molecule to target positive cells as assayed by CD₅₀ values.

In certain embodiments, the cell-targeted molecule of the present invention comprises 1) a binding region capable of specifically binding at least one extracellular target biomolecule and 2) a Shiga toxin effector polypeptide region having an amino-terminus; wherein the amino-terminus of the Shiga toxin effector polypeptide is at and/or proximal to an amino-terminus of a polypeptide component of the cell-targeted molecule; and wherein the cell-targeted molecule is capable when introduced to cells of exhibiting cytotoxicity that is greater than that of a second cell-targeted molecule comprising the binding region and the Shiga toxin effector polypeptide region oriented such that the binding region is linked, either directly or indirectly, to the amino-terminus of the Shiga toxin effector polypeptide region. In certain further embodiments, the cell-targeted molecule of the present invention exhibits cytotoxicity that is greater by 3-fold, 4-fold, 5-fold, 6-fold, 7-fold, 8-fold, 9-fold, 10-fold than the cytotoxicity of the second cell-targeted molecule. In certain further embodiments, the cell-targeted molecule of the present invention exhibits cytotoxicity that is greater by 3-fold, 4-fold, 5-fold, 6-fold, 7-fold, 8-fold, 9-fold, 10-fold than the cytotoxicity of the second cell-targeted molecule as assayed by CD50 values, such as, e.g., by using a cell-kill assay described herein. In certain further embodiments, the cytotoxicity of the cell-targeted molecule of the present invention to target positive cells is 3-fold, 4-fold, 5-fold, 6-fold, 7-fold, 8-fold, 9-fold, 10-fold or greater than the cytotoxicity of the second cell-targeted molecule to target positive cells as assayed by CD₅₀ values.

In certain embodiments, the binding region of the cell-targeted molecule of the present invention comprises an immunoglobulin-type binding region. In certain embodiments, the immunoglobulin-type binding region is linked, either directly or indirectly, to the Shiga toxin effector polypeptide region by at least one covalent bond which is not a disulfide bond. In certain further embodiments, the immunoglobulin-type binding region is fused, either directly or indirectly, to the carboxy terminus of the Shiga toxin effector polypeptide to form a single, continuous polypeptide.

In certain embodiments, the binding region of the cell-targeted molecule of the present invention comprises an immunoglobulin-type binding region comprising a polypeptide selected from the group consisting of: single-domain antibody fragment, single-chain variable fragment, antibody variable fragment, complementary determining region 3 fragment, constrained FR3-CDR3-FR4 polypeptide, Fd fragment, antigen-binding fragment, fibronectin-derived 10^{th} fibronectin type III domain, tenascin type III domain, ankyrin repeat motif domain, low-density-lipoprotein-receptor-derived A-domain, lipocalin, Kunitz domain, Protein-A-derived Z domain, gamma-B crystalline-derived domain, ubiquitin-derived domain, Sac7d-derived polypeptide, Fyn-derived SH2 domain, miniprotein, C-type lectin-like domain scaffold, engineered antibody mimic, and any genetically manipulated counterparts of any of the foregoing which retain binding functionality. In certain embodiments, the binding region of the cell-targeted molecule of the present invention comprises the immunoglobulin-type binding region which is capable of binding to an extracellular target biomolecule selected from the group consisting of: CD20, CD22, CD40, CD74, CD79, CD25, CD30, HER2/neu/ErbB2, EGFR, EpCAM, EphB2, prostate-specific membrane antigen, Cripto, CDCP1, endoglin, fibroblast activated protein, Lewis-Y, CD19, CD21, CS1/SLAMF7, CD33, CD52, CD133, CEA, gpA33, mucin, TAG-72, tyrosine-protein kinase transmembrane receptor, carbonic anhydrase IX, folate binding protein, ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANK, FAP, tenascin, CD64, mesothelin, BRCA1, MART-1/MelanA, gp100, tyrosinase, TRP-1, TRP-2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen, prostate specific antigen, prostate stem cell antigen, human aspartyl (asparaginyl) beta-hydroxylase, EphA2, HER3/ErbB-3, MUC1, MART-1/MelanA, gp100, tyrosinase associated antigen, HPV-E7, Epstein-Barr virus antigen, Bcr-Abl, alpha-fetoprotein antigen, 17-A1, bladder tumor antigen, CD38, CD15, CD23, CD53, CD88, CD129, CD183, CD191, CD193, CD244, CD294, CD305, C3AR, FceRIa, galectin-9, mrp-14, PD-L1 (programmed death-ligand 1), Siglec-8, Siglec-10, CD49d, CD13, CD44, CD54, CD63, CD69, CD123, TLR4, FceRIa, IgE, CD107a, CD203c, CD14, CD68, CD80, CD86, CD105, CD115, F4/80, ILT-3, galectin-3, CD11a-c, GITRL, MHC class I molecule, MHC class II molecule, CD284 (TLR4), CD107-Mac3, CD195 (CCR5), HLA-DR, CD16/32, CD282 (TLR2), and any immunogenic fragment of any of the foregoing. In certain further embodiments, the binding region of the cell-targeted molecule of the present invention comprises the immunoglobulin-type binding region comprising or consisting essentially of the polypeptide sequence of amino acids 269-508 of any one of SEQ ID NOs: 4, 8, 12, or 16.

In certain embodiments, the Shiga toxin effector polypeptide region of the cell-targeted molecule of the present invention comprises or consists essentially of the polypeptide represented by the amino acid sequence selected from the group consisting of: 1) amino acids 75 to 251 of SEQ ID NO:1, SEQ ID NO:2, or SEQ ID NO:3; 2) amino acids 1 to 241 of SEQ ID NO:1, SEQ ID NO:2, or SEQ ID NO:3; and 3) amino acids 1 to 251 of SEQ ID NO:1, SEQ ID NO:2, or SEQ ID NO:3.

In certain embodiments, the Shiga toxin effector polypeptide region of the cell-targeted molecule of the present invention comprises 1) a Shiga toxin A1 fragment region having a carboxy-terminus and 2) a disrupted furin-cleavage motif at the carboxy-terminus of the Shiga toxin A1 fragment region. In certain further embodiments, the disrupted furin-cleavage motif comprises one or more mutations in the minimal, furin-cleavage motif relative to a wild-type, Shiga toxin A Subunit. In certain embodiments, the disrupted furin-cleavage motif is not an amino terminal truncation of sequences that overlap with part or all of at least one amino acid residue of the minimal furin-cleavage site. In certain embodiments, the mutation in the minimal, furin-cleavage motif is an amino acid deletion, insertion, and/or substitution of at least one amino acid residue in the R/Y-x-x-R furin cleavage motif. In certain further embodiments, the disrupted furin-cleavage motif comprises at least one mutation relative to a wild-type, Shiga toxin A Subunit, the mutation altering at least one amino acid residue in the region natively positioned 1) at 248-251 of the A Subunit of Shiga-like toxin 1 (SEQ ID NO: 1) or Shiga toxin (SEQ ID NO: 2), or 2) at 247-250 of the A Subunit of Shiga-like toxin 2 (SEQ ID NO:3).

In certain embodiments, the Shiga toxin effector polypeptide region of the cell-targeted molecule of the present invention comprises or consists essentially of the polypeptide represented by the amino acid sequence selected from the group consisting of: 1) amino acids 75 to 246 of SEQ ID NO:3; 2) amino acids 75 to 247 of SEQ ID NO:1 or SEQ ID NO:2; 3) amino acids 1 to 241 of SEQ ID NO:1, SEQ ID NO:2, or SEQ ID NO:3; 4) amino acids 1 to 246 of SEQ ID NO:3; and 5) amino acids 1 to 247 of SEQ ID NO:1 or SEQ ID NO:2.

In certain embodiments, the cell-targeted molecule comprises a binding region and/or molecular moiety located carboxy-terminal to the carboxy-terminus of the Shiga toxin A1 fragment region; wherein the mass of the binding region and/or molecular moiety is at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater.

In certain embodiments, the cell-targeted molecule comprises a binding region with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the cell-targeted molecule retains the appropriate level of the Shiga toxin biological activity noted herein (e.g., cytotoxicity and/or intracellular routing). In certain embodiments, the binding region is comprised within a relatively large, molecular moiety comprising such as, e.g., a molecular moiety with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the cell-targeted molecule retains the appropriate level of the Shiga toxin biological activity noted herein.

In certain embodiments, the cell-targeted molecule comprises a binding region and/or molecular moiety located carboxy-terminal to the carboxy-terminus of the Shiga toxin A1 fragment region. In certain further embodiments, the binding region and/or molecular moiety has a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the cell-targeted molecule retains the appropriate level of the Shiga toxin biological activity noted herein.

In certain embodiments, the cell-targeted molecule is capable when introduced to cells of exhibiting cytotoxicity that is in a range of from 0.1-fold to 1.5-fold of the cytotoxicity exhibited by a second cell-targeted molecule comprising the binding region and a second, Shiga toxin effector polypeptide region consisting of a wild-type, Shiga toxin A1 fragment, and wherein the binding region is positioned within the second cell targeted molecule carboxy-terminal to the wild-type, Shiga toxin A1 fragment. This means the second cell-targeted molecule comprises the same binding region as the cell-targeted molecule of the present invention but instead of comprising the same Shiga toxin effector polypeptide of the cell-targeted molecule of the present invention, the second cell-targeted molecule comprises a wild-type, Shiga toxin effector polypeptide comprising a Shiga toxin A1 fragment region having a carboxy-terminus and a wild-type furin-cleavage site at the carboxy-terminus of the A1 fragment region of the wild-type, Shiga toxin effector polypeptide (e.g. the polypeptide represented by amino acids 1-251 of SEQ ID NO:1 or SEQ ID NO:2, or amino acids 1-250 of SEQ ID NO:3). In certain further embodiments, the cell-targeted molecule of the present invention is capable when introduced to cells of exhibiting cytotoxicity that is in a range of from 0.1-fold, 0.5-fold, or 0.75-fold to 1.2-fold, 1.5-fold, 1.75-fold, 2-fold, 3-fold, 4-fold, or 5-fold of the cytotoxicity exhibited by a second cell-targeted molecule comprising the binding region and a second, Shiga toxin effector polypeptide region consisting of a wild-type, Shiga toxin A1 fragment, and wherein the binding region is positioned within the second cell targeted molecule carboxy-terminal to the wild-type, Shiga toxin A1 fragment.

In certain embodiments, the cell-targeted molecule is capable when introduced to cells of exhibiting cytotoxicity equivalent to a second cell-targeted molecule comprising the binding region and a Shiga toxin effector polypeptide region consisting of a wild-type, Shiga toxin A1 fragment, wherein the binding region is linked to the second cell-targeted molecule at a position carboxy-terminal to the wild-type, Shiga toxin A1 fragment. This means the second cell-targeted molecule comprises the same binding region as the cell-targeted molecule of the present invention but instead of comprising the same Shiga toxin effector polypeptide of the cell-targeted molecule of the present invention, the second cell-targeted molecule comprises a wild-type, Shiga toxin effector polypeptide comprising a Shiga toxin A1 fragment region having a carboxy-terminus and a wild-type furin-cleavage site at the carboxy-terminus of the A1 fragment region of the wild-type, Shiga toxin effector polypeptide (e.g. the polypeptide represented by amino acids 1-251 of SEQ ID NO:1 or SEQ ID NO:2, or amino acids 1-250 of SEQ ID NO:3). The meaning of “equivalent” is the cell-targeted molecule of the present invention exhibits an empirically measured level of cytotoxicity, as measured by an appropriate quantitative assay with reproducibility, which is reproducibly within 10% or less of the activity of the second cell-targeted molecule as the reference molecule.

In certain embodiments, the cell-targeted molecule of the present invention is capable of exhibiting improved, in vivo tolerability compared to the in vivo tolerability of a second cell-targeted molecule comprising the binding region and a Shiga toxin effector polypeptide region consisting of a wild-type, Shiga toxin A1 fragment, wherein the binding region is positioned within the second cell-targeted molecule carboxy-terminal to the wild-type, Shiga toxin A1 fragment. This means the second cell-targeted molecule comprises the same components linked together in the same manner except for the Shiga toxin effector polypeptide of the second cell-targeted molecule consists of a wild-type, Shiga toxin A1 polypeptide region.

In certain embodiments, the cell-targeted molecule of the present invention is capable of exhibiting improved, in vivo tolerability compared to the in vivo tolerability of a second cell-targeted molecule comprising the binding region and a Shiga toxin effector polypeptide region consisting of a wild-type, Shiga toxin A1 fragment, wherein the binding region is linked to the second cell targeted molecule at a position carboxy-terminal to the wild-type, Shiga toxin A1 fragment. This means the second cell-targeted molecule comprises the same components linked together in the same manner except for the Shiga toxin effector polypeptide of the second cell-targeted molecule consists of a wild-type, Shiga toxin A1 polypeptide region.

In certain embodiments, the cell-targeted molecule of the present invention comprises a carboxy-terminal endoplasmic reticulum retention/retrieval signal motif. In certain further embodiments, the cell-targeted molecule of the present invention comprises a carboxy-terminal endoplasmic reticulum retention/retrieval signal motif selected from the group consisting of: KDEL, HDEF, HDEL, RDEF, RDEL, WDEL, YDEL, HEEF, HEEL, KEEL, REEL, KAEL, KCEL, KFEL, KGEL, KHEL, KLEL, KNEL, KQEL, KREL, KSEL, KVEL, KWEL, KYEL, KEDL, KIEL, DKEL, FUEL, KDEF, KKEL, HADL, HAEL, HIEL, HNEL, HTEL, KTEL, HVEL, NDEL, QDEL, REDL, RNEL, RTDL, RTEL, SDEL, TDEL, SKEL, STEL, and EDEL.

In certain embodiments, the cell-targeted molecule of the present invention comprises a Shiga toxin effector polypeptide region having a mutation relative to a naturally occurring A Subunit of a member of the Shiga toxin family which changes the enzymatic activity of the Shiga toxin effector region. In certain further embodiments, the mutation is selected from at least one amino acid residue deletion, insertion, or substitution that reduces or eliminates cytotoxicity of the Shiga toxin effector polypeptide region. In certain further embodiments, the cell-targeted molecule comprises a mutation which reduces or eliminates catalytic activity but retains other Shiga toxin effector functions, such as, e.g., promoting cellular internalization and/or directing intracellular routing. In certain further embodiments of the cell-targeted molecule of the invention, the Shiga toxin effector polypeptide region comprises a mutation relative to a naturally occurring A Subunit of a member of the Shiga toxin family which reduces or eliminates the enzymatic activity of the Shiga toxin effector polypeptide but does not reduce the subcellular routing to the cytosol, of at least a part of the Shiga toxin effector polypeptide, below the subcellular routing level of a wild-type, Shiga toxin effector polypeptide. In certain embodiments, the mutation is selected from at least one amino acid residue substitution, such as, e.g., A231E, R75A, Y77S, Y114S, E167D, R170A, R176K and/or W203A in SEQ ID NO:1, SEQ ID NO:2, or SEQ ID NO:3.

In certain embodiments, the Shiga toxin effector polypeptide region of the cell-targeted molecule of the present invention comprises 1) a Shiga toxin A1 fragment region having a carboxy-terminus and 2) a disrupted furin-cleavage motif at the carboxy-terminus of the Shiga toxin A1 fragment region. In certain further embodiments, the disrupted furin-cleavage motif comprises one or more mutations in the minimal, furin-cleavage motif relative to a wild-type, Shiga toxin A Subunit. In certain embodiments, the disrupted furin-cleavage motif is not an amino terminal truncation of sequences that overlap with part or all of at least one amino acid residue of the minimal furin-cleavage site. In certain embodiments, the mutation in the minimal, furin-cleavage motif is an amino acid deletion, insertion, and/or substitution of at least one amino acid residue in the R/Y-x-x-R furin cleavage motif. In certain further embodiments, the disrupted furin-cleavage motif comprises at least one mutation relative to a wild-type, Shiga toxin A Subunit, the mutation altering at least one amino acid residue in the region natively positioned 1) at 248-251 of the A Subunit of Shiga-like toxin 1 (SEQ ID NO: 1) or Shiga toxin (SEQ ID NO: 2), or 2) at 247-250 of the A Subunit of Shiga-like toxin 2 (SEQ ID NO:3). In certain further embodiments, the mutation is an amino acid residue substitution of an arginine residue with a non-positively charged, amino acid residue.

In certain embodiments, the cell-targeted molecule of the present invention comprises 1) a binding region comprising one or more polypeptides and capable of specifically binding at least one extracellular target biomolecule, 2) a Shiga toxin effector polypeptide region comprising a Shiga toxin A1 fragment region having a carboxy-terminus, and c) a disrupted furin-cleavage motif at the carboxy-terminus of the A1 fragment region; wherein the binding region is positioned within the cell-targeted molecule carboxy-terminal to the Shiga toxin A1 fragment region; and wherein the cell-targeted molecule is capable when introduced to cells of exhibiting cytotoxicity that is in a range of from 0.1-fold to 1.5-fold of the cytotoxicity exhibited by a second cell-targeted molecule comprising the binding region and a second, Shiga toxin effector polypeptide region consisting of a wild-type, Shiga toxin A1 fragment, and wherein the binding region is linked to the second cell targeted molecule carboxy-terminal to the wild-type, Shiga toxin A1 fragment. In certain further embodiments, the cell-targeted molecule of the present invention is capable when introduced to cells of exhibiting cytotoxicity that is in a range of from 0.1-fold, 0.5-fold, or 0.75-fold to 1.2-fold, 1.5-fold, 1.75-fold, 2-fold, 3-fold, 4-fold, or 5-fold of the cytotoxicity exhibited by a second cell-targeted molecule comprising the binding region and a second, Shiga toxin effector polypeptide region consisting of a wild-type, Shiga toxin A1 fragment, and wherein the binding region is positioned within the second cell targeted molecule carboxy-terminal to the wild-type, Shiga toxin A1 fragment.

In certain embodiments, the cell-targeted molecule of the present invention comprises 1) a binding region comprising one or more polypeptides and capable of specifically binding at least one extracellular target biomolecule, 2) a Shiga toxin effector polypeptide region comprising a Shiga toxin A1 fragment region having a carboxy-terminus, and c) a disrupted furin-cleavage motif at the carboxy-terminus of the A1 fragment region; wherein the binding region is linked to the cell-targeted molecule carboxy-terminal to the Shiga toxin A1 fragment region; and wherein the cell-targeted molecule is capable when introduced to cells of exhibiting cytotoxicity equivalent to a second cell-targeted molecule comprising the binding region and a Shiga toxin effector polypeptide region consisting of a wild-type, Shiga toxin A1 fragment, wherein the binding region is linked to the second cell-targeted molecule at a position carboxy-terminal to the wild-type, Shiga toxin A1 fragment.

In certain embodiments, the cell-targeted molecule of the present invention comprises 1) a binding region comprising one or more polypeptides and capable of specifically binding at least one extracellular target biomolecule, 2) a Shiga toxin effector polypeptide region comprising a Shiga toxin A1 fragment region having a carboxy-terminus, and c) a disrupted furin-cleavage motif at the carboxy-terminus of the A1 fragment region; wherein the binding region is positioned within the cell-targeted molecule carboxy-terminal to the Shiga toxin A1 fragment region; and wherein the cell-targeted molecule is capable when introduced to cells of exhibiting cytotoxicity that is in a range of from 0.1-fold to 1.5-fold of the cytotoxicity exhibited by a second cell-targeted molecule comprising the binding region and a second, Shiga toxin effector polypeptide region consisting of a wild-type, Shiga toxin A1 fragment, and wherein the binding region is linked to the second cell targeted molecule carboxy-terminal to the wild-type, Shiga toxin A1 fragment. In certain further embodiments, the cell-targeted molecule of the present invention is capable when introduced to cells of exhibiting cytotoxicity that is in a range of from 0.1-fold, 0.5-fold, or 0.75-fold to 1.2-fold, 1.5-fold, 1.75-fold, 2-fold, 3-fold, 4-fold, or 5-fold of the cytotoxicity exhibited by a second cell-targeted molecule comprising the binding region and a second, Shiga toxin effector polypeptide region consisting of a wild-type, Shiga toxin A1 fragment, and wherein the binding region is positioned within the second cell targeted molecule carboxy-terminal to the wild-type, Shiga toxin A1 fragment. In certain further embodiments, the cell-targeted molecule is capable when introduced to cells of exhibiting cytotoxicity that is greater than that of a third cell-targeted molecule having an amino-terminus, and comprising the binding region and the Shiga toxin effector polypeptide region which is not positioned at or proximal to the amino-terminus of the third cell-targeted molecule. In certain further embodiments, the Shiga toxin effector polypeptide region of the cell-targeted molecule of the present invention comprises or consists essentially of amino acids 2-242, 2-243, 2-244, 2-245, 2-246, 2-247, 2-248, 2-249, 2-250, 2-251, or 2-252 of any one of SEQ ID NOs: 32-35. In certain further embodiments, the binding region of the cell-targeted molecule of the present invention comprises the immunoglobulin-type binding region comprising or consisting essentially of the polypeptide sequence of amino acids 269-508 of any one of SEQ ID NOs: 4, 8, 12, or 16.

In certain embodiments, the cell-targeted molecule of the present invention comprises 1) a binding region comprising one or more polypeptides and capable of specifically binding at least one extracellular target biomolecule, 2) a Shiga toxin effector polypeptide region comprising a Shiga toxin A1 fragment region having a carboxy-terminus, and c) a disrupted furin-cleavage motif at the carboxy-terminus of the A1 fragment region; wherein the binding region is positioned within the cell-targeted molecule carboxy-terminal to the Shiga toxin A1 fragment region; and wherein the cell-targeted molecule is capable when introduced to cells of exhibiting cytotoxicity that is in a range of from 0.1-fold to 1.5-fold of the cytotoxicity exhibited by a second cell-targeted molecule comprising the binding region and a second, Shiga toxin effector polypeptide region consisting of a wild-type, Shiga toxin A1 fragment, and wherein the binding region is linked to the second cell targeted molecule carboxy-terminal to the wild-type, Shiga toxin A1 fragment. In certain further embodiments, the cell-targeted molecule of the present invention is capable when introduced to cells of exhibiting cytotoxicity that is in a range of from 0.1-fold, 0.5-fold, or 0.75-fold to 1.2-fold, 1.5-fold, 1.75-fold, 2-fold, 3-fold, 4-fold, or 5-fold of the cytotoxicity exhibited by a second cell-targeted molecule comprising the binding region and a second, Shiga toxin effector polypeptide region consisting of a wild-type, Shiga toxin A1 fragment, and wherein the binding region is positioned within the second cell targeted molecule carboxy-terminal to the wild-type, Shiga toxin A1 fragment. In certain further embodiments, the cell-targeted molecule is capable when introduced to cells of exhibiting cytotoxicity that is greater than that of a third cell-targeted molecule having an amino-terminus, and comprising the binding region and the Shiga toxin effector polypeptide region which is not positioned at or proximal to the amino-terminus of the third cell-targeted molecule.

In certain embodiments, the cell-targeted molecule of the present invention comprises 1) a binding region comprising one or more polypeptides and capable of specifically binding at least one extracellular target biomolecule, 2) a Shiga toxin effector polypeptide region having an amino-terminus and comprising a Shiga toxin A1 fragment region having a carboxy-terminus, and c) a disrupted furin-cleavage motif at the carboxy-terminus of the A1 fragment region; wherein the binding region is linked, either directly or indirectly, to the cell-targeted molecule carboxy-terminal to the Shiga toxin A1 fragment region; and wherein the cell-targeted molecule is capable of exhibiting cytotoxicity equivalent to a second cell-targeted molecule comprising the binding region and a Shiga toxin effector polypeptide region consisting of a wild-type, Shiga toxin A1 fragment and wherein the binding region is linked to the second cell targeted molecule at a position carboxy-terminal to the wild-type, Shiga toxin A1 fragment. In certain further embodiments, the cell-targeted molecule is capable when introduced to cells of exhibiting cytotoxicity that is greater than that of a third cell-targeted molecule having an amino-terminus, and comprising the binding region and the Shiga toxin effector polypeptide region which is not positioned at or proximal to the amino-terminus of the third cell-targeted molecule. In certain further embodiments, the Shiga toxin effector polypeptide region of the cell-targeted molecule of the present invention comprises or consists essentially of amino acids 2-242, 2-243, 2-244, 2-245, 2-246, 2-247, 2-248, 2-249, 2-250, 2-251, or 2-252 of any one of SEQ ID NOs: 32-35. In certain further embodiments, the binding region of the cell-targeted molecule of the present invention comprises the immunoglobulin-type binding region comprising or consisting essentially of the polypeptide sequence of amino acids 269-508 of any one of SEQ ID NOs: 4, 8, 12, or 16.

In certain embodiments, the cell-targeted molecule of the present invention comprises 1) a binding region comprising one or more polypeptides and capable of specifically binding at least one extracellular target biomolecule, 2) a Shiga toxin effector polypeptide region having an amino-terminus and comprising a Shiga toxin A1 fragment region having a carboxy-terminus, and c) a disrupted furin-cleavage motif at the carboxy-terminus of the A1 fragment region; wherein the binding region is linked, either directly or indirectly, to the cell-targeted molecule carboxy-terminal to the Shiga toxin A1 fragment region; and wherein the cell-targeted molecule is capable of exhibiting cytotoxicity equivalent to a second cell-targeted molecule comprising the binding region and a Shiga toxin effector polypeptide region consisting of a wild-type, Shiga toxin A1 fragment and wherein the binding region is linked to the second cell targeted molecule at a position carboxy-terminal to the wild-type, Shiga toxin A1 fragment. In certain further embodiments, the cell-targeted molecule is capable when introduced to cells of exhibiting cytotoxicity that is greater than that of a third cell-targeted molecule having an amino-terminus, and comprising the binding region and the Shiga toxin effector polypeptide region which is not positioned at or proximal to the amino-terminus of the third cell-targeted molecule. In certain further embodiments, the cell-targeted molecule of the present invention exhibits cytotoxicity that is greater by 3-fold, 4-fold, 5-fold, 6-fold, 7-fold, 8-fold, 9-fold, 10-fold than the cytotoxicity of the second cell-targeted molecule. In certain further embodiments, the cell-targeted molecule of the present invention exhibits cytotoxicity that is greater by 3-fold, 4-fold, 5-fold, 6-fold, 7-fold, 8-fold, 9-fold, 10-fold than the cytotoxicity of the second cell-targeted molecule as assayed by CD50 values, such as, e.g., by using a cell-kill assay described herein. In certain further embodiments, the cytotoxicity of the cell-targeted molecule of the present invention to target positive cells is 3-fold, 4-fold, 5-fold, 6-fold, 7-fold, 8-fold, 9-fold, 10-fold or greater than the cytotoxicity of the second cell-targeted molecule to target positive cells as assayed by CD50 values. In certain further embodiments, the Shiga toxin effector polypeptide region of the cell-targeted molecule of the present invention comprises or consists essentially of amino acids 2-242, 2-243, 2-244, 2-245, 2-246, 2-247, 2-248, 2-249, 2-250, 2-251, or 2-252 of any one of SEQ ID NOs: 32-35. In certain further embodiments, the binding region of the cell-targeted molecule of the present invention comprises the immunoglobulin-type binding region comprising or consisting essentially of the polypeptide sequence of amino acids 269-508 of any one of SEQ ID NOs: 4, 8, 12, or 16.

In certain embodiments, the cell-targeted molecule of the present invention comprises 1) a binding region comprising one or more polypeptides and capable of specifically binding at least one extracellular target biomolecule, 2) a Shiga toxin effector polypeptide region comprising a Shiga toxin A1 fragment region having a carboxy-terminus, and 3) a disrupted furin-cleavage motif at the carboxy-terminus of the A1 fragment region; wherein the binding region is linked, either directly or indirectly, to the cell-targeted molecule carboxy-terminal to the Shiga toxin A1 fragment region; and wherein the cell-targeted molecule is capable when introduced to cells of exhibiting cytotoxicity that is in a range of from 0.1-fold to 1.5-fold of the cytotoxicity exhibited by a second cell-targeted molecule comprising the binding region and a second, Shiga toxin effector polypeptide region consisting of a wild-type, Shiga toxin A1 fragment, and wherein the binding region is linked to the second cell targeted molecule carboxy-terminal to the wild-type, Shiga toxin A1 fragment. In certain further embodiments, the cell-targeted molecule of the present invention is capable when introduced to cells of exhibiting cytotoxicity that is in a range of from 0.1-fold, 0.5-fold, or 0.75-fold to 1.2-fold, 1.5-fold, 1.75-fold, 2-fold, 3-fold, 4-fold, or 5-fold of the cytotoxicity exhibited by a second cell-targeted molecule comprising the binding region and a second, Shiga toxin effector polypeptide region consisting of a wild-type, Shiga toxin A1 fragment, and wherein the binding region is positioned within the second cell targeted molecule carboxy-terminal to the wild-type, Shiga toxin A1 fragment. In certain further embodiments, the cell-targeted molecule is capable of exhibiting improved, in vivo tolerability compared to in vivo tolerability of the second cell-targeted molecule. In certain further embodiments, the Shiga toxin effector polypeptide region of the cell-targeted molecule of the present invention comprises or consists essentially of amino acids 2-242, 2-243, 2-244, 2-245, 2-246, 2-247, 2-248, 2-249, 2-250, 2-251, or 2-252 of any one of SEQ ID NOs: 32-35. In certain further embodiments, the binding region of the cell-targeted molecule of the present invention comprises the immunoglobulin-type binding region comprising or consisting essentially of the polypeptide sequence of amino acids 269-508 of any one of SEQ ID NOs: 4, 8, 12, or 16. In certain further embodiments, the cell-targeted molecule of the present invention comprises or consists essentially of the polypeptide shown in any one of SEQ ID NOs: 32-35.

In certain embodiments, the cell-targeted molecule of the present invention comprises 1) a binding region comprising one or more polypeptides and capable of specifically binding at least one extracellular target biomolecule, 2) a Shiga toxin effector polypeptide region having an amino-terminus and comprising a Shiga toxin A1 fragment region having a carboxy-terminus, and 3) a disrupted furin-cleavage motif at the carboxy-terminus of the A1 fragment region; wherein the binding region is linked, either directly or indirectly, to the cell-targeted molecule carboxy-terminal to the Shiga toxin A1 fragment region; and wherein the cell-targeted molecule is capable of exhibiting cytotoxicity equivalent to a second cell-targeted molecule comprising the binding region and a Shiga toxin effector polypeptide region consisting of a wild-type, Shiga toxin A1 fragment and wherein the binding region is linked to the second cell targeted molecule at a position carboxy-terminal to the wild-type, Shiga toxin A1 fragment. In certain further embodiments, the cell-targeted molecule is capable of exhibiting improved, in vivo tolerability compared to in vivo tolerability of the second cell-targeted molecule. In certain further embodiments, the Shiga toxin effector polypeptide region of the cell-targeted molecule of the present invention comprises or consists essentially of amino acids 2-242, 2-243, 2-244, 2-245, 2-246, 2-247, 2-248, 2-249, 2-250, 2-251, or 2-252 of any one of SEQ ID NOs: 32-35. In certain further embodiments, the binding region of the cell-targeted molecule of the present invention comprises the immunoglobulin-type binding region comprising or consisting essentially of the polypeptide sequence of amino acids 269-508 of any one of SEQ ID NOs: 4, 8, 12, or 16. In certain further embodiments, the cell-targeted molecule of the present invention comprises or consists essentially of the polypeptide shown in any one of SEQ ID NOs: 32-35.

For certain embodiments of the cell-targeted molecule of the present invention, administration of the cell-targeted molecule to a first population of cells whose members are physically coupled to extracellular target biomolecules of the binding region of the cell-targeted molecule, and a second population of cells whose members are not physically coupled to any extracellular target biomolecule of the binding region, the cytotoxic effect of the cell-targeted molecule to members of said first population of cells relative to members of said second population of cells is at least 3-fold greater.

In certain embodiments, the cell-targeted molecule of the present invention comprises or consists essentially of the polypeptide shown in any one of SEQ ID NOs: 4-31.

In certain embodiments of the cell-targeted molecule of the present invention, the binding region does not comprise a ligand. In certain embodiments of the cell-targeted molecule of the present invention, the binding region does not comprise a chemokine or a TNF-related apoptosis-inducing ligand (TRAIL) nor a receptor binding fragment thereof. In certain embodiments of the cell-targeted molecule of the present invention, the binding region does not comprise a human chemokine or human TRAIL nor a receptor binding fragment thereof. In certain embodiments of the cell-targeted molecule of the present invention, the immunoglobulin-type binding region does not comprise a ligand nor a receptor binding fragment thereof. In certain embodiments of the cell-targeted molecule of the present invention, the immunoglobulin-type binding region does not comprise a chemokine or a TNF-related apoptosis-inducing ligand (TRAIL) nor a receptor binding fragment thereof. In certain embodiments of the cell-targeted molecule of the present invention, the binding region does not comprise a human CC chemokine nor a receptor binding fragment thereof. In certain embodiments of the cell-targeted molecule of the present invention, the binding region does not comprise the human CC chemokine CCL2 (see Bose S, Cho J et al., Arch Pharm Res 36: 1039-50 (2013)). In certain embodiments of the cell-targeted molecule of the present invention, the binding region does not comprise the human, CC chemokine CCL2, nor a receptor binding fragment thereof, and a carboxy-terminal, Shiga toxin effector polypeptide consisting of amino acids 75-247 of StxA. In certain embodiments of the cell-targeted molecule of the present invention, the binding region does not comprise the human, CC chemokine CCL2, nor a receptor binding fragment thereof, fused to a carboxy-terminal, Shiga toxin effector polypeptide consisting of amino acids 75-247 of StxA (SEQ ID NO:2). In certain embodiments of the cell-targeted molecule of the present invention, the binding region does not comprise the human TRAIL nor a receptor binding fragment thereof.

In certain embodiments, the cell-targeted molecule of the present invention does not comprise a carboxy-terminal, binding region comprising a fragment of an immune cell surface receptor. In certain embodiments of the cell-targeted molecule of the present invention, the binding region does not comprise a fragment of a human, immune cell surface co-receptor. In certain further embodiments of the cell-targeted molecule of the present invention, the binding region does not comprise a fragment of human CD4, a type-I transmembrane glycoprotein. In certain embodiments, the cell-targeted molecules of the present invention do not comprise a Shiga toxin effector polypeptide comprising amino acids 1-247 of SEQ ID NO:2, 45-247 of SEQ ID NO:2, and/or 75-247 of SEQ ID NO:2 fused to a carboxy-terminal, binding region comprising a fragment of human CD4 corresponding to amino acid residues 19-183.

In certain embodiments of the cell-targeted molecule (e.g., a protein) of the present invention, the Shiga toxin effector polypeptide does not consist of the polypeptide shown in SEQ ID NO:2 further comprising both of the following amino acid residue substitutions: R248H and R251H. In certain embodiments of the cell-targeted molecule, the Shiga toxin effector polypeptide does not comprise both of the following amino acid residue substitutions: R248H and R251H. In certain embodiments of the cell-targeted molecule, the Shiga toxin effector polypeptide does not consist of the polypeptide shown in SEQ ID NO:1 further comprising both of the following amino acid residue substitutions: R248G and R251G. In certain embodiments of the cell-targeted molecule, the Shiga toxin effector polypeptide does not comprise both of the following amino acid residue substitutions: R248G and R251G. In certain embodiments of the cell-targeted molecule, the Shiga toxin effector polypeptide does not consist of the polypeptide shown in SEQ ID NO:1 further comprising all of the following amino acid residue substitutions: A246G, S247A, A253G, and S254A. In certain embodiments of the cell-targeted molecule, the Shiga toxin effector polypeptide does not comprise all of the following amino acid residue substitutions: A246G, S247A, A253G, and S254A. In certain embodiments of the cell-targeted molecule, the Shiga toxin effector polypeptide does not consist of the polypeptide shown in SEQ ID NO:1 further comprising all of the following amino acid residue substitutions: A246G, S247A, R248G, R251G, A253G, and S254A. In certain embodiments of the cell-targeted molecule, the Shiga toxin effector polypeptide does not comprise all of the following amino acid residue substitutions: A246G, S247A, R248G, R251G, A253G, and S254A. In certain embodiments of the cell-targeted molecule, the Shiga toxin effector polypeptide does not consist of the polypeptide shown in SEQ ID NO:2 further comprising the deletion of the region natively positioned at 247-252. In certain embodiments of the cell-targeted molecule, the Shiga toxin effector polypeptide does not comprise a Shiga toxin effector polypeptide comprising the deletion of the region natively positioned at 247-252. In certain embodiments of the cell-targeted molecule, the Shiga toxin effector polypeptide does not consist of the polypeptide shown in SEQ ID NO:2 further comprising both of the following deletions: 245-247 and 253-255. In certain embodiments of the cell-targeted molecule, the Shiga toxin effector polypeptide does not comprise both of the following deletions: 245-247 and 253-255.

The present invention also provides pharmaceutical compositions comprising a cell-targeted molecule of the present invention (e.g., a protein of the present invention) and at least one pharmaceutically acceptable excipient or carrier; and the use of such a cell-targeted molecule or a composition comprising it in the methods of the present invention as further described herein.

The present invention also provides diagnostic compositions, each comprising 1) a cell-targeted molecule (e.g., a protein) of the present invention and 2) a detection promoting agent; and the use of such a diagnostic composition in the methods of the present invention as further described herein. Among certain embodiments of the present invention is a diagnostic composition comprising a protein of the present invention further comprising a detection promoting agent for the collection of information, such as diagnostically useful information about a cell type, tissue, organ, disease, disorder, condition, patient, and/or group of patients.

Beyond the cell-targeted molecules of the present invention, polynucleotides capable of encoding a cell-targeted molecule (e.g., a protein) of the present invention of the present invention, are within the scope of the present invention, as well as expression vectors which comprise a polynucleotide of the present invention and host cells comprising an expression vector of the present invention. Host cells comprising an expression vector may be used, e.g., in methods for producing a molecule (e.g., a cell-targeted molecule) of the present invention, or a polypeptide component or fragment thereof, by recombinant expression.

The present invention also encompasses any composition of matter of the present invention which is immobilized on a solid substrate. Such arrangements of the compositions of matter of the present invention may be utilized, e.g., in methods of screening molecules as described herein.

Beyond the compositions of matter of the present invention, the present invention is directed to a variety of methods, such as, e.g., methods which use a composition of matter of the invention and/or methods which create a composition of matter of the invention.

Among certain embodiments of the present invention are methods of conferring improved cytotoxicity to a cell-targeted molecule comprising a binding region and a Shiga toxin effector polypeptide region, wherein each method comprises the step of arranging the binding region and Shiga toxin effector polypeptide region such that the amino-terminus of the Shiga toxin effector polypeptide is at and/or more proximal to an amino terminus of a polypeptide component of the cell-targeted molecule. For example, certain embodiments of the present invention are methods of conferring improved cytotoxicity to a cell-targeted molecule, wherein each method comprises the step of arranging the binding region more proximally to the carboxy-terminus of the cell-targeted molecule and/or to the carboxy-terminus of the cell-targeted molecule. In another example, certain embodiments of the present invention are methods of conferring improved cytotoxicity to a cell-targeted molecule having a polypeptide component, wherein each method comprises the step of arranging the Shiga toxin effector polypeptide within the cell-targeted molecule more proximally to an amino-terminus of a polypeptide component of the cell-targeted molecule and/or to the amino-terminus of a polypeptide component of the cell-targeted molecule.

Among certain embodiments of the present invention is a method of conferring improved cytotoxicity to a cell-targeted molecule comprising a binding region and a Shiga toxin effector polypeptide region comprising a polypeptide derived from the amino acid sequence of the A Subunit of at least one member of the Shiga toxin family, the method comprising the step of arranging the binding region within the cell-targeted molecule carboxy-terminal to the Shiga toxin effector polypeptide. In certain further embodiments, the binding region comprises or consists of an immunoglobulin-type binding region comprising one or more polypeptides and capable of specifically binding at least one extracellular target biomolecule. In certain further embodiments, the arranging step comprises linking the binding region to the cell-targeted molecule at a position carboxy-terminal to the carboxy-terminus of the Shiga toxin effector polypeptide. In certain further embodiments, the arranging step comprises fusing the binding region to the cell-targeted molecule at a position carboxy-terminal to the carboxy-terminus of the Shiga toxin effector polypeptide. In certain further embodiments, the cell-targeted molecule consists of a single, continuous polypeptide. The present invention also encompasses any cell-targeted molecule created using this method which is capable when introduced to cells of exhibiting a cytotoxicity greater than that of a second cell-targeted molecule comprising the binding region and a Shiga toxin effector polypeptide region consisting of a wild-type, Shiga toxin A1 fragment and wherein the binding region is linked to the second cell targeted molecule at a position carboxy-terminal to the wild-type, Shiga toxin A1 fragment, such as, e.g., cell-targeted molecules created using the method that exhibit a 4-fold, 5-fold, 6-fold, 9-fold, or greater cytotoxicity as compared to the cytotoxicity of the second cell-targeted molecule or parental cell-targeted molecule. In certain further embodiments, the cell-targeted molecule of the present invention exhibits cytotoxicity that is greater by 3-fold, 4-fold, 5-fold, 6-fold, 7-fold, 8-fold, 9-fold, 10-fold than the cytotoxicity of the second cell-targeted molecule. In certain further embodiments, the cell-targeted molecule of the present invention exhibits cytotoxicity that is greater by 3-fold, 4-fold, 5-fold, 6-fold, 7-fold, 8-fold, 9-fold, 10-fold than the cytotoxicity of the second cell-targeted molecule as assayed by CD50 values, such as, e.g., by using a cell-kill assay described herein. In certain further embodiments, the cytotoxicity of the cell-targeted molecule of the present invention to target positive cells is 3-fold, 4-fold, 5-fold, 6-fold, 7-fold, 8-fold, 9-fold, 10-fold or greater than the cytotoxicity of the second cell-targeted molecule to target positive cells as assayed by CD50 values.

Among certain embodiments of the present invention is a method of conferring improved cytotoxicity to a cell-targeted molecule comprising a binding region and a Shiga toxin effector polypeptide region comprising a polypeptide derived from the amino acid sequence of the A Subunit of at least one member of the Shiga toxin family, the method comprising the step of arranging the Shiga toxin effector polypeptide within the cell-targeted molecule more proximally to an amino-terminus of a polypeptide component of the cell-targeted molecule and/or to an amino-terminus of a polypeptide component of the cell-targeted molecule. In certain further embodiments, the arranging step comprises linking the binding region to the cell-targeted molecule at a position carboxy-terminal to the carboxy-terminus of the Shiga toxin effector polypeptide. In certain further embodiments, the binding region comprises or consists of an immunoglobulin-type binding region comprising one or more polypeptides and capable of specifically binding at least one extracellular target biomolecule. In certain further embodiments, the arranging step comprises fusing the binding region to the cell-targeted molecule at a position carboxy-terminal to the carboxy-terminus of the Shiga toxin effector polypeptide. In certain further embodiments, the cell-targeted molecule consists of a single, continuous polypeptide. The present invention also encompasses any cell-targeted molecule created using this method which is capable when introduced to cells of exhibiting a cytotoxicity greater than that of a second cell-targeted molecule comprising the binding region and a Shiga toxin effector polypeptide region consisting of a wild-type, Shiga toxin A1 fragment and wherein the binding region is linked to the second cell targeted molecule at a position carboxy-terminal to the wild-type, Shiga toxin A1 fragment, such as, e.g., cell-targeted molecules created using the method that exhibits a 4-fold, 5-fold, 6-fold, 9-fold, or greater cytotoxicity as compared to the cytotoxicity of the second cell-targeted molecule or parental cell-targeted molecule. In certain further embodiments, the cell-targeted molecule of the present invention exhibits cytotoxicity that is greater by 3-fold, 4-fold, 5-fold, 6-fold, 7-fold, 8-fold, 9-fold, 10-fold than the cytotoxicity of the second cell-targeted molecule. In certain further embodiments, the cell-targeted molecule of the present invention exhibits cytotoxicity that is greater by 3-fold, 4-fold, 5-fold, 6-fold, 7-fold, 8-fold, 9-fold, 10-fold than the cytotoxicity of the second cell-targeted molecule as assayed by CD50 values, such as, e.g., by using a cell-kill assay described herein. In certain further embodiments, the cytotoxicity of the cell-targeted molecule of the present invention to target positive cells is 3-fold, 4-fold, 5-fold, 6-fold, 7-fold, 8-fold, 9-fold, 10-fold or greater than the cytotoxicity of the second cell-targeted molecule to target positive cells as assayed by CD₅₀ values.

Among certain embodiments of the present invention is a method for improving the in vivo tolerability and/or in vitro stability of a molecule comprising 1) a Shiga toxin effector polypeptide comprising i) a polypeptide derived from the amino acid sequence of the A Subunit of at least one member of the Shiga toxin family and ii) Shiga toxin A1 fragment region having a carboxy-terminus and a furin-cleavage site at and/or proximal to the carboxy-terminus of the A1 fragment region; and 2) a heterologous, binding region capable of specifically binding at least one extracellular target biomolecule which is linked to the cell-targeted molecule carboxy-terminal to the Shiga toxin effector polypeptide; the method comprising the step of disrupting a furin-cleavage motif comprising the furin-cleavage site. In certain further embodiments of this method, the disrupting step involves creating a mutation, truncation, and/or modification of the functional group of an amino acid residue that reduces the protease-cleavage sensitivity of the carboxy-terminus of the Shiga toxin effector polypeptide. In certain embodiments of this method, the heterologous, binding region sterically covers the carboxy-terminus of the A1 fragment region. In certain further embodiments, the binding region comprises or consists of an immunoglobulin-type binding region comprising one or more polypeptides and capable of specifically binding at least one extracellular target biomolecule. The present invention also encompasses any molecule created using this method which is capable of exhibiting improved in vivo tolerability as compared to a parental molecule comprising an undisrupted, furin-cleavage site and/or furin-cleavage motif at and/or proximal to the carboxy-terminus of the A1 fragment region.

Among certain embodiments of the present invention is a method for improving the in vivo tolerability and/or in vitro stability of a molecule comprising 1) a Shiga toxin effector polypeptide comprising i) a polypeptide derived from the amino acid sequence of the A Subunit of at least one member of the Shiga toxin family and ii) Shiga toxin A1 fragment region having a carboxy-terminus and a furin-cleavage site at and/or proximal to the carboxy-terminus of the A1 fragment region; and 2) a heterologous, binding region capable of specifically binding at least one extracellular target biomolecule which is linked, either directly or indirectly, to the carboxy-terminus of the Shiga toxin effector polypeptide; the method comprising the step of disrupting a furin-cleavage motif comprising the furin-cleavage site. In certain further embodiments of this method, the disrupting step involves creating a mutation, truncation, and/or modification of the functional group of an amino acid residue that reduces the protease-cleavage sensitivity of the carboxy-terminus of the Shiga toxin effector polypeptide. In certain embodiments of this method, the heterologous, binding region sterically covers the carboxy-terminus of the A1 fragment region. The present invention also encompasses any molecule created using this method which is capable of exhibiting improved in vivo tolerability as compared to a parental molecule comprising an undisrupted furin-cleavage motif at and/or proximal to the carboxy-terminus of the A1 fragment region.

Additionally, the present invention provides methods of killing cell(s) comprising the step of contacting a cell(s) with a cell-targeted molecule (e.g., a protein) of the present invention or a pharmaceutical thereof. In certain embodiments of the methods of the present invention are methods of killing cell(s) comprising the step of contacting a cell(s) with a protein of the present invention or a pharmaceutical composition comprising a protein of the invention. In certain embodiments, the step of contacting the cell(s) occurs in vitro. In certain other embodiments, the step of contacting the cell(s) occurs or in vivo. In further embodiments of the cell killing methods, the method is capable of selectively killing cell(s) and/or cell types preferentially over other cell(s) and/or cell types when contacting a mixture of cells which differ with respect to the extracellular presence and/or expression level of an extracellular target biomolecule of the binding region of the protein.

The present invention further provides methods of treating diseases, disorders, and/or conditions in patients comprising the step of administering to a patient in need thereof a therapeutically effective amount of a cell-targeted molecule (e.g., a protein) of the present invention or a pharmaceutical composition of the present invention. In certain embodiments, the disease, disorder, or condition to be treated using a method of the invention is selected from: a cancer, tumor, growth abnormality, immune disorder, or microbial infection. In certain embodiments of these methods, the cancer to be treated is selected from the group consisting of: bone cancer, breast cancer, central/peripheral nervous system cancer, gastrointestinal cancer, germ cell cancer, glandular cancer, head-neck cancer, hematological cancer, kidney-urinary tract cancer, liver cancer, lung/pleura cancer, prostate cancer, sarcoma, skin cancer, and uterine cancer. In certain embodiments of these methods, the immune disorder to be treated is an immune disorder associated with a disease selected from the group consisting of: amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriatic arthritis, rheumatoid arthritis, scleroderma, septic shock, Sjorgren's syndrome, ulcerative colitis, and vasculitis.

Among certain embodiments of the present invention is the use of any composition of the present invention (e.g., a cell-targeted molecule, protein, and/or pharmaceutical composition of the present invention) for the treatment or prevention of a cancer, tumor, growth abnormality, immune disorder and/or microbial infection is within the scope of the present invention. Among certain embodiments of the present invention is a protein of the present invention and/or a pharmaceutical composition thereof for the treatment or prevention of a cancer, tumor, growth abnormality, immune disorder, and/or microbial infection. Among certain embodiments of the present invention is the use of a cell-targeted molecule of the present invention and/or pharmaceutical composition thereof in the manufacture of a medicament for the treatment or prevention of a cancer, tumor, growth abnormality, immune disorder, or microbial infection.

Certain embodiments of the cell-targeted molecules of the present invention (e.g., proteins of the present invention) may be utilized for the delivery of additional exogenous material into a cell physically coupled with an extracellular target biomolecule of the binding region of a cell-targeted molecule of the present invention. Additionally, the present invention provides a method for delivering exogenous material to the inside of a cell(s) comprising contacting the cell(s), either in vitro or in vivo, with a cell-targeted molecule, pharmaceutical composition, and/or diagnostic composition of the present invention. The present invention further provides a method for delivering exogenous material to the inside of a cell(s) in a patient, the method comprising the step of administering to the patient a cell-targeted molecule of the present invention (with or without cytotoxic activity), wherein the target cell(s) is physically coupled with an extracellular target biomolecule of the binding region of the cell-targeted molecule.

The use of any composition of the present invention (e.g., a cell-targeted molecule, protein, pharmaceutical composition, and/or diagnostic composition of the present invention) for the diagnosis, prognosis, and/or characterization of a disease, disorder, and/or condition is within the scope of the present invention. Among certain embodiments of the present invention is a method of using a cell-targeted molecule (e.g., a protein) of the present invention comprising a detection promoting agent and/or composition of the present invention (e.g. a diagnostic composition of the present invention) for the collection of information useful in the diagnosis, prognosis, or characterization of a disease, disorder, or condition. For example, a diagnostic composition of the present invention may be used to detect a cell in vivo by administering to a mammalian subject a composition comprising a cell-targeted molecule of the present invention which comprises a detection promoting agent and detecting the presence of the cell-targeted molecule either in vitro or in vivo.

Among certain embodiments of the present invention is the method of detecting a cell using a cell-targeted molecule (e.g., a protein) and/or diagnostic composition of the present invention comprising the steps of 1) contacting a cell with the cell-targeted molecule and/or diagnostic composition and 2) detecting the presence of said cell-targeted molecule and/or diagnostic composition. In certain embodiments, the step of contacting the cell(s) occurs in vitro. In certain embodiments, the step of contacting the cell(s) occurs in vivo. In certain embodiments, the step of detecting the cell(s) occurs in vitro. In certain embodiments, the step of detecting the cell(s) occurs in vivo. In certain further embodiments, the method involves the detection of the location of the cell-targeted molecule and/or diagnostic composition in an organism using one or more imaging procedures after the step of administrating the cell-targeted molecule and/or diagnostic composition to said organism. The information collected using methods of the present invention may regard the presence of a cell physically coupled with an extracellular target of the binding region of the cell-targeted molecule of the present invention and may be useful in the diagnosis, prognosis, characterization, and/or treatment of a disease, disorder, or condition. For example, cell-targeted molecules of the invention which incorporate detection promoting agents as described herein may be used to characterize diseases as potentially treatable by a related pharmaceutical composition of the present invention. For example, certain cell-targeted molecules (e.g. proteins) and compositions (e.g. pharmaceutical compositions and diagnostic compositions) of the present invention, as well as methods of the present invention may be used to determine if a patient belongs to a group that responds to a pharmaceutical composition of the present invention.

Among certain embodiments of the present invention are kits comprising a composition of matter of the present invention, and optionally, instructions for use, additional reagent(s), and/or pharmaceutical delivery device(s).

These and other features, aspects and advantages of the present invention will become better understood with regard to the following description, appended claims, and accompanying figures. The aforementioned elements of the invention may be individually combined or removed freely in order to make other embodiments of the invention, without any statement to object to such combination or removal hereinafter.

### BRIEF DESCRIPTION OF THE FIGURES

FIG. 1 shows the general arrangement of the polypeptide components of exemplary, cell-targeted molecules of the present invention with “N” and “C” denoting an amino-terminus and carboxy-terminus, respectively, of the cell-targeted molecule or a polypeptide component of the cell-targeted molecule which comprises a Shiga toxin effector polypeptide region. Certain embodiments of the cell-targeted molecules of the present invention comprise a protease-cleavage resistant, Shiga toxin effector polypeptide region (i.e. a Shiga toxin A Subunit effector polypeptide comprising a disrupted furin-cleavage motif) linked, proximal to its carboxy terminus, with a relatively large, binding region (e.g., sizes ranging from 28 to 50 kiloDaltons (kDa)).

FIG. 2 graphically shows SLT-1A::αCD38scFv exhibited improved cytotoxicity as compared to the reverse orientation αCD38scFv::SLT-1A. The percent viability of cells was plotted over the logarithm to base 10 of the cytotoxic protein concentration administered to the cells in nanomolar (nM).

FIG. 3 graphically shows improved target cell-type specific cytotoxicity of SLT-1A::αHER2scFv as compared to the reverse orientation αHER2scFv::SLT-1A. The percent viability of cells was plotted over the logarithm to base 10 of the cytotoxic protein concentration administered to the cells in nanomolar (nM).

FIG. 4 shows microscopy images of the subcellular localization of αHER2scFv::SLT-1A and SLT-1A::αHER2scFv during the intoxication process. The images show both cell-targeted molecules entered target cells within one hour of administration to target positive cells with no apparent difference in cellular internalization behavior by this assay.

FIG. 5 graphically shows SLT-1A::αCD19scFv exhibited improved target cell-type specific cytotoxicity as compared to the reverse orientation αCD19scFv::SLT-1A. The percent viability of cells was plotted over the logarithm to base 10 of the cytotoxic protein concentration administered to the cells in nanomolar (nM).

FIG. 6 graphically shows SLT-1A::αCD74scFv exhibited improved cytotoxicity as compared to the reverse orientation αCD74scFv::SLT-1A. The percent viability of cells was plotted over the logarithm to base 10 of the cytotoxic protein concentration administered to the cells in nanomolar (nM).

FIG. 7 shows the furin-cleavage resistance of the exemplary, cytotoxic protein (SLT-1A-FR::scFv-1), which comprises a Shiga toxin effector polypeptide comprising a disrupted furin-cleavage motif, as compared to a nearly identical, cytotoxic protein comprising a Shiga toxin effector polypeptide comprising a wild-type, furin-cleavage site. FIG. 7 shows a Coomassie-stained, polyacrylamide gel after electrophoresis of protein samples treated with either purified, recombinant, human furin or various negative control conditions. The lanes of the gel are numbered, and the figure legend indicates pre-treatment conditions of each protein sample prior to loading sample to the gel: the temperature in degrees Celsius (° C.), the pre-treatment duration in hours (denoted by “hrs”), and whether any furin was added by denoting the amount of furin activity units (U) per microgram (μg) of sample protein (labeled “U/μg furin”) or “no furin” for zero U/μg furin. The lane marked “L” shows the migration pattern of a protein molecular weight ladder, and the approximate size of each ladder protein band is labeled in kDa. The figure legend indicates which Shiga toxin effector polypeptide was present in each protein sample per lane, either 1) a wild-type furin-cleavage site (WT) or 2) a disrupted furin-cleavage motif (FR). The treated samples were subjected to 0.5 furin activity units per microgram of protein (U/μg furin) at 30° C. for 25 hours (hrs). FIG. 7 shows SLT-1A-FR::scFv-1 was resistant to proteolytic cleavage under the conditions of 0.5 furin activity units per microgram of SLT-1A-FR::scFv-1 at 30° C.

FIG. 8 shows the furin-cleavage resistance at multiple temperatures of the exemplary, cytotoxic protein (SLT-1A-FR::scFv-2), which comprises a Shiga toxin effector polypeptide comprising a disrupted furin-cleavage motif. FIG. 8 shows a Coomassie-stained, polyacrylamide gel after electrophoresis of protein samples treated with either purified, recombinant, human furin or no furin. The lanes of the gel are numbered, and the figure legend indicates pre-treatment conditions of each protein sample prior to loading sample to the gel: the temperature in ° C., the pre-treatment duration in hours (denoted by “hrs”), and whether any furin was added by denoting the amount of furin activity units per microgram of sample cytotoxic protein (labeled “U/μg furin”) or “no furin” for zero U/μg furin. The lane marked “L” shows the migration pattern of a protein molecular weight ladder, and the approximate size of each ladder protein band is labeled in kDa. FIG. 8 shows SLT-1A-FR::scFv-2 was resistant to proteolytic cleavage under the conditions of 0.5 furin activity units per microgram of SLT-1A-FR::scFv-2 at temperatures ranging from 4 to 37° C.

FIG. 9 graphically shows that the exemplary, protease-cleavage resistant, cytotoxic protein SLT-1A-FR::scFv-1 exhibited cell-targeted cytotoxicity comparable to a nearly identical, cytotoxic protein comprising a Shiga toxin effector polypeptide with a wild-type, furin-cleavage site. The percent viability of target positive cells was plotted over the logarithm to base 10 of the cytotoxic protein concentration administered to the cells in nanomolar (nM).

FIG. 10 graphically shows that the exemplary, protease-cleavage resistant, cytotoxic protein SLT-1A-FR::scFv-1 exhibited non-targeted cytotoxicity comparable to a nearly identical, cytotoxic protein comprising a Shiga toxin effector polypeptide with a wild-type, furin-cleavage site. SLT-1A-FR::scFv-1 also showed non-targeted cytotoxicity comparable to an untargeted, wild-type, Shiga toxin A Subunit construct. The percent viability of target negative cells was plotted over the logarithm to base 10 of the cytotoxic protein concentration administered to the cells in nanomolar (nM).

FIG. 11 shows the improved survival of mice administered repeat doses of the exemplary, protease-cleavage resistant, cytotoxic protein SLT-A1-FR::scFv-1 as compared to the protease-cleavage sensitive SLT-A1-WT::scFv-1. FIG. 11 shows Kaplan-Meier survival plots of mice administered 2.5 milligram (mg) per kilogram (kg) of body mass per injection (denoted by “mg/kg/injection”) of protease-cleavage sensitive SLT-1A-WT::scFv-1 or the exemplary, cell-targeted molecule SLT-1A-FR::scFv-1 for a total of three injections. The y-axis shows the percent survival of mice within a dosage group, and the x-axis shows the time in days. Mice exhibited superior tolerability to the protease-cleavage resistant SLT-1A-FR::scFv-1 over time as compared to their tolerability of the protease-cleavage sensitive SLT-1A-WT::scFv-1.

FIG. 12 shows the exemplary, cytotoxic protein SLT-1A-FR::scFv-2 inhibited the growth of target positive, human tumor cells in vivo in a murine xenograft model of human cancer. FIG. 12 shows the tumor burden over time as assayed by bioluminescence per individual mouse based on the expression of a luciferase reporter by human tumor cells grafted into the mouse. An individual mouse is represented by each symbol plotted on the graph, i.e. open triangle, filled triangle, open circle, or filled square. The y-axis shows the total bioluminescence signal of an individual mouse, which represents that mouse's tumor burden, in millions of photons per second (photons/sec), and the x-axis shows the injection dose, which ranged from 0 to 2 milligrams (mg) of SLT-1A-FR::scFv-2 per kilogram (kg) of body mass per injection (denoted by “mg/kg/injection”). The four, x-axis, dose groups correspond to four groups of mice, each group containing ten mice. The dose groups differed by the dosage of cytotoxic protein administered: Group #1—mice received zero mg of SLT-1A-1-R::scFv-2, Group #2—mice received 0.05 mg of SLT-1A-1-R::scFv-2 per kg of body mass, Group #3—mice received 0.5 mg of SLT-1A-FR::scFv-2 per kg of body mass, and Group #4—mice received 2 mg of SLT-1A-FR::scFv-2 per kg of body mass. The experiment was run for at least 4 weeks. FIG. 12 shows both the dosage- and time-dependence of the inhibition of human tumor cell growth exhibited by the exemplary, cell-targeted molecule SLT-1A-FR::scFv-2.

### DETAILED DESCRIPTION

The present invention is described more fully hereinafter using illustrative, non-limiting embodiments, and references to the accompanying figures. This invention may, however, be embodied in many different forms and should not be construed as to be limited to the embodiments set forth below. Rather, these embodiments are provided so that this disclosure is thorough and conveys the scope of the invention to those skilled in the art.

In order that the present invention may be more readily understood, certain terms are defined below. Additional definitions may be found within the detailed description of the invention.

As used in the specification and the appended claims, the terms “a,” “an” and “the” include both singular and the plural referents unless the context clearly dictates otherwise.

As used in the specification and the appended claims, the term “and/or” when referring to two species, A and B, means at least one of A and B. As used in the specification and the appended claims, the term “and/or” when referring to greater than two species, such as A, B, and C, means at least one of A, B, or C, or at least one of any combination of A, B, or C (with each species in singular or multiple possibility).

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout this specification, the term “including” is used to mean “including but not limited to.” “Including” and “including but not limited to” are used interchangeably.

The term “amino acid residue” or “amino acid” includes reference to an amino acid that is incorporated into a protein, polypeptide, or peptide. The term “polypeptide” includes any polymer of amino acids or amino acid residues. The term “polypeptide sequence” refers to a series of amino acids or amino acid residues which physically compose a polypeptide. A “protein” is a macromolecule comprising one or more polypeptides or polypeptide “chains.” A “peptide” is a small polypeptide of sizes less than a total of 15-20 amino acid residues. The term “amino acid sequence” refers to a series of amino acids or amino acid residues which physically comprise a peptide or polypeptide depending on the length. Unless otherwise indicated, polypeptide and protein sequences disclosed herein are written from left to right representing their order from an amino-terminus to a carboxy terminus.

The terms “amino acid,” “amino acid residue,” “amino acid sequence,” or polypeptide sequence include naturally occurring amino acids (including L and D isosteriomers) and, unless otherwise limited, also include known analogs of natural amino acids that can function in a similar manner as naturally occurring amino acids, such as selenocysteine, pyrrolysine, N-formylmethionine, gamma-carboxyglutamate, hydroxyprolinehypusine, pyroglutamic acid, and selenomethionine. The amino acids referred to herein are described by shorthand designations as follows in Table A:

TABLE A
Amino Acid Nomenclature

| Name | 3-letter | 1-letter |
|---|---|---|
| Alanine | Ala | A |
| Arginine | Arg | R |
| Asparagine | Asn | N |
| Aspartic Acid or Aspartate | Asp | D |
| Cysteine | Cys | C |
| Glutamic Acid or Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The phrase “conservative substitution” with regard to a polypeptide, refers to a change in the amino acid composition of the polypeptide that does not substantially alter the function and structure of the overall polypeptide (see Creighton, Proteins: Structures and Molecular Properties (W. H. Freeman and Company, New York (2nd ed., 1992)).

As used herein, the terms “expressed,” “expressing,” or “expresses,” and grammatical variants thereof, refer to translation of a polynucleotide or nucleic acid into a polypeptide or protein. The expressed polypeptides or proteins may remain intracellular, become a component of the cell surface membrane or be secreted into an extracellular space.

As used herein, cells which express a significant amount of an extracellular target biomolecule at least one cellular surface are “target positive cells” or “target+ cells” and are cells physically coupled to the specified extracellular target biomolecule.

As used herein, the symbol “α” is shorthand for an immunoglobulin-type binding region capable of binding to the biomolecule following the symbol. The symbol “α” is used to refer to the functional characteristic of an immunoglobulin-type binding region based on its capability of binding to the biomolecule following the symbol.

The symbol “::” means the polypeptide regions before and after it are physically linked together to form a continuous polypeptide.

The term “selective cytotoxicity” with regard to the cytotoxic activity of a cell-targeted molecule (e.g., a cytotoxic protein of the present invention) refers to the relative levels of cytotoxicity between a targeted cell population and a non-targeted bystander cell population, which can be expressed as a ratio of the half-maximal cytotoxic concentration (CD₅₀) for a targeted cell type over the CD₅₀ for an untargeted cell type to show preferentiality of cell killing of the targeted cell type.

For purposes of the present invention, the term “effector” means providing a biological activity, such as cytotoxicity, biological signaling, enzymatic catalysis, subcellular routing, and/or intermolecular binding resulting in the recruitment of factor(s), and/or allosteric effect(s).

For purposes of the present invention, the phrase “derived from” means that the polypeptide region comprises amino acid sequences originally found in a protein and which may now comprise additions, deletions, truncations, or other alterations from the original sequence such that overall function and structure are substantially conserved. The skilled worker will be able to identify a parental molecule from which a polypeptide region was derived using techniques known in the art, e.g., protein sequence alignment software.

For purposes of the present invention, the phrase “Shiga toxin effector polypeptide,” “Shiga toxin A Subunit effector polypeptide,” “Shiga toxin effector region,” or “Shiga toxin effector polypeptide region” refers to a polypeptide derived from a Shiga toxin A Subunit of a member of the Shiga toxin family that is capable of exhibiting at least one Shiga toxin function. Shiga toxin functions include, e.g., promoting cell entry, deforming lipid membranes, stimulating clathrin-mediated endocytosis, directing its own subcellular routing, directing its own retrograde transport, avoiding intracellular degradation, catalytically inactivating ribosomes, effectuating cytotoxicity, and effectuating cytostatic effects.

For purposes of the present invention, a Shiga toxin effector function is a biological activity conferred by a polypeptide region derived from a Shiga toxin A Subunit. Non-limiting examples of Shiga toxin effector functions include cellular internalization, subcellular routing, catalytic activity, and cytotoxicity. Shiga toxin catalytic activities include, for example, ribosome inactivation, protein synthesis inhibition, N-glycosidase activity, polynucleotide:adenosine glycosidase activity, RNAase activity, and DNAase activity. RIPs can depurinate nucleic acids, polynucleosides, polynucleotides, rRNA, ssDNA, dsDNA, mRNA (and polyA), and viral nucleic acids (Barbieri L et al., Biochem J 286: 1-4 (1992); Barbieri L et al., Nature 372: 624 (1994); Ling J et al., FEBS Lett 345: 143-6 (1994); Barbieri L et al., Biochem J 319: 507-13 (1996); Roncuzzi L, Gasperi-Campani A, FEBS Lett 392: 16-20 (1996); Stirpe F et al., FEBS Lett 382: 309-12 (1996); Barbieri L et al., Nucleic Acids Res 25: 518-22 (1997); Wang P, Tumer N, Nucleic Acids Res 27: 1900-5 (1999); Barbieri L et al., Biochim Biophys Acta 1480: 258-66 (2000); Barbieri L et al., J Biochem 128: 883-9 (2000); Bagga S et al., J Biol Chem 278: 4813-20 (2003); Picard D et al., J Biol Chem 280: 20069-75 (2005)). Some RIPs show antiviral activity and superoxide dismutase activity (Erice A et al., Antimicrob Agents Chemother 37: 835-8 (1993); Au T et al., FEBS Lett 471: 169-72 (2000); Parikh B, Tumer N, Mini Rev Med Chem 4: 523-43 (2004); Sharma N et al., Plant Physiol 134: 171-81 (2004)). Shiga toxin catalytic activities have been observed both in vitro and in vivo. Assays for Shiga toxin effector activity can measure various activities, such as, e.g., protein synthesis inhibitory activity, depurination activity, inhibition of cell growth, cytotoxicity, supercoiled DNA relaxation activity, and/or nuclease activity.

As used herein, the retention of Shiga toxin effector function refers to a level of Shiga toxin functional activity, as measured by an appropriate quantitative assay with reproducibility comparable to a wild-type Shiga toxin effector region control. For ribosome inhibition, Shiga toxin effector function is exhibiting an IC₅₀ of 10,000 picomolar (pM) or less. For cytotoxicity in a target positive cell kill assay, Shiga toxin effector function is exhibiting a CD₅₀ of 1,000 nanomolar (nM) or less, depending on the cell type and its expression of the appropriate extracellular target biomolecule.

For purposes of the claimed invention and with regard to a Shiga toxin protein sequence, the term “wild-type” generally refers to a naturally occurring, Shiga toxin protein sequence(s) found in a living species, such as, e.g., a pathogenic bacterium, wherein that Shiga toxin protein sequence(s) is one of the most frequently occurring variants. This is in contrast to infrequently occurring Shiga toxin protein sequences that, while still naturally occurring, are found in less than one percent of individual organisms of a given species out of individual organisms of that same species when sampling a statistically powerful number of naturally occurring individual organisms of that species which comprise at least one Shiga toxin protein variant. A clonal expansion of a natural isolate outside its natural environment (regardless of whether the isolate is an organism or molecule comprising biological sequence information) does not alter the naturally occurring requirement as long as the clonal expansion does not introduce new sequence variety not present in naturally occurring populations of that species and/or does not change the relative proportions of sequence variants to each other.

As used herein, the retention of “significant” Shiga toxin effector function refers to a level of Shiga toxin functional activity, as measured by an appropriate quantitative assay with reproducibility comparable to a wild-type Shiga toxin effector polypeptide control. For in vitro ribosome inhibition, significant Shiga toxin effector function is exhibiting an IC₅₀ of 300 pM or less depending on the source of the ribosomes (e.g. bacteria, archaea, or eukaryote (algae, fungi, plants, or animals)) This is significantly greater inhibition as compared to the approximate IC₅₀ of 100,000 pM for the catalytically inactive SLT-1A 1-251 double mutant (Y77S/E167D). For cytotoxicity in a target positive cell kill assay in laboratory cell culture, significant Shiga toxin effector function is exhibiting a CD₅₀ of 100, 50, or 30 nM or less, depending on the cell line and its expression of the appropriate extracellular target biomolecule. This is significantly greater cytotoxicity to the appropriate target cell line as compared to the SLT-1A component alone, without a cell targeting binding region, which has a CD₅₀ of 100-10,000 nM, depending on the cell line.

For some samples, accurate values for either IC₅₀ or CD₅₀ might be unobtainable due to the inability to collect the required data points for an accurate curve fit. Inaccurate IC₅₀ and/or CD₅₀ values should not be considered when determining significant Shiga toxin effector function activity. Data insufficient to accurately fit a curve as described in the analysis of the data from exemplary, Shiga toxin effector function assays, such as, e.g., assays described in the Examples, should not be considered as representative of actual Shiga toxin effector function. For example, theoretically, neither an IC₅₀ nor CD₅₀ can be determined if greater than 50% ribosome inhibition or cell death, respectively, does not occur in a concentration series for a given sample.

The failure to detect activity in Shiga toxin effector function may be due to improper expression, polypeptide folding, and/or polypeptide stability rather than a lack of cell entry, subcellular routing, and/or enzymatic activity. Assays for Shiga toxin effector functions may not require much of the molecule of the invention to measure significant amounts of Shiga toxin effector function activity. To the extent that an underlying cause of low or no effector function is determined empirically to relate to protein expression or stability, one of skill in the art may be able to compensate for such factors using protein chemistry and molecular engineering techniques known in the art, such that a Shiga toxin functional effector activity may be restored and measured. As examples, improper cell-based expression may be compensated for by using different expression control sequences; improper polypeptide folding and/or stability may benefit from stabilizing terminal sequences, or compensatory mutations in non-effector regions which stabilize the three-dimensional structure of the protein, etc. When new assays for individual Shiga toxin functions become available, Shiga toxin effector polypeptides may be analyzed for any level of those Shiga toxin effector functions, such as for being within a certain-fold activity of a wild-type, Shiga toxin effector polypeptide.

Certain Shiga toxin effector functions are not easily measurable, e.g. subcellular routing functions. Currently there is no routine, quantitative assay to distinguish whether the failure of a Shiga toxin effector polypeptide to be cytotoxic is due to improper subcellular routing, but at a time when tests are available, then Shiga toxin effector polypeptides may be analyzed for any significant level of subcellular routing as compared to the appropriate wild-type Shiga toxin effector region.

It should be noted that even if the cytotoxicity of a Shiga toxin effector polypeptide is reduced relative to wild-type, in practice, applications using attenuated Shiga toxin effector polypeptides may be equally or more effective than those using wild-type Shiga toxin effector polypeptides because the highest potency variants might exhibit undesirable effects which are minimized in reduced potency variants. Wild-type Shiga toxin effector polypeptides are very potent, being able to kill with only one molecule reaching the cytosol or perhaps forty molecules being internalized. Shiga toxin effector polypeptides with even considerably reduced Shiga toxin effector functions, such as, e.g., subcellular routing or cytotoxicity, as compared to wild-type Shiga toxin effector polypeptides may still be potent enough for practical applications involving targeted cell killing and/or specific cell detection.

For purposes of the claimed invention, the term “equivalent” with regard to cytotoxicity means an empirically measured level of cytotoxicity, as measured by an appropriate quantitative assay with reproducibility, which is reproducibly within 10% or less of the activity of the reference molecule (e.g., the second cell-targeted molecule or third cell-targeted molecule).

For purposes of the claimed invention, the term “associated” or “association” with regard to two molecular components refers to the state of the two components being joined, attached, connected, linked, or otherwise coupled to form a single molecule and includes covalent and/or non-covalent associations.

For purposes of the present invention, the term “linked” refer to two or more molecular components associated by one or more atomic interactions such that a single molecule is formed and wherein the atomic interactions includes at least one covalent bond. For purposes of the present invention, the term “linking” refers to the act of creating a linked molecule as described above.

For purposes of the present invention, the term “fused” refers to two or more proteinaceous components associated by at least one covalent bond which is a peptide bond. Non-limiting examples of two proteinaceous components fused together include, e.g., an amino acid, peptide, or polypeptide fused to a polypeptide via a peptide bond such that the resulting molecule is a single, continuous polypeptide. For purposes of the present invention, the term “fusing” refers to the act of creating a fused molecule as described above, such as, e.g., a fusion protein generated from the recombinant fusion of genetic regions.

For purposes of the present invention, the term “heterologous” means of a different source than a Shiga holotoxin, e.g. a heterologous molecular moiety or polypeptide is one that is not natively found as part of or linked to a naturally occurring, A Subunit of a native, Shiga toxin expressed by a naturally occurring bacterial species.

For purposes of the present invention, the phrase “proximal to an amino-terminus” with reference to the position of a Shiga toxin effector polypeptide region within a cell-targeted molecule of the present invention (e.g., a protein of the present invention) refers to at least one amino acid residue of the Shiga toxin effector polypeptide region being within 1, 2, 3, 4, 5, 6, 7, 8, 9, 10, 11, 12 or more, e.g., up to 18-20 amino acid residues, of an amino-terminus of the cell-targeted molecule, as long as the cell-targeted molecule retains the appropriate level of activity noted herein (e.g., a certain level of cytotoxic potency). In other words, amino acid residue(s) amino-terminal to the Shiga toxin effector polypeptide within the cell-targeted molecule of the present invention should not reduce any Shiga toxin effector activity (e.g., by sterically hindering a structure(s) near the amino-terminus of the Shiga toxin effector polypeptide region) such that an activity of the cell-targeted molecule (e.g., cytotoxicity) is reduced below the appropriate activity level noted herein.

For purposes of the present invention, the phrase “more proximal to an amino-terminus” with reference to the position of a Shiga toxin effector polypeptide region within a cell-targeted molecule of the present invention (e.g., a protein of the present invention) as compared to another polypeptide component (e.g., a cell-targeting, binding region) refers to a position wherein at least one amino acid residue of the amino-terminus of the Shiga toxin effector polypeptide is closer to the amino-terminus of a polypeptide component of a cell-targeted molecule of the present invention.

For purposes of the present invention, the term “Shiga toxin A1 fragment region” refers to a polypeptide region consisting essentially of a Shiga toxin A1 fragment and/or derived from a Shiga toxin A1 fragment of a Shiga toxin.

For purposes of the present invention, the phrase “carboxy terminus region of a Shiga toxin A1 fragment” refers to a polypeptide region derived from a naturally occurring Shiga toxin A1 fragment, the region beginning with a hydrophobic residue (e.g., V236 of StxA-A1 and SLT-1A1, and V235 of SLT-2A1) that is followed by a hydrophobic residue and the region ending with the furin-cleavage site conserved among Shiga toxin A1 fragment polypeptides and ending at the junction between the A1 fragment and the A2 fragment in native, Shiga toxin A Subunits. For purposes of the present invention, the carboxy-terminal region of a Shiga toxin A1 fragment includes a peptidic region derived from the carboxy terminus of a Shiga toxin A1 fragment polypeptide, such as, e.g., a peptidic region comprising or consisting essentially of the carboxy terminus of a Shiga toxin A1 fragment. Non-limiting examples of peptidic regions derived from the carboxy terminus of a Shiga toxin A1 fragment include the amino acid residue sequences natively positioned from position 236 to position 239, 240, 241, 242, 243, 244, 245, 246, 247, 248, 249, 250, or 251 in Stx1A (SEQ ID NO:2) or SLT-1A (SEQ ID NO:1); and from position 235 to position 239, 240, 241, 242, 243, 244, 245, 246, 247, 248, 249, or 250 in SLT-2A (SEQ ID NO:3).

For purposes of the present invention, the terms “terminus,” “amino-terminus,” or “carboxy-terminus” with regard to a cell-targeted molecule refers generally to the last amino acid residue of a polypeptide chain of the cell-targeted molecule (e.g., a single, continuous polypeptide chain). A cell-targeted molecule may comprise more than one polypeptides or proteins, and, thus, a cell-targeted molecule of the present invention may comprise multiple amino-terminals and carboxy-terminals. For example, the “amino-terminus” of a cell-targeted molecule may be defined by the first amino acid residue of a polypeptide chain representing the amino-terminal end of the polypeptide, which is generally characterized by a starting, amino acid residue which does not have a peptide bond with any amino acid residue involving the primary amino group of the starting amino acid residue or involving the equivalent nitrogen for starting amino acid residues which are members of the class of N-alkylated alpha amino acid residues. Similarly, the “carboxy-terminus” of a cell-targeted molecule may be defined by the last amino acid residue of a polypeptide chain representing the carboxyl-terminal end of the polypeptide, which is generally characterized by a final, amino acid residue which does not have any amino acid residue linked by a peptide bond to the alpha-carbon of its primary carboxyl group.

For purposes of the present invention, the terms “terminus,” “amino-terminus,” or “carboxy-terminus” with regard to a polypeptide region refers to the regional boundaries of that region, regardless of whether additional amino acid residues are linked by peptide bonds outside of that region. In other words, the terminals of the polypeptide region regardless of whether that region is fused to other peptides or polypeptides. For example, a fusion protein comprising two proteinaceous regions, e.g., a binding region polypeptide and a Shiga toxin effector region polypeptide, may have a Shiga toxin effector region with a carboxy-terminus ending at amino acid residue 251 of the fusion protein despite a peptide bond to an amino acid residue at position 252 of the fusion protein representing the beginning of another proteinaceous region, e.g., the binding region. In this example, the carboxy-terminus of the Shiga toxineffector region refers to residue 251, which is not a terminus of the fusion protein but rather represents an internal regional boundary. Thus, for polypeptide regions, the terms “terminus,” “amino-terminus,” and “carboxy-terminus” are used to refer to the boundaries of polypeptide regions, whether the boundary is a physically terminal or an internal position embedded within a larger polypeptide chain.

For purposes of the present invention, the phrase “proximal to the carboxy terminus of an A1 fragment polypeptide” with regard to a linked molecular moiety or binding region refers to being within 1, 2, 3, 4, 5, 6, 7, 8, 9, 10, 11, or 12 amino acid residues from the amino acid residue defining the last residue of the Shiga toxin A1 fragment polypeptide.

For purposes of the present invention, the phrase “sterically covers the carboxy terminus of the A1 fragment-derived region” includes any molecular moiety of a size of 4.5 kDa or greater (e.g., an immunoglobulin-type binding region) covalently linked to an amino acid residue in the carboxy terminus of the A1 fragment-derived region, such as, e.g., the amino acid residue derived from the amino acid residue natively positioned at any one of positions 236 to 251 in Stx1A (SEQ ID NO:2) or SLT-1A (SEQ ID NO:1) or from 235 to 250 in SLT-2A (SEQ ID NO:3). For purposes of the present invention, the phrase “sterically covers the carboxy terminus of the A1 fragment-derived region” also includes any molecular moiety of a size of 4.5 kDa or greater (e.g., an immunoglobulin-type binding region) covalently linked to an amino acid residue in the carboxy terminus of the A1 fragment-derived region, such as, e.g., the amino acid residue carboxy-terminal to the last amino acid A1 fragment-derived region and/or the Shiga toxin effector polypeptide. For purposes of the present invention, the phrase “sterically covers the carboxy terminus of the A1 fragment-derived region” also includes any molecular moiety of a size of 4.5 kDa or greater (e.g., an immunoglobulin-type binding region) physically preventing cellular recognition of the carboxy terminus of the A1 fragment-derived region, such as, e.g. recognition by the ERAD machinery.

For purposes of the present invention, a binding region, such as, e.g., an immunoglobulin-type binding region, that comprises a polypeptide comprising at least forty amino acids and that is linked (e.g., fused) to the carboxy terminus of the Shiga toxin effector polypeptide region comprising an A1 fragment-derived region is a molecular moiety which is “sterically covering the carboxy-terminus of the A1 fragment-derived region.”

For purposes of the present invention, a binding region, such as, e.g., an immunoglobulin-type binding region, that comprises a polypeptide comprising at least forty amino acids and that is linked (e.g., fused) to the carboxy terminus of the Shiga toxin effector polypeptide region comprising an A1 fragment-derived region is a molecular moiety “encumbering the carboxy-terminus of the A1 fragment-derived region.”

For purposes of the claimed invention, the phrase “furin-cleavage motif at the carboxy terminus of the A1 fragment region” refers to a specific, furin-cleavage motif conserved among Shiga toxin A Subunits and bridging the junction between the A1 fragment and the A2 fragment in native, Shiga toxin A Subunits.

For purposes of the present invention, the phrase “furin-cleavage site proximal to the carboxy terminus of the A1 fragment region” refers to any identifiable, furin-cleavage site having an amino acid residue within a distance of less than 1, 2, 3, 4, 5, 6, 7, or more amino acid residues of the amino acid residue defining the last amino acid residue in the A1 fragment region.

The effectiveness and potency of immunotoxins and ligand-toxin fusions as cytotoxic molecules is influenced by the densities of their target antigen(s) on a target cell surface (see e.g. Decket T et al., Blood 103: 2718-26 (2004); Du X et al., Blood 111: 338-43 (2008); Baskar S et al., mAbs 4: 349-61 (2012)), epitope location (Press O et al., J Immunol 141: 4410-7 (1988); Godal A et al., In J Cancer 52: 631-5 (1992); Yazdi P et al., Cancer Res 55: 3763-71 (1995)), rate of internalization of the surface bound cytotoxic molecule (see e.g. Du X et al., Cancer Res 68: 6300-5 (2008)), and the intracellular itinerary (Tortorella L et al., PLoS One 7: e47320 (2012)).

The cell surface representation and/or density of a given extracellular target biomolecule may influence the applications for which certain cell-targeted molecules of the present invention may be most suitably used. Differences in cell surface representation and/or density of a given target biomolecule between cells may alter the internalization and/or cytotoxicity of a given cell-targeted molecule of the invention both quantitatively and qualitatively. The cell surface representation and/or density of a given target biomolecule can vary greatly among target biomolecule positive cells or even on the same cell at different points in the cell cycle or cell differentiation. The total cell surface representation of a given target biomolecule on a particular cell or population of cells may be determined using methods known to the skilled worker, such as the fluorescence-activated cell sorting (FACS) flow cytometry methods.

### Introduction

The present invention solves problems for engineering potently cytotoxic cell-targeted molecules which comprise Shiga-toxin-Subunit-A-derived regions for cytotoxicity linked to heterologous, binding regions for cell targeting, e.g. fusion proteins which retain robust, Shiga toxin A Subunit functionalities, such as efficient intracellular routing to the cytosol. For better optimized cytotoxic potency of such cell-targeted molecules, the intracellular routing of the Shiga toxin effector polypeptide region must be sufficient to permit enzymatically active, Shiga-toxin-Subunit-A-derived polypeptide(s) to efficiently reach the cytosolic compartment and inactivate ribosomes to potently cause cell death. The present invention is based on the observation that a particular structural relationship between the Shiga-toxin-Subunit-A-derived toxin region and a heterologous binding region in a cell-targeted molecule can impact the cytotoxic potency of the cell-targeted molecule, possibly due to better optimization of intracellular routing in certain structural arrangements.

As described in more detail in the Examples below, having the Shiga-toxin-Subunit-A-derived toxin region oriented proximal to an amino-terminus relative to a heterologous, cell-targeting, binding region resulted in higher cytotoxic effects, i.e. better optimized cytotoxic potencies. The Examples demonstrate that orienting the Shiga-toxin-Subunit-A-derived toxin region proximal to an amino-terminus of a polypeptide component of a cell-targeted molecule resulted in significantly more robust cell kill (4-fold to 9-fold depending on the exemplary molecule) than the identical components arranged with the Shiga toxin effector region more distally from any amino-terminus of the molecule. The present invention provides a specific way of engineering cell-targeted molecules to accomplish optimized Shiga toxin cytotoxic potency by arranging the Shiga toxin effector region proximal to an amino-terminus of a polypeptide component of the cell-targeted molecule. The linking of heterologous, cell-targeting, binding regions with Shiga toxin-Subunit A polypeptide regions in this optimized orientation allows for the engineering of more potent cell-type specific targeting of Shiga toxin cytotoxicity as well as more efficient intracellular routing of Shiga toxin effector polypeptides, such as, e.g., to the endoplasmic reticulum and/or cytosol.

Previously, Shiga toxin A Subunit fusion constructs were shown to be cytotoxic and presumably capable of self-directing their own intracellular routing to deliver an enzymatically active toxin fragment to the cytosol (Al-Jaufy, Infect Immun 62: 956-60 (1994); Al-Jaufy, Infect Immun 63: 3073-8 (1995); Su H, Protein Expr Purif 66: 149-57 (2009)); however, none of these constructs comprised immunoglobulin-type binding regions. When multiple cytotoxic proteins were created and tested with Shiga toxin derived regions linked to immunoglobulin derived targeting regions at their amino-terminals, these engineered proteins did not display the expected levels of cytotoxicity (Examples, infra). However, these lower-cytotoxicity proteins exhibited cell surface binding and entry, as well as similar in vitro enzymatic activities and similar binding affinities as compared to more cytotoxic variants with the same polypeptide regions linked in a different configuration (Examples, infra). Thus, the differences in cytotoxic potency between orientation variants were not predictable based on the functional characteristics of their proteinaceous components in biochemical assays and/or the fundamental, functional characteristics of the cytotoxic proteins in various biochemical and cellular assays (e.g., Shiga toxin enzymatic activity, target cell binding affinity, and cellular internalization).

As described further in the Examples, the potencies of cytotoxic, cell-targeted molecules were better optimized (i.e. increased) by changing the orientation such that immunoglobulin-type, binding regions were linked to carboxy-proximal regions of Shiga toxin effector polypeptide regions. However, the orientation of engineering did not alter the functional characteristics of the proteinaceous components with regard to the catalytic activity of the Shiga toxin-derived region and the binding kinetics of the immunoglobulin-type, binding region. The sensitivity of the cytotoxicity (and presumably intracellular routing) of Shiga-toxin-based constructs with heterologous binding regions to the orientation of engineering its proteinaceous components is unexpected and remains unexplained. The improved cytotoxicity for certain orientation constructs could not be explained by differences in the Shiga-toxin-based construct's target binding characteristics, cell binding characteristics, or catalytic activities.

The cell-targeted molecules of the present invention (e.g., cytotoxic proteins comprising immunoglobulin-type, binding regions linked carboxy-terminal to Shiga toxin effector polypeptide regions) may optionally comprise furin-cleavage resistant, Shiga toxin A Subunit effector polypeptides which confer certain improvement(s) over cell-targeted molecules comprising protease-cleavage sensitive, Shiga toxin effector polypeptides without sacrificing the better optimized cytotoxicity observed of furin-cleavage sensitive constructs, i.e. cytotoxicity equivalent to a cell-targeted molecule comprising a wild-type, Shiga toxin effector polypeptide oriented more proximally to an amino-terminus of the construct than the cell-targeting, binding region moiety.

### I. The General Structure of the Cell-Targeted Molecules of the Present Invention

The present invention provides various cell-targeted molecules, each molecule comprising 1) a binding region for cell-targeting and 2) a Shiga toxin effector polypeptide region for cellular internalization, intracellular routing, and/or cell killing. The linking of cell-targeting, binding regions with polypeptides derived from Shiga toxin A Subunits enables the engineering of cell-type specific targeting of potent Shiga toxin cytotoxicity. Certain embodiments of the cell-targeted molecule of the present invention comprise an amino-terminal and/or amino-terminus proximal, Shiga toxin A Subunit effector polypeptide region (derived from at least one Shiga toxin A Subunit of a member of the Shiga toxin family) linked to a binding region that can bind specifically to at least one extracellular target biomolecule in physical association with a cell, such as a target biomolecule expressed on the surface of a cell. Certain further embodiments comprise a binding region linked more proximally to the carboxy-terminus of the Shiga toxin effector polypeptide region than to the amino-terminus of the Shiga toxin effector region. Certain further embodiments comprise a binding region linked to the carboxy-terminus of the Shiga toxin effector polypeptide region. This general structure is modular in that any number of diverse binding regions may be linked to various, Shiga toxin A Subunit effector polypeptides to produce variations of the same, general, cell-targeted molecule of the present invention.

### A. Binding Regions

The binding region of a cell-targeted molecule of the present invention comprises a peptide or polypeptide region capable of binding specifically to an extracellular target biomolecule. Binding region may comprise one or more various peptidic or polypeptide moieties, such as randomly generated peptide sequences, naturally occurring ligands or derivatives thereof, immunoglobulin derived domains, synthetically engineered scaffolds as alternatives to immunoglobulin domains, and the like.

There are numerous binding regions known in the art that are useful for targeting polypeptides to specific cell-types via their binding characteristics, such as ligands, monoclonal antibodies, engineered antibody derivatives, and engineered alternatives to antibodies.

According to one specific, but non-limiting aspect, the binding region of the molecule of the invention comprises a naturally occurring ligand or derivative thereof that retains binding functionality to an extracellular target biomolecule, commonly a cell surface receptor. For example, various cytokines, growth factors, and hormones known in the art may be used to target the cell-targeted molecule to the cell-surface of specific cell types expressing a cognate cytokine receptor, growth factor receptor, or hormone receptor. Certain non-limiting examples of ligands include epidermal growth factors, fibroblast growth factors, vascular endothelial growth factors, interleukins (such as IL-2, IL-6, and IL-23), and B-cell activating factor (BAFF).

According to certain other embodiments, the binding region comprises a synthetic ligand capable of binding an extracellular target biomolecule (see e.g. Liang S et al., J Mol Med 84: 764-73 (2006); Ahmed S et al., Anal Chem 82: 7533-41 (2010); Kaur K et al., Methods Mol Biol 1248: 239-47 (2015)).

In certain embodiments, the cell-targeted molecule of the present invention comprises a cell-targeting carrier, such as, e.g., a liposome, polymer, nanocarrier, microsphere, nanosphere, dendrimer, polymeric micelle, and/or other silicon or carbon materials including nanotubes, nanorods and nanohorns, magnetic nanoparticles, microemulsions, etc. (Sinha R et al., Molecular Cancer Therapeutics 5: 1909-17 (2006); L Brinton et al., Journal of the National Cancer Institute 100: 1643-8 (2008); Tanaka T et al., Biomed Micro Devices 11: 49-63 (2009)). In certain embodiments, the cell-targeted molecule of the present invention comprises a binding region which is part of a cell-targeting carrier. The association of a polypeptide component of the cell-targeted molecule of the present invention to a cell-targeting carrier may be accomplished using techniques known to the skilled worker, such as, e.g., by using covalent bonds and/or encapsulation.

In certain embodiments, the binding region of a cell-targeted molecule of the present invention comprises one or more polypeptides capable of selectively and specifically binding an extracellular target biomolecule.

### Immunoglobulin-Type Binding Regions

The binding region of a cell-targeted molecule of the present invention (e.g. a cell-targeted, fusion protein of the present invention) may comprise an immunoglobulin-type binding region comprising one or more polypeptides capable of selectively and specifically binding an extracellular target biomolecule. There are numerous, immunoglobulin-type polypeptides contemplated as binding regions of the cell-targeted molecules of the present invention.

The term “immunoglobulin-type binding region” as used herein refers to a polypeptide region capable of binding one or more target biomolecules, such as an antigen or epitope Immunoglobulin-type binding regions are functionally defined by their ability to bind to target molecules Immunoglobulin-type binding regions are commonly derived from antibody or antibody-like structures; however, alternative scaffolds from other sources are contemplated as being including within the scope of the term as used herein, as long as those scaffolds support display screening for binding affinity, such as, e.g., by phage display or directed evolution, to find novel binding region variants which bind with high-affinity to diverse target biomolecules.

There are numerous immunoglobulin-derived binding regions and non-immunoglobulin derived but immunoglobulin-like in their flexibility and toleration of mutations while maintaining a relatively constant structural framework.

Non-limiting, structural examples of immunoglobulin-type binding regions include: autonomous V_{H} domains, single-domain antibody (sdAb) fragments, nanobodies, heavy-chain antibody domains derived from camelid V_{H}H fragments or V_{H} domain fragments, heavy-chain antibody domains derived from cartilaginous fish, immunoglobulin new antigen receptors (IgNARs), V_{NAR} fragments, single-chain variable fragments (scFvs), antibody variable fragments (Fvs), a complementary determining region 3 (CDR3) fragments, constrained FR3-CDR3-FR4 (FR3-CDR3-FR4) polypeptides, Fd fragments, small modular immunopharmaceutical (SMIP) domains, antigen-binding fragments (Fabs), fibronectin-derived 10^{th} fibronectin type III domains (10Fn3) (e.g. monobodies), tenascin type III domains (e.g. TNfn3), ankyrin repeat motif domains (ARD), low-density-lipoprotein-receptor-derived A-domains (A domains of LDLRs or LDLR-As), lipocalins (anticalins), Kunitz domains, Protein-A-derived Z domains, gamma-B crystalline-derived domains, ubiquitin-derived domains, Sac7d-derived polypeptides (affitins), Fyn-derived SH2 domains, miniproteins, C-type lectin-like domain scaffolds, and engineered antibody mimics.

In certain embodiments, the binding region of the cell-targeted molecule of the present invention is selected from the group which includes: autonomous V_{H} domains, single-domain antibody domains (sdAbs), nanobodies, heavy-chain antibody domains derived from camelids (V_{H}H fragments or V_{H} domain fragments), bivalent nanobodies, heavy-chain antibody domains derived from cartilaginous fishes, immunoglobulin new antigen receptors (IgNARs), V_{NAR} fragments, single-chain variable (scFv) fragments, multimerizing scFv fragments (diabodies, triabodies, tetrabodies), bispecific tandem scFv fragments, disulfide stabilized antibody variable (Fv) fragments, disulfide stabilized antigen-binding (Fab) fragments consisting of the V_{L}, V_{H}, C_{L} and C_{H}1 domains, divalent F(ab′)2 fragments, Fd fragments consisting of the heavy chain and C_{H}1 domains, single chain Fv-C_{H}3 minibodies, bispecific minibodies, dimeric C_{H}2 domain fragments (C_{H}2D), Fc antigen binding domains (Fcabs), isolated complementary determining region 3 (CDR3) fragments, constrained framework region 3, CDR3, framework region 4 (FR3-CDR3-FR4) polypeptides, small modular immunopharmaceutical (SMIP) domains, scFv-Fc fusions, multimerizing scFv fragments (diabodies, triabodies, tetrabodies), disulfide stabilized antibody variable (Fv) fragments, disulfide stabilized antigen-binding (Fab) fragments consisting of the V_{L}, V_{H}, C_{L} and C_{H}1 domains, bivalent nanobodies, bivalent minibodies, bivalent F(ab′)₂ fragments (Fab dimers), bispecific tandem V_{H}H fragments, bispecific tandem scFv fragments, bispecific nanobodies, bispecific minibodies, and any genetically manipulated counterparts of the foregoing that retain its paratope and binding function (see Ward E et al., Nature 341: 544-6 (1989); Davies J, Riechmann L, Biotechnology (NY) 13: 475-9 (1995); Reiter Y et al., Mol Biol 290: 685-98 (1999); Riechmann L, Muyldermans S, J Immunol Methods 231: 25-38 (1999); Tanha J et al., J Immunol Methods 263: 97-109 (2002); Vranken W et al., Biochemistry 41: 8570-9 (2002); Jespers L et al., J Mol Biol 337: 893-903 (2004); Jespers L et al., Nat Biotechnol 22: 1161-5 (2004); To R et al., J Biol Chem 280: 41395-403 (2005); Saerens D et al., Curr Opin Pharmacol 8: 600-8 (2008); Dimitrov D, MAbs 1: 26-8 (2009); Weiner L, Cell 148: 1081-4 (2012); Ahmad Z et al., Clin Dev Immunol 2012: 980250 (2012)).

There are a variety of binding regions comprising polypeptides derived from the constant regions of immunoglobulins, such as, e.g., engineered dimeric Fc domains, monomeric Fcs (mFcs), scFv-Fcs, V_{H}H-Fcs, C_{H}2 domains, monomeric C_{H}3s domains (mC_{H}3s), synthetically reprogrammed immunoglobulin domains, and/or hybrid fusions of immunoglobulin domains with ligands (Hofer T et al., Proc Natl Acad Sci U.S.A. 105: 12451-6 (2008); Xiao J et al., J Am Chem Soc 131: 13616-13618 (2009); Xiao X et al., Biochem Biophys Res Commun 387: 387-92 (2009); Wozniak-Knopp G et al., Protein Eng Des Sel 23 289-97 (2010); Gong R et al., PLoS ONE 7: e42288 (2012); Wozniak-Knopp G et al., PLoS ONE 7: e30083 (2012); Ying T et al., J Biol Chem 287: 19399-408 (2012); Ying T et al., J Biol Chem 288: 25154-64 (2013); Chiang M et al., J Am Chem Soc 136: 3370-3 (2014); Rader C, Trends Biotechnol 32: 186-97 (2014); Ying T et al., Biochimica Biophys Acta 1844: 1977-82 (2014)).

In accordance with certain other embodiments, the immunoglobulin-type binding region comprises an engineered, alternative scaffold to immunoglobulin domains. Engineered alternative scaffolds are known in the art which exhibit similar functional characteristics to immunoglobulin-derived structures, such as high-affinity and specific binding of target biomolecules, and may provide improved characteristics to immunoglobulin domains, such as, e.g., greater stability or reduced immunogenicity. Generally, alternative scaffolds to immunoglobulins are less than 20 kilodaltons, consist of a single polypeptide chain, lack cysteine residues, and exhibit relatively high thermodynamic stability.

For example, numerous alternative scaffolds have been identified which bind to the extracellular receptor HER2 (see e.g. Wikman M et al., Protein Eng Des Sel 17: 455-62 (2004); Orlova A et al. Cancer Res 66: 4339-8 (2006); Ahlgren S et al., Bioconjug Chem 19: 235-43 (2008); Feldwisch J et al., J Mol Biol 398: 232-47 (2010); U.S. Pat. Nos. 5,578,482; 5,856,110; 5,869,445; 5,985,553; 6,333,169; 6,987,088; 7,019,017; 7,282,365; 7,306,801; 7,435,797; 7,446,185; 7,449,480; 7,560,111; 7,674,460; 7,815,906; 7,879,325; 7,884,194; 7,993,650; 8,241,630; 8,349,585; 8,389,227; 8,501,909; 8,512,967; 8,652,474; and U.S. patent application 2011/0059090).

In accordance with certain other embodiments, the binding region includes engineered, alternative scaffolds to immunoglobulin domains that exhibit similar functional characteristics, such as high-affinity and specific binding of target biomolecules, and enables the engineering of improved characteristics, such as greater stability or reduced immunogenicity. For certain embodiments of the cell-targeted molecules of the present invention, the binding region is selected from the group which includes engineered Armadillo repeat polypeptides (ArmRPs), engineered, fibronectin-derived, 10^{th} fibronectin type III (10Fn3) domain (monobodies, AdNectins™, or AdNexins™); engineered, tenascin-derived, tenascin type III domain (Centryns™); engineered, ankyrin repeat motif containing polypeptide (DARPins™); engineered, low-density-lipoprotein-receptor-derived, A domain (LDLR-A) (Avimers™); lipocalin (anticalins); engineered, protease inhibitor-derived, Kunitz domain; engineered, Protein-A-derived, Z domain (Affibodies™); engineered, gamma-B crystalline-derived scaffold or engineered, ubiquitin-derived scaffold (Affilins); Sac7d-derived polypeptides (Nanoffitins® or affitins); engineered, Fyn-derived, SH2 domain (Fynomers®); and engineered antibody mimic and any genetically manipulated counterparts of the foregoing that retains its binding functionality (Worn A, Plückthun A, J Mol Biol 305: 989-1010 (2001); Xu L et al., Chem Biol 9: 933-42 (2002); Wikman M et al., Protein Eng Des Sel 17: 455-62 (2004); Binz H et al., Nat Biotechnol 23: 1257-68 (2005); Holliger P, Hudson P, Nat Biotechnol 23: 1126-36 (2005); Gill D, Damle N, Curr Opin Biotech 17: 653-8 (2006); Koide A, Koide S, Methods Mol Biol 352: 95-109 (2007); Byla P et al., J Biol Chem 285: 12096 (2010); Zoller F et al., Molecules 16: 2467-85 (2011); Alfarano P et al., Protein Sci 21: 1298-314 (2012); Madhurantakam C et al., Protein Sci 21: 1015-28 (2012); Varadamsetty G et al., J Mol Biol 424: 68-87 (2012)).

Immunoglobulin-type structures share the feature of representing general scaffolds which may be readily engineered by varying amino acid residues to alter target molecule specificity, such as, e.g. via display screening methods like phage display (see e.g. Binz H et al., Nat Biotechnol 23: 1257-1268 (2005); Binz H, Plückthun A, Curr Opin Biotechnol 16: 459-69 (2005); Holliger P, Hudson P, Nat Biotechnol 23: 1126-36 (2005); Chao G et al., Nat Protoc 1: 755-68 (2006); Paschke M, Appl Microbiol Biotechnol 70: 2-11 (2006); Zahnd C et al., Nat Methods 4: 269-79 (2007); Skerra A, Curr Opin Biotechnol 18: 295-304 (2007); Hackel B et al., J Mol Biol 381: 1238-52 (2008); Skerra A, FEBS J 275: 2677-83 (2008); Saerens D et al., J Immunol Methods 329: 138-50 (2008); Bloom L, Calabro V, Drug Discov Today 14: 949-55 (2009); Gebauer M, Skerra A, Curr Opin Chem Biol 13: 245-55 (2009); Liao H et a., J Biol Chem 284: 17512-20 (2009); Lloyd C et al., Protein Eng Des Sel 22: 159-68 (2009); Hackle B, Wittrup K, Protein Eng Des Sel 23: 211-9 (2010); Hackel B et al., J Mol Biol 401: 84-96 (2010); Lipovsek D, Protein Eng Des Sel 24 3-9 (2011); Tillib S et al., Acta Naturae 2: 85-93 (2010); Wojcik J et al., Nat Struct Mol Biol 17: 519-27 (2010); Zahnd C et al., Cancer Res 70: 1595-605 (2010); Boersma Y, Plückthun A, Curr Opin Biotechnol 22: 849-57 (2011); Bradbury A et al., Nat Biotechnol 29: 245-54 (2011); Gebauer M, Skerra A, Methods Enzymol 503: 157-88 (2012); Geyer C et al., Methods Mol Biol 901: 11-32 (2012); Jacobs S et al., Protein Eng Des Sel 25: 107-17 (2012); Koide S et al., Methods Enzymol 503: 135-56 (2012); Ribosome Display and Related Technologies—Methods and Protocols, Methods in Molecular Biology, vol. 805: pp. 161-190 (Eds. Douthwaite J, Jackson R. Humana Press 2012); Zou Y, Marks J, Methods Enzymol 502: 43-66 (2012); Chen T et al., Methods Enzymol 523: 303-26 (2013); WO 2015/120058 and references therein).

Immunoglobulin (Ig) proteins have a structural domain known as an Ig domain. Ig domains range in length from about 70-110 amino acid residues and possess a characteristic Ig-fold, in which typically 7 to 9 antiparallel beta strands arrange into two beta sheets which form a sandwich-like structure. The Ig fold is stabilized by hydrophobic amino acid interactions on inner surfaces of the sandwich and highly conserved disulfide bonds between cysteine residues in the strands. Ig domains may be variable (IgV or V-set), constant (IgC or C-set) or intermediate (IgI or I-set). Some Ig domains may be associated with a complementarity determining region (CDR), also called a “complementary determining region,” which is important for the specificity of antibodies binding to their epitopes.

Ig-like domains, which commonly share a general beta-sheet structure described above and which are generally characterized by a disulfide bridge packed with a tryptophan residue, are also found in non-immunoglobulin proteins. Non-immunoglobulin proteins comprising Ig-like domains are classified as members of the Ig superfamily of proteins. The Ig superfamily is a heterogeneous group of proteins, some of which cannot currently be engineered to bind new antigens nor provide scaffolds that allow for successful library screening for high-affinity binding to multiple, diverse target biomolecules. The HUGO Gene Nomenclature Committee (HGNC) provides a list of members of the Ig-like domain containing family.

An immunoglobulin binding region generally comprises three or more CDRs. As used herein, the term “heavy chain variable (V_{H}) domain” or “light chain variable (V_{L}) domain” respectively refer to any antibody V_{H} or V_{L} domain (e.g. a human V_{H} or V_{L} domain) as well as any derivative thereof retaining at least qualitative antigen binding ability of the corresponding native antibody (e.g. a humanized V_{H} or V_{L} domain derived from a native murine V_{H} or V_{L} domain). A V_{H} or V_{L} domain consists of a “framework” region interrupted by the three CDRs or ABRs. The framework regions serve to align the CDRs for specific binding to an epitope of an antigen. From amino-terminus to carboxyl-terminus, both V_{H} and V_{L} domains comprise the following framework (FR) and CDR regions: FR1, CDR1, FR2, CDR2, FR3, CDR3, and 1-R4. For camelid V_{H}H fragments, IgNARs of cartilaginous fish, V_{NAR} fragments, and derivatives thereof, there is a single heavy chain variable domain comprising the same basic arrangement: FR1, CDR1, FR2, CDR2, FR3, CDR3, and FR4.

An immunoglobulin-type binding region may be a polypeptide sequence of an antibody or antigen-binding fragment thereof wherein the amino acid sequence has been varied from that of a native antibody or an Ig-like domain of a non-immunoglobulin protein, for example by molecular engineering or selection by library screening. Because of the relevance of recombinant DNA techniques and in vitro library screening in the generation of immunoglobulin-type binding regions, antibodies can be redesigned to obtain desired characteristics, such as smaller size, cell entry, or other therapeutic improvements. The possible variations are many and may range from the changing of just one amino acid to the complete redesign of, for example, a variable region. Typically, changes in the variable region will be made in order to improve the antigen-binding characteristics, improve variable region stability, or reduce the potential for certain immunogenic responses to the molecule of administration to a subject.

In certain embodiments, the immunoglobulin-type binding region is derived from an immunoglobulin binding region, such as an antibody paratope capable of binding an extracellular target biomolecule. In certain other embodiments, the immunoglobulin-type binding region comprises an engineered polypeptide not derived from any immunoglobulin domain but which functions like an immunoglobulin binding region by providing high-affinity binding to an extracellular target biomolecule. This engineered polypeptide may optionally include polypeptide scaffolds comprising or consisting essentially of complementary determining regions from immunoglobulins as described herein.

Among certain embodiments of the present invention, the immunoglobulin-type binding region is derived from a nanobody or single domain immunoglobulin-derived region V_{H}H. Generally, nanobodies are constructed from fragments of naturally occurring single, monomeric variable domain antibodies (sdAbs) of the sort found in camelids and cartilaginous fishes (Chondrichthyes). Nanobodies are engineered from these naturally occurring antibodies by truncating the single, monomeric variable domain to create smaller and more stable molecules, such as, e.g., IgNAR, V_{H}H, V_{NAR}, and autonomous V_{H} domain constructs. Due to their small size, nanobodies are able to bind to antigens that are not accessible to whole antibodies.

Despite the presence of the word “immunoglobulin” in the phrase immunoglobulin-type, the phrase “immunoglobulin-type binding region” does not necessarily include all structures with an Ig Superfamily domain, such as, e.g., a polypeptide from certain, immune cell, surface receptors with Ig-like domains, unless that structure can serve as a framework for screening to find novel binding region variants which bind with high-affinity to multiple, diverse target biomolecules.

Any of the above binding regions may be used as a component of the cell-targeted molecules of the present invention as long as the binding region component has a dissociation constant of 10⁻⁵ to 10⁻¹² moles per liter, preferably less than 200 nanomolar (nM), towards an extracellular target biomolecule.

### Extracellular Target Biomolecules

The binding region of the cell-targeted molecule of the present invention (e.g., a cytotoxic protein of the present invention) comprises a peptide or polypeptide region capable of binding specifically to an extracellular target biomolecule, preferably which is physically-coupled to the surface of a cell type of interest, such as a cancer cell, tumor cell, plasma cell, infected cell, or host cell harboring an intracellular pathogen.

The term “target biomolecule” refers to a biological molecule, commonly a protein or a protein modified by post-translational modifications, such as glycosylation, which is capable of being bound by a binding region to target a protein to a specific cell-type or location within an organism. Extracellular target biomolecules may include various epitopes, including unmodified polypeptides, polypeptides modified by the addition of biochemical functional groups, and glycolipids (see e.g. U.S. Pat. No. 5,091,178; EP 2431743). It is desirable that an extracellular target biomolecule be endogenously internalized or be readily forced to internalize upon interaction with the cell-targeted molecule of the invention.

For purposes of the present invention, the term “extracellular” with regard to modifying a target biomolecule refers to a biomolecule that has at least a portion of its structure exposed to the extracellular environment. Extracellular target biomolecules include cell membrane components, transmembrane spanning proteins, cell membrane-anchored biomolecules, cell-surface-bound biomolecules, and secreted biomolecules.

With regard to the present invention, the phrase “physically coupled” when used to describe a target biomolecule means both covalent and/or non-covalent intermolecular interactions that couple the target biomolecule, or a portion thereof, to the outside of a cell, such as a plurality of non-covalent interactions between the target biomolecule and the cell where the energy of each single interaction is on the order of about 1-5 kiloCalories (e.g. electrostatic bonds, hydrogen bonds, Van der Walls interactions, hydrophobic forces, etc.). All integral membrane proteins can be found physically coupled to a cell membrane, as well as peripheral membrane proteins. For example, an extracellular target biomolecule might comprise a transmembrane spanning region, a lipid anchor, a glycolipid anchor, and/or be non-covalently associated (e.g. via non-specific hydrophobic interactions and/or lipid binding interactions) with a factor comprising any one of the foregoing.

Extracellular target biomolecules of the binding region of the cell-targeted molecules of the present invention may include biomarkers over-proportionately or exclusively present on cancer cells, immune cells, and cells infected with intracellular pathogens, such as viruses, bacteria, fungi, prions, or protozoans.

The binding regions of the cell-targeted molecules of the present invention may be designed or selected based on numerous criteria, such as the cell-type specific expression of their target biomolecules and/or the physical localization of their target biomolecules with regard to specific cell types. For example, certain cell-targeted molecules of the present invention comprise binding domains capable of binding cell-surface targets which are expressed exclusively by only one cell-type to the cell surface.

Among certain embodiments of the present invention, the cell-targeted molecules of the invention comprise a binding region derived from an immunoglobulin-type polypeptide selected for specific and high-affinity binding to a surface antigen on the cell surface of a cancer cell, where the antigen is restricted in expression to cancer cells (see Glokler J et al., Molecules 15: 2478-90 (2010); Liu Y et al., Lab Chip 9: 1033-6 (2009)). In accordance with other embodiments, the binding region is selected for specific and high-affinity binding to a surface antigen on the cell surface of a cancer cell, where the antigen is over-expressed or preferentially expressed by cancer cells as compared to non-cancer cells. Some representative target biomolecules include, but are not limited to, the following enumerated targets associated with cancers and/or specific immune cell types.

Many immunoglobulin-type binding regions that recognize epitopes associated with cancer cells exist in the prior art, such as binding regions that target (alternative names are indicated in parentheses) annexin AI, B3 melanoma antigen, B4 melanoma antigen, CD2, CD3, CD4, CD20 (B-lymphocyte antigen protein CD20), CD22, CD25 (interleukin-2 receptor IL2R), CD30 (TNFRSF8), CD38 (cyclic ADP ribose hydrolase), CD40, CD44 (hyaluronan receptor), ITGAV (CD51), CD66, CD71 (transferrin receptor), CD73, CD74 (HLA-DR antigens-associated invariant chain), CD79, CD98, endoglin (END or CD105), CD106 (VCAM-1), chemokine receptor type 4 (CDCR-4, fusin, CD184), CD200, insulin-like growth factor 1 receptor (CD221), mucin1 (MUC1, CD227), basal cell adhesion molecule (B-CAM or CD239), CD248 (endosialin or TEM1), carcinoembryonic antigen protein (CEA), chondroitin sulfate proteoglycan 4 (CSP4, MCSP, or NG2), tumor necrosis factor receptor 10b (TNFRSF10B, CD262), tumor necrosis factor receptor 13B (TNFRSF13B, TACI, CD276), vascular endothelial growth factor receptor 2 (KDR, CD309), epithelial cell adhesion molecule (EpCAM, CD326), human epidermal growth factor receptor 2 (HER2/Neu/ErbB2/CD340), cancer antigen 15-3 (CA15-3), cancer antigen 19-9 (CA 19-9), cancer antigen 125 (CA125, MUC16), CA242, carcinoembryonic antigen-related cell adhesion molecules (e.g. CEACAM3 (CD66d) and CEACAM5), carcinoembryonic antigen protein (CEA), chondroitin sulfate proteoglycan 4 (CSP4, MCSP, NG2), CTLA4, DLL4, epidermal growth factor receptor (EGFR/ErbB1), folate receptor (FOLR), G-28, ganglioside GD2, ganglioside GD3, HLA-Dr10, HLA-DRB, human epidermal growth factor receptor 1 (HER1), Ephrin type-B receptor 2 (EphB2), epithelial cell adhesion molecule (EpCAM), fibroblast activation protein (FAP/seprase), insulin-like growth factor 1 receptor (IGF1R), interleukin 2 receptor (IL-2R), interleukin 6 receptor (IL-6R), integrins alpha-V beta-3 (αvβ₃), integrins alpha-V beta-5 (αvβ5), integrins alpha-5 beta-1 (α₅β₁), L6, MPG, melanoma-associated antigen 1 protein (MAGE-1), melanoma-associated antigen 3 (MAGE-3), mesothelin (MSLN), MPG, MS4A, p21, p97, polio virus receptor-like 4 (PVRL4), protease-activated-receptors (such as PAR1), prostate-specific membrane antigen protein (PSMA), trophoblast glycoprotein (TPGB), and tumor-associated calcium signal transducers (TACSTDs) (see e.g. Lui B et al., Cancer Res 64: 704-10 (2004); Novellino L et al., Cancer Immunol Immunother 54: 187-207 (2005); Bagley R et al., Int J Oncol 34: 619-27 (2009); Gerber H et al., mAbs 1: 247-53 (2009); Beck A et al., Nat Rev Immunol 10: 345-52 (2010); Andersen J et al., J Biol Chem 287: 22927-37 (2012); Nolan-Stevaux O et al., PLoS One 7: e50920 (2012); Rust S et al., Mol Cancer 12: 11 (2013)). This list of target biomolecules is intended to be non-limiting. It will be appreciated by the skilled worker that any desired target biomolecule associated with a cancer cell or other desired cell type may be used to design or select a binding region to be coupled with the Shiga toxin effector region to produce a cell-targeted molecule of the invention.

Examples of other target biomolecules which are strongly associated with cancer cells and immunoglobulin-type binding regions known to bind them include BAGE proteins (B melanoma antigens), basal cell adhesion molecules (BCAMs or Lutheran blood group glycoproteins), bladder tumor antigen (BTA), cancer-testis antigen NY-ESO-1, cancer-testis antigen LAGE proteins, CD19 (B-lymphocyte antigen protein CD19), CD21 (complement receptor-2 or complement 3d receptor), CD26 (dipeptidyl peptidase-4, DPP4, or adenosine deaminase complexing protein 2), CD33 (sialic acid-binding immunoglobulin-type lectin-3), CD52 (CAMPATH-1 antigen), CD56 (neural cell adhesion molecule or NCAM), CD133 (prominin-1), CS1 (SLAM family number 7 or SLAMF7), cell surface A33 antigen protein (gpA33), Epstein-Barr virus antigens, GAGE/PAGE proteins (melanoma associated cancer/testis antigens), hepatocyte growth factor receptor (HGFR or c-Met), MAGE proteins, melanoma antigen recognized by T-cells 1 protein (MART-1/MelanA, MARTI), mucins (such as MUC1 and cancer antigen 125 (CA-125)), Preferentially Expressed Antigen of Melanoma (PRAME) proteins, prostate specific antigen protein (PSA), prostate stem cell antigen protein (PSCA), Receptor for Advanced Glycation Endproducts (RAGE), tumor-associated glycoprotein 72 (TAG-72), tyrosine-protein kinase transmembrane receptor (ROR1 or NTRKR1), vascular endothelial growth factor receptors (VEGFRs), and Wilms' tumor antigen.

Examples of other target biomolecules which are strongly associated with cancer cells are carbonic anhydrase IX (CA9/CAIX), claudin proteins (CLDN3, CLDN4), ephrin type-A receptor 3 (EphA3), folate binding proteins (FBPs and folate receptors), ganglioside GM2, insulin-like growth factor receptors, integrins (such as CD11a-c), receptor activator of nuclear factor kappa B (RANK), receptor tyrosine-protein kinase erB-3, tumor necrosis factor receptor 10A (TRAIL-R1/DR4), tumor necrosis factor receptor 10B (TRAIL-R2), tenascin C, and CD64 (FcγRI) (see Hough C et al., Cancer Res 60: 6281-7 (2000); Thepen T et al., Nat Biotechnol 18: 48-51 (2000); Pastan I et al., Nat Rev Cancer 6: 559-65 (2006); Pastan, Annu Rev Med 58: 221-37 (2007); Fitzgerald D et al., Cancer Res 71: 6300-9 (2011); Scott A et al., Cancer Immun 12: 14-22 (2012)). This list of target biomolecules is intended to be non-limiting.

In addition, there are numerous other examples of contemplated, target biomolecules such as melanocyte protein PMEL (gp100), human tyrosinase, tyrosinase-related protein 1 (TYRP1 or TRP1), tyrosinase-related protein 2 (TRP-2), lysophosphatidlglycerol acyltransferase 1 (LPGAT1/IAA0205), SART proteins, ADP-ribosyltransferases (ART1, ART4), human aspartyl (asparaginyl) beta-hydroxylase (HAAH), ephrin type-A receptor 2 (EphA2), receptor tyrosine-protein kinase erbB-3, tyrosinase associated antigen (TAA), break point cluster region-c-abl oncogene (Bcr-Abl) proteins, ADAM metalloproteinases (e.g. ADAM-9, ADAM-10, ADAM-12, ADAM-15, ADAM-17), ADP-ribosyltransferases (ART1, ART4), antigen F4/80, bone marrow stroma antigens (BST1, BST2), break point cluster region-c-abl oncogene (BCR-ABL) proteins, C3aR (complement component 3a receptors), CD7, CD13, CD14, CD15 (Lewis X or stage-specific embryonic antigen 1), CD23 (FC epsilon RII), CD49d, CD53, CD54 (intercellular adhesion molecule 1), CD63 (tetraspanin), CD69, CD80, CD86, CD88 (complement component 5a receptor 1), CD115 (colony stimulating factor 1 receptor), CD123 (interleukin-3 receptor), CD129 (interleukin 9 receptor), CD183 (chemokine receptor CXCR3), CD191 (CCR1), CD193 (CCR3), CD195 (chemokine receptor CCR5), CD203c, CD225 (interferon-induced transmembrane protein 1), CD244 (Natural Killer Cell Receptor 2B4), CD282 (toll-like receptor 2 (TLR2)), CD284 (Toll-like receptor 4 (TLR4)), CD294 (GPR44), CD305 (leukocyte-associated immunoglobulin-like receptor 1), ephrin type-A receptor 2 (EphA2), FceRIa, galectin-9, alpha-fetoprotein antigen 17-A1 protein, human aspartyl (asparaginyl) beta-hydroxylase (HAAH), immunoglobulin-like transcript ILT-3, lysophosphatidlglycerol acyltransferase 1 (LPGAT1/IAA0205), lysosome-associated membrane proteins (LAMPs, such as CD107), melanocyte protein PMEL (gp100), myeloid-related protein-14 (mrp-14), programmed death-ligand 1 (PD-L1), receptor tyrosine-protein kinase erbB-3, SART proteins, scavenger receptors (such as CD64 and CD68), Siglecs (sialic acid-binding immunoglobulin-type lectins), syndecans (such as SDC1 or CD138), tyrosinase, tyrosinase-related protein 1 (TRP-1), tyrosinase-related protein 2 (TRP-2), tyrosinase associated antigen (TAA), APO-3, BCMA, CD2, CD3 (T-cell co-receptor), CD4, CD8, CD18, CD27, CD28, CD29, CD41, CD49, CD90, CD95 (Fas), CD103, CD104, CD134 (OX40), CD137 (4-1BB), CD152 (CTLA-4), chemokine receptors, complement proteins, cytokine receptors, histocompatibility proteins, ICOS, interferon-alpha, interferon-beta, c-myc, osteoprotegerin, PD-1, RANK, TACI, TNF receptor superfamily member (TNF-R1, TNFR-2), Apo2/TRAIL-R1, TRAIL-R2, TRAIL-R3, and TRAIL-R4 (see Cheever M et al., Clin Cancer Res 15: 5323-37 (2009); Scott A et al., Cancer Immun 12: 14 (2012)), for target biomolecules and note the target molecules described therein are non-limiting examples). It will be appreciated by the skilled worker that any desired target biomolecule may be used to design or select a binding region to be coupled with the Shiga toxin effector region to produce a cell-targeted molecule of the invention.

In certain embodiments, the binding region comprises or consists essentially of an immunoglobulin-type polypeptide selected for specific and high-affinity binding to the cellular surface of a cell type of the immune system. For example, immunoglobulin-type binding domains are known that bind to CD1, CD2, CD3, CD4, CD5, CD6, CD7, CD8, CD9, CD10, CD11, CD12, CD13, CD14, CD15, CD16, CD17, CD18, CD19, CD20, CD21, CD22, CD23, CD24, CD25, CD26, CD27, CD28, CD29, CD30, CD31, CD33, CD34, CD35, CD36, CD37, CD38, CD40, CD41, CD56, CD61, CD62, CD66, CD95, CD117, CD123, CD235, CD146, CD326, interleukin-2 receptor (IL-2R), receptor activator of nuclear factor kappa B (RANKL), SLAM-associated protein (SAP), and TNFSF18 (tumor necrosis factor ligand 18 or GITRL).

In certain embodiments, the binding region binds with high-affinity to the target biomolecule which is a chemokine receptor selected from the following CXCR-1, CXCR-2, CXCR-3 A, CXCR3B, CXCR-4, CXCR-5, CCR-I, CCR-2A, CCR-2B, CCR-3, CCR-4, CCR-5, CCR-6, CCR-7, CCR-8, CCR-9, CCR-10, CX3CR-1, XCR1, CXCR-6, CXCR-7, chemokine binding protein-2 (CCBP2, D6 receptor), and Duffy antigen/chemokine receptor (DARC, Fy glycoprotein, FY, CD234). For more non-limiting target biomolecules, see Table 15 in the Examples below.

The general structure of the cell-targeted molecules of the present invention is modular, in that various, diverse, cell-targeting binding regions, such as, e.g., immunoglobulin-type polypeptides, may be used with the same Shiga toxin effector region to provide for diverse targeting of various extracellular target biomolecules and thus targeting of cytotoxicity, cytostasis, diagnostic agents, and/or exogenous material delivery to various diverse cell types while also providing more optimized cytotoxic potency and/or improvements in protease-cleavage resistance, increased molecular stability, and improved in vivo tolerability.

B. Shiga Toxin Effector Regions Derived from A Subunits of Members of the Shiga Toxin Family

The cell-targeted molecule of the present invention comprises a Shiga toxin effector polypeptide region wherein the amino-terminus of the Shiga toxin effector polypeptide is the amino-terminus of or is proximal to an amino-terminus of a polypeptide component of the cell-targeted molecule.

As used herein, a “Shiga toxin effector region,” “Shiga toxin effector polypeptide,” “Shiga toxin effector polypeptide region,” “Shiga toxin A Subunit effector region,” “Shiga toxin A Subunit effector polypeptide,” or “Shiga toxin A Subunit effector polypeptide region” of a cell-targeted molecule of the present invention refers to a polypeptide derived from a Shiga toxin A Subunit from at least one member of the Shiga toxin family.

A member of the Shiga toxin family refers to any member of a family of naturally occurring protein toxins which are structurally and functionally related, notably toxins isolated from S. dysenteriae and E. coli (Johannes L, Römer W, Nat Rev Microbiol 8: 105-16 (2010)). For example, the Shiga toxin family encompasses true Shiga toxin (Stx) isolated from S. dysenteriae serotype 1, Shiga-like toxin 1 variants (SLT1 or Stx1 or SLT-1 or Slt-I) isolated from serotypes of enterohemorrhagic E. coli, and Shiga-like toxin 2 variants (SLT2 or Stx2 or SLT-2) isolated from serotypes of enterohemorrhagic E. coli. SLT1 differs by only one residue from Stx, and both have been referred to as Verocytotoxins or Verotoxins (VTs) (O'Brien A et al., Curr Top Microbiol Immunol 180: 65-94 (1992)). Although SLT1 and SLT2 variants are only about 53-60% similar to each other at the amino acid sequence level, they share mechanisms of enzymatic activity and cytotoxicity common to the members of the Shiga toxin family (Johannes L, Römer W, Nat Rev Microbiol 8: 105-16 (2010)). Over 39 different Shiga toxins have been described, such as the defined subtypes Stx1a, Stx1c, Stx1d, and Stx2a-g (Scheutz F et al., J Clin Microbiol 50: 2951-63 (2012)). Members of the Shiga toxin family are not naturally restricted to any bacterial species because Shiga toxin encoding genes can spread among bacterial species via horizontal gene transfer (Strauch E et al., Infect Immun 69: 7588-95 (2001); Bielaszewska M et al., Appl Environ Micrbiol 73: 3144-50 (2007); Zhaxybayeva O, Doolittle W, Curr Biol 21: R242-6 (2011)). As an example of interspecies transfer, a Shiga toxin was discovered in a strain of A. haemolyticus isolated from a patient (Grotiuz G et al., J Clin Microbiol 44: 3838-41 (2006)). Once a Shiga toxin encoding polynucleotide enters a new subspecies or species, the Shiga toxin amino acid sequence is presumed to be capable of developing slight sequence variations due to genetic drift and/or selective pressure while still maintaining a mechanism of cytotoxicity common to members of the Shiga toxin family (see Scheutz, J Clin Microbiol 50: 2951-63 (2012)).

For purposes of the present invention, the phrase “Shiga toxin effector region” refers to a polypeptide region derived from a Shiga toxin A Subunit of a member of the Shiga toxin family that is capable of exhibiting at least one Shiga toxin function. Shiga toxin functions include, e.g., cell entry, lipid membrane deformation, directing subcellular routing, avoiding degradation, catalytically inactivating ribosomes, effectuating cytotoxicity, and effectuating cytostatic effects.

Shiga toxin effector regions of the cell-targeted molecules of the present invention comprise or consist essentially of a polypeptide derived from a Shiga toxin A Subunit dissociated from any form of its native Shiga toxin B Subunit. In addition, certain embodiments of the cell-targeted molecules of the present invention do not comprise any polypeptide comprising or consisting essentially of a functional binding domain of a native Shiga toxin B subunit. Rather, the Shiga toxin A Subunit derived regions are functionally associated, linked, and/or fused with heterologous, binding regions to effectuate cell targeting.

In certain embodiments, a Shiga toxin effector region of the invention may comprise or consist essentially of a full length Shiga toxin A Subunit (e.g. SLT-1A (SEQ ID NO:1), StxA (SEQ ID NO:2), or SLT-2A (SEQ ID NO:3)), noting that naturally occurring Shiga toxin A Subunits may comprise precursor forms containing signal sequences of about 22 amino acids at their amino-terminals which are removed to produce mature Shiga toxin A Subunits and are recognizable to the skilled worker. In other embodiments, the Shiga toxin effector region of the invention comprises or consists essentially of a truncated Shiga toxin A Subunit which is shorter than a full-length Shiga toxin A Subunit.

Shiga-like toxin 1 A Subunit truncations are catalytically active, capable of enzymatically inactivating ribosomes in vitro, and cytotoxic when expressed within a cell (LaPointe P et al., J Biol Chem 280: 23310-18 (2005)). The smallest Shiga toxin A Subunit fragment exhibiting full enzymatic activity is a polypeptide composed of residues 1-239 of Slt1A (LaPointe P et al., J Biol Chem 280: 23310-18 (2005)). Although the smallest fragment of the Shiga toxin A Subunit reported to retain substantial catalytic activity was residues 75-247 of StxA (Al-Jaufy A et al., Infect Immun 62: 956-60 (1994)), a StxA truncation expressed de novo within a eukaryotic cell requires only up to residue 240 to reach the cytosol and exert catalytic inactivation of ribosomes (LaPointe P et al., J Biol Chem 280: 23310-18 (2005)).

Shiga toxin effector regions may commonly be smaller than the full length A subunit. It is preferred that the Shiga toxin effector region maintain the polypeptide region from amino acid position 77 to 239 (SLT-1A (SEQ ID NO:1) or StxA (SEQ ID NO:2)) or the equivalent in other A Subunits of members of the Shiga toxin family (e.g. 77 to 238 of (SEQ ID NO:3)). For example, in certain embodiments of the invention, a Shiga toxin effector region derived from SLT-1A may comprise or consist essentially of amino acids 75 to 251 of SEQ ID NO:1, 1 to 241 of SEQ ID NO:1, 1 to 251 of SEQ ID NO:1, or amino acids 1 to 261 of SEQ ID NO:1. Among certain other embodiments, a Shiga toxin effector region derived from StxA may comprise or consist essentially of amino acids 75 to 251 of SEQ ID NO:2, 1 to 241 of SEQ ID NO:2, 1 to 251 of SEQ ID NO:2, or amino acids 1 to 261 of SEQ ID NO:2. Among certain other embodiments, a Shiga toxin effector region derived from SLT-2 may comprise or consist essentially of amino acids 75 to 251 of SEQ ID NO:3, 1 to 241 of SEQ ID NO:3, 1 to 251 of SEQ ID NO:3, or amino acids 1 to 261 of SEQ ID NO:3.

In certain embodiments of the invention, one or more amino acid residues may be mutated, inserted, or deleted in order to increase the enzymatic activity of the Shiga toxin effector region. For example, mutating residue-position alanine-231 in Stx1A to glutamate increased its enzymatic activity in vitro (Suhan M, Hovde C, Infect Immun 66: 5252-9 (1998)).

In certain embodiments of the invention, one or more amino acid residues may be mutated, inserted, or deleted in order to decrease the catalytic and/or cytotoxic activity of the Shiga toxin effector region.

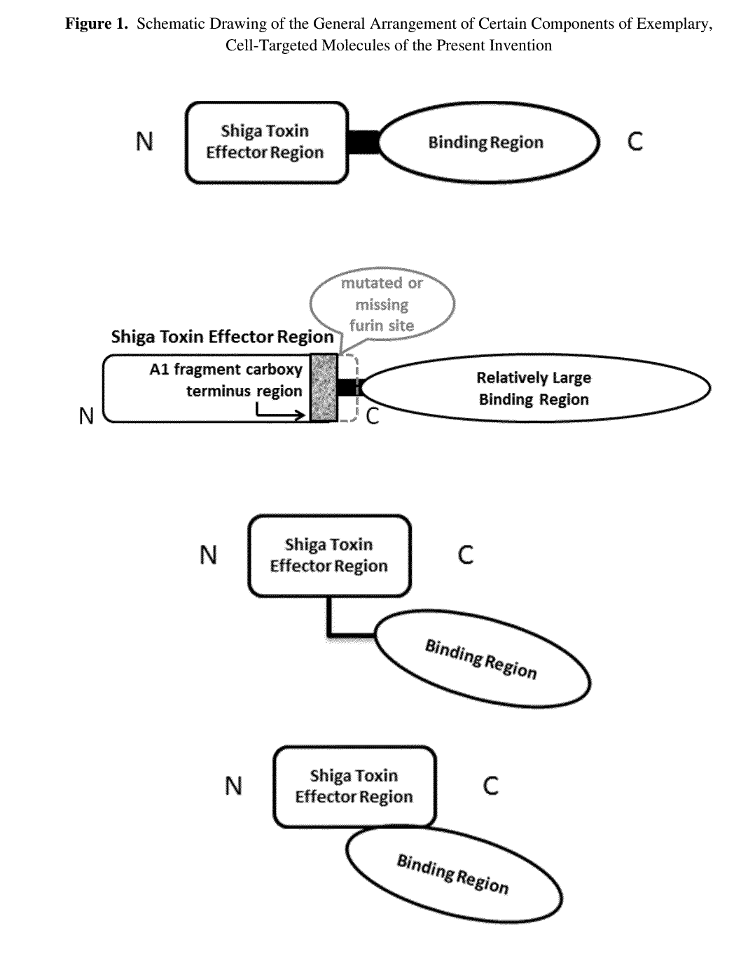

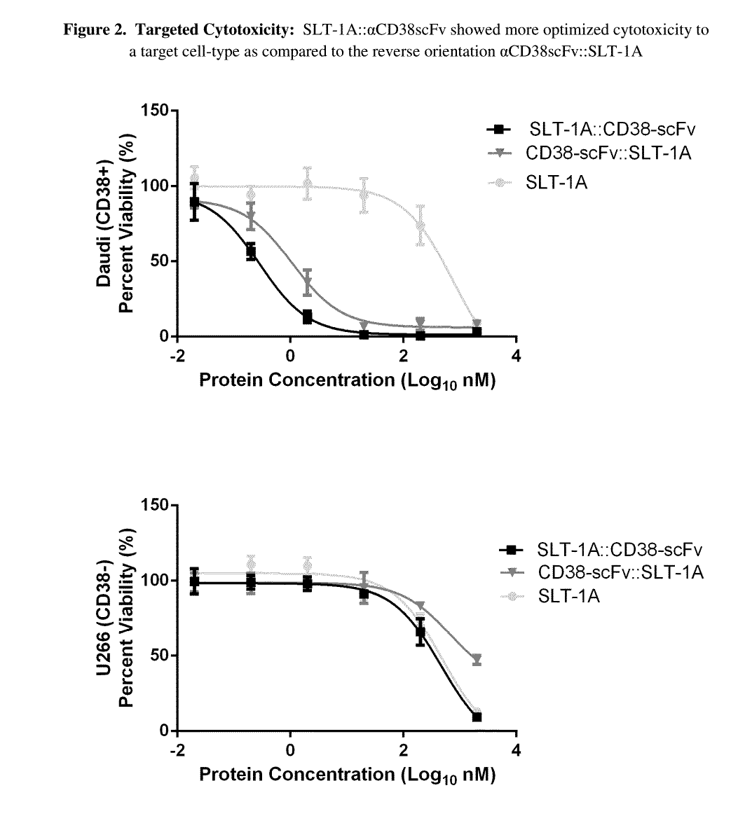

In certain embodiments of the cell-targeted molecules of the present invention, the arrangement of the Shiga toxin effector region within the cell-targeted molecule is limited to being at and/or proximal to the amino-terminus of a polypeptide component of the cell-targeted molecule (see FIG. 1). For example, certain embodiments of the cell-targeted molecule of the present invention comprise 1) a binding region oriented within the cell-targeted molecule at a position carboxy-terminal to the Shiga toxin effector region, 2) a binding region associated with the Shiga toxin effector region at a position distal from the amino terminus of the Shiga toxin effector region (e.g., distances of 50, 100, 200, or 250 amino acid residues or greater), 3) a binding region not sterically covering the amino-terminus of the Shiga toxin effector region and/or 4) a binding region not sterically hindering a structure(s) near the amino-terminus of the Shiga toxin effector polypeptide region (see e.g. FIG. 1). In certain embodiments of the cell-targeted molecules of the present invention, the specific order or orientation of the Shiga toxin effector region and binding region is fixed such that the binding region is located within the cell-targeted molecules more proximal to the carboxy-terminus of the Shiga toxin effector region than to the amino-terminus of the Shiga toxin effector region.

### C. Disrupted Furin-Cleavage Motifs at the Carboxy-Terminus of Shiga Toxin A1 Fragment Regions of Shiga Toxin Effector Polypeptide Regions

In certain embodiments, the cell-targeted molecule of the present invention comprises a protease-cleavage resistant, Shiga toxin A Subunit effector region, such as, e.g., a Shiga toxin effector region more resistant to furin-cleavage in vitro and/or in vivo as compared to a wild-type, Shiga toxin A Subunit. In general, the protease-cleavage sensitivity of a cell-targeted molecule of the present invention is tested by comparing it to the same cell-targeted molecule having its furin-cleavage resistant, Shiga toxin effector polypeptide replaced with a wild-type, Shiga toxin effector polypeptide comprising a Shiga toxin A1 fragment.

In certain embodiments, the cell-targeted molecule of the present invention comprises a Shiga toxin A Subunit effector polypeptide region comprising a Shiga toxin A1 fragment region comprising a disrupted furin-cleavage motif at the carboxy terminus of the Shiga toxin A1 fragment region (such as a disrupted furin-cleavage site located at the carboxy-terminus of a Shiga toxin A1 fragment region). In certain embodiments, the cell-targeted molecule of the present invention comprises a Shiga toxin A Subunit effector polypeptide region which is more furin-cleavage resistant than a wild-type, Shiga toxin A Subunit and/or a wild-type, Shiga toxin A1 fragment. In certain embodiments, the cell-targeted molecule of the present invention comprises a furin-cleavage resistant, Shiga toxin effector region and a binding region linked carboxy-terminal to the disrupted furin-cleavage site of the disrupted furin-cleavage motif located at the carboxy-terminus of the Shiga toxin A1 fragment region of the Shiga toxin effector polypeptide.

For certain, cytotoxic, protease-cleavage resistant, cell-targeted molecules of the present invention that comprise a toxic component (e.g., a toxin-derived region), the cell-targeted molecule may exhibit reduced non-specific toxicity as compared to more protease-cleavage sensitive variants, which have greater propensity to break apart and thereby release the toxic component, especially when administered to living materials, such as, e.g., a cell, tissue, and/or an organism. Furthermore, certain protease-cleavage resistant, cell-targeted molecules of the present invention may exhibit increased, in vivo, half-lives after administration to living materials (e.g., certain vertebrates) as compared to more protease-cleavage sensitive variants based on the protease-cleavage resistance conferred to the cell-targeted molecule by the disrupted furin-cleavage motif at the carboxy terminus of the Shiga toxin A1 fragment region.

Shiga toxin A Subunits of members of the Shiga toxin family comprise a conserved, furin-cleavage site at the carboxy-terminals of their A1 fragment regions important for Shiga toxin function. Certain, cytotoxic, cell-targeted molecules of the present invention each comprise a Shiga toxin effector polypeptide comprising a disrupted furin-cleavage motif at the carboxy terminus of the A1 fragment region of the Shiga toxin effector polypeptide. Furin-cleavage site motifs and furin-cleavage sites can be identified by the skilled worker and/or by using the information herein.

For purposes of the present invention, the term “furin-cleavage motif” refers to a polypeptide consisting essentially of a twenty, amino acid residue, consensus polypeptide sequence (P14 to P6′) as described herein, which comprises 1) a minimal, furin-cleavage motif P4 to P1, 2) a core, furin-cleavage motif P6 to P2′, and 3) two, flanking, polypeptide regions P14 to P7 and P3′ to P6′. For purposes of the claimed invention, the term “furin-cleavage site” refers to a minimal, furin-cleavage consensus site R/Y-x-x-R in the protease sensitive loop of Shiga toxin A Subunits.

For purposes of the present invention, a “disrupted furin-cleavage motif” is an alteration to one or more amino acid residues derived from the 20 amino acid residue region which is a furin-cleavage motif found in native, Shiga toxin A Subunits at the junction between the Shiga toxin A1 fragment and A2 fragment regions and positioned such that furin-cleavage of a Shiga toxin A Subunit results in the production of the A1 and A2 fragments; wherein the disrupted furin-cleavage motif exhibits reduced furin cleavage compared to a reference molecule comprising a wild-type, Shiga toxin A1 fragment region fused to a carboxy-terminal polypeptide of a size large enough to readily monitor furin cleavage using the appropriate assay. A reduction in furin cleavage may be determined by the skilled worker using assays known in the art and/or described herein.

For example, a reduction in furin cleavage of one molecule compared to a reference molecule may be determined using an in vitro, furin-cleavage assay described in the Examples below, conducted using the same conditions, and then performing a quantitation of the band density of any fragments resulting from cleavage. In certain embodiments of the molecules of the present invention, the disrupted furin-cleavage motif exhibits a reduction in in vitro furin cleavage of 30%, 40%, 50%, 60%, 70%, 80%, 90%, 95%, 97%, 98% or greater compared to a reference molecule comprising a wild-type, Shiga toxin A1 fragment fused at its carboxy terminus to a polypeptide, such as, e.g., the reference molecule SLT-1A-WT::scFv-1 described in the Examples.

The model of Shiga toxin cytotoxicity is that intracellular proteolytic processing of Shiga toxin A Subunits by furin in intoxicated cells is essential for 1) liberation of the A1 fragment from the rest of the Shiga holotoxin, 2) escape of the A1 fragment from the endoplasmic reticulum by exposing a hydrophobic domain in the carboxy terminus of the A1 fragment, and 3) enzymatic activation of the A1 fragment (see Johannes L, Römer W, Nat Rev Microbiol 8: 105-16 (2010)). The efficient liberation of the Shiga toxin A1 fragment from the A2 fragment and the rest of the components of the Shiga holotoxin in the endoplasmic reticulum of intoxicated cells is essential for efficient intracellular routing to the cytosol, maximal enzymatic activity, efficient ribosome inactivation, and achieving optimal cytotoxicity, i.e. comparable to a wild-type Shiga toxin.

During Shiga toxin intoxication, the A Subunit is proteolytically cleaved by furin at the carboxy bond of a conserved arginine residue (e.g. the arginine residue at position 251 in StxA and SLT-1A and the arginine residue at position 250 in Stx2A and SLT-2A) (Garred Ø et al., Exp Cell Res 218: 39-49 (1995); Garred Ø et al., J Biol Chem 270: 10817-21 (1995); Faqerquist C, Sultan O, J Biomed Biotechnol 2010: 123460 (2010)). Furin cleavage of Shiga toxin A Subunits occurs in endosomal and/or Golgi compartments (Garred Ø et al., J Biol Chem 270: 10817-21 (1995); Kurmanova A et al., Biochem Biophys Res Commun 357: 144-9 (2007)).

Furin is a specialized serine endoprotease which is expressed by a wide variety of cell types, in all human tissues examined, and by most animal cells (see Shiryaev S et al., J Biol Chem 282: 20847-53 (2007)). Furin cleaves polypeptides comprising accessible motifs often centered on the minimal, dibasic, consensus motif R-x-(R/K/x)-R (Thomas G, Nat Rev Mol Cell Biol 3: 735-66 (2002); Tian S, Biochem Insights 2: 9-20 (2009)). The A Subunits of members of the Shiga toxin family comprise a conserved, surface-exposed, extended loop structure (e.g. 242-261 in StxA and SLT-1A, and 241-260 in SLT-2) with a conserved S-R/Y-x-x-R motif which is cleaved by furin (Garred Ø et al., Exp Cell Res 218: 39-49 (1995); Garred Ø et al., J Biol Chem 270: 10817-21 (1995); Kurmanova A et al., Biochem Biophys Res Commun 357: 144-9 (2007); Faqerquist C, Sultan O, J Biomed Biotechnol 2010: 123460 (2010)). The surface exposed, extended loop structure positioned at amino acid residues 242-261 in StxA is required for furin-induced cleavage of StxA, including features flanking the minimal, furin-cleavage motif R-x-x-R (Kurmanova A et al., Biochem Biophys Res Commun 357: 144-9 (2007)).

Consensus motifs in substrates cleaved by furin have been identified with some degree of specificity. A furin-cleavage site motif has been described which comprises a region of 20 amino acid residues which can be labeled P14 through P6′ (Tian S, Biochem Insights 2: 9-20 (2009); Tian S, Jianhua W, Int J Biol Sci 6: 89-95 (2010); Tian S et al., Int J Mol Sci 12: 1060-5 (2011); Tian S et al., Sci Rep 2: 261 (2012)) using the nomenclature described in Schechter I, Berger, A, Biochem Biophys Res Commun 32: 898-902 (1968). According to this nomenclature, the cleavage site is at the carboxy bond of the amino acid reside designated P1 and the residues are numbered P2, P3, P4, etc., in the direction going toward the amino-terminus from this reference P1 residue. The residues going toward the carboxy terminus from the P1 reference residue are numbered with the prime notation P2′, P3′, P4′, etc.

A general, furin-cleavage site is often described by the consensus motif R-x-x-R which corresponds to P4-P3-P2-P1; where “R” represents an arginine residue (see Table A, supra), a dash “-” represents a peptide bond, and a lowercase “x” represents any amino acid residue (Schalken J et al., J Clin Invest 80: 1545-9 (1987); Bresnahan P et al., J Cell Biol 111: 2851-9 (1990); Hatsuzawa K et al., J Biol Chem 265: 22075-8 (1990); Wise R et al., Proc Natl Acad Sci U.S.A. 87: 9378-82 (1990); Molloy S et al., J Biol Chem 267: 16396-402 (1992)). However, other residues and positions may help to further define furin-cleavage motifs (Hosaka M et al., J Biol Chem 266: 12127-30 (1991); Oda K et al., Biochem Biophys Res Commun 179: 1181-6 (1991); Leduc R et al., J Biol Chem 267: 14304-8 (1992); Watanabe T et al., J Biol Chem 267: 8270-4 (1992)). A slightly more refined furin-cleavage site motif is often reported as the consensus motif R-x-[K/R]-R (where a forward slash “/” means “or” and divides alternative amino acid residues at the same position), which corresponds to P4-P3-P2-P1, because it was observed that furin has a strong preference for cleaving substrates containing this motif (see Rockwell N et al., Chem Rev 102: 4525-48 (2002); Remade A et al., J Biol Chem 283: 20897-906 (2008); Tian S, Biochem Insights 2: 9-20 (2009); Tian S, Jianhua W, Int J Biol Sci 6: 89-95 (2010); Tian S et al., Int J Mol Sci 12: 1060-5 (2011); Tian S et al., Sci Rep 2: 261 (2012)).

Consistent with this, many furin inhibitors comprise peptides comprise the motif R-x-x-R (see e.g. Misumi Y et al., Biochem Biophys Res Commun 171: 236-42 (1990); Hallenberger S et al., Nature 360: 358-61 (1992); Garten W et al., Biochimie 76: 217-25 (1994); Angliker H, J Med Chem 38: 4014-8 (1995); Van Rompaey L et al., Biochem J 326: 507-514 (1997); Cameron A et al., J Biol Chem 275: 36741-9 (2000); Jean F et al., Proc Natl Acad Sci U.S.A. 97: 2864-9 (2000); Basak A, Lazure C, Biochem J 373: 231-9 (2003); Kacprzak M et al., J Biol Chem 279: 36788-94 (2004)). An example of a synthetic inhibitor of furin is R-V-K-R (see e.g. Henrich S et al., Nat Struct Biol 10: 520-6 (2003)). In general, a polypeptide comprising a surface accessible, dibasic amino acid motif with two positively charged, amino acids separated by two amino acid residues may be predicted to be a furin-cleavage sensitive with cleavage occurring at the carboxy bond of the last basic amino acid in the motif (Rockwell N et al., Chem Rev 102: 4525-48 (2002); Remade A et al., J Biol Chem 283: 20897-906 (2008)).

In addition to the minimal, furin-cleavage site of R-x-x-R, a larger, furin-cleavage site motif has been described with certain amino acid residue preferences at certain positions. By comparing various known furin substrates, certain physicochemical properties have been characterized for the amino acids residues in a 20 amino acid residue long, furin-cleavage site motif. The P6 to P2′ region of the furin-cleavage motif delineates the core furin-cleavage site which physically interacts with the enzymatic domain of furin. The two flanking regions P14 to P7 and P3′ to P6′ are often hydrophilic being rich in polar, amino acid residues to increase the surface accessibility of the core furin-cleavage site located between them.

In general, the furin-cleavage motif region from position P5 to P1 tends to comprise amino acid residues with a positive charge and/or high isoelectric points. In particular, the P1 position, which marks the position of furin proteolysis, is generally occupied by an arginine but other positively charged, amino acid residues may occur in this position. Positions P2 and P3 tend to be occupied by flexible, amino acid residues, and in particular P2 tends to be occupied by arginine, lysine, or sometimes by very small and flexible amino acid residues like glycine. The P4 position tends to be occupied by positively charged, amino acid residues in furin substrates. However, if the P4 position is occupied by an aliphatic, amino acid residue, then the lack of a positively charged, functional group can be compensated for by a positively charged residue located at position(s) P5 and/or P6 (Tian S, Jianhua W, Int J Biol Sci 6: 89-95 (2010)). Positions P1′ and P2′ are commonly occupied by aliphatic and/or hydrophobic amino acid residues, with the P1′ position most commonly being occupied by a serine (Tian S, Biochem Insights 2: 9-20 (2009); Tian S et al., Sci Rep 2: 261 (2012)).

The two, hydrophilic, flanking regions tend to be occupied by amino acid residues which are polar, hydrophilic, and/or have smaller amino acid functional groups; however, in certain verified furin substrates, the flanking regions of the core furin-cleavage motif do not contain any consensus, hydrophilic, amino acid residues (see Tain S, Biochem Insights 2: 9-20 (2009)). In the furin-cleavage motifs of some viral proteins, positions P3′ to P6′ are occupied by amino acid residues with small, hydrophobic, functional groups, such as, e.g., alanines, glycines, and prolines (Tian S, Biochem Insights 2: 9-20 (2009); Tian S et al., Sci Rep 2: 261 (2012)). Although not required for furin proteolysis, the presence of positively charged, amino acid residue(s) at position P5 and/or P6 might increase furin-cleavage efficiency. In Shiga toxin A Subunits, the conserved furin-cleavage motif located at the junction of the Shiga toxin A1 fragment and A2 fragment might have optimized competing functions, such as, e.g., balancing efficient furin-cleavage with exposing an unstructured, hydrophobic patch at the carboxy terminus of the A1 fragment after cleavage.

The 20 amino acid residue furin-cleavage motif found in native, Shiga toxin A Subunits at the junction between the Shiga toxin A1 fragment and A2 fragment furin-cleavage motif is well characterized in certain Shiga toxins. For example, in StxA (SEQ ID No:2) and SLT-1A (SEQ ID NO:1), this furin-cleavage motif is natively positioned from L238 to F257, and in SLT-2A (SEQ ID NO:3), this furin-cleavage motif is natively positioned from V237 to Q256. Based on amino acid homology, experiment, and/or furin-cleavage assays described herein, the skilled worker can identify furin-cleavage motifs in other native, Shiga toxin A Subunits, where the motifs are predicted to result in the production of A1 and A2 fragments after furin cleavage of those Shiga toxin A Subunits by an intoxicated eukaryotic cell.

Alterations to an amino acid residue in the furin-cleavage motif include various mutations as well as post-translation modifications, such as, e.g., glycosylation and the like which involve linking a bulky molecule to the functional group of an amino acid residue. A mutation to an amino acid residue in the furin-cleavage motif includes a deletion, insertion, inversion, substitution, and/or carboxy-terminal truncation of the furin-cleavage motif. Because it has been disrupted, certain disrupted furin-cleavage motifs may not be easily recognizable as being related to any furin-cleavage motif; however, the carboxy terminus of the Shiga toxin A1 fragment region will be recognizable and will define where the furin-cleavage motif would be located were it not disrupted. For example, a disrupted furin-cleavage motif may comprise less than the twenty, amino acid residues of the furin-cleavage motif and representing a carboxy-terminal truncation as compared to a Shiga toxin A Subunit and/or Shiga toxin A1 fragment.

For purposes of the present invention with regard to a furin-cleavage site or furin-cleavage motif, the term “disruption” or “disrupted” refers to an alteration from the naturally occurring furin-cleavage site, such as, e.g., a mutation, which results in a reduction in furin-cleavage at the site as compared to a wild-type Shiga toxin A Subunit. Because the furin-cleavage motif is comprised of about 20 amino acid residues, in theory, mutations, deletions, or insertions involving one or more of any one of these 20 positions can result in a reduction of furin-cleavage sensitivity (Tian S et al., Sci Rep 2: 261 (2012)). The disruption may or may not increase resistance to other proteases.

Examples of types of mutations which can disrupt a furin-cleavage site and furin-cleavage motif are amino acid residue deletions, insertions, inversions, and/or substitutions, including substitutions with non-standard amino acids and/or non-natural amino acids. In addition, furin-cleavage sites and furin-cleavage motifs can be disrupted by mutations comprising the modification of an amino acid by the addition of a covalently-linked chemical structure which masks at least one amino acid in the site or motif, see, e.g. PEGylation (see Zhang C et al., BioDrugs 26: 209-15 (2012) and small molecule adjuvants (Flower D, Expert Opin Drug Discov 7: 807-17 (2012)).

Mutating one or both of the two arginine residues in the minimal, furin consensus site R-x-x-R to alanine will disrupt a furin-cleavage motif and prevent furin-cleavage at that site (see e.g. Duda A et al., J Virol 78: 13865-70 (2004)). Similarly, amino acid residue substitutions of one or both of the arginine residues in the minimal furin-cleavage motif R-x-x-R to any non-conservative amino acid residue known to the skilled worker will reduced the furin-cleavage sensitivity of the motif. In particular, amino acid residue substitutions of arginine to any non-basic amino acid residue which lacks a positive charge, such as, e.g., A, G, P, S, T, D, E, Q, N, C, I, L, M, V, F, W, and Y, will result in a disrupted furin-cleavage motif. Furthermore, deletions within the furin-cleavage motif of the minimal furin-cleavage site or the core, furin-cleavage motif will reduce the furin-cleavage sensitivity of the furin-cleavage motif.

In certain embodiments of the molecules of the present invention, the disrupted furin-cleavage motif comprises a disruption in terms of existence, position, or functional group of one or both of the consensus amino acid residues P1 and P4, such as, e.g., the amino acid residues in positions 1 and 4 of the minimal furin-cleavage motif R/Y-x-x-R.

In certain embodiments, the disrupted furin-cleavage motif comprises a disruption in the spacing between the consensus amino acid residues P4 and P1 in terms of the number of intervening amino acid residues being other than two, and, thus, changing either P4 and/or P1 into a different position and eliminating the P4 and/or P1 designations.

Certain furin-cleavage motif disruptions are indicated herein by reference to specific amino acid positions of native Shiga toxin A Subunits provided in the Sequence Listing, noting that naturally occurring Shiga toxin A Subunits may comprise precursor forms containing signal sequences of about 22 amino acids at their amino-terminals which are removed to produce mature Shiga toxin A Subunits and are recognizable to the skilled worker. Further, certain furin-cleavage motif disruptions comprising mutations are indicated herein by reference to specific amino acids (e.g. R for an arginine residue) natively present at specific positions within native Shiga toxin A Subunits (e.g. R251 for the arginine residue at position 251 from the amino-terminus) followed by the amino acid with which that residue has been substituted in the particular mutation under discussion (e.g. R251A represents the amino acid substitution of alanine for arginine at amino acid residue 251 from the amino-terminus).

In certain embodiments, the disrupted furin-cleavage motif comprises one or more amino acid residue substitutions, as compared to a wild-type, Shiga toxin A Subunit. In certain further embodiments, the disrupted furin-cleavage motif comprises one or more amino acid residue substitutions within the minimal furin-cleavage site R/Y-x-x-R, such as, e.g., for StxA and SLT-1A derived Shiga toxin effector polypeptides, the natively positioned amino acid residue R248 substituted with any non-positively charged, amino acid residue and/or R251 substituted with any non-positively charged, amino acid residue; and for SLT-2A derived Shiga toxin effector polypeptides, the natively positioned amino acid residue Y247 substituted with any non-positively charged, amino acid residue and/or R250 substituted with any non-positively charged, amino acid residue. In further certain embodiments, the disrupted furin-cleavage motif comprises an un-disrupted, minimal furin-cleavage site R/Y-x-x-R but instead comprises a disrupted flanking region, such as, e.g., amino acid residue substitutions in one or more amino acid residues in the furin-cleavage motif flanking regions natively position at, e.g., 241-247 and/or 252-259.

In certain embodiments, the disruption comprises a deletion, insertion, inversion, and/or mutation of at least one amino acid residue within the protease motif region. In certain embodiments, a protease-cleavage resistant, Shiga toxin effector polypeptide may comprise a disruption of the amino acid sequence natively positioned at 249-251 of the A Subunit of Shiga-like toxin 1 (SEQ ID NO:1) or Shiga toxin (SEQ ID NO:2), or at 247-250 of the A Subunit of Shiga-like toxin 2 (SEQ ID NO:3) or the equivalent position in a conserved Shiga toxin effector polypeptide and/or non-native Shiga toxin effector polypeptide sequence. In certain further embodiments, protease-cleavage resistant, Shiga toxin effector polypeptides comprise a disruption which comprises a deletion of at least one amino acid within the protease motif region. In certain further embodiments, protease-cleavage resistant, Shiga toxin effector polypeptides comprise a disruption which comprises an insertion of at least one amino acid within the protease motif region. In certain further embodiments, the protease-cleavage resistant, Shiga toxin effector polypeptides comprise a disruption which comprises an inversion of amino acids, wherein at least one inverted amino acid is within the protease motif region. In certain further embodiments, the protease-cleavage resistant, Shiga toxin effector polypeptides comprise a disruption which comprises a mutation, such as an amino acid substitution to a non-standard amino acid or an amino acid with a chemically modified side chain. Examples of single amino acid substitutions are provided in the Examples.

In certain embodiments, the protease-cleavage resistant, Shiga toxin effector polypeptides comprise a disruption which comprises an amino acid substitution within a protease motif region, where in the substitution occurs at the natively positioned amino acid selected from the group consisting of: 247 of SEQ ID NO:3, 248 of SEQ ID NO:1 or SEQ ID NO:2, 250 of SEQ ID NO:3, 251 of SEQ ID NO:1 or SEQ ID NO:2, or the equivalent position in a conserved Shiga toxin effector polypeptide and/or non-native Shiga toxin effector polypeptide sequence. In certain further embodiments, the substitution is to any non-conservative amino acid and the substitution occurs at the natively positioned amino acid residue selected from the group consisting of: 247 of SEQ ID NO:3, 248 of SEQ ID NO:1 or SEQ ID NO:2, 250 of SEQ ID NO:3, 251 of SEQ ID NO:1 or SEQ ID NO:2, or the equivalent position in a conserved Shiga toxin effector polypeptide and/or non-native Shiga toxin effector polypeptide sequence. In certain further embodiments, the mutation comprises an amino acid substitution selected from the group consisting of: R247A, R248A, R250A R251A, or the equivalent position in a conserved Shiga toxin effector polypeptide and/or non-native Shiga toxin effector polypeptide sequence.

In certain embodiments of the molecules of the present invention, the disrupted furin-cleavage motif comprises the deletion of nine, ten, eleven, or more of the carboxy-terminal amino acid residues within the furin-cleavage motif. In these embodiments, the disrupted furin-cleavage motif will not comprise a furin-cleavage site or a minimal furin-cleavage motif. In other words, certain embodiments lack a furin-cleavage site at the carboxy terminus of the A1 fragment region.

In certain embodiments, a molecule of the present invention comprises a Shiga toxin effector polypeptide comprising a disrupted furin-cleavage motif comprising a mutation in the surface-exposed, protease sensitive loop conserved among Shiga toxin A Subunits. For example, in StxA and SLT-1A, this protease-sensitive loop is natively positioned from position 242 to position 261, and in SLT-2A, this loop is natively positioned from position 241 to position 260. Based on polypeptide sequence homology, the skilled worker can identify this conserved, protease-sensitive loop in other Shiga toxin A Subunits. In certain further embodiments, a molecule of the present invention comprises a Shiga toxin effector polypeptide comprising a disrupted furin-cleavage motif comprising a mutation in this protease-sensitive loop of Shiga toxin A Subunits, the mutation which reduce the surface accessibility of certain amino acid residues within the loop such that furin-cleavage sensitivity is reduced.

In certain embodiments, a molecule of the present invention comprises the disrupted furin-cleavage motif comprising the amino acid residue substitution of one or both of the arginine residues in the minimal, cleavage site consensus motif with A, G, or H. In certain further embodiments, the disrupted furin-cleavage motif comprises a deletion of the region natively positioned at 247-252 in StxA (SEQ ID NO:2) and SLT-1A (SEQ ID NO:3), or the region natively positioned at 246-251 in SLT-2A (SEQ ID NO:3); a deletion of the region natively positioned at 244-246 in StxA (SEQ ID NO:2) and SLT-1A (SEQ ID NO:3), or the region natively positioned at 243-245 in SLT-2A (SEQ ID NO:3); or a deletion of the region natively positioned at 253-259 in StxA (SEQ ID NO:2) and SLT-1A (SEQ ID NO:3), or the region natively positioned at 252-258 in SLT-2A (SEQ ID NO:3). Certain further embodiments comprise the disrupted furin-cleavage motif comprising a combination of any of the aforementioned mutations, where possible.

In certain embodiments, the disrupted furin-cleavage motif comprises a carboxy-terminal truncation as compared to a wild-type, Shiga toxin A Subunit, the truncation which results in the deletion of one or more amino acid residues within the furin-cleavage motif. In certain further embodiments, the disrupted furin-cleavage motif comprises the carboxy-terminal truncation which deletes one or more amino acid residues within the minimal cleavage site Y/R-x-x-R, such as, e.g., for StxA and SLT-1A derived Shiga toxin effector polypeptides, truncations ending at the natively amino acid residue position 250, 249, 248, 247, 246, 245, 244, 243, 242, 241, 240, or less; and for SLT-2A derived Shiga toxin effector polypeptides, truncations ending at the natively amino acid residue position 249, 248, 247, 246, 245, 244, 243, 242, 241, or less.

In certain embodiments, the disrupted furin-cleavage motif comprises the mutation that is a partial, carboxy-terminal truncation of the furin-cleavage motif; however, certain molecules of the present invention do not comprise the disrupted furin-cleavage motif which is a complete, carboxy-terminal truncation of the entire, 20-amino-acid-long, furin-cleavage motif. For example, certain, cytotoxic, cell-targeted molecules of the present invention comprise a Shiga toxin effector polypeptide comprising the disrupted furin-cleavage motif comprising a partial, carboxy-terminal truncation of the A1 fragment region up to native position 240 in StxA (SEQ ID NO:2) or SLT-1A (SEQ ID NO:1) but not a carboxy-terminal truncation at position 239 or less. Similarly, certain, cytotoxic, cell-targeted molecules of the present invention comprise a Shiga toxin effector polypeptide comprising the disrupted furin-cleavage motif comprising a partial, carboxy-terminal truncation of the A1 fragment region up to native position 239 in SLT-2A (SEQ ID NO:3) but not a carboxy-terminal truncation at position 238 or less. In the largest carboxy-terminal truncation mutations comprising the disrupted furin-cleavage motif, positions P14 and P13 of the furin-cleavage motif are still present.

In certain embodiments, the disrupted furin-cleavage motif comprises both an amino acid residue substitution within the furin-cleavage motif and a carboxy-terminal truncation as compared to a wild-type, Shiga toxin A Subunit. In certain further embodiments, the disrupted furin-cleavage motif comprises both an amino acid residue substitution within the minimal furin-cleavage site R/Y-x-x-R and a carboxy-terminal truncation as compared to a wild-type, Shiga toxin A Subunit, such as, e.g., for StxA and SLT-1A derived Shiga toxin effector polypeptides, truncations ending at the natively amino acid residue position 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue R248 and/or R251 substituted with any non-positively charged, amino acid residue where appropriate; and for SLT-2A derived Shiga toxin effector polypeptides, truncations ending at the natively amino acid residue position 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue Y247 and/or R250 substituted with any non-positively charged, amino acid residue where appropriate. In certain embodiments, the truncated Shiga toxin effector polypeptide comprising a disrupted furin-cleavage motif also comprises the furin-cleavage motif, amino acid residues at positions P9, P8, and/or P7 in order to maintain optimal cytotoxicity.

In certain embodiments, the disrupted furin-cleavage motif comprises one or more internal amino acid residue deletions, as compared to a wild-type, Shiga toxin A Subunit. In certain further embodiments, the disrupted furin-cleavage motif comprises one or more amino acid residue deletions within the minimal furin-cleavage site R/Y-x-x-R. For example, StxA and SLT-1A derived Shiga toxin effector polypeptides comprising internal deletions of the natively positioned amino acid residues R248 and/or R251, which may be combined with deletions of surrounding residues such as, e.g., 249, 250, 247, 252, etc.; and SLT-2A derived Shiga toxin effector polypeptides comprising internal deletions of the natively positioned amino acid residues Y247 and/or R250, which may be combined with deletions of surrounding residues such as, e.g., 248, 249, 246, 251, etc. In certain further embodiments, the disrupted furin-cleavage motif comprises a deletion of four, consecutive, amino acid residues which deletes the minimal furin-cleavage site R/Y-x-x-R, such as, e.g., StxA and SLT-1A derived Shiga toxin effector polypeptides lacking R248-R251 and SLT-2A derived Shiga toxin effector polypeptides lacking Y247-R250. In certain further embodiments, the disrupted furin-cleavage motif comprises one or more amino acid residue deletions in the amino acid residues flanking the core furin-cleavage motif, such as, e.g., a deletion of 244-247 and/or 252-255 in SLT-1A or StxA. In certain further embodiments, the disrupted furin-cleavage motif comprises an internal deletion of the entire surface-exposed, protease-cleavage sensitive loop as compared to a wild-type, Shiga toxin A Subunit, such as, e.g., for StxA and SLT-1A derived Shiga toxin effector polypeptides, a deletion of natively positioned amino acid residues 241-262; and for SLT-2A derived Shiga toxin effector polypeptides, a deletion of natively positioned amino acid residues 240-261.

In certain embodiments, the disrupted furin-cleavage motif comprises both an internal amino acid residue deletion within the furin-cleavage motif and a carboxy-terminal truncation as compared to a wild-type, Shiga toxin A Subunit. In certain further embodiments, the disrupted furin-cleavage motif comprises both an amino acid residue deletion within the minimal furin-cleavage site R/Y-x-x-R and a carboxy-terminal truncation as compared to a wild-type, Shiga toxin A Subunit. For example, Shiga toxin effector polypeptides with a disrupted furin-cleavage motif may comprise deletions of the natively positioned amino acid residues 248-249 and/or 250-251 in a truncated StxA or SLT-1A polypeptide or the amino acid residues 247-248 and/or 249-250 in a truncated SLT-2A. In certain further embodiments, the disrupted furin-cleavage motif comprises a deletion of four, consecutive, amino acid residues which deletes the minimal furin-cleavage site R/Y-x-x-R and a carboxy-terminal truncation as compared to a wild-type, Shiga toxin A Subunit, such as, e.g., for StxA and SLT-1A derived Shiga toxin effector polypeptides, truncations ending at the natively amino acid residue position 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and lacking R248-R251; and for SLT-2A derived Shiga toxin effector polypeptides, truncations ending at the natively amino acid residue position 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and lacking Y247-R250.

In certain embodiments, the disrupted furin-cleavage motif comprises both an amino acid residue deletion and an amino acid residue substitution as compared to a wild-type, Shiga toxin A Subunit. In certain further embodiments, the disrupted furin-cleavage motif comprises one or more amino acid residue deletions and substitutions within the minimal furin-cleavage site R/Y-x-x-R, such as, e.g., for StxA and SLT-1A derived Shiga toxin effector polypeptides, the natively positioned amino acid residue R248 substituted with any non-positively charged, amino acid residue and/or R251 substituted with any non-positively charged, amino acid residue; and for SLT-2A derived Shiga toxin effector polypeptides, the natively positioned amino acid residue Y247 substituted with any non-positively charged, amino acid residue and/or R250 substituted with any non-positively charged, amino acid residue.

In certain embodiments, the disrupted furin-cleavage motif comprises an amino acid residue deletion and an amino acid residue substitution as well as a carboxy-terminal truncation as compared to a wild-type, Shiga toxin A Subunit. In certain further embodiments, the disrupted furin-cleavage motif comprises one or more amino acid residue deletions and substitutions within the minimal furin-cleavage site R/Y-x-x-R, such as, e.g., for StxA and SLT-1A derived Shiga toxin effector polypeptides, the natively positioned amino acid residue R248 substituted with any non-positively charged, amino acid residue and/or R251 substituted with any non-positively charged, amino acid residue; and for SLT-2A derived Shiga toxin effector polypeptides, the natively positioned amino acid residue Y247 substituted with any non-positively charged, amino acid residue and/or R250 substituted with any non-positively charged, amino acid residue.

In certain further embodiments, the disrupted furin-cleavage motif comprises both an amino acid substitution within the minimal furin-cleavage site R/Y-x-x-R and a carboxy-terminal truncation as compared to a wild-type, Shiga toxin A Subunit, such as, e.g., for StxA and SLT-1A derived Shiga toxin effector polypeptides, truncations ending at the natively amino acid position 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue R248 and/or R251 substituted with any non-positively charged, amino acid residue where appropriate; and for SLT-2A derived Shiga toxin effector polypeptides, truncations ending at the natively amino acid position 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue Y247 and/or R250 substituted with any non-positively charged, amino acid residue where appropriate.

In certain embodiments, the disrupted furin-cleavage motif comprises an insertion of one or more amino acid residues as compared to a wild-type, Shiga toxin A Subunit as long as the inserted amino residue(s) does not create a de novo furin-cleavage site. In certain embodiments, the insertion of one or more amino acid residues disrupts the natural spacing between the arginine residues in the minimal, furin-cleavage site R/Y-x-x-R, such as, e.g., StxA and SLT-1A derived polypeptides comprising an insertion of one or more amino acid residues at 249 or 250 and thus between R248 and R251; or SLT-2A derived polypeptides comprising an insertion of one or more amino acid residues at 248 or 249 and thus between Y247 and R250.

In certain embodiments, the disrupted furin-cleavage motif comprises both an amino acid residue insertion and a carboxy-terminal truncation as compared to a wild-type, Shiga toxin A Subunit. In certain embodiments, the disrupted furin-cleavage motif comprises both an amino acid residue insertion and an amino acid residue substitution as compared to a wild-type, Shiga toxin A Subunit. In certain embodiments, the disrupted furin-cleavage motif comprises both an amino acid residue insertion and an amino acid residue deletion as compared to a wild-type, Shiga toxin A Subunit.

In certain embodiments, the disrupted furin-cleavage motif comprises an amino acid residue deletion, an amino acid residue insertion, and an amino acid residue substitution as compared to a wild-type, Shiga toxin A Subunit.

In certain embodiments, the disrupted furin-cleavage motif comprises an amino acid residue deletion, insertion, substitution, and carboxy-terminal truncation as compared to a wild-type, Shiga toxin A Subunit.

In certain embodiments, the Shiga toxin effector polypeptide comprising a disrupted furin-cleavage motif is directly fused by a peptide bond to a molecular moiety comprising an amino acid, peptide, and/or polypeptide wherein the fused structure involves a single, continuous polypeptide. In these fusion embodiments, the amino acid sequence following the disrupted furin-cleavage motif should not create a de novo, furin-cleavage site at the fusion junction.

Shiga toxin A Subunits might have other furin-cleavage motifs besides the furin-cleavage motif in the highly conserved, surface-exposed loop structure and natively positioned in the region from L238 to F257 in StxA and SLT-1A and from V237 to Q256 in SLT-2A. For example, StxA and SLT-1A comprise a furin-cleavage motif around the natively positioned amino acid residue region 220 to 223. However, there is no evidence this second furin site in Shiga toxin A Subunits is cleaved in vivo. On the contrary, in vitro treatment of Stx2 holotoxin with human furin did not produce cleavage at any other R-x-x-R motif in the A Subunit (e.g. the motif natively positioned from amino acid residue 179 to 222) beside at Arg250, which suggests that other potential dibasic sites within Shiga toxin A Subunits are not accessible to furin (Faqerquist C, Sultan O, J Biomed Biotechnol 2010: 123460 (2010)). Although disrupting other cleavage sites besides might be engineered, e.g., the furin-cleavage motif at L238 to F257 in StxA1 and SLT-1A, disrupting the furin-cleavage motif natively positioned in the region from 220 to 223 in SLT-1A may reduce its cytotoxic activity below a reasonable activity (see e.g. Lea N et al., Microbiology 145: 999-1004 (1999)) and would provide little benefit related to protease-resistance if the protease site in the 220 to 223 region is not protease accessible.

Optionally, a cell-targeted molecule of the invention may further comprise a carboxy-terminal endoplasmic retention/retrieval signal motif, such as KDEL.

D. Molecular Moieties Associated with the Cell-Targeted Molecule at Positions Carboxy-Terminal to the Carboxy-Terminus of the Shiga Toxin A1 Fragment Region

Certain molecules of the present invention comprise a molecular moiety associated with the carboxy-terminus of the Shiga toxin effector polypeptide at positions carboxy-terminal to the carboxy terminus of the Shiga toxin A1 fragment region. The present invention enables the attachment of relatively large, molecular moieties carboxy terminal to the carboxy-terminals of the Shiga toxin A1 fragment regions of furin-cleavage resistant, Shiga toxin effector polypeptides without any loss in Shiga toxin effector cytotoxicity as compared to furin-cleavable, Shiga toxin effector polypeptides.

The term “molecular moiety” encompasses polypeptides, proteins, cytotoxic agents, polynucleotides, detection promoting agents, small molecule chemotherapeutic agents, polysaccharides, lipids, and other biomolecules whether naturally occurring or synthetic. In certain embodiments, the molecular moiety comprises the binding region of the cell-targeted molecule.

Furin proteolysis of the Shiga toxin A Subunit within an intoxicated cell provides for at least three events: exposure of the carboxy terminus of the Shiga toxin A1 fragment, liberation of the A1 fragment from all other molecular moieties, and translocation of the A1 fragment from the endoplasmic reticulum to the cytosol. The dissociation of the A1 fragment from the A2 fragment and the rest of the Shiga holotoxin is required for the translocation of the A1 fragment from the lumen of the endoplasmic reticulum to the cytosol and the only component of the Shiga holotoxin that reaches the cytosolic compartment is the A1 fragment (LaPointe P et al., J Biol Chem 280: 23310-18 (2005); Tam P, Lingwood C, Microbiology 153: 2700-10 (2007); Li S et al., PLoS One 7: e41119 (2012)).

One critical function of furin cleavage during Shiga toxin intoxication appears to be the exposure of the carboxy terminus of the Shiga toxin A1 fragment. Exposure of the carboxy terminus of the A1 fragment in the endoplasmic reticulum of an intoxicated cell is thought to be required for optimal subcellular routing and cytotoxicity. When a Shiga toxin A Subunit derived structure cannot expose the carboxy terminus of an A1 fragment in the endoplasmic reticulum of an intoxicated cell, then the cytotoxic effect of that structure is reduced (Burgess B, Roberts L, Mol Microbiol 10: 171-9 (1993); Garred Ø et al., Exp Cell Res 218: 39-49 (1995); Lea N et al., Microbiology 145: 999-1004 (1999); Kurmanova A et al., Biochem Biophys Res Commun 357: 144-9 (2007)). This can be explained by the persistence of one or more molecular moieties sterically covering the carboxy terminus of the Shiga toxin A1 fragment resulting in the perturbation of the normally efficient intracellular routing of the A1 fragment to the cytosol. Shiga toxin A Subunit derived structures which lack furin proteolytic processing fail to efficiently reach the cytosol of intoxicated cells (Garred Ø et al., J Biol Chem 270: 10817-21 (1995); Garred Ø et al., Exp Cell Res 218: 39-49 (1995); Lea N et al., Microbiology 145: 999-1004 (1999)).

Another critical function of furin cleavage during Shiga toxin intoxication is liberation of the Shiga toxin A1 fragment from the rest of the Shiga holotoxin. Liberation of the A1 fragment in the endoplasmic reticulum of an intoxicated cell is thought to be required for optimal subcellular routing and cytotoxicity. When a Shiga toxin A1 fragment cannot be furin-cleaved and liberated in the endoplasmic reticulum of an intoxicated cell, then the cytotoxic effect is reduced (Burgess B, Roberts L, Mol Microbiol 10: 171-9 (1993); Garred Ø et al., Exp Cell Res 218: 39-49 (1995); Lea N et al., Microbiology 145: 999-1004 (1999); Kurmanova A et al., Biochem Biophys Res Commun 357: 144-9 (2007)). Again, this can be explained by the persistence of one or more molecular moieties associated with the carboxy terminus of the Shiga toxin A1 fragment resulting in the perturbation of the normally efficient intracellular routing of the A1 fragment to the cytosol.

For maximal Shiga toxin cytotoxicity, models suggest that it is essential that the Shiga toxin A1 fragment is liberated from all molecular moieties associated with and/or sterically covering its carboxy-terminus for efficient cytosolic routing, optimal proteasome evasion, optimal catalytic structure formation, and maximal enzymatic activation (Garred Ø et al., Exp Cell Res 218: 39-49 (1995); Garred Ø et al., J Biol Chem 270: 10817-21 (1995); Lea N et al., Microbiology 145: 999-1004 (1999); LaPointe P et al., J Biol Chem 280: 23310-18 (2005); Yu M, Haslam D, Infect Immun 73: 2524-32 (2005); Kurmanova A et al., Biochem Biophys Res Commun 357: 144-9 (2007); Smith M et al., Infect Immun 77: 2730-40 (2009); Di R et al., Toxicon 57: 535-39 (2011); Li S et al., PLoS One 7: e41119 (2012)). For example, the Shiga toxin A2 fragment is fused to the A1 fragment in wild-type, Shiga holotoxins and the pentamer of Shiga toxin B-Subunits is bound to the carboxy terminus of the A2 fragment (Fraser M et al., Nat Struct Biol 1: 59-64 (1994)). Similarly, maximal Shiga toxin cytotoxicity might require the liberation of the A1 fragment from all molecular moieties associated with its carboxy terminus, such as, e.g., moieties at least as large as the A2 fragment (4.5-4.7 kDa) and of the mass of the remainder of the Shiga holotoxin (42.7-43.2 kDa).

Relatedly, maximal, Shiga toxin cytotoxicity might require the liberation of the A1 fragment from all carboxy-terminal moieties which sterically cover the carboxy terminus of the A1 fragment as this region must be exposed for efficient translocation to the cytosol (see Suhan M, Hovde C, Infect Immun 66: 5252-9 (1998); LaPointe P et al., J Biol Chem 280: 23310-18 (2005); Yu M, Haslam D, Infect Immun 73: 2524-32 (2005); Li S et al., PLoS One 7: e41119 (2012)).

In addition, for maximal Shiga toxin cytotoxicity it might be important to liberate the A1 fragment from molecular moieties comprising cell-targeting binding domains which bind cellular membrane components, like Shiga toxin B Subunits which bind gangliosides in lipid bilayer membranes. It is possible that when the A1 fragment is covalently attached to a cell-targeting moiety bound with high affinity to an endoplasmic membrane target, then the Shiga toxin A1 fragment remains tethered to the lipid membrane of the endoplasmic reticulum in a way that perturbs mechanisms and events required for efficient A1 fragment liberation and/or translocation to the cytosol.

The present invention provides exemplary structures demonstrating that the functions of furin-cleavage of Shiga toxin A Subunits in the models described above are not required for wild-type levels of Shiga cytotoxicity exhibited by synthetic cell-targeted molecules (see Examples, infra). Apparently, the carboxy terminus of the Shiga toxin A1 fragment does not need to be exposed for efficient intracellular routing to the cytosol, and, apparently, the liberation of the A1 fragment from all other molecular moieties is not required for maximal, Shiga toxin cytotoxicity. Thus, the furin-cleavage motif of Shiga toxin A Subunits may be disrupted in cell-targeted molecules without sacrificing any cytotoxicity despite the presence of a molecular moiety located with the cell-targeted molecule carboxy terminal to the Shiga toxin effector polypeptide region.

Certain molecules of the present invention comprise a molecular moiety associated with the carboxy terminus of the Shiga toxin effector polypeptide. In certain further embodiments, the association comprises a covalent bond linking the carboxy terminus of the Shiga toxin effector polypeptide, either directly or indirectly, with the molecular moiety. In certain further embodiments, the association comprises the peptide bond which fuses the carboxy terminus of the Shiga toxin effector polypeptide with one or more amino acid residues of the molecular moiety. In certain further embodiments, the Shiga toxin effector polypeptide and the molecular moiety are fused to form a single, continuous polypeptide such that the Shiga toxin effector polypeptide is physically located within the continuous polypeptide amino-terminal to the molecular moiety.

The size of the molecular moiety may vary. Molecular moieties of the molecules of the present invention include: moieties large enough to sterically cover the carboxy terminus of a Shiga toxin A1 fragment, moieties of any size comprising binding regions capable of binding lipid membrane bound targets, moieties of any size which provide a well-structured, tertiary polypeptide structure proximal to the carboxy-terminal region of the Shiga toxin A1 fragment of the invention, moieties of any size which are more polar and hydrophilic than the carboxy terminus of Shiga toxin A1 fragments, and any moiety equal or greater than the size of a native, Shiga toxin A Subunit (approximately 28 kDa). A molecular moiety of a size equal to or greater than 28 kDa is referred to herein as “relatively large.”

In certain embodiments, a molecule of the invention may comprise the molecular moiety comprising a peptide. In certain embodiments, a molecule of the invention may comprise the molecular moiety having a mass of 1.5 kDa or greater. In certain embodiments, a molecule of the invention may comprise the molecular moiety that has a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the molecule retains the appropriate Shiga toxin biological activity noted herein.

In certain embodiments, the molecular moiety has a mass of about 4.5 kDa or another equivalent mass of a Shiga toxin A2 fragment. It was unexpected that a moiety of this size can remain attached to the carboxy terminus of the Shiga toxin A1 fragment without disrupting the efficiency of sub-cellular routing and ribosome inactivation within intoxicated cells.

In certain embodiments, the molecular moiety has a mass of about 7.6 kDa or another equivalent mass of a Shiga toxin B Subunit. It was unexpected that a moiety of this size can remain attached to the carboxy terminus of the Shiga toxin A1 fragment without disrupting the efficiency of sub-cellular routing and ribosome inactivation within intoxicated cells.

In certain embodiments, the molecular moiety has a mass of about 6-10 kDa or greater and comprises a binding region comprising an antibody mimetic or alternative antibody format, such as, e.g., engineered Armadillo repeat polypeptides (ArmRPs), engineered, fibronectin-derived, 10^{th} fibronectin type III (10Fn3) domain (monobodies, AdNectins™, or AdNexins™); engineered, ankyrin repeat motif containing polypeptide (DARPins™); engineered, low-density-lipoprotein-receptor-derived, A domain (LDLR-A) (Avimers™); engineered, Protein-A-derived, Z domain (Affibodies™); engineered, gamma-B crystalline-derived scaffold or engineered, ubiquitin-derived scaffold (Affilins); and Sac7d-derived polypeptides (Nanoffitins® or affitins).

In certain embodiments, the molecular moiety has a mass of about 11 kDa or more and comprises a binding region comprising an immunoglobulin domain(s) and which specifically binds an extracellular target biomolecule with high affinity, such as, e.g., a V_{HH} or nanobody. In certain further embodiments, the molecular moiety has a mass of about 24 kDa or more and comprises a binding region comprising an immunoglobulin domain and which specifically binds an extracellular target biomolecule with high affinity, such as, e.g., a scFv.

In certain embodiments, the molecular moiety has a mass of about 12 kDa or another equivalent mass of a Shiga toxin A2 fragment and B Subunit complex. It was unexpected that a moiety of this size can remain attached to the carboxy terminus of the Shiga toxin A1 fragment without disrupting the efficiency of sub-cellular routing and ribosome inactivation within intoxicated cells.

In certain embodiments, the molecular moiety has a mass of about 28 kDa. It was unexpected that a moiety of this size can remain attached to the carboxy terminus of the Shiga toxin A1 fragment without disrupting the efficiency of sub-cellular routing and ribosome inactivation within intoxicated cells; however, the Examples herein demonstrate that a furin-cleavage resistant molecule comprising a Shiga toxin A1 fragment fused to a 28 kDa molecular moiety did not exhibit any apparent disruption in sub-cellular routing, ribosome inhibition, or cytotoxicity.

In certain embodiments, the relatively large, molecular moiety has a mass of about 39 kDa or another equivalent mass of a Shiga toxin B Subunit pentamer. It was unexpected that a moiety of this size can remain attached to the carboxy terminus of the Shiga toxin A1 fragment without disrupting the efficiency of sub-cellular routing and ribosome inactivation within intoxicated cells.

In certain embodiments, the relatively large, molecular moiety has a mass of about 43.2 kDa or another equivalent mass of a Shiga toxin A2 fragment and B Subunit pentamer complex. It was unexpected that a moiety of this size can remain attached to the carboxy terminus of the Shiga toxin A1 fragment without disrupting the efficiency of sub-cellular routing and ribosome inactivation within intoxicated cells.

In certain embodiments, the molecular moiety is branched. In certain embodiments, the molecule moiety is non-proteinaceous. In certain embodiments, the molecular moiety is a cytotoxic agent or detection promoting agent, such as agents described herein.

In certain embodiments, the molecular moiety sterically covers the carboxy-terminus of the Shiga toxin A1 fragment polypeptide of the Shiga toxin effector polypeptide of the invention. For purposes of the present invention, “sterically cover” or “sterically covering” refers to a moiety covalently attached directly to the carboxy terminal region of the Shiga toxin A1 fragment polypeptide of the Shiga toxin effector polypeptide of the invention. In certain embodiments, the molecular moiety sterically covers the carboxy terminal region of the Shiga toxin A1 fragment polypeptide of the Shiga toxin effector polypeptide of the invention such that the hydrophobic region within the carboxy-terminal region of the Shiga toxin A1 fragment polypeptide of the Shiga toxin effector polypeptide of the invention remain buried and is not surface exposed in the endoplasmic reticulum, thereby keeping the carboxy terminus of the A1 fragment region covered and preventing cellular recognition of the carboxy terminus of the A1 fragment-derived region, such as, e.g. recognition by the ERAD machinery.

In certain embodiments, the molecular moiety comprises a polypeptide which is more polar and hydrophilic than the carboxy-terminal region of a Shiga toxin A1 fragment such that the hydrophobic region within the carboxy-terminal region of the Shiga toxin A1 fragment polypeptide of the Shiga toxin effector polypeptide of the invention remain buried and is not surface exposed in the endoplasmic reticulum, thereby keeping the carboxy terminus of the A1 fragment region covered and preventing cellular recognition of the carboxy terminus of the A1 fragment-derived region, such as, e.g. recognition by the ERAD machinery.

In certain embodiments, the molecular moiety comprises a binding region capable of specifically binding at least one target biomolecule which is membrane bound in the endoplasmic reticulum membrane.

In certain embodiments, the molecular moiety comprises a binding region capable of specifically binding at least one extracellular target biomolecule.

### E. Endoplasmic Reticulum Retention/Retrieval Signal Motif of a Member of the KDEL Family

For purposes of the present invention, the phrase “endoplasmic reticulum retention/retrieval signal motif,” KDEL-type signal motif, or signal motif refers to any member of the KDEL family capable of functioning within a eukaryotic cell to promote subcellular localization of a protein to the endoplasmic reticulum via KDEL receptors.

The carboxy-terminal lysine-asparagine-glutamate-leucine (KDEL) sequence is a canonical, endoplasmic reticulum retention and retrieval signal motif for soluble proteins in eukaryotic cells and is recognized by KDEL receptors (see, Capitani M, Sallese M, FEBS Lett 583: 3863-71 (2009), for review). The KDEL family of signal motifs includes many KDEL-like motifs, such as HDEL, RDEL, WDEL, YDEL, HEEL, KEEL, REEL, KFEL, KIEL, DKEL, KKEL, HNEL, HTEL, KTEL, and HVEL, all of which are found at the carboxy-terminals of proteins which are known to be residents of the lumen of the endoplasmic reticulum of organisms throughout multiple phylogenetic kingdoms (Munro S, Pelham H, Cell 48: 899-907 (1987); Raykhel I et al., J Cell Biol 179: 1193-204 (2007)). The KDEL signal motif family includes at least 46 polypeptide variants shown using synthetic constructs (Raykhel, J Cell Biol 179: 1193-204 (2007)). Additional KDEL signal motifs include ALEDEL, HAEDEL, HLEDEL, KLEDEL, IRSDEL, ERSTEL, and RPSTEL (Alanen H et al., J Mol Biol 409: 291-7 (2011)). A generalized consensus motif representing the majority of KDEL signal motifs has been described as [KRHQSA]-[DENQ]-E-L (Hulo N et al., Nucleic Acids Res 34: D227-30 (2006)).

Proteins containing KDEL family signal motifs are bound by KDEL receptors distributed throughout the Golgi complex and transported to the endoplasmic reticulum by a microtubule-dependent mechanism for release into the lumen of the endoplasmic reticulum (Griffiths G et al., J Cell Biol 127: 1557-74 (1994); Miesenböck G, Rothman J, J Cell Biol 129: 309-19 (1995)). KDEL receptors dynamically cycle between the Golgi complex and endoplasmic reticulum (Jackson M et al., EMBO J 9: 3153-62 (1990); Schutze M et al., EMBO J 13: 1696-1705 (1994)).

For purposes of the present invention, the members of the KDEL family include synthetic signal motifs able to function within a eukaryotic cell to promote subcellular localization of a protein to the endoplasmic reticulum via KDEL receptors. In other words, some members of the KDEL family might not occur in nature or have yet to be observed in nature but have or may be constructed and empirically verified using methods known in the art; see e.g., Raykhel I et al., J Cell Biol 179: 1193-204 (2007).

As a component of certain embodiments of the molecules of the invention, the KDEL-type signal motif is physically located, oriented, or arranged within the molecule such that it is on a carboxy-terminal of a polypeptide and/or protein component of the molecule.

In the cell-targeted molecules of the present invention, the binding regions, Shiga toxin effector polypeptide regions, optional molecular moieties, optional additional exogenous material, detection promoting agents, and/or optional KDEL-type signal motif peptides, may be directly linked to each other and/or suitably linked to each other indirectly via one or more linkers well known in the art and/or described herein, such as, e.g., proteinaceous linkers capable of being genetically fused between other proteinaceous components of the cell-targeted molecule of the present invention.

F. Linkages Connecting Polypeptide Components of the Invention and/or their Subcomponents

The cell-targeted molecules of the present invention may consist of a single polypeptide, multiple polypeptides in association with each other, a branched polypeptide, and/or one or more non-polypeptide moieties.

Individual peptide, polypeptide, and/or protein components of the invention, e.g. immunoglobulin-type binding regions and Shiga toxin effector regions (which may be cytotoxic and/or harbor one or more mutations altering, reducing, or eliminating catalytic activity and/or cytotoxicity), may be suitably linked to each other via one or more linkers well known in the art and/or described herein. Individual polypeptide subcomponents of the binding regions, e.g. CDR, ABR, V_{H}H regions, heavy chain variable regions (V_{H}), light chain variable regions (V_{L}), IgNAR regions, and/or V_{NAR} regions, may be suitably linked to each other via one or more linkers well known in the art and/or described herein (see e.g. Weisser N, Hall J, Biotechnol Adv 27: 502-20 (2009); Chen X et al., Adv Drug Deliv Rev 65: 1357-69 (2013)). Polypeptide components of the invention, e.g., multi-chain binding regions, may be suitably linked to each other or other peptide and/or polypeptide components of the invention via one or more linkers well known in the art. Peptide components of the invention, e.g., KDEL family endoplasmic reticulum retention/retrieval signal motifs, may be suitably linked to another component of the invention via one or more linkers, such as a proteinaceous linker, which are well known in the art.

Suitable linkers are generally those which allow each polypeptide component of the invention to fold with a three-dimensional structure very similar to the polypeptide components produced individually without any linker or other component. Suitable linkers include single amino acids, peptides, polypeptides, and linkers lacking any of the aforementioned such as, e.g., various non-proteinaceous carbon chains, whether branched or cyclic (see e.g. Alley S et al., Bioconjug Chem 19: 759-65 (2008) Ducry L, Stump B, Bioconjug Chem 21: 5-13 (2010); Chen X et al., Adv Drug Deliv Rev 65: 1357-69 (2013)).

Suitable linkers may be proteinaceous and comprise one or more amino acids, peptides, and/or polypeptides. Proteinaceous linkers are suitable for both recombinant fusion proteins and chemically linked conjugates. A proteinaceous linker typically has from about 2 to about 50 amino acid residues, such as, e.g., from about 5 to about 30 or from about 6 to about 25 amino acid residues. The length of the linker selected will depend upon a variety of factors, such as, e.g., the desired property or properties for which the linker is being selected (see e.g. Chen X et al., Adv Drug Deliv Rev 65: 1357-69 (2013)).

Suitable linkers may be non-proteinaceous, such as, e.g. chemical linkers (see e.g. Dosio F et al., Toxins 3: 848-83 (2011); Feld J et al., Oncotarget 4: 397-412 (2013)). Various non-proteinaceous linkers known in the art may be used to link binding regions to the Shiga toxin effector polypeptide regions, such as linkers commonly used to conjugate immunoglobulin-derived polypeptides to heterologous polypeptides. For example, polypeptide components of the cell-targeted molecules of the present invention may be linked using the functional side chains of their amino acid residues and carbohydrate moieties such as, e.g., a carboxy, amine, sulfhydryl, carboxylic acid, carbonyl, hydroxyl, and/or cyclic ring group. For example, disulfide bonds and thioether bonds may be used to link two or more polypeptides (see e.g. Fitzgerald D et al., Bioconjugate Chem 1: 264-8 (1990); Pasqualucci L et al., Haematologica 80: 546-56 (1995)). In addition, non-natural amino acid residues may be used with other functional side chains, such as ketone groups (see e.g. Axup J et al., Proc Natl Acad Sci USA 109: 16101-6 (2012); Sun S et al., Chembiochem July 18 (2014); Tian F et al., Proc Natl Acad Sci USA 111: 1766-71 (2014)). Examples of non-proteinaceous chemical linkers include but are not limited to N-succinimidyl (4-iodoacetyl)-aminobenzoate, S—(N-succinimidyl) thioacetate (SATA), N-succinimidyl-oxycarbonyl-cu-methyl-a-(2-pyridyldithio) toluene (SMPT), N-succinimidyl 4-(2-pyridyldithio)-pentanoate (SPP), succinimidyl 4-(N-maleimidomethyl) cyclohexane carboxylate (SMCC or MCC), sulfosuccinimidyl (4-iodoacetyl)-aminobenzoate, 4-succinimidyl-oxycarbonyl-α-(2-pyridyldithio) toluene, sulfosuccinimidyl-6-(α-methyl-α-(pyridyldithiol)-toluamido) hexanoate, N-succinimidyl-3-(-2-pyridyldithio)-proprionate (SPDP), succinimidyl 6(3(-(-2-pyridyldithio)-proprionamido) hexanoate, sulfosuccinimidyl 6(3(-(-2-pyridyldithio)-propionamido) hexanoate, maleimidocaproyl (MC), maleimidocaproyl-valine-citrulline-p-aminobenzyloxycarbonyl (MC-vc-PAB), 3-maleimidobenzoic acid N-hydroxysuccinimide ester (MBS), alpha-alkyl derivatives, sulfoNHS-ATMBA (sulfosuccinimidyl N-[3-(acetylthio)-3-methylbutyryl-beta-alanine]), sulfodicholorphenol, 2-iminothiolane, 3-(2-pyridyldithio)-propionyl hydrazide, Ellman's reagent, dichlorotriazinic acid, and S-(2-thiopyridyl)-L-cysteine (see e.g. Thorpe P et al., Eur J Biochem 147: 197-206 (1985); Thorpe P et al., Cancer Res 47: 5924-31 (1987); Thorpe P et al., Cancer Res 48: 6396-403 (1988); Grossbard M et al., Blood 79: 576-85 (1992); Lui C et al., Proc Natl Acad Sci USA 93: 8618-23 (1996); Doronina S et al., Nat Biotechnol 21: 778-84 (2003); Feld J et al., Oncotarget 4: 397-412 (2013)).

Suitable linkers, whether proteinaceous or non-proteinaceous, may include, e.g., protease sensitive, environmental redox potential sensitive, pH sensitive, acid cleavable, photocleavable, and/or heat sensitive linkers (see e.g. Dosio F et al., Toxins 3: 848-83 (2011); Chen X et al., Adv Drug Deliv Rev 65: 1357-69 (2013); Feld J et al., Oncotarget 4: 397-412 (2013)).

Proteinaceous linkers may be chosen for incorporation into recombinant fusion protein embodiments of the cell-targeted molecules of the present invention. For example, the component polypeptides of the cell-targeted molecules of the present invention or their subcomponents may be joined by one or more linkers comprising one or more amino acids, peptides, and/or polypeptides. For recombinant fusion protein embodiments of the cell-targeted molecules of the invention, linkers typically comprise about 2 to 50 amino acid residues, preferably about 5 to 30 amino acid residues (Argos P, J Mol Biol 211: 943-58 (1990); Williamson M, Biochem J 297: 240-60 (1994); George R, Heringa J, Protein Eng 15: 871-9 (2002); Kreitman R, AAPS J 8: E532-51 (2006)). Commonly, proteinaceous linkers comprise a majority of amino acid residues with polar, uncharged, and/or charged residues, such as, e.g., threonines, prolines, glutamines, glycines, and alanines (see e.g. Huston J et al. Proc Natl Acad Sci USA 85: 5879-83 (1988); Pastan I et al., Annu Rev Med 58: 221-37 (2007); Li J et al., Cell Immunol 118: 85-99 (1989); Cumber A et al. Bioconj Chem 3: 397-401 (1992); Friedman P et al., Cancer Res 53: 334-9 (1993); Whitlow M et al., Protein Engineering 6: 989-95 (1993); Siegall C et al., J Immunol 152: 2377-84 (1994); Newton et al. Biochemistry 35: 545-53 (1996); Ladurner et al. J Mol Biol 273: 330-7 (1997); Kreitman R et al., Leuk Lymphoma 52: 82-6 (2011); U.S. Pat. No. 4,894,443). Non-limiting examples of proteinaceous linkers include alanine-serine-glycine-glycine-proline-glutamate (ASGGPE), valine-methionine (VM), alanine-methionine (AM), AM(G_{2 to 4}S)ₓAM where G is glycine, S is serine, and x is an integer from 1 to 10.

Proteinaceous linkers may be selected based upon the properties desired. Proteinaceous linkers may be chosen by the skilled worker with specific features in mind, such as to optimize one or more of the fusion molecule's folding, stability, expression, solubility, pharmacokinetic properties, pharmacodynamic properties, and/or the activity of the fused domains in the context of a fusion construct as compared to the activity of the same domain by itself. For example, proteinaceous linkers may be selected based on flexibility, rigidity, and/or cleavability (see e.g. Chen X et al., Adv Drug Deliv Rev 65: 1357-69 (2013)). The skilled worker may use databases and linker design software tools when choosing linkers. Certain linkers may be chosen to optimize expression (see e.g. Turner D et al., J Immunol Methods 205: 43-54 (1997)). Certain linkers may be chosen to promote intermolecular interactions between identical polypeptides or proteins to form homomultimers or different polypeptides or proteins to form heteromultimers. For example, proteinaceous linkers may be selected which allow for desired non-covalent interactions between polypeptide components of cell-targeted molecules of the present invention, such as, e.g., interactions related to the formation dimers and other higher order multimers (see e.g. U.S. Pat. No. 4,946,778).

Flexible proteinaceous linkers are often greater than 12 amino acid residues long and rich in small, non-polar amino acid residues, polar amino acid residues, and/or hydrophilic amino acid residues, such as, e.g., glycines, serines, and threonines (see e.g. Bird R et al., Science 242: 423-6 (1988); Friedman P et al., Cancer Res 53: 334-9 (1993); Siegall C et al., J Immunol 152: 2377-84 (1994)). Flexible proteinaceous linkers may be chosen to increase the spatial separation between components and/or to allow for intramolecular interactions between components. For example, various “GS” linkers are known to the skilled worker and are composed of multiple glycines and/or one or more serines, sometimes in repeating units, such as, e.g., (GₓS)ₙ, (SₓG)ₙ, (GGGGS)ₙ, and (G)ₙ. in which x is 1 to 6 and n is 1 to 30 (see e.g. WO 96/06641). Non-limiting examples of flexible proteinaceous linkers include GKSSGSGSESKS, GSTSGSGKSSEGKG, GSTSGSGKSSEGSGSTKG, GSTSGSGKPGSGEGSTKG, EGKSSGSGSESKEF, SRSSG, and SGSSC.

Rigid proteinaceous linkers are often stiff alpha-helical structures and rich in proline residues and/or one or more strategically placed prolines (see Chen X et al., Adv Drug Deliv Rev 65: 1357-69 (2013)). Rigid linkers may be chosen to prevent intramolecular interactions between components.

Suitable linkers may be chosen to allow for in vivo separation of components, such as, e.g., due to cleavage and/or environment-specific instability (see Dosio F et al., Toxins 3: 848-83 (2011); Chen X et al., Adv Drug Deliv Rev 65: 1357-69 (2013)). In vivo cleavable proteinaceous linkers are capable of unlinking by proteolytic processing and/or reducing environments often at a specific site within an organism or inside a certain cell type (see e.g. Doronina S et al., Bioconjug Chem 17: 144-24 (2006); Erickson H et al., Cancer Res 66: 4426-33 (2006)). In vivo cleavable proteinaceous linkers often comprise protease sensitive motifs and/or disulfide bonds formed by one or more cysteine pairs (see e.g. Pietersz G et al., Cancer Res 48: 4469-76 (1998); The J et al., J Immunol Methods 110: 101-9 (1998); see Chen X et al., Adv Drug Deliv Rev 65: 1357-69 (2013)). In vivo cleavable proteinaceous linkers can be designed to be sensitive to proteases that exist only at certain locations in an organism, compartments within a cell, and/or become active only under certain physiological or pathological conditions (such as, e.g., proteases with abnormally high levels, proteases overexpressed at certain disease sites, and proteases specifically expressed by a pathogenic microorganism). For example, there are proteinaceous linkers known in the art which are cleaved by proteases present only intracellularly, proteases present only within specific cell types, and proteases present only under pathological conditions like cancer or inflammation, such as, e.g., R-x-x-R motif and AMGRSGGGCAGNRVGSSLSCGGLNLQAM.

In certain embodiments of the cell-targeted molecules of the present invention, a linker may be used which comprises one or more protease sensitive sites to provide for cleavage by a protease present within a target cell. In certain embodiments of the cell-targeted molecules of the invention, a linker may be used which is not cleavable to reduce unwanted toxicity after administration to a vertebrate organism (see e.g. Polson A et al., Cancer Res 69: 2358-64 (2009)).

Suitable linkers may include, e.g., protease sensitive, environmental redox potential sensitive, pH sensitive, acid cleavable, photocleavable, and/or heat sensitive linkers, whether proteinaceous or non-proteinaceous (see Chen X et al., Adv Drug Deliv Rev 65: 1357-69 (2013)).

Suitable cleavable linkers may include linkers comprising cleavable groups which are known in the art such as, e.g., linkers noted by Zarling D et al., J Immunol 124: 913-20 (1980); Jung S, Moroi M, Biochem Biophys Acta 761: 152-62 (1983); Bouizar Z et al., Eur J Biochem 155: 141-7 (1986); Park L et al., J Biol Chem 261: 205-10 (1986); Browning J, Ribolini A, J Immunol 143: 1859-67 (1989); Joshi S, Burrows R, J Biol Chem 265: 14518-25 (1990).

Suitable linkers may include pH sensitive linkers. For example, certain suitable linkers may be chosen for their instability in lower pH environments to provide for dissociation inside a subcellular compartment of a target cell. For example, linkers that comprise one or more trityl groups, derivatized trityl groups, bismaleimideothoxy propane groups, adipic acid dihydrazide groups, and/or acid labile transferrin groups, may provide for release of components of the invention, e.g. a polypeptide component, in environments with specific pH ranges (see e.g. Welhöner H et al., J Biol Chem 266: 4309-14 (1991); Fattom A et al., Infect Immun 60: 584-9 (1992)). Certain linkers may be chosen which are cleaved in pH ranges corresponding to physiological pH differences between tissues, such as, e.g., the pH of tumor tissue is lower than in healthy tissues (see e.g. U.S. Pat. No. 5,612,474).

Photocleavable linkers are linkers that are cleaved upon exposure to electromagnetic radiation of certain wavelength ranges, such as light in the visible range (see e.g. Goldmacher V et al., Bioconj Chem 3: 104-7 (1992)). Photocleavable linkers may be used to release a component of a cell-targeted molecule of the invention, e.g. a polypeptide component, upon exposure to light of certain wavelengths. Non-limiting examples of photocleavable linkers include a nitrobenzyl group as a photocleavable protective group for cysteine, nitrobenzyloxycarbonyl chloride cross-linkers, hydroxypropylmethacrylamide copolymer, glycine copolymer, fluorescein copolymer, and methylrhodamine copolymer (Hazum E et al., Pept Proc Eur Pept Symp, 16th, Brunfeldt K, ed., 105-10 (1981); Senter et al., Photochem Photobiol 42: 231-7 (1985); Yen et al., Makromol Chem 190: 69-82 (1989); Goldmacher V et al., Bioconj Chem 3: 104-7 (1992)). Photocleavable linkers may have particular uses in linking components to form cell-targeted molecules of the present invention designed for treating diseases, disorders, and conditions that can be exposed to light using fiber optics.

In certain embodiments of the cell-targeted molecules of the present invention, a cell-targeting binding region is linked to a Shiga toxin effector polypeptide region using any number of means known to the skilled worker, including both covalent and noncovalent linkages (see e.g. Chen X et al., Adv Drug Deliv Rev 65: 1357-69 (2013); Behrens C, Liu B, MAbs 6: 46-53 (2014)).

In certain embodiments of the cell-targeted molecules of the present invention, the cell-targeted molecule comprises a binding region which is a scFv with a linker connecting a heavy chain variable (V_{H}) domain and a light chain variable (V_{L}) domain. There are numerous linkers known in the art suitable for this purpose, such as, e.g., the 15-residue (Gly4Ser)₃ peptide. Suitable scFv linkers which may be used in forming non-covalent multivalent structures include GGS, GGGS (Gly3Ser or G3S), GGGGS (Gly4Ser or G4S), GGGGSGGG, GGSGGGG, GSTSGGGSGGGSGGGGSS, and GSTSGSGKPGSSEGSTKG (Plückthun A, Pack P, Immunotechnology 3: 83-105 (1997); Atwell J et al., Protein Eng 12: 597-604 (1999); Wu A et al., Protein Eng 14: 1025-33 (2001); Yazaki P et al., J Immunol Methods 253: 195-208 (2001); Carmichael J et al., J Mol Biol 326: 341-51 (2003); Arndt M et al., FEBS Lett 578: 257-61 (2004); Bie C et al., World J Hepatol 2: 185-91 (2010)).

Suitable methods for linkage of components of the cell-targeted molecules of the present invention may be by any method presently known in the art for accomplishing such, as long as the attachment does not substantially impede the binding function of the binding region, the cellular internalization of the cell-targeted molecule, and/or desired Shiga toxin effector function(s) of the Shiga toxin effector polypeptide region as measured by an appropriate assay, including assays as described herein.

### II. General Functions of the Cell-Targeted Molecules of the Present Invention

The present invention provides various cell-targeted molecules, each comprising 1) a binding region for cell targeting and 2) a Shiga toxin effector region, which is located proximal to an amino-terminus of a polypeptide of the cell-targeted molecule. The linking of cell-targeting binding regions carboxy-terminal to Shiga toxin effector regions enables the engineering of cell-targeted molecules with more optimized Shiga toxin cytotoxic potency (greater cytotoxic potency) as compared to related molecules where the Shiga toxin effector region is not proximal to an amino-terminus of the molecule. As described in more detail in the Examples below, positioning the Shiga toxin effector region within the cell-targeted molecule at and/or proximal to an amino-terminus of a polypeptide component of the cell-targeted molecule resulted in significantly more robust cell kill (4-fold to 9-fold) than the identical proteinaceous components arranged with the amino-terminus of the Shiga toxin effector region physically covered or sterically blocked by another polypeptide region.

For certain embodiments, the cell-targeted molecules of the present invention are capable of binding extracellular target biomolecules associated with the cell surface of particular cell types and entering those cells. Once internalized within a targeted cell type, certain embodiments of the cell-targeted molecules of the invention are capable of routing a Shiga toxin effector polypeptide fragment into the cytosol of the target cell. Once in the cytosol of a targeted cell type, the Shiga toxin effector polypeptides of certain embodiments of the cell-targeted molecules of the invention are capable of enzymatically inactivating ribosomes, interfering with cell homeostasis, and eventually killing the cell, such as via apoptosis. Furthermore, certain embodiments of the cell-targeted molecules of the present invention can kill target cells with more optimized cytotoxic potency than related cell-target molecules, such as, e.g., with a 4-fold to 9-fold or greater level of cytotoxicity.

The design of the cell-targeted molecules of the present invention is modular in that any number of diverse binding regions can be used to target this potent Shiga toxin cytotoxicity to various, diverse cell types. Alternatively, nontoxic variants of the cell-targeted molecules of the present invention may be used to deliver additional exogenous materials into target cells, such as, e.g., cytotoxic agents, antigens, nucleic acids, or detection promoting agents to label the interiors of target cells for diagnostic information collection functions.

The present invention also provides certain embodiments of the cell-targeted molecules of the present invention which exhibit improvements over certain cell-targeted molecules comprising protease-cleavage sensitive, Shiga toxin effector polypeptide regions. By disrupting a conserved, furin-cleavage site natively positioned in Shiga toxin A Subunits between the Shiga toxin A1 and A2 fragments, certain cell-targeted molecules of the present invention may be designed to have improved characteristics, such as, e.g., increased molecular stability, improved in vivo tolerability; and maximal cytotoxicity—equivalent to a nearly identical, cell-targeted molecule comprising a wild-type, Shiga toxin effector polypeptide region (see Examples, infra).

The cell-targeted molecules of the present invention, whether comprising a protease-sensitive or protease-cleavage resistant, Shiga toxin effector region, have uses, e.g., for targeted cell killing, delivering exogenous materials into specific cell types, obtaining diagnostic information, and as therapeutics for the treatment of a variety of diseases, disorders, and conditions, including, inter alia, cancers, immune disorders, and microbial infections.

### A. Cell Kill Via Targeted Shiga Toxin Cytotoxicity

It would be desirable to have cytotoxic, cell-targeted molecules comprising Shiga toxin A Subunit derived components which are as cytotoxic as possible. Because members of the Shiga toxin family are adapted to killing eukaryotic cells, cell-targeted molecules designed using Shiga toxin effector regions can have potent cell-kill activity. The A Subunits of members of the Shiga toxin family comprise enzymatic domains capable of killing a eukaryotic cell once in the cell's cytosol. However, the arrangement of a Shiga toxin effector region within a cell-targeted molecule might perturb that Shiga toxin effector region's potential for effectuating potent cytotoxicity, such as, e.g., by perturbing its ability to reach the cytosol efficiently. For example, the amino to carboxy position of a Shiga toxin A Subunit effector region with respect to a cell-targeting binding region was discovered to have a surprising effect on cytotoxic potency of 4-fold to 9-fold or greater (see Examples, infra).

For many applications, the optimal cytotoxic potency is the highest potency achievable, such as, e.g., to provide for more effective therapeutic windows. Certain embodiments of the cytotoxic cell-targeted molecules of the present invention take advantage of the high cytotoxic potency of polypeptides derived from Shiga toxin A Subunits by providing more optimized potency with as little reduction as possible in the cytotoxic potency of their Shiga toxin effector region in the context of a cell-targeted molecule. Unbound by theory, the orientation of engineering of the proteinaceous components of a cell-targeted molecule of the present invention optimizes the Shiga toxin effector region's cytotoxic potency by improving intracellular routing of the Shiga toxin effector polypeptide to the cytosol via functionalities native to Shiga toxin effector polypeptides.

In certain embodiments, the cell-targeted molecule of the present invention exhibits cytotoxicity with better optimized, cytotoxic potency, such as, e.g., being capable of exhibiting a cytotoxicity 3-fold, 4-fold, 5-fold, 6-fold, 7-fold, 8-fold, 9-fold, 10-fold or greater that the cytotoxicity of a related cell-targeted molecule lacking an amino-terminal and/or amino-terminus proximal, Shiga toxin effector polypeptide region.

The capacity of a cell-targeted molecule of the present invention to cause cell death, e.g. its cytotoxicity, cytotoxic potency, and cytotoxic effect, may be measured using any one or more of a number of assays well known in the art. According to the present invention, the relative cytotoxicity or cytotoxic potency exhibited by two different molecules may be quantified in terms of the ratio (a/b) of (a) the CD₅₀ value of the first molecule to a sample from a population of cells to (b) the CD₅₀ value of the second molecule towards a sample from the same population of cells.

For certain embodiments of the cell-targeted molecules of the present invention, after contacting a cell physically coupled with an extracellular target biomolecule of the binding region of the cell-targeted molecule of the invention (target positive cell) with the cell-targeted molecule, the cell-targeted molecule is capable of causing death of the target positive cell. For certain embodiments of the cell-targeted molecules of the present invention, administration of the cell-targeted molecule of the present invention to a cells physically coupled with an extracellular target biomolecule of the binding region of the cell-targeted molecule of the invention, the cell-targeted molecule is capable of causing death of the cells with a cytotoxic effect 3-fold, 4-fold, 5-fold, 6-fold, 7-fold, 8-fold, 9-fold, 10-fold, or greater than a second cell-targeted molecule comprising the same binding region and the same Shiga toxin effector polypeptide region which is not positioned at or proximal to an amino-terminus of the second cell-targeted molecule.

A cytotoxic effect may be determined as a CD₅₀ value measured using a cell type 1) whose members are physically linked to a target biomolecule of the binding reigon of the cell-targeted molecule to be tested, 2) which expresses the target biomolecule of the binding region of the cell-targeted molecule to be tested, 3) which expresses a significant amount of the target biomolecule of the binding reigon of the cell-targeted molecule to be tested, and/or 4) which is known to be or shown to be target biomolecule positive. Thus, certain cell-targeted molecules of the present invention exhibit a cytotoxic effect 3-fold, 4-fold, 5-fold, 6-fold, 7-fold, 8-fold, 9-fold, 10-fold, or greater than certain, second cell-targeted molecules as evidenced by the ratio (a/b)-fold of (a) the CD₅₀ value of the cell-targeted molecule of the present invention to a sample from a population of cells to (b) the CD₅₀ value of the second cell-targeted molecule towards a sample from the same population of cells.

In certain embodiments, the cytotoxicity of the cell-targeted molecule of the present invention to a population of target positive cells is 3-fold, 4-fold, 5-fold, 6-fold, 7-fold, 8-fold, 9-fold, 10-fold or greater than the cytotoxicity of the second cell-targeted molecule, as described herein, to a second population of target positive cells as assayed by CD₅₀ values.

For certain embodiments of the cell-targeted molecules of the present invention, after contacting a cell physically coupled with an extracellular target biomolecule of the binding region of a cell-targeted molecule of the invention with the cell-targeted molecule, the cell-targeted molecule is capable of causing death of the cell with a cytotoxic effect 3-fold, 4-fold, 5-fold, 6-fold, 7-fold, 8-fold, 9-fold, 10-fold, or greater than a second cell-targeted molecule comprising the same binding region and the same Shiga toxin effector polypeptide region but wherein the Shiga toxin effector polypeptide region is not proximal to an amino-terminus of a polypeptide component of the second cell-targeted molecule. A cytotoxic effect may be determined as a CD₅₀ value measured using a cell type 1) whose members are physically linked to a target biomolecule of the binding reigon of the cell-targeted molecule to be tested, 2) which expresses the target biomolecule of the binding reigon of the cell-targeted molecule to be tested, 3) which expresses a significant amount of the target biomolecule of the binding reigon of the cell-targeted molecule to be tested, and/or 4) which is known to be or shown to be target biomolecule positive. Thus, certain cell-targeted molecules of the present invention exhibit a cytotoxic effect 3-fold, 4-fold, 5-fold, 6-fold, 7-fold, 8-fold, 9-fold, 10-fold, or greater than certain, second cell-targeted molecules as evidenced by the ratio (a/b)-fold of (a) the CD₅₀ value of the cell-targeted molecule of the present invention to a sample from a population of cells to (b) the CD₅₀ value of the second cell-targeted molecule towards a sample from the same population of cells.

For certain embodiments of the cell-targeted molecules of the present invention, after contacting a cell physically coupled with an extracellular target biomolecule of the binding region of a cell-targeted molecule of the invention with the cell-targeted molecule, the cell-targeted molecule is capable of causing death of the cell with a cytotoxic effect 3-fold, 4-fold, 5-fold, 6-fold, 7-fold, 8-fold, 9-fold, 10-fold, or greater than a second cell-targeted molecule comprising the same binding region and the same Shiga toxin effector polypeptide region linked in the same way but wherein the binding region is located amino-terminal to the Shiga toxin effector polypeptide region within a polypeptide component of the second cell-targeted molecule. For certain embodiments of the cytotoxic proteins of the present invention, after contacting a cell physically coupled with an extracellular target biomolecule of the immunoglobulin-type binding region of a cytotoxic protein of the invention with the cytotoxic protein, the cytotoxic protein is capable of causing death of the cell with a cytotoxic effect 3-fold, 4-fold, 5-fold, 6-fold, 7-fold, 8-fold, 9-fold, 10-fold, or greater than a second protein comprising the same immunoglobulin-type binding region and the same Shiga toxin effector polypeptide region linked in the same way but wherein the immunoglobulin-type binding region is amino-terminal to the Shiga toxin effector polypeptide region in the second molecule.

In certain embodiments, the cell-targeted molecules of the present invention comprise furin-cleavage resistant, Shiga toxin A Subunit effector polypeptide regions and exhibit equivalent cytotoxic potency to related cell-targeted molecules with wild-type, Shiga toxin effector polypeptide regions. For certain embodiments of the protease-cleavage resistant, cell-targeted molecules of the present invention, upon contacting a cell physically coupled with an extracellular target biomolecule of the binding region of the cell-targeted molecule of the invention (target positive cell) with the cell-targeted molecule, the cell-targeted molecule is capable of causing death of the target positive cell. For certain embodiments of the protease-cleavage resistant, cell-targeted molecules of the present invention, after contacting a cell physically coupled with an extracellular target biomolecule of the binding region of the cell-targeted molecule of the invention with the cell-targeted molecule, the cell-targeted molecule is capable of causing death of the cell with a cytotoxic effect equivalent to the same cell-targeted molecule but which comprises a furin-cleavable, wild-type, Shiga toxin effector polypeptide, such as, e.g., a wild-type, Shiga toxin A1 fragment. For certain embodiments of the protease-cleavage resistant, cell-targeted molecules of the present invention, after contacting a cell physically coupled with an extracellular target biomolecule of the binding region of the cell-targeted molecule of the invention with the cell-targeted molecule, the cell-targeted molecule is capable of causing death of the cell with 1) a cytotoxic effect 3-fold, 4-fold, 5-fold, 6-fold, 7-fold, 8-fold, 9-fold, 10-fold, or greater than a second cell-targeted molecule comprising the same binding region and the same Shiga toxin effector polypeptide region but wherein the Shiga toxin effector polypeptide region is not proximal to an amino-terminus of a polypeptide component of the second cell-targeted molecule and 2) a cytotoxic effect equivalent to the same cell-targeted molecule but which comprises a furin-cleavable, wild-type, Shiga toxin effector polypeptide, such as, e.g., a wild-type, Shiga toxin A1 fragment. For certain embodiments of the cell-targeted molecules of the present invention, after contacting a cell physically coupled with an extracellular target biomolecule of the binding region of a cell-targeted molecule of the invention with the cell-targeted molecule, the cell-targeted molecule is capable of causing death of the cell with 1) a cytotoxic effect 3-fold, 4-fold, 5-fold, 6-fold, 7-fold, 8-fold, 9-fold, 10-fold, or greater than a second cell-targeted molecule comprising the same binding region and the same Shiga toxin effector polypeptide region linked in the same way but wherein the binding region is located amino-terminal to the Shiga toxin effector polypeptide region within a polypeptide component of the second cell-targeted molecule and 2) a cytotoxic effect equivalent to the same cell-targeted molecule but which comprises a furin-cleavable, wild-type, Shiga toxin effector polypeptide, such as, e.g., a wild-type, Shiga toxin A1 fragment. For certain embodiments of the cytotoxic proteins of the present invention, after contacting a cell physically coupled with an extracellular target biomolecule of the immunoglobulin-type binding region of a cytotoxic protein of the invention with the cytotoxic protein, the cytotoxic protein is capable of causing death of the cell with 1) a cytotoxic effect 3-fold, 4-fold, 5-fold, 6-fold, 7-fold, 8-fold, 9-fold, 10-fold, or greater than a second protein comprising the same immunoglobulin-type binding region and the same Shiga toxin effector polypeptide region linked in the same way but wherein the immunoglobulin-type binding region is amino-terminal to the Shiga toxin effector polypeptide region in the second molecule and 2) a cytotoxic effect equivalent to the same cell-targeted molecule but which comprises a furin-cleavable, wild-type, Shiga toxin effector polypeptide, such as, e.g., a wild-type, Shiga toxin A1 fragment.

For purposes of the present invention, the exhibition of “equivalent” Shiga toxin effector cytotoxicity compared to the cytotoxicity of a reference cell-targeted molecule comprising 1) the same immunoglobulin-type binding region and 2) a wild-type, Shiga toxin A1 fragment polypeptide region oriented amino-terminal to the binding region refers to a level of cytotoxicity within ten percent or less, as measured by an appropriate quantitative assay with reproducibility comparable to cytotoxicity measurements of a cell-targeted molecule comprising a Shiga toxin effector polypeptide region comprising a full-length, Shiga toxin A1 fragment. For cytotoxicity in a target-positive cell kill assay in laboratory cell culture, “equivalent” cytotoxicity is typically a CD₅₀ value within ten percent of the CD₅₀ value of a reference protein (such as, e.g., a “second cell-targeted molecule” or “third-cell targeted molecule), which comprises 1) an identical immunoglobulin-type binding region to the cell-targeted molecule of interest and 2) a wild-type, Shiga toxin A1 fragment polypeptide located amino-terminal to the binding region; and wherein the binding region and the wild-type, Shiga toxin A1 fragment polypeptide are linked together with an identical linkage structure as in the protein of interest.

Furthermore, if a cell-targeted molecule of the present invention exhibits cytotoxicity equivalent to a reference molecule comprising a wild-type, Shiga toxin A1 fragment polypeptide, then the cell-targeted molecule of the invention exhibits the Shiga toxin effector function of subcellular routing at an activity level equivalent to the subcellular routing activity level of that reference molecule, e.g. a sub-cellular routing activity equivalent to a reference cell-targeted molecule comprising an amino-terminus proximal, furin-cleavable, wild-type, Shiga toxin A1 fragment.

Cell kill may be accomplished using a cell-targeted molecule of the present invention under varied conditions of target cells, such as an ex vivo manipulated target cell, a target cell cultured in vitro, a target cell within a tissue sample cultured in vitro, or a target cell in vivo.

The expression of the target biomolecule need not be native in order for targeted cell killing by a cell-targeted molecule of the present invention. Cell surface expression of the target biomolecule could be the result of an infection, the presence of a pathogen, and/or the presence of an intracellular microbial pathogen. Expression of a target biomolecule could be artificial such as, for example, by forced or induced expression after infection with a viral expression vector, see e.g. adenoviral, adeno-associated viral, and retroviral systems. An example of inducing expression of a target biomolecule is the upregulation of CD38 expression of cells exposed to retinoids, like all-trans retinoic acid and various synthetic retinoids, or any retinoic acid receptor (RAR) agonist (Drach J et al., Cancer Res 54: 1746-52 (1994); Uruno A et al., J Leukoc Biol 90: 235-47 (2011)). In another example, CD20, HER2, and EGFR expression may be induced by exposing a cell to ionizing radiation (Wattenberg M et al., Br J Cancer 110: 1472-80 (2014)).

### B. Selective Cytotoxicity Among Cell Types

Cell-targeted molecules designed using Shiga toxin effector polypeptide regions can have potent cell-kill activity. By targeting the delivery of enzymatically active, Shiga toxin polypeptides using high-affinity, target binding regions to specific cell types, this potent cell-kill activity can be restricted to preferentially killing selected cell types. The present invention provides various cytotoxic, cell-targeted molecules which exhibit selective cytotoxicity to certain a cell-type(s) in the presence of another cell-type(s).

In certain embodiments, upon administration of the cell-targeted molecule of the present invention to a mixture of cell types, the cell-targeted molecule is capable of selectively killing those cells which are physically coupled with a certain extracellular target biomolecule (e.g., target positive cells) compared to cell types not physically coupled with any extracellular target biomolecule specifically bound by the binding region of that cell-targeted molecule (e.g., target negative cells). In certain embodiments, the cell-targeted molecule of the present invention is capable of selectively or preferentially causing the death of a specific cell type within a mixture of two or more different cell types. This enables the targeting of cytotoxic activity to specific cell types with a high preferentiality, such as a 3-fold cytotoxic effect, over “bystander” cell types that do not express the target biomolecule. Alternatively, the expression of the target biomolecule of the binding region may be non-exclusive to one cell type if the target biomolecule is expressed in low enough amounts and/or physically coupled in low amounts with cell types that are not to be targeted. This enables the targeted cell-killing of specific cell types with a high preferentiality, such as a 3-fold cytotoxic effect, over “bystander” cell types that do not express significant amounts of the target biomolecule or are not physically coupled to significant amounts of the target biomolecule.

In certain embodiments of the cell-targeted molecules of the present invention, administration of the cell-targeted molecule of the invention to two populations of cell types which differ with respect to the presence and/or polypeptide sequence of an extracellular target biomolecule, the cell-targeted molecule is capable of causing cell death as defined by the half-maximal cytotoxic concentration (CD₅₀) on a population of target cells, whose members express an extracellular target biomolecule of the binding region of the cell-targeted molecule, e.g., at a dose at least three-times lower than the CD₅₀ dose of the same cell-targeted molecule to a population of cells whose members do not express an extracellular target biomolecule of the binding region of the cell-targeted molecule.

In certain embodiments, the cytotoxic activity of a cell-targeted molecule of the present invention toward populations of cell types physically coupled with an extracellular target biomolecule is at least 3-fold higher than the cytotoxic activity toward populations of cell types not physically coupled with any extracellular target biomolecule bound specifically by that cell-targeted molecule of the invention. According to the present invention, selective cytotoxicity may be quantified in terms of the ratio (a/b) of (a) cytotoxicity towards a population of cells of a specific cell type physically coupled with a target biomolecule of the binding region to (b) cytotoxicity towards a population of cells of a cell type not physically coupled with a target biomolecule of the binding region. In certain embodiments, the cytotoxicity ratio is indicative of selective cytotoxicity which is at least 3-fold, 5-fold, 10-fold, 15-fold, 20-fold, 25-fold, 30-fold, 40-fold, 50-fold, 75-fold, 100-fold, 250-fold, 500-fold, 750-fold, 1000-fold, or higher for populations of cells or cell types physically coupled with a target biomolecule of the binding region compared to populations of cells or cell types not physically coupled with a target biomolecule of the binding region.

This preferential cell-killing function allows a target cell to be killed by certain cell-targeted molecules of the present invention under varied conditions and in the presence of non-targeted bystander cells, such as ex vivo manipulated mixtures of cell types, in vitro cultured tissues with mixtures of cell types, or in vivo in the presence of multiple cell types (e.g. in situ or in a native location within a multicellular organism).

In certain embodiments, the proteins of the present invention are capable of selectively or preferentially causing the death of a specific cell type within a mixture of two or more different cell types. This enables the targeted cytotoxic activity to specific cell types with a high preferentiality, such as a 3-fold cytotoxic effect, over “bystander” cell types that do not express extracellular target bound specifically by the binding region of that cytotoxic protein of the invention. Alternatively, the expression of an extracellular target biomolecule may be non-exclusive to one cell type if the extracellular target biomolecule is expressed in low enough amounts by cell types that are not to be targeted. This enables the preferential cell-killing of only those cell type(s) expressing the highest amounts of extracellular target biomolecules, such as a 3-fold cytotoxic effect, over “bystander” cell types that do not express significant amounts of the target biomolecule bound specifically by that cytotoxic protein and/or are not physically coupled to significant amounts of extracellularly exposed target biomolecule bound specifically by that cytotoxic protein.

The cell-targeted molecules of the present invention are useful for the elimination of populations of specific cell types. For example, the cell-targeted molecules of the present invention (e.g., cytotoxic, fusion proteins of the present invention) are useful for the treatment of certain tumors, cancers, and/or other growth abnormalities by eliminating “target biomolecule+” cells that express elevated levels of target biomolecule at one or more cellular surfaces.

In certain embodiments, the cytotoxic activity of a cell-targeted molecule of the present invention toward populations of cell types physically coupled with a certain extracellular target biomolecule is at least 3-fold higher than the cytotoxic activity toward populations of cell types not physically coupled with significant amounts of an extracellular target biomolecule bound specifically by the binding region of that particular cell-targeted molecule of the invention. According to the present invention, selective cytotoxicity may be quantified in terms of the ratio (a/b) of (a) cytotoxicity towards a population of cells physically coupled with a significant amount of an extracellular target biomolecule bound by the binding region of the cell-targeted molecule of the invention to (b) cytotoxicity towards a population of cells of a cell type not physically coupled with a significant amount of any extracellular target biomolecule bound specifically by the binding region of that particular cytotoxic of the invention. In certain embodiments, the cytotoxicity ratio is indicative of selective cytotoxicity which is at least 3-fold, 5-fold, 10-fold, 15-fold, 20-fold, 25-fold, 30-fold, 40-fold, 50-fold, 75-fold, 100-fold, 250-fold, 500-fold, 750-fold, 1000-fold or higher for populations of cells or cell types expressing an extracellular target biomolecule or physically coupled with an extracellular target biomolecule bound by the binding region of the cell-targeted molecule of the invention compared to populations of cells or cell types which do not express an extracellular target biomolecule or that are not physically coupled with significant amounts of an extracellular target biomolecule bound specifically the binding region of that particular cell-targeted molecule of the invention. For example, administration of certain cell-targeted molecules of the present invention to two different populations of cells which differ with respect to the presence and/or polypeptide sequence of an extracellular target biomolecule, the cell-targeted molecule of the invention is capable of causing cell death to the cell-types physically coupled with an extracellular target biomolecule bound by the cell-targeted molecule's binding region, e.g., at a CD₅₀ at least three times or less than the CD₅₀ of binding to cell types that are not physically coupled with an extracellular target biomolecule bound by the cell-targeted molecule's binding region or to cell types that are physically coupled only with forms of that extracellular target biomolecule which comprise sequence variations or mutations which disrupt binding specificity by the binding region of that cell-targeted molecule.

Levels of extracellular target biomolecules on the surface of cells may be determined using various methods known to the skilled worker, such as, e.g., FACS methods. As used herein, a significant amount of an extracellular target biomolecule expressed at a cellular surface is greater than 10,000, 20,000, 30,000, 40,000, 50,000, 60,000, or 70,000 mean fluorescence intensity (MFI) by FACS analysis depending on the cell type.

In certain embodiments of the cell-targeted molecules of the present invention, administration of the cell-targeted molecule to two different populations of cell types, the cell-targeted molecule is capable of causing cell death as defined by the half-maximal cytotoxic concentration (CD₅₀) on a first cell population, whose members express a certain target biomolecule at a cellular surface, at a dose at least three-times lower than the CD₅₀ dose of the same cell-targeted molecule to a second population of cells whose members do not express that target biomolecule, do not express a significant amount of that target biomolecule, or are not exposing a significant amount of that target biomolecule bound by the cell-targeted molecule's binding region.

C. Reducing Protease-Cleavage Sensitivity of Shiga Toxin A Subunit Effector Polypeptide Regions of Cell-Targeted Molecules while Maintaining Efficient Intracellular Routing and Potent Cytotoxicity

The disruption of the furin-cleavage motif at the carboxy terminus of the Shiga toxin A1 fragment region in Shiga toxin A Subunit effector regions of cell-targeted molecules of the present invention enables the engineering of cell-targeted molecules with improved toxicity profiles after administration to a vertebrate while maintaining Shiga toxin cytotoxic potency comparable to cell-targeted molecules comprising furin-cleavage sensitive, Shiga toxin effector regions but (see Examples, infra). Certain cell-targeted molecules of the present invention exhibit reduced deleterious effects (e.g. non-specific toxicities and/or general systemic toxicity) after administration to vertebrates as compared to related cell-targeted molecules comprising wild-type, Shiga toxin effector polypeptides and, additionally, can exhibit improved stability during production, storage, and administration.

Previously, it was believed that cytotoxic Shiga toxin A Subunit constructs comprising Shiga toxin A1 fragment catalytic regions must maintain or somehow compensate for the naturally occurring proteolytic processing by furin within intoxicated cells in order to preserve efficient and potent cytotoxicity. Unexpectedly, it was discovered that the furin-cleavage event was not required for potent cytotoxicity because potent Shiga toxin cytotoxicity at the level of a wild-type, Shiga toxin control construct was achieved in the absence of a target-cell-mediated, furin-cleavage event at the carboxy terminus of the Shiga toxin A1 fragment despite the presence of a relatively large (greater than 28 kDa), immunoglobulin-type, binding region moiety on the carboxy terminus (see Examples, infra). The lack of a furin-cleavage event within the intoxicated cell was expected to interfere with the efficient liberation of a Shiga toxin A1 fragment and, thus, result in the continued linkage of a relatively large, carboxy-terminal, binding region to the Shiga toxin A1 fragment region. However despite this expectation, potent Shiga toxin cytotoxicity was achieved with furin-cleavage deficient, Shiga toxin A Subunit constructs comprising relatively large, carboxy-terminal, binding regions and lacking any apparent compensatory feature(s), such as, e.g. an engineered, alternative protease site.

These results are surprising because the optimal Shiga toxin intoxication process was thought to require liberation of the Shiga toxin A1 fragments from all other large molecular moieties and exposure of the carboxy terminus of the A1 fragment to efficiently retrotranslocate liberated A1 fragments from the endoplasmic reticulum to the cytosol where the A1 fragments can form an enzymatically active structure that catalytically inactivates the intoxicated cell's ribosomes. In particular, the persistence and/or inefficient release of a molecular moiety covering the carboxy terminus of the Shiga toxin A1 fragment was expected to interfere with the Shiga toxin A1 fragment's natural mechanism of efficiently gaining access to the cytosol involving the exposure of the A1 fragment's hydrophobic carboxy terminus domain recognized by the ERAD system (see Di R et al., Toxicon 57: 535-39 (2011); Li S et al., PLoS One 7: e41119 (2012)). For example, the persistence of a molecular moiety covering the carboxy terminus of the Shiga toxin A1 fragment was expected to disrupt the accessibility of the carboxy terminus of the Shiga toxin A1 fragment to the ERAD machinery in the endoplasmic reticulum and efficiently gaining access to the cytosol where it forms an enzymatically active structure. Unexpectedly, this is found to be incorrect because efficient and potent Shiga toxin cytotoxicity was achieved in the absence of a target-cell-mediated, furin-cleavage event at the carboxy terminus of the Shiga toxin A1 fragment despite the presence of a large, carboxy-terminal, cell-targeting moiety (see Examples, infra).

Alternatively, the lack of an intoxicated-cell-mediated, furin-cleavage event may be hypothetically compensated for. A non-limiting example of a potential, compensatory approach is engineering a heterologous and/or ectopic protease site that can functionally substitute for the lack of the native, Shiga toxin, furin-cleavage event, such as, e.g., adding a heterologous and/or ectopic protease site cleaved by an intracellular protease of the target cell. In this approach, the Shiga toxin A1 fragment-like polypeptide can be designed to intracellularly dissociate from one or more other components of the chimeric construct by the time the constructed molecule reaches the endoplasmic reticulum of an intoxicated cell such that in the endoplasmic reticulum the carboxy terminus of the Shiga toxin A1 fragment-like polypeptide becomes exposed for recognition by the ERAD machinery.

This proposed approach for designing Shiga toxin A Subunit effector polypeptides that compensates for the lack of an intoxicated-cell-mediated, furin-cleavage events are hypothetical. The proposed approach could significantly alter the efficiency and potency of cytotoxicity as compared to a molecule comprising a wild-type, Shiga toxin A Subunit or Shiga toxin A Subunit construct comprising only wild-type sequences which include the furin-cleavage site naturally occurring at the carboxy terminus of the A1 fragment region. In addition, only certain variants which rely on target cell endoproteases, might allow for a relatively large, cell-targeting, binding region moiety to be fused carboxy-terminal to the Shiga toxin effector polypeptide. However, currently no compensatory approach relying on a target cell endoprotease other than furin is known which can provide fully compensatory cytotoxicity equivalent to furin cleavage, and alternative cellular proteases like calpain have shown to be less efficient in facilitating Shiga toxin cytotoxicity (Garred Ø et al., Exp Cell Res 218: 39-49 (1995); Garred Ø et al., J Biol Chem 270: 10817-21 (1995); Kurmanova, Biochem Biophys Res Commun 357: 144-9 (2007)).

The cytotoxic proteins of the present invention that comprise Shiga toxin effector polypeptides comprising disrupted furin-cleavage motifs all exhibit reduced sensitivity to cleavage by furin (see Examples, infra). Because the minimal, furin-cleavage, R/Y-x-x-R motif is shared by multiple proteases, such as by highly promiscuous proteases (e.g. trypsin), certain disrupted furin-cleavage motifs of the Shiga toxin effector polypeptides of the present invention are expected to exhibit reduced sensitivity to cleavage by multiple proteases besides just furin (see e.g. Kurmanova A et al., Biochem Biophys Res Commun 357: 144-9 (2007)). For example, the proprotein convertase class of peptidases includes at least seven members in humans, PC1, PC2, PC3, PC4, PACE4, PC5, PC6, and PC7 (Fugere M, Day R, Trends Pharmacol Sci 26: 294-301 (2005)), many of which are known to cleave their substrates at single or pairs of basic residues, such as, e.g., one or more arginine residues (Seidah N, Ann N Y Acad Sci 1220: 149-61 (2011)). The disruption of the furin-cleavage motif at the carboxy terminus of the Shiga toxin A1 fragment region in a Shiga toxin A Subunit effector polypeptide reduces furin cleavage at the motif and might reduce cleavage by other proteases beside furin, such as, e.g., trypsin and extracellular proteases common in the vascular system of vertebrates.

Certain cell-targeted molecules of the present invention display optimized cytotoxic potency as compared to cell-targeted molecules comprising protease-cleavage sensitive, Shiga toxin effector polypeptides despite the presence within the cell-targeted molecules of the present invention of a relatively large, immunoglobulin-type, binding region moiety fused to the carboxy terminus of the Shiga toxin effector polypeptide that cannot be released by furin cleavage inside an intoxicated target cell.

D. Improved In Vivo Tolerability and Stability Due to Protease-Cleavage Resistance while Retaining Wild-Type Cytotoxicity

In certain embodiments, the cell-targeted molecules of the present invention exhibit increased stability and/or improved, in vivo tolerability as compared to related, furin-cleavage sensitive, cell-targeted molecule variants. The increased stability can be exhibited in vitro and/or in vivo.

The stability of a therapeutic or diagnostic molecule over time is an important feature and can affect for which applications the molecule may be practically employed. Molecular stability includes in vitro and in vivo, such as, e.g., stability within an organism after administration and during storage over a range of temperatures and concentrations. For certain immunotoxins or ligand-toxin fusions, the stability of the linkage between the toxin and other components can affect the amount of non-specific toxicity caused by the release of untargeted toxin over time within the body of an organism.

Certain cell-targeted molecules of the present invention (useful as therapeutics and/or diagnostics) exhibit reduced non-specific toxicity in vivo, manifested as improved, in vivo tolerability as compared to more protease-cleavage sensitive variants. In vivo tolerability can be determined by the skilled worker using techniques known in the art and/or described herein. In addition to assessing in vivo tolerability using mortality, signs of morbidity may be used for assessing in vivo tolerability, such as, e.g., aspects of body weight, physical appearance, measureable clinical signs, unprovoked behavior, and responses to external stimuli (see e.g. Morton D, Griffiths P, Vet Rec 116: 431-43 (1985); Montgomery C, Cancer Bull 42: 230-7 (1990); Ullman-Culleré M, Foltz C, Lab Anim Sc 49: 319-23 (1999); Clingerman K, Summers L, J Am Assoc Lab Anim Sci 51: 31-6 (2012)). Euthanasia may be used in response to signs of morbidity and/or morbundity and, thus, create a mortality time-point. For example, a decrease in body weight of 15-20% in 2-3 days can be used as a sign of morbidity in rodents and as a justification for euthanization (see e.g. Institute of Laboratory Animal Research 2011. Guide for the care and use of laboratory animals, 8th ed., Washington, D.C., U.S.: National Academies Press (2011)).

For purposes of the claimed invention, the term “improved, in vivo tolerability” refers to a reproducible and statistically significant decrease in the toxicity and/or general deleterious effect of the cell-targeted molecule on the health or survival of a whole organism after receiving administration of the cell-targeted molecule, such as a decrease of 20%, 25%, 30%, 40%, 50%, 60%, 70%, 80%, 90% or greater, preferably 50% or greater, of the improved cell-targeted molecule to a mammalian organism as compared to a reference cell-targeted molecule when compared using the same assay under the same conditions, e.g. the same species, the same dose and cumulative dosage, the same dosing schedule, and the same duration for time-points of observation and/or measurement. A decrease in toxicity or general deleterious effect can be measured by a decrease in mortality and/or morbidity over specific time duration.

As shown in the Examples, a decrease in toxicity could represent 100% survival at a given time duration for mammals receiving the cell-targeted molecule with improved, in vivo tolerability as compared to 100% mortality at the same time-point for mammals receiving the reference cell-targeted molecule. Mortality may be due to death or euthanasia for compassionate reasons as mentioned above. Generally, the dosing schedule is two to three doses per week for 2, 3, 4, or more weeks, where each dose is around 0.001 to 10 mg of molecule per kg body weight.

The improved, in vivo tolerability observed for exemplary cell-targeted molecules of the present invention suggests that much higher doses of these cell-targeted molecules may be safely administered to mammals as compared to the doses of related cell-targeted molecules comprising a furin-cleavage sensitive, Shiga toxin effector polypeptide. Certain cytotoxic cell-targeted molecules of the present invention might exhibit reduced non-specific toxicity as compared to more protease-cleavage sensitive variants because the protease-cleavage resistance serves to protect and preserve the linkage between the Shiga toxin effector component and the cell-targeting binding region.

In addition, certain cell-targeted molecules of the invention exhibit increased half-lives, both in vitro and/or in vivo, as compared to more protease-cleavage sensitive variants. Molecular stability can be assayed by determining the half-life of a molecule of interest with regard to the association of its components. Certain embodiments of the cell-targeted molecules of the present invention will have longer half-lives as compared to furin-cleavage sensitive variants, especially with regard to the continued association of the Shiga toxin effector component and one or more other components. For example, certain embodiments of the cell-targeted molecules of the invention will have longer half-lives with regard to the continued association of the Shiga toxin effector component and another component, e.g. a cell-targeting moiety, as compared to a furin-cleavage sensitive variant wherein the furin-cleavage sensitive site(s) lies between those two components.

Maximal, Shiga toxin cytotoxicity was believed to require the cleavage of Shiga toxin A Subunit, exposure in the endoplasmic reticulum of a hydrophobic region proximal to the carboxy terminus of the A1 fragment, and the liberation of the A1 fragment from the rest of the holotoxin, all of which might result in multiple structural and functional changes to the A1 fragment. In addition, it is believed that optimal intracellular transport of Shiga toxin A1 fragments to the cytosol requires the same events: A Subunit cleavage, exposure of the A1 fragment carboxy terminus, and the liberation of the A1 fragment from all other molecular moieties. In the absence of furin-cleavage of the Shiga toxin A Subunit, sub-cellular routing of Shiga toxin catalytic domains can occur but is suboptimal, less efficient, and results in reductions in the efficacy of ribosome inhibition (Garred Ø et al., Exp Cell Res 218: 39-49 (1995); Lea N et al., Microbiology 145: 999-1004 (1999); Kurmanova A et al., Biochem Biophys Res Commun 357: 144-9 (2007)). Because furin proteolytic processing of Shiga toxin A Subunits in intoxicated vertebrate cells is critical for maximal cytotoxicity, it is important when designing cytotoxic molecules derived from Shiga toxin A Subunits to maintain or compensate for this naturally occurring proteolytic processing in order to preserve maximal, Shiga toxin cytotoxicity.

The liberation of the Shiga toxin A1 fragment from all other moieties may be required both for 1) exposing the carboxy terminus of the A1 fragment for recognition by cellular factors within the endoplasmic reticulum of intoxicated cells and 2) maximizing catalytic activity.

The liberation of the Shiga toxin A1 fragment is required to expose the carboxy terminus of the A1 fragment. The hydrophobic region around 224 to 241 in the carboxy-terminal region of the A1 fragment of StxA is believed to play a role in the retrotranslocation of the A1 fragment from the lumen of the endoplasmic reticulum to the cytosol (Suhan M, Hovde C, Infect Immun 66: 5252-9 (1998); LaPointe P et al., J Biol Chem 280: 23310-18 (2005)). Several amino residues in this hydrophobic region become more surface accessible after cleavage of Shiga toxin A Subunits in both Stx1A and Stx2A (Di R et al., Toxicon 57: 535-39 (2011)). Thus, the liberation of the Shiga toxin A1 fragment and the exposure of its carboxy-terminal hydrophobic region might trigger the transport of the A1 fragment from the endoplasmic reticulum to the cytosol (Suhan M, Hovde C, Infect Immun 66: 5252-9 (1998); LaPointe P et al., J Biol Chem 280: 23310-18 (2005); Di R et al., Toxicon 57: 535-39 (2011)). In addition, the carboxy terminus of the A1 fragment may function as a ligand recognized and bound by an endoplasmic reticular receptor, other than a chaperone protein, which contributes to the efficient retrotranslocation of the A1 fragment (LaPointe P et al., J Biol Chem 280: 23310-18 (2005)).

Structural changes which enhance cytotoxicity of the Shiga toxin A1 fragment could occur after liberation of the A1 fragment from all other moieties. The free Shiga toxin A1 fragment might exhibit optimal catalytic activity, such as, e.g., by exposing certain catalytic regions buried in the Shiga holotoxin structure (see Tesh V et al., Infect Immun 61: 3392-402 (1993); Di R et al., Toxicon 57: 535-39 (2011)). Shiga toxin catalytic activation after proteolytic cleavage and exposure to reducing conditions or enhancement of Shiga toxin toxicity after proteolytic processing and exposure to reducing conditions are most likely the result of separation of the A1 fragment from the A2 fragment (Tesh V et al., Infect Immun 61: 3392-402 (1993)). Structural changes to the Shiga toxin A1 fragment after dissociation from the rest of the Shiga holotoxin may relate to functional changes, such as, e.g. the ability to form a newly folded structure which is more catalytically active after being unfolded by the ERAD machinery and translocated to the cytosol, the ability of the cytosolic A1 fragment to evade degradation by the proteasome, and the ability to form structures with more open catalytic active sites and/or binding clefts which enhances enzymatic activity (Smith M et al., Infect Immun 77: 2730-40 (2009); Di R et al., Toxicon 57: 535-39 (2011)). Thus, the liberation of the Shiga toxin A1 fragment from all other moieties may be required for maximal Shiga toxin cytotoxicity due to structural and functional changes which enhance the sub-cellular routing of the A1 fragment to the cytosol of intoxicated cells, enzymatic activity of the A1 fragment in the cytosol of intoxicated cells, and persistence of the A1 fragment in the cytosol of intoxicated cells.

The dissociation of the Shiga toxin A1 fragment from the A2 fragment and the rest of the Shiga holotoxin is required for the translocation of the A1 fragment from the lumen of the endoplasmic reticulum to the cytosol (LaPointe P et al., J Biol Chem 280: 23310-18 (2005); Li S et al., PLoS One 7: e41119 (2012)). The liberation of the A1 fragment exposes a hydrophobic domain which triggers a series of complex steps: 1) recognition of the A1 fragment by the endoplasmic-reticulum-associated degradation (ERAD) system, 2) unfolding, 3) retrotranslocation across the endoplasmic reticulum membrane, and 4) refolding to a catalytic formation in the cytosol (Li S et al., PLoS One 7: e41119 (2012)).

First, the carboxy terminus of the Shiga toxin A1 fragment, which is exposed after furin cleavage and liberation from the rest of the Shiga holotoxin, is recognized by the ERAD system. The ERAD system identifies terminally misfolded proteins in the ER, tags them with polyubiquitin, and exports them to the cytosol for proteasomal destruction (Smith M et al., Science 334: 1086-90 (2011)). The A1 fragments of Shiga toxins exploit the ERAD pathway to gain access to the cytosol perhaps by mimicking an unfolded ERAD substrate via a locally misfolded, polypeptide region, comprising a patch of relatively hydrophobic amino acid residues, located on the carboxy-terminals of A1 fragments created by furin cleavage (LaPointe P et al., J Biol Chem 280: 23310-18 (2005); Yu M, Haslam D, Infect Immun 73: 2524-32 (2005); Li S et al., PLoS One 7: e41119 (2012)). The partially unfolded, hydrophobic patch of amino acid residues near the carboxy terminus of Shiga toxin A1 fragments exposed by furin cleavage can be recognized by endoplasmic reticulum chaperone proteins of the ERAD system LaPointe P et al., J Biol Chem 280: 23310-18 (2005); Yu M, Haslam D, Infect Immun 73: 2524-32 (2005); Li S et al., PLoS One 7: e41119 (2012)).

When a Shiga toxin A1 fragment first enters the cytosol of an intoxicated eukaryotic cell, it is believed to be polyubiqutinated and in a substantially disordered conformation as a result of being unfolded, thus A1 fragments must both avoid proteasomal degradation and refold into a catalytically active conformation in order to exert their cytotoxic catalytic activity (Tam P, Lingwood C, Microbiology 153: 2700-10 (2007); Li S et al., PLoS One 7: e41119 (2012)). Once in the cytosol, an active Shiga toxin A1 fragment can irreversibly cripple one eukaryotic ribosome after another via the A1 fragment's potent enzymatic activity at a rate of approximately 700 ribosomes per minute (Brigotti M et al., Toxicon 35:1431-1437 (1997); Tam P, Lingwood C, Microbiology 153: 2700-10 (2007)). After a threshold number of ribosomes is inactivated, an intoxicated host cell is predicted to experience sufficient reduction in protein synthesis to induce cell death via apoptosis (Iordanov M et al., Mol Cell Biol 17: 3373-81 (1997); Smith W et al., Infect Immun 71: 1497-504 (2003); Lee S et al., Cell Microbiol 10: 770-80 (2008); Tesh V, Future Microbiol 5: 431-53 (2010)).

Intracellular, furin cleavage of the Shiga toxin A Subunit between the A1 and A2 fragments is important for maximal Shiga toxin cytotoxicity. Experiments have shown maximal Shiga holotoxin cytotoxicity requires 1) the minimal furin-cleavage site R/Y-x-x-R located between the A1 and A2 fragments in the Shiga toxin A Subunit; 2) certain amino acid residues in the surface-exposed, extended loop structure in the Shiga toxin A Subunit comprising the minimal furin-cleavage site; and 3) the cellular expression of furin by intoxicated vertebrate cells.

Human cells lacking furin are protected against Shiga toxin cytotoxicity, and these same furin-deficient cells can be made Shiga toxin sensitive by the forced expression of furin (Garred Ø et al., J Biol Chem 270: 10817-21 (1995); Kurmanova A et al., Biochem Biophys Res Commun 357: 144-9 (2007)).

Furin was shown to be necessary for maximal Shiga toxin cytotoxicity in certain human cancer cells (Garred Ø et al., J Biol Chem 270: 10817-21 (1995); Kurmanova A et al., Biochem Biophys Res Commun 357: 144-9 (2007)). Shiga toxins with disrupted furin-cleavage sites and/or mutations in conserved, surface-exposed, extended loop structures show reduced cytotoxicity. Disrupting the S-R/Y-x-x-R furin-cleavage motif in the surface-exposed, extended loop of Shiga toxin A Subunits with amino acid residue substitutions or deletions resulted in less efficient cleavage of the A Subunits and less efficient ribosome inhibition in vertebrate cells (Burgess B, Roberts L, Mol Microbiol 10: 171-9 (1993); Garred Ø et al., Exp Cell Res 218: 39-49 (1995); Lea N et al., Microbiology 145: 999-1004 (1999); Kurmanova A et al., Biochem Biophys Res Commun 357: 144-9 (2007)). The disruption of the furin-cleavage motif in the A Subunit of SLT-1 reduced its ribosome inhibition activity by 60-fold (Lea N et al., Microbiology 145: 999-1004 (1999)). In addition, disruption of the flanking regions of the furin-cleavage motif without disrupting the minimal furin-cleavage motif R-x-x-R also reduced the ribosome inhibition activity of Stx (Kurmanova A et al., Biochem Biophys Res Commun 357: 144-9 (2007)).

The dissociation of the Shiga toxin A1 fragment from the A2 fragment is required for activation of the catalytic domain of the A1 fragment (Garred Ø et al., Exp Cell Res 218: 39-49 (1995); Garred Ø et al., J Biol Chem 270: 10817-21 (1995); Kurmanova A et al., Biochem Biophys Res Commun 357: 144-9 (2007)). The catalytic domains of Shiga toxins are inactive before furin cleavage possibly because the A2 portion of the A Subunit occludes the active site cleft of the A1 portion, with methionine-260 of the A2 portion protruding into and blocking the active site of the A1 portion (Lea N et al., Microbiology 145: 999-1004 (1999); see also Fraser M et al., Nat Struct Biol 1: 59-64 (1994)).

The dissociation of the A1 fragment from the A2 fragment might be required for activation of the catalytic domain of the A1 fragment (Garred Ø et al., Exp Cell Res 218: 39-49 (1995); Garred Ø et al., J Biol Chem 270: 10817-21 (1995); Kurmanova A et al., Biochem Biophys Res Commun 357: 144-9 (2007)). The catalytic domains of Shiga toxins might be inactive before furin cleavage because the catalytic domain is sterically blocked (Lea N et al., Microbiology 145: 999-1004 (1999); see also Fraser M et al., Nat Struct Biol 1: 59-64 (1994)).

E. Delivery of Additional Exogenous Material into the Interior of a Target Cell

In addition to direct cell killing, cell-targeted molecules of the present invention optionally may be used for delivery of additional exogenous materials into the interiors of target cells. The delivery of additional exogenous materials may be used, e.g., for cytotoxic, cytostatic, information gathering, and/or diagnostic functions. Non-toxic variants of the cytotoxic, cell-targeted molecules of the invention, or optionally toxic variants, may be used to deliver additional exogenous materials to and/or label the interiors of cells physically coupled with an extracellular target biomolecule of the binding region of the cell-targeted molecule of the invention. Various types of cells and/or cell populations which express target biomolecules to at least one cellular surface may be targeted by the cell-targeted molecules of the invention for receiving exogenous materials. The functional components of the present invention are modular, in that various Shiga toxin effector regions and additional exogenous materials may be linked to various binding regions to provide diverse applications, such as non-invasive in vivo imaging of tumor cells.

Because the cell-targeted molecules of the present invention, whether toxic or nontoxic, and catalytically inactive forms thereof, are capable of entering cells physically coupled with an extracellular target biomolecule recognized by its binding region, certain embodiments of the cell-targeted molecules of the invention may be used to deliver additional exogenous materials into the interior of targeted cell types. In one sense, the entire cell-targeted molecule is an exogenous material which will enter the cell; thus, the “additional” exogenous materials are heterologous materials linked to but other than the core of the cell-targeted molecule, i.e. the minimal required components of the cell-targeted molecule of the present invention being a Shiga toxin A Subunit effector polypeptide and a binding region capable of specifically binding an extracellular target biomolecule.

“Additional exogenous material” as used herein refers to one or more molecules, often not generally present within a native target cell, where the cell-targeted molecules of the present invention may be used to specifically transport such material to the interior of a cell. Non-limiting examples of additional exogenous materials are cytotoxic agents, peptides, polypeptides, proteins, polynucleotides, detection promoting agents, and small molecule chemotherapeutic agents.

In certain embodiments of the cell-targeted molecules of the present invention for delivery of additional exogenous material, the additional exogenous material is a cytotoxic agent, such as, e.g., a small molecule chemotherapeutic agent, cytotoxic antibiotic, alkylating agent, antimetabolite, topoisomerase inhibitor, and/or tubulin inhibitor. Non-limiting examples of cytotoxic agents include aziridines, cisplatins, tetrazines, procarbazine, hexamethylmelamine, vinca alkaloids, taxanes, camptothecins, etoposide, doxorubicin, mitoxantrone, teniposide, novobiocin, aclarubicin, anthracyclines, actinomycin, bleomycin, plicamycin, mitomycin, daunorubicin, epirubicin, idarubicin, dolastatins, maytansines, docetaxel, adriamycin, calicheamicin, auristatins, pyrrolobenzodiazepine, carboplatin, 5-fluorouracil (5-FU), capecitabine, mitomycin C, paclitaxel, 1,3-Bis(2-chloroethyl)-1-nitrosourea (BCNU), rifampicin, cisplatin, methotrexate, and gemcitabine.

In certain embodiments, the additional exogenous material comprises a protein or polypeptide comprising an enzyme. In certain other embodiments, the additional exogenous material is a nucleic acid, such as, e.g. a ribonucleic acid that functions as a small inhibiting RNA (siRNA) or microRNA (miRNA). In certain embodiments, the additional exogenous material is an antigen, such as antigens derived from bacterial proteins, viral proteins, proteins mutated in cancer, proteins aberrantly expressed in cancer, or T-cell complementary determining regions. For example, exogenous materials include antigens, such as those characteristic of antigen-presenting cells infected by bacteria, and T-cell complementary determining regions capable of functioning as exogenous antigens.

### F. Information Gathering for Diagnostic Functions

Certain cell-targeted molecules of the present invention have uses in the in vitro and/or in vivo detection of specific cells, cell types, and/or cell populations. In certain embodiments, the cytotoxic cell-targeted molecules described herein are used for both diagnosis and treatment, or for diagnosis alone. When the same cytotoxic cell-targeted molecule is used for both diagnosis and treatment, the cytotoxic cell-targeted molecule variant which incorporates a detection promoting agent for diagnosis may be rendered non-toxic by catalytic inactivation of a Shiga toxin effector region via one or more amino acid substitutions, including exemplary substitutions described herein. Catalytically inactive forms of the cytotoxic cell-targeted molecules of the invention that are conjugated to detection promoting agents optionally may be used for diagnostic functions, such as for companion diagnostics used in conjunction with a therapeutic regimen comprising the same or a related binding region.

The ability to conjugate detection promoting agents known in the art to various cell-targeted molecules of the invention provides useful compositions for the detection of cancer, tumor, immune, and infected cells. These diagnostic embodiments of the cell-targeted molecules of the invention may be used for information gathering via various imaging techniques and assays known in the art. For example, diagnostic embodiments of the cell-targeted molecules of the present invention may be used for information gathering via imaging of intracellular organelles (e.g. endocytotic, Golgi, endoplasmic reticulum, and cytosolic compartments) of individual cancer cells, immune cells, or infected cells in a patient or biopsy sample.

Various types of information may be gathered using the diagnostic embodiments of the cell-targeted molecules of the present invention whether for diagnostic uses or other uses. This information may be useful, for example, in diagnosing neoplastic cell types, determining therapeutic susceptibilities of a patient's disease, assaying the progression of antineoplastic therapies over time, assaying the progression of immunomodulatory therapies over time, assaying the progression of antimicrobial therapies over time, evaluating the presence of infected cells in transplantation materials, evaluating the presence of unwanted cell types in transplantation materials, and/or evaluating the presence of residual tumor cells after surgical excision of a tumor mass.

For example, subpopulations of patients might be ascertained using information gathered using the diagnostic variants of the cell-targeted molecules of the present invention, and then individual patients could be categorized into subpopulations based on their unique characteristic(s) revealed using those diagnostic embodiments. For example, the effectiveness of specific pharmaceuticals or therapies might be one type of criterion used to define a patient subpopulation. For example, a non-toxic diagnostic variant of a particular cytotoxic cell-targeted molecule of the invention may be used to differentiate which patients are in a class or subpopulation of patients predicted to respond positively to a cytotoxic variant of the same cell-targeted molecule of the invention. Accordingly, associated methods for patient identification, patient stratification and diagnosis using cell-targeted molecules of the invention and/or their non-toxic variants are considered to be within the scope of the present invention.

### III. Examples of Specific Structural Variations of the Cell-Targeted Molecules of the Present Invention

Among certain embodiments, the cell-targeted molecules of the present invention comprise the Shiga toxin effector region comprising or consisting essentially of amino acids 75 to 251 of SLT-1A (SEQ ID NO:1), StxA (SEQ ID NO:2), and/or SLT-2A (SEQ ID NO:3). Further embodiments are cell-targeted molecules in which the Shiga toxin effector region comprises or consists essentially of amino acids 1 to 241 of SLT-1A (SEQ ID NO:1), StxA (SEQ ID NO:2), and/or SLT-2A (SEQ ID NO:3). Further embodiments are cell-targeted molecules in which the Shiga toxin effector region comprises or consists essentially of amino acids 1 to 251 of SLT-1A (SEQ ID NO:1), StxA (SEQ ID NO:2), and/or SLT-2A (SEQ ID NO:3). Further embodiments are cell-targeted molecules in which the Shiga toxin effector region comprises or consists essentially of amino acids 1 to 261 of SLT-1A (SEQ ID NO:1), StxA (SEQ ID NO:2), and/or SLT-2A (SEQ ID NO:3).

In certain embodiments, the cell-targeted molecules of the present invention comprise the protease-cleavage resistant, Shiga toxin effector region comprising a mutation in the minimal, furin-cleavage motif altering at least one amino acid residue in the R/Y-x-x-R furin cleavage motif between the carboxy-terminus of a Shiga toxin A1 fragment region and the binding region. In certain further embodiments, the protease-cleavage resistant, Shiga toxin effector region comprises the mutation altering at least one amino acid residue in the region 1) natively positioned at amino acid residues 248 to 251 of the A Subunit of Shiga-like toxin 1 (SEQ ID NO: 1) or Shiga toxin (SEQ ID NO: 2), or 2) natively positioned at amino acid residues 247 to 250 of the A Subunit of Shiga-like toxin 2 (SEQ ID NO:3). In certain other embodiments, the protease-cleavage resistant, Shiga toxin effector region comprises the deletion of the region 1) natively positioned at amino acid residues 248 to 251 of the A Subunit of Shiga-like toxin 1 (SEQ ID NO: 1) or Shiga toxin (SEQ ID NO: 2), or 2) natively positioned at amino acid residues 247 to 250 of the A Subunit of Shiga-like toxin 2 (SEQ ID NO:3).

In certain embodiments, the cell-targeted molecule of the present invention comprises the protease-cleavage resistant, Shiga toxin effector region comprising the polypeptide represented by amino acids 2 to 252 of any one of SEQ ID NOs: 32-35.

In certain embodiments, the cell-targeted molecules of the present invention comprise the immunoglobulin-type binding region comprising or consisting essentially of amino acids 269-508 of SEQ ID NO:4, which exhibits high affinity binding specifically to human CD38. Further embodiments are the cell-targeted molecules comprising or consisting essentially of any one of the polypeptides shown in SEQ ID NOs: 4-7.

In certain embodiments, the cell-targeted molecules of the present invention comprise the immunoglobulin-type binding region comprising or consisting essentially of amino acids 269-512 of SEQ ID NO:8, which exhibits high affinity binding specifically to human HER2. Further embodiments are the cell-targeted molecules comprising or consisting essentially of any one of the polypeptides shown in SEQ ID NOs: 8-11.

In certain embodiments, the cell-targeted molecules comprise the immunoglobulin-type binding region comprising or consisting essentially of amino acids 269-516 of SEQ ID NO:12, which exhibits high affinity binding specifically to human CD19. Further embodiments are the cell-targeted molecules comprising or consisting essentially of any one of the polypeptides shown in SEQ ID NOs: 12-15.

In certain embodiments, the cell-targeted molecules comprise the immunoglobulin-type binding region comprising or consisting essentially of amino acids 269-518 of SEQ ID NO:16, which exhibits high affinity binding specifically to human CD74. Further embodiments are the cell-targeted molecules comprising or consisting essentially of any one of the polypeptides shown in SEQ ID NOs: 16-19.

Among certain embodiments of the present invention, the immunoglobulin-type binding region is derived from a nanobody or single domain immunoglobulin-derived V_{H}H or V_{H} region. Generally, nanobodies are constructed from fragments of naturally occurring single, monomeric variable domain antibodies (sdAbs) of the sort found in camelids and cartilaginous fishes (Chondrichthyes). Nanobodies are engineered from these naturally occurring antibodies by truncating the single, monomeric variable domain to create a smaller and more stable molecule. Due to their small size, nanobodies are able to bind to antigens that are not accessible to whole antibodies.

Among certain embodiments of the cell-targeted molecules of the present invention, the binding region is a single domain immunoglobulin-derived region V_{H}H which exhibits high affinity binding specifically to HER2, such as derived from a single-domain variable region of the camelid antibody (V_{H}H) protein 5F7, as described in U.S. Patent Application Publication 2011/0059090. In certain further embodiments, the cell-targeted molecules of the present invention comprise the immunoglobulin-type binding region comprising or consisting essentially of amino acids 268-385 of SEQ ID NO:20, which exhibits high affinity binding specifically to human HER2. Further embodiments are the cell-targeted molecules comprising or consisting essentially of any one of the polypeptides shown in SEQ ID NOs: 20-27.

For certain embodiments, the cell-targeted molecule comprises or consists essentially of the polypeptide shown in any one of SEQ ID NOs: 4-7. These CD38 binding, cell-targeted molecules may be used to treat and/or diagnose hematological cancers and/or immune disorders, such as, e.g., leukemia, myeloma, lymphoma, post-transplant lymphoproliferations, heavy chain disease, monoclonal gammopathy, monoclonal immunoglobulin deposition disease, myelodysplastic syndromes (MDS), smoldering multiple myeloma, and Waldenstrom macroglobulinemia.

For certain embodiments, the cell-targeted molecule comprises or consists essentially of the polypeptide shown in any one of SEQ ID NOs: 8-11, 20-27, 32, and 35. These HER-2 binding, cell-targeted molecules may be used to treat and/or diagnose cancers, such as, e.g., breast cancer, cervical cancer, colorectal cancer, endometrial cancer, esophageal cancer, gastrointestinal cancer (e.g. colorectal, esophageal, and gastric), head and neck cancer (e.g. oropharynx), kidney-urinary tract cancer (e.g. bladder and uterine), lung/pleura cancer, ovarian cancer, pancreatic cancer, prostate cancer, and testicular cancer.

For certain embodiments, the cell-targeted molecule comprises or consists essentially of the polypeptide shown in any one of SEQ ID NOs: 12-15 and 33. These CD19 binding, cell-targeted molecules may be used to treat and/or diagnose B-cell- or antibody-mediated diseases or disorders, such as for example leukemia, lymphoma, myeloma, amyloidosis, ankylosing spondylitis, asthma, Crohn's disease, diabetes, graft rejection, graft-versus-host disease, Hashimoto's thyroiditis, hemolytic uremic syndrome, Human Immunodeficiency Virus-related diseases (HIV) related diseases, lupus erythematosus, multiple sclerosis, polyarteritis nodosa, polyarthritis, psoriasis, psoriatic arthritis, rheumatoid arthritis, scleroderma, septic shock, Sjorgren's syndrome, ulcerative colitis, and vasculitis.

For certain embodiments, the cell-targeted molecule comprises or consists essentially of the polypeptide shown in any one of SEQ ID NOs: 16-19 and 34. These CD74 binding, cell-targeted molecules may be used to treat and/or diagnose B-cell-, plasma cell-, or antibody-mediated disease or disorder, such as for example leukemia, lymphoma, myeloma, amyloidosis, ankylosing spondylitis, asthma, Crohn's disease, diabetes, graft rejection, graft-versus-host disease, Hashimoto's thyroiditis, hemolytic uremic syndrome, Human Immunodeficiency Virus-related diseases (HIV) related diseases, lupus erythematosus, multiple sclerosis, polyarteritis nodosa, polyarthritis, psoriasis, psoriatic arthritis, rheumatoid arthritis, scleroderma, septic shock, Sjorgren's syndrome, ulcerative colitis, vasculitis, bone cancer, breast cancer, cervical cancer, head-neck cancer (e.g. oropharynx), gastrointestinal cancer (e.g. esophageal and colorectal), kidney-urinary tract cancer (e.g. bladder), lung/pleura cancer, ovarian cancer, leukemia, lymphoma, melanoma, myeloma, amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis, psoriasis, psoriatic arthritis, rheumatoid arthritis, scleroderma, septic shock, Sjorgren's syndrome, ulcerative colitis, and/or vasculitis.

For certain embodiments, the cell-targeted molecule comprises or consists essentially of the polypeptide shown in any one of SEQ ID NOs: 28-31. These CD20-binding, cell-targeted molecule embodiments may be used to treat and/or diagnose B-cell-, plasma cell-, or antibody-mediated disease or disorder, such as for example leukemia, lymphoma, myeloma, amyloidosis, ankylosing spondylitis, asthma, Crohn's disease, diabetes, graft rejection, graft-versus-host disease, Hashimoto's thyroiditis, hemolytic uremic syndrome, Human Immunodeficiency Virus-related diseases (HIV) related diseases, lupus erythematosus, multiple sclerosis, polyarteritis nodosa, polyarthritis, psoriasis, psoriatic arthritis, rheumatoid arthritis, scleroderma, septic shock, Sjorgren's syndrome, ulcerative colitis, vasculitis, bone cancer, breast cancer, cervical cancer, head-neck cancer (e.g. oropharynx), gastrointestinal cancer (e.g. esophageal and colorectal), kidney-urinary tract cancer (e.g. bladder), lung/pleura cancer, ovarian cancer, leukemia, lymphoma, melanoma, myeloma, amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis, psoriasis, psoriatic arthritis, rheumatoid arthritis, scleroderma, septic shock, Sjorgren's syndrome, ulcerative colitis, and/or vasculitis.

It is within the scope of the present invention to use fragments, variants, and/or derivatives of the polypeptides of the cell-targeted molecules of the present invention which contain a functional extracellular target biomolecule binding site, and even more preferably capable of binding the target biomolecule with high affinity (e.g. as shown by K_{D}). For example, while the invention provides polypeptide sequences that can bind to CD19, CD20, CD38, CD74, and HER2, any binding region that binds to an extracellular target biomolecule physically-linked to a cell (e.g. expressed on a cell surface) with a dissociation constant of 10⁻⁵ to 10⁻¹² moles per liter, preferably less than 200 nM, may be substituted for use in making cell-targeted molecules and methods of the present invention.

IV. Variations in the Polypeptide Sequences of the Cell-Targeted Molecules of the Present Invention which Maintain Overall Structure and Function

The skilled worker will recognize that variations may be made to the cell-targeted molecules of the present invention (and polynucleotides encoding them) without diminishing their biological activities, e.g. by maintaining the overall structure and function of the cell-targeted molecule of the invention. For example, some modifications may facilitate expression, purification, pharmacokinetic properties, and/or immunogenicity. Such modifications are well known to the skilled worker and include, for example, a methionine added at the amino-terminus to provide an initiation site, additional amino acids placed on either terminus to create conveniently located restriction sites or termination codons, and biochemical affinity tags fused to either terminus to provide for convenient detection and/or purification.

Also contemplated herein is the inclusion of additional amino acid residues at the amino and/or carboxy termini, such as sequences for epitope tags or other moieties. The additional amino acid residues may be used for various purposes including, e.g., to facilitate cloning, expression, post-translational modification, synthesis, purification, detection, and/or administration. Non-limiting examples of epitope tags and moieties are: chitin binding protein domains, enteropeptidase cleavage sites, Factor Xa cleavage sites, FIAsH tags, FLAG tags, green fluorescent proteins (GFP), glutathione-S-transferase moieties, HA tags, maltose binding protein domains, myc tags, polyhistidine tags, ReAsH tags, strep-tags, strep-tag II, TEV protease sites, thioredoxin domains, thrombin cleavage site, and VS epitope tags.

In certain of the above embodiments, the cell-targeted molecule of the present invention is a variant in which there are one or more conservative amino acid substitutions introduced into the polypeptide region(s). As used herein, the term “conservative substitution” denotes that one or more amino acids are replaced by another, biologically similar amino acid residue. Examples include substitution of amino acid residues with similar characteristics, e.g. small amino acids, acidic amino acids, polar amino acids, basic amino acids, hydrophobic amino acids and aromatic amino acids (see, for example, Table B below). An example of a conservative substitution with a residue normally not found in endogenous, mammalian peptides and proteins is the conservative substitution of an arginine or lysine residue with, for example, ornithine, canavanine, aminoethylcysteine, or another basic amino acid. For further information concerning phenotypically silent substitutions in peptides and proteins (see e.g. Bowie J et al., Science 247: 1306-10 (1990)).

TABLE B
Examples of Conservative Amino Acid Substitutions

| I | II | III | IV | V | VI | VII | VIII | IX | X | XI | XII | XIII | XIV |
|---|---|---|---|---|---|---|---|---|---|---|---|---|---|
| A | D | H | C | F | N | A | C | F | A | C | A | A | D |
| G | E | K | I | W | Q | G | M | H | C | D | C | C | E |
| P | Q | R | L | Y | S | I | P | W | F | E | D | D | G |
| S | N | | M | | T | L | | Y | G | H | G | E | K |
| T | | | V | | | V | | | H | K | N | G | P |
| | | | | | | | | | I | N | P | H | Q |
| | | | | | | | | | L | Q | S | K | R |
| | | | | | | | | | M | R | T | N | S |
| | | | | | | | | | R | S | V | Q | T |
| | | | | | | | | | T | T | | R | |
| | | | | | | | | | V | | | S | |
| | | | | | | | | | W | | | P | |
| | | | | | | | | | Y | | | T | |

In the conservative substitution scheme in Table B, exemplary conservative substitutions of amino acids are grouped by physicochemical properties—I: neutral, hydrophilic; II: acids and amides; III: basic; IV: hydrophobic; V: aromatic, bulky amino acids, VI hydrophilic uncharged, VII aliphatic uncharged, VIII non-polar uncharged, IX cycloalkenyl-associated, X hydrophobic, XI polar, XII small, XIII turn-permitting, and XIV flexible. For example, conservative amino acid substitutions include the following: 1) S may be substituted for C; 2) M or L may be substituted for F; 3) Y may be substituted for M; 4) Q or E may be substituted for K; 5) N or Q may be substituted for H; and 6) H may be substituted for N.

In certain embodiments, a cell-targeted molecule of the present invention may comprise functional fragments or variants of a polypeptide region of the invention that have, at most, 20, 15, 10, 9, 8, 7, 6, 5, 4, 3, 2, or 1 amino acid substitution(s) compared to a polypeptide sequence recited herein, as long as the substituted polypeptide region retains measurable biological activity alone or as a component of a cell-targeted molecule of the invention. Variants of cell-targeted molecules of the invention are within the scope of the invention as a result of changing a polypeptide of the cell-targeted molecule of the invention by altering one or more amino acids or deleting or inserting one or more amino acids, such as within the binding region or the Shiga toxin effector region, in order to achieve desired properties, such as changed cytotoxicity, changed cytostatic effects, changed immunogenicity, and/or changed serum half-life. A polypeptide of a cell-targeted molecule of the invention may further be with or without a signal sequence.

In certain embodiments, a cell-targeted molecule of the present invention shares at least 85%, 90%, 95%, 96%, 97%, 98%, 99% or more amino acid sequence identity to any one of the amino acid sequences of a protein recited herein, as long as it retains measurable biological activity, such as cytotoxicity, extracellular target biomolecule binding, enzymatic catalysis, or subcellular routing. The binding region may differ from the amino acid sequences of a protein recited herein, as long as it retains binding functionality to its extracellular target biomolecule. Binding functionality will most likely be retained if the amino acid sequences of the CDRs or ABRs are identical. For example, a protein is within the claim scope that comprises or consists essentially of 85% amino acid identity to a protein recited herein which for the purposes of determining the degree of amino acid identity, the amino acid residues that form the CDRs or ABRs are disregarded. Binding functionality can be determined by the skilled worker using standard techniques.

The invention further provides variants of the cell-targeted molecules of the present invention, wherein the Shiga toxin effector region differs from a naturally occurring Shiga toxin A Subunit by up to 1, 2, 3, 4, 5, 6, 7, 8, 9, 10, 15, 20, 25, 30, 35, 40 or more amino acid residues (but by no more than that which retains at least 85%, 90%, 95%, 99% or more amino acid sequence identity). Thus, a polypeptide region derived from an A Subunit of a member of the Shiga toxin family may comprise additions, deletions, truncations, or other alterations from the original sequence as long as at least 85%, 90%, 95%, 99% or more amino acid sequence identity is maintained to a naturally occurring Shiga toxin A Subunit.

In certain of the above embodiments, the polypeptide sequences of the protease-cleavage resistant, Shiga toxin effector polypeptide component of a molecule of the present invention is varied by one or more conservative amino acid substitutions as long as the Shiga toxin effector polypeptide retains a disrupted furin-cleavage motif and as long as the Shiga toxin effector polypeptide exhibits, alone and/or as a component of a cell-targeted molecule, one or more Shiga toxin effector functions selected from one or more of the following: intracellular routing, catalytic activity, and/or cytotoxicity. In certain of the above embodiments, the polypeptide sequences of the cell-targeted molecules of the present invention are varied by one or more conservative amino acid substitutions introduced into a polypeptide region(s) the Shiga toxin effector polypeptide region retains a disrupted furin-cleavage motif and as long as the binding region retains extracellular target biomolecule binding specificity.

Accordingly, in certain embodiments, the Shiga toxin effector region comprises or consists essentially of amino acid sequences having at least 55%, 60%, 65%, 70%, 75%, 80%, 85%, 90%, 95%, 97%, 98%, 99%, 99.5% or 99.7% overall sequence identity to a naturally occurring Shiga toxin A Subunit, such as SLT-1A (SEQ ID NO:1), Stx (SEQ ID NO:2), and/or SLT-2A (SEQ ID NO:3).

Optionally, either a full length or a truncated version of the Shiga toxin A Subunit may comprise one or more mutations (e.g. substitutions, deletions, insertions or inversions). In certain embodiments, it is preferred that the Shiga toxin effector region has sufficient sequence identity to a naturally occurring Shiga toxin A Subunit to retain cytotoxicity after entry into a cell, either by well-known methods of host cell transformation, transfection, infection or induction, or by internalization mediated by the cell-targeting, binding region linked with the Shiga toxin effector region. The most critical residues for enzymatic activity and/or cytotoxicity in the Shiga toxin A Subunits have been mapped to the following residue-positions: aspargine-75, tyrosine-77, glutamate-167, arginine-170, and arginine-176 among others (Di R, Toxicon 57: 535-39 (2011)). In any one of the embodiments of the invention, the Shiga toxin effector region may preferably but not necessarily maintain one or more conserved amino acids at positions, such as those found at positions 77, 167, 170, and 176 in StxA, SLT-1A, or the equivalent conserved position in other members of the Shiga toxin family which are typically required for cytotoxic activity. The capacity of a cytotoxic cell-targeted molecule of the invention to cause cell death, e.g. its cytotoxicity, may be measured using any one or more of a number of assays well known in the art.

In certain embodiments, the Shiga toxin effector region may be altered to change its enzymatic activity and/or cytotoxicity as long as the Shiga toxin effector region retains one or more other Shiga toxin effector functions. This change may or may not result in a change in the cytotoxicity of a cell-targeted molecule of which the altered Shiga toxin effector region is a component. Possible alterations include mutations to the Shiga toxin effector region selected from the group consisting of: a truncation, deletion, inversion, insertion, rearrangement, and substitution.

The catalytic and/or cytotoxic activity of the A Subunits of members of the Shiga toxin family may be reduced or eliminated by mutation or truncation. The positions labeled tyrosine-77, glutamate-167, arginine-170, tyrosine-114, and tryptophan-203 have been shown to be important for the catalytic activity of Stx, Stx1, and Stx2 (Hovde C et al., Proc Natl Acad Sci USA 85: 2568-72 (1988); Deresiewicz R et al., Biochemistry 31: 3272-80 (1992); Deresiewicz R et al., Mol Gen Genet 241: 467-73 (1993); Ohmura M et al., Microb Pathog 15: 169-76 (1993); Cao C et al., Microbiol Immunol 38: 441-7 (1994); Suhan M, Hovde C, Infect Immun 66: 5252-9 (1998)). Mutating both glutamate-167 and arginine-170 eliminated the enzymatic activity of Slt-I A1 in a cell-free ribosome inactivation assay (LaPointe P et al., J Biol Chem 280: 23310-18 (2005)). In another approach using de novo expression of Slt-I A1 in the endoplasmic reticulum, mutating both glutamate-167 and arginine-170 eliminated Slt-I A1 fragment cytotoxicity at that expression level (LaPointe P et al., J Biol Chem 280: 23310-18 (2005)). A truncation analysis demonstrated that a fragment of StxA from residues 75 to 268 still retains significant enzymatic activity in vitro (Haddad J et al., J Bacteriol 175: 4970-8 (1993)). A truncated fragment of Slt-I A1 containing residues 1-239 displayed significant enzymatic activity in vitro and cytotoxicity by de novo expression in the cytosol (LaPointe P et al., J Biol Chem 280: 23310-18 (2005)). Expression of a Slt-I A1 fragment truncated to residues 1-239 in the endoplasmic reticulum was not cytotoxic because it could not retrotranslocate into the cytosol (LaPointe P et al., J Biol Chem 280: 23310-18 (2005)).

The most critical residues for enzymatic activity and/or cytotoxicity in the Shiga toxin A Subunits have been mapped to the following residue-positions: aspargine-75, tyrosine-77, glutamate-167, arginine-170, and arginine-176 among others (Di R, Toxicon 57: 535-39 (2011)). In particular, a double-mutant construct of Stx2A containing glutamate-E167-to-lysine and arginine-176-to-lysine mutations was completely inactivated; whereas, many single mutations in Stx1 and Stx2 showed a 10-fold reduction in cytotoxicity. Further, truncation of Stx1A to 1-239 or 1-240 reduced its cytotoxicity, and similarly, truncation of Stx2A to a conserved hydrophobic residue reduced its cytotoxicity.

The most critical residues for binding eukaryotic ribosomes and/or eukaryotic ribosome inhibition in the Shiga toxin A Subunit have been mapped to the following residue-positions arginine-172, arginine-176, arginine-179, arginine-188, tyrosine-189, valine-191, and leucine-233 among others (McCluskey A et al., PLoS One 7: e31191 (2012).

Shiga-like toxin 1 A Subunit truncations are catalytically active, capable of enzymatically inactivating ribosomes in vitro, and cytotoxic when expressed within a cell (LaPointe P et al., J Biol Chem 280: 23310-18 (2005)). The smallest Shiga toxin A Subunit fragment exhibiting full enzymatic activity is a polypeptide composed of residues 1-239 of Slt1A (LaPointe P et al., J Biol Chem 280: 23310-18 (2005)). Although the smallest fragment of the Shiga toxin A Subunit reported to retain substantial catalytic activity was residues 75-247 of StxA (Al-Jaufy, Infect Immun 62: 956-60 (1994)), a StxA truncation expressed de novo within a eukaryotic cell requires only up to residue 240 to reach the cytosol and exert catalytic inactivation of ribosomes (LaPointe P et al., J Biol Chem 280: 23310-18 (2005)).

In certain embodiments derived from SLT-1A (SEQ ID NO:1) or StxA (SEQ ID NO:2), these changes include substitution of the asparagine at position 75, tyrosine at position 77, tyrosine at position 114, glutamate at position 167, arginine at position 170, arginine at position 176, and/or substitution of the tryptophan at position 203. Examples of such substitutions will be known to the skilled worker based on the prior art, such as asparagine at position 75 to alanine, tyrosine at position 77 to serine, substitution of the tyrosine at position 114 to serine, substitution of the glutamate at position 167 to aspartate, substitution of the arginine at position 170 to alanine, substitution of the arginine at position 176 to lysine, and/or substitution of the tryptophan at position 203 to alanine.

Cell-targeted molecules of the present invention may optionally be conjugated to one or more additional agents, such as, e.g., therapeutic and/or diagnostic agents known in the art, including such agents as described herein.

### V. Production, Manufacture, and Purification of a Cell-Targeted Molecule of the Present Invention

The cell-targeted molecules of the present invention may be produced using biochemical engineering techniques well known to those of skill in the art. For example, cytotoxic of the invention may be manufactured by standard synthetic methods, by use of recombinant expression systems, or by any other suitable method. The cell-targeted molecules of the invention may be produced as fusion proteins, chemically coupled conjugates, and/or combinations thereof, such as, e.g., a fusion protein component covalently coupled to one or more components. Thus, the cell-targeted molecules of the invention may be synthesized in a number of ways, including, e.g. methods comprising: (1) synthesizing a polypeptide or polypeptide component of a cell-targeted molecule of the invention using standard solid-phase or liquid-phase methodology, either stepwise or by fragment assembly, and isolating and purifying the final polypeptide compound product; (2) expressing a polynucleotide that encodes a polypeptide or polypeptide component of a cell-targeted molecule of the invention in a host cell and recovering the expression product from the host cell or host cell culture; or (3) cell-free in vitro expression of a polynucleotide encoding a polypeptide or polypeptide component of a cell-targeted molecule of the invention, and recovering the expression product; or by any combination of the methods of (1), (2) or (3) to obtain fragments of the peptide component, subsequently joining (e.g. ligating) the fragments to obtain the peptide component, and recovering the peptide component. For example, polypeptide and/or peptide components may be ligated together using coupling reagents, such as, e.g., N,N′-dicyclohexycarbodiimide and N-ethyl-5-phenyl-isoxazolium-3′-sulfonate (Woodward's reagent K).

It may be preferable to synthesize a polypeptide or polypeptide component of a cell-targeted molecule of the present invention by means of solid-phase or liquid-phase peptide synthesis. Cell-targeted molecules of the invention may suitably be manufactured by standard synthetic methods. Thus, peptides may be synthesized by, e.g. methods comprising synthesizing the peptide by standard solid-phase or liquid-phase methodology, either stepwise or by fragment assembly, and isolating and purifying the final peptide product. In this context, reference may be made to WO 1998/11125 or, inter alia, Fields G et al., Principles and Practice of Solid-Phase Peptide Synthesis (Synthetic Peptides, Grant G, ed., Oxford University Press, U.K., 2nd ed., 2002) and the synthesis examples therein.

Cell-targeted molecules of the present invention may be prepared (produced and purified) using recombinant techniques well known in the art. In general, methods for preparing polypeptides by culturing host cells transformed or transfected with a vector comprising the encoding polynucleotide and recovering the polypeptide from cell culture are described in, e.g. Sambrook J et al., Molecular Cloning: A Laboratory Manual (Cold Spring Harbor Laboratory Press, NY, U.S., 1989); Dieffenbach C et al., PCR Primer: A Laboratory Manual (Cold Spring Harbor Laboratory Press, N.Y., U.S., 1995). Any suitable host cell may be used to produce a cell-targeted molecule of the invention. Host cells may be cells stably or transiently transfected, transformed, transduced or infected with one or more expression vectors which drive expression of a polypeptide of the invention. In addition, a protein of the invention may be produced by modifying the polynucleotide encoding the protein of the invention that result in altering one or more amino acids or deleting or inserting one or more amino acids in order to achieve desired properties, such as changed cytotoxicity, changed cytostatic effects, changed immunogenicity, and/or changed serum half-life.

There are a wide variety of expression systems which may be chosen to produce a cell-targeted molecule of the present invention. For example, host organisms for expression of cell-targeted molecules of the invention include prokaryotes, such as E. coli and B. subtilis, eukaryotic cells, such as yeast and filamentous fungi (like S. cerevisiae, P. pastoris, A. awamori, and K. lactis), algae (like C. reinhardtii), insect cell lines, mammalian cells (like CHO cells), plant cell lines, and eukaryotic organisms such as transgenic plants (like A. thaliana and N. benthamiana).

Accordingly, the present invention also provides methods for producing a cell-targeted molecule of the present invention according to above recited methods and using (i) a polynucleotide encoding part or all of a cell-targeted molecule of the invention or a polypeptide component thereof, (ii) an expression vector comprising at least one polynucleotide of the invention capable of encoding part or all of a cell-targeted molecule of the invention or a polypeptide component thereof when introduced into a suitable host cell or cell-free expression system, and/or (iii) a host cell comprising a polynucleotide or expression vector of the invention.

When a polypeptide or protein is expressed using recombinant techniques in a host cell or cell-free system, it is advantageous to separate (or purify) the desired polypeptide or protein away from other components, such as host cell factors, in order to obtain preparations that are of high purity or are substantially homogeneous. Purification can be accomplished by methods well known in the art, such as centrifugation techniques, extraction techniques, chromatographic and fractionation techniques (e.g. size separation by gel filtration, charge separation by ion-exchange column, hydrophobic interaction chromatography, reverse phase chromatography, chromatography on silica or cation-exchange resins such as DEAE and the like, chromatofocusing, and Protein A Sepharose chromatography to remove contaminants), and precipitation techniques (e.g. ethanol precipitation or ammonium sulfate precipitation). Any number of biochemical purification techniques may be used to increase the purity of a cell-targeted molecule of the invention. In certain embodiments, the cell-targeted molecules of the invention may optionally be purified in homo-multimeric forms (e.g., a protein complex of two or more identical proteins) or in hetero-multimeric forms (e.g., a protein complex of two or more non-identical proteins).

In the Examples, below, are descriptions of non-limiting examples of methods for producing a cell-targeted molecule of the present invention, as well as specific but non-limiting aspects of protein production for certain, disclosed, cytotoxic, cell-targeted, fusion proteins.

### VI. Pharmaceutical and Diagnostic Compositions Comprising a Cell-Targeted Molecule of the Present Invention

The present invention provides cell-targeted molecules for use, alone or in combination with one or more additional therapeutic agents, in a pharmaceutical composition, for treatment or prophylaxis of conditions, diseases, disorders, or symptoms described in further detail below (e.g. cancers, malignant tumors, non-malignant tumors, growth abnormalities, immune disorders, and microbial infections). The present invention further provides pharmaceutical compositions comprising a cell-targeted molecule of the invention, or a pharmaceutically acceptable salt or solvate thereof, according to the invention, together with at least one pharmaceutically acceptable carrier, excipient, or vehicle. In certain embodiments, the pharmaceutical composition of the invention may comprise homo-multimeric and/or hetero-multimeric forms of the cell-targeted molecules of the invention. The pharmaceutical compositions will be useful in methods of treating, ameliorating, or preventing a disease, condition, disorder, or symptom described in further detail below. Each such disease, condition, disorder, or symptom is envisioned to be a separate embodiment with respect to uses of a pharmaceutical composition according to the invention. The invention further provides pharmaceutical compositions for use in at least one method of treatment according to the invention, as described in more detail below.

As used herein, the terms “patient” and “subject” are used interchangeably to refer to any organism, commonly vertebrates such as humans and animals, which presents symptoms, signs, and/or indications of at least one disease, disorder, or condition. These terms include mammals such as the non-limiting examples of primates, livestock animals (e.g. cattle, horses, pigs, sheep, goats, etc.), companion animals (e.g. cats, dogs, etc.) and laboratory animals (e.g. mice, rabbits, rats, etc.).

As used herein, “treat,” “treating,” or “treatment” and grammatical variants thereof refer to an approach for obtaining beneficial or desired clinical results. The terms may refer to slowing the onset or rate of development of a condition, disorder or disease, reducing or alleviating symptoms associated with it, generating a complete or partial regression of the condition, or some combination of any of the above. For the purposes of this invention, beneficial or desired clinical results include, but are not limited to, reduction or alleviation of symptoms, diminishment of extent of disease, stabilization (e.g. not worsening) of state of disease, delay or slowing of disease progression, amelioration or palliation of the disease state, and remission (whether partial or total), whether detectable or undetectable. “Treat,” “treating,” or “treatment” can also mean prolonging survival relative to expected survival time if not receiving treatment. A subject (e.g. a human) in need of treatment may thus be a subject already afflicted with the disease or disorder in question. The terms “treat,” “treating,” or “treatment” includes inhibition or reduction of an increase in severity of a pathological state or symptoms relative to the absence of treatment, and is not necessarily meant to imply complete cessation of the relevant disease, disorder, or condition. With regard to tumors and/or cancers, treatment includes reductions in overall tumor burden and/or individual tumor size.

As used herein, the terms “prevent,” “preventing,” “prevention” and grammatical variants thereof refer to an approach for preventing the development of, or altering the pathology of, a condition, disease, or disorder. Accordingly, “prevention” may refer to prophylactic or preventive measures. For the purposes of this invention, beneficial or desired clinical results include, but are not limited to, prevention or slowing of symptoms, progression or development of a disease, whether detectable or undetectable. A subject (e.g. a human) in need of prevention may thus be a subject not yet afflicted with the disease or disorder in question. The term “prevention” includes slowing the onset of disease relative to the absence of treatment, and is not necessarily meant to imply permanent prevention of the relevant disease, disorder or condition. Thus “preventing” or “prevention” of a condition may in certain contexts refer to reducing the risk of developing the condition, or preventing or delaying the development of symptoms associated with the condition.

As used herein, an “effective amount” or “therapeutically effective amount” is an amount or dose of a composition (e.g. a therapeutic composition or agent) that produces at least one desired therapeutic effect in a subject, such as preventing or treating a target condition or beneficially alleviating a symptom associated with the condition. The most desirable therapeutically effective amount is an amount that will produce a desired efficacy of a particular treatment selected by one of skill in the art for a given subject in need thereof. This amount will vary depending upon a variety of factors understood by the skilled worker, including but not limited to the characteristics of the therapeutic cell-targeted molecule of the present invention or composition thereof (including activity, pharmacokinetics, pharmacodynamics, and bioavailability), the physiological condition of the subject (including age, sex, disease type, disease stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a cell-targeted molecule of the present invention, or composition thereof, and adjusting the dosage accordingly (see e.g. Remington: The Science and Practice of Pharmacy (Gennaro A, ed., Mack Publishing Co., Easton, Pa., U.S., 19th ed., 1995)).

Diagnostic compositions of the present invention comprise a cell-targeted molecule of the invention and one or more detection promoting agents. Various detection promoting agents are known in the art, such as isotopes, dyes, colorimetric agents, contrast enhancing agents, fluorescent agents, bioluminescent agents, and magnetic agents. These agents may be incorporated into the cell-targeted molecule of the invention at any position. The incorporation of the agent may be via an amino acid residue(s) of the cell-targeted molecule of the invention or via some type of linkage known in the art, including via linkers and/or chelators. The incorporation of the agent is in such a way to enable the detection of the presence of the diagnostic composition in a screen, assay, diagnostic procedure, and/or imaging technique.

When producing or manufacturing a diagnostic composition of the present invention, a cell-targeted molecule of the invention may be directly or indirectly linked to one or more detection promoting agents. There are numerous detection promoting agents known to the skilled worker which can be operably linked to the cell-targeted molecules of the invention for information gathering methods, such as for diagnostic and/or prognostic applications to diseases, disorders, or conditions of an organism (see e.g. Cai W et al., J Nucl Med 48: 304-10 (2007); Nayak T, Brechbiel M, Bioconjug Chem 20: 825-41 (2009); Paudyal P et al., Oncol Rep 22: 115-9 (2009); Qiao J et al., PLoS ONE 6: e18103 (2011); Sano K et al., Breast Cancer Res 14: R61 (2012)). For example, detection promoting agents include image enhancing contrast agents, such as fluorescent dyes (e.g. Alexa680, indocyanine green, and Cy5.5), isotopes and radionuclides, such as ¹¹C, ¹³N, ¹⁵O, ¹⁸F, ³²P, ⁵¹Mn, ⁵²mMn, ⁵²Fe, ⁵⁵Co, ⁶²Cu, ⁶⁴Cu, ⁶⁷Cu, ⁶⁷Ga, ⁶⁸Ga, ⁷²As, ⁷³Se, ⁷⁵Br, ⁷⁶Br, ⁸²mRb, ⁸³Sr, ⁸⁶Y, ⁹⁰Y, ⁸⁹Zr, ⁹⁴mTc, ⁹⁴Tc, ⁹⁹mTc, ¹¹⁰In, ¹¹¹In, ¹²⁰I, ¹²³I, ¹²⁴I, ¹²⁵I, ¹³¹I, ¹⁵⁴Gd, ¹⁵⁵Gd, ¹⁵⁶Gd, ¹⁵⁷Gd, ¹⁵⁸Gd, ¹⁷⁷Lu, ¹⁸⁶Re, ¹⁸⁸Re, and ²²³R, paramagnetic ions, such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III), metals, such as lanthanum (III), gold (III), lead (II), and bismuth (III), ultrasound contrast enhancing agents, such as liposomes, radiopaque agents, such as barium, gallium, and thallium compounds. Detection promoting agents may be incorporated directly or indirectly by using an intermediary functional group, such as chelators like 2-benzyl DTPA, PAMAM, NOTA, DOTA, TETA, analogs thereof, and functional equivalents of any of the foregoing (see Leyton J et al., Clin Cancer Res 14: 7488-96 (2008)).

There are numerous standard techniques known to the skilled worker for incorporating, affixing, and/or conjugating various detection promoting agents to molecules, especially to immunoglobulins and immunoglobulin-derived domains (Wu A, Methods 65: 139-47 (2014)). Similarly, there are numerous imaging approaches known to the skilled worker, such as non-invasive in vivo imaging techniques commonly used in the medical arena, for example: computed tomography imaging (CT scanning), optical imaging (including direct, fluorescent, and bioluminescent imaging), magnetic resonance imaging (MRI), positron emission tomography (PET), single-photon emission computed tomography (SPECT) ultrasound, and x-ray computed tomography imaging (see Kaur S et al., Cancer Lett 315: 97-111 (2012), for review).

Production or Manufacture of a Pharmaceutical and/or Diagnostic Composition Comprising a Cell-Targeted Molecule of the Present Invention

Pharmaceutically acceptable salts or solvates of any of the cell-targeted molecules of the present invention are likewise within the scope of the present invention.

The term “solvate” in the context of the present invention refers to a complex of defined stoichiometry formed between a solute (in casu, a polypeptide compound or pharmaceutically acceptable salt thereof according to the invention) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

Cell-targeted molecules of the present invention, or salts thereof, may be formulated as pharmaceutical compositions prepared for storage or administration, which typically comprise a therapeutically effective amount of a compound of the invention, or a salt thereof, in a pharmaceutically acceptable carrier. The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences (Mack Publishing Co. (A. Gennaro, ed., 1985). As used herein, “pharmaceutically acceptable carrier” includes any and all physiologically acceptable, i.e. compatible, solvents, dispersion media, coatings, antimicrobial agents, isotonic, and absorption delaying agents, and the like. Pharmaceutically acceptable carriers or diluents include those used in formulations suitable for oral, rectal, nasal or parenteral (including subcutaneous, intramuscular, intravenous, intradermal, and transdermal) administration. Exemplary pharmaceutically acceptable carriers include sterile aqueous solutions or dispersions and sterile powders for the extemporaneous preparation of sterile injectable solutions or dispersions. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyloleate. Proper fluidity can be maintained, for example, by the use of coating materials, such as lecithin, by the maintenance of the required particle size in the case of dispersions, and by the use of surfactants. In certain embodiments, the carrier is suitable for intravenous, intramuscular, subcutaneous, parenteral, spinal or epidermal administration (e.g. by injection or infusion). Depending on selected route of administration, the cell-targeted molecule of the invention or other pharmaceutical component may be coated in a material intended to protect the compound from the action of low pH and other natural inactivating conditions to which the active cell-targeted molecule of the invention may encounter when administered to a patient by a particular route of administration.

The formulations of the pharmaceutical compositions of the present invention may conveniently be presented in unit dosage form and may be prepared by any of the methods well known in the art of pharmacy. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of a pen. Compositions may be formulated for any suitable route and means of administration. Subcutaneous or transdermal modes of administration may be particularly suitable for therapeutic cell-targeted molecules described herein.

The pharmaceutical compositions of the present invention may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. Isotonic agents, such as sugars, sodium chloride, and the like into the compositions, may also be desirable. In addition, prolonged absorption of the injectable pharmaceutical form may be brought about by the inclusion of agents which delay absorption such as, aluminum monostearate and gelatin.

A pharmaceutical composition of the present invention also optionally includes a pharmaceutically acceptable antioxidant. Exemplary pharmaceutically acceptable antioxidants are water soluble antioxidants such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propylgallate, alpha-tocopherol, and the like; and metal chelating agents, such as citric acid, ethylenediamine tetraacetic acid (EDTA), sorbitol, tartaric acid, phosphoric acid, and the like.

In another aspect, the present invention provides pharmaceutical compositions comprising one or a combination of different cell-targeted molecules of the invention, or an ester, salt or amide of any of the foregoing, and at least one pharmaceutically acceptable carrier.

Therapeutic compositions are typically sterile and stable under the conditions of manufacture and storage. The composition may be formulated as a solution, microemulsion, liposome, or other ordered structure suitable to high drug concentration. The carrier may be a solvent or dispersion medium containing, for example, water, alcohol such as ethanol, polyol (e.g. glycerol, propylene glycol, and liquid polyethylene glycol), or any suitable mixtures. The proper fluidity may be maintained, for example, by the use of a coating such as lecithin, by the maintenance of the required particle size in the case of dispersion and by use of surfactants according to formulation chemistry well known in the art. In certain embodiments, isotonic agents, e.g. sugars, polyalcohols such as mannitol, sorbitol, or sodium chloride may be desirable in the composition. Prolonged absorption of injectable compositions may be brought about by including in the composition an agent that delays absorption for example, monostearate salts and gelatin.

Solutions or suspensions used for intradermal or subcutaneous application typically include one or more of: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and tonicity adjusting agents such as, e.g., sodium chloride or dextrose. The pH can be adjusted with acids or bases, such as hydrochloric acid or sodium hydroxide, or buffers with citrate, phosphate, acetate and the like. Such preparations may be enclosed in ampoules, disposable syringes or multiple dose vials made of glass or plastic.

Sterile injectable solutions may be prepared by incorporating a cell-targeted molecule of the invention in the required amount in an appropriate solvent with one or a combination of ingredients described above, as required, followed by sterilization microfiltration. Dispersions may be prepared by incorporating the active cell-targeted molecule compound into a sterile vehicle that contains a dispersion medium and other ingredients, such as those described above. In the case of sterile powders for the preparation of sterile injectable solutions, the methods of preparation are vacuum drying and freeze-drying (lyophilization) that yield a powder of the active ingredient in addition to any additional desired ingredient from a sterile-filtered solution thereof.

When a therapeutically effective amount of a cell-targeted molecule of the invention is designed to be administered by, e.g. intravenous, cutaneous or subcutaneous injection, the binding agent will be in the form of a pyrogen-free, parenterally acceptable aqueous solution. Methods for preparing parenterally acceptable cell-targeted molecule solutions, taking into consideration appropriate pH, isotonicity, stability, and the like, are within the skill in the art. A preferred pharmaceutical composition for intravenous, cutaneous, or subcutaneous injection will contain, in addition to binding agents, an isotonic vehicle such as sodium chloride injection, Ringer's injection, dextrose injection, dextrose and sodium chloride injection, lactated Ringer's injection, or other vehicle as known in the art. A pharmaceutical composition of the present invention may also contain stabilizers, preservatives, buffers, antioxidants, or other additives well known to those of skill in the art.

As described elsewhere herein, a cell-targeted molecule of the present invention or composition thereof (e.g. pharmaceutical or diagnostic composition) may be prepared with carriers that will protect the cell-targeted molecule and/or composition against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art (see e.g. Sustained and Controlled Release Drug Delivery Systems (Robinson J, ed., Marcel Dekker, Inc., NY, U.S., 1978)).

In certain embodiments, the composition of the present invention (e.g. pharmaceutical or diagnostic composition) may be formulated to ensure a desired distribution in vivo. For example, the blood-brain barrier excludes many large and/or hydrophilic compounds. To target a therapeutic cell-targeted molecule or composition of the invention to a particular in vivo location, it can be formulated, for example, in liposomes which may comprise one or more moieties that are selectively transported into specific cells or organs, thus enhancing targeted drug delivery. Exemplary targeting moieties include folate or biotin; mannosides; antibodies; surfactant protein A receptor; p120 catenin and the like.

Pharmaceutical compositions include parenteral formulations designed to be used as implants or particulate systems. Examples of implants are depot formulations composed of polymeric or hydrophobic components such as emulsions, ion exchange resins, and soluble salt solutions. Examples of particulate systems are dendrimers, liposomes, microspheres, microparticles, nanocapsules, nanoparticles, nanorods, nanospheres, polymeric micelles, and nanotubes (see e.g. Honda M et al., Int J Nanomedicine 8: 495-503 (2013); Sharma A et al., Biomed Res Int 2013: 960821 (2013); Ramishetti S, Huang L, Ther Deliv 3: 1429-45 (2012)). Controlled release formulations may be prepared using polymers sensitive to ions, such as, e.g. liposomes, polaxamer 407, and hydroxyapatite. Particulate and polymer formulations may comprise a plasma membrane permeability altering agent(s), such as, e.g., various peptides and proteins like cytolysins, toxin-derived agents, virus-derived agents, synthetic biomimetic peptides, and chemical agents (see e.g. Varkouhi A et al., J Control Release 151: 220-8 (2011); Pirie C et al., Mol Cancer Ther 12: 1774-82 (2013)).

### VII. Polynucleotides, Expression Vectors, and Host Cells

Beyond the cell-targeted molecules of the present invention, the polynucleotides which encode such cell-targeted molecules, or functional portions thereof, are within the scope of the present invention. The term “polynucleotide” is equivalent to the term “nucleic acids” both of which include polymers of deoxyribonucleic acids (DNAs), polymers of ribonucleic acids (RNAs), analogs of these DNAs or RNAs generated using nucleotide analogs, and derivatives, fragments and homologs thereof. The polynucleotide of the invention may be single-, double-, or triple-stranded. Disclosed polynucleotides are specifically disclosed to include all polynucleotides capable of encoding an exemplary protein of the invention, for example, taking into account the wobble known to be tolerated in the third position of RNA codons, yet encoding for the same amino acid as a different RNA codon (see Stothard P, Biotechniques 28: 1102-4 (2000)).

In one aspect, the invention provides polynucleotides which encode a protein of the invention, or a fragment or derivative thereof. The polynucleotides may include, e.g., nucleic acid sequence encoding a polypeptide at least 50%, 55%, 60%, 65%, 70%, 75%, 80%, 85%, 90%, 95%, 99% or more, identical to a polypeptide comprising one of the amino acid sequences of the protein of the invention. The invention also includes polynucleotides comprising nucleotide sequences that hybridize under stringent conditions to a polynucleotide which encodes a protein of the invention, or a fragment or derivative thereof, or the antisense or complement of any such sequence.

Derivatives or analogs of the polynucleotides (or proteins) of the invention include, inter alia, polynucleotide (or polypeptide) molecules having regions that are substantially homologous to the polynucleotides or proteins of the invention, e.g. by at least about 45%, 50%, 70%, 80%, 95%, 98%, or even 99% identity (with a preferred identity of 80-99%) over a polynucleotide or polypeptide sequence of the same size or when compared to an aligned sequence in which the alignment is done by a computer homology program known in the art. An exemplary program is the GAP program (Wisconsin Sequence Analysis Package, Version 8 for UNIX, Genetics Computer Group, University Research Park, Madison, Wis., U.S.) using the default settings, which uses the algorithm of Smith T, Waterman M, Adv. Appl. Math. 2: 482-9 (1981). Also included are polynucleotides capable of hybridizing to the complement of a sequence encoding the proteins of the invention under stringent conditions (see e.g. Ausubel F et al., Current Protocols in Molecular Biology (John Wiley & Sons, New York, N.Y., U.S., 1993)), and below. Stringent conditions are known to those skilled in the art and may be found in Current Protocols in Molecular Biology (John Wiley & Sons, NY, U.S., Ch. Sec. 6.3.1-6.3.6 (1989)).

The present invention further provides expression vectors that comprise the polynucleotides within the scope of the present invention. The polynucleotides capable of encoding the proteins of the invention may be inserted into known vectors, including bacterial plasmids, viral vectors and phage vectors, using material and methods well known in the art to produce expression vectors. Such expression vectors will include the polynucleotides necessary to support production of contemplated proteins of the invention within any host cell of choice or cell-free expression systems (e.g. pTxb1 and pIVEX2.3 described in the Examples below). The specific polynucleotides comprising expression vectors for use with specific types of host cells or cell-free expression systems are well known to one of ordinary skill in the art, can be determined using routine experimentation, or may be purchased.

The term “expression vector,” as used herein, refers to a polynucleotide, linear or circular, comprising one or more expression units. The term “expression unit” denotes a polynucleotide segment encoding a polypeptide of interest and capable of providing expression of the nucleic acid segment in a host cell. An expression unit typically comprises a transcription promoter, an open reading frame encoding the polypeptide of interest, and a transcription terminator, all in operable configuration. An expression vector contains one or more expression units. Thus, in the context of the present invention, an expression vector encoding a protein of the invention comprising a single polypeptide chain (e.g. a scFv genetically recombined with a Shiga toxin effector region) includes at least an expression unit for the single polypeptide chain, whereas a protein of the invention comprising, e.g. two or more polypeptide chains (e.g. one chain comprising a V_{L} domain and a second chain comprising a V_{H} domain linked to a toxin effector region) includes at least two expression units, one for each of the two polypeptide chains of the protein. For expression of multi-chain proteins of the invention, an expression unit for each polypeptide chain may also be separately contained on different expression vectors (e.g. expression may be achieved with a single host cell into which expression vectors for each polypeptide chain has been introduced).

Expression vectors capable of directing transient or stable expression of polypeptides and proteins are well known in the art. The expression vectors generally include, but are not limited to, one or more of the following: a heterologous signal sequence or peptide, an origin of replication, one or more marker genes, an enhancer element, a promoter, and a transcription termination sequence, each of which is well known in the art. Optional regulatory control sequences, integration sequences, and useful markers that can be employed are known in the art.

The term “host cell” refers to a cell which can support the replication or expression of the expression vector. Host cells may be prokaryotic cells, such as E. coli or eukaryotic cells (e.g. yeast, insect, amphibian, bird, or mammalian cells). Creation and isolation of host cell lines comprising a polynucleotide of the invention or capable of producing a protein of the invention can be accomplished using standard techniques known in the art.

Cell-targeted molecules within the scope of the present invention may be variants or derivatives of the proteins described herein that are produced by modifying the polynucleotide encoding a disclosed protein of the invention by altering one or more amino acids or deleting or inserting one or more amino acids that may render it more suitable to achieve desired properties, such as more optimal expression by a host cell.

### VIII. Molecules of the Present Invention Immobilized on Solid Substrates

Certain embodiments of the present invention include a molecule of the present invention (e.g. a protease-cleavage resistant, cytotoxic molecule or cell-targeted molecule) or any effector fragment thereof, immobilized on a solid substrate. Solid substrates contemplated herein include, but are not limited to, microbeads, nanoparticles, polymers, matrix materials, microarrays, microtiter plates, or any solid surface known in the art (see e.g. U.S. Pat. No. 7,771,955). In accordance with these embodiments, a molecule of the present invention may be covalently or non-covalently linked to a solid substrate, such as, e.g., a bead, particle, or plate, using techniques known to the skilled worker. Immobilized molecules of the invention may be used for screening applications using techniques known in the art (see e.g. Bradbury A et al., Nat Biotechnol 29: 245-54 (2011); Sutton C, Br J Pharmacol 166: 457-75 (2012); Diamante L et al., Protein Eng Des Sel 26: 713-24 (2013); Houlihan G et al., J Immunol Methods 405: 47-56 (2014)).

Non-limiting examples of solid substrates to which a molecule of the invention may be immobilized on include: microbeads, nanoparticles, polymers, nanopolymers, nanotubes, magnetic beads, paramagnetic beads, superparamagnetic beads, streptavidin coated beads, reverse-phase magnetic beads, carboxy terminated beads, hydrazine terminated beads, silica (sodium silica) beads and iminodiacetic acid (IDA)-modified beads, aldehyde-modified beads, epoxy-activated beads, diaminodipropylamine (DADPA)-modified beads (beads with primary amine surface group), biodegradable polymeric beads, polystyrene substrates, amino-polystyrene particles, carboxyl-polystyrene particles, epoxy-polystyrene particles, dimethylamino-polystyrene particles, hydroxy-polystyrene particles, colored particles, flow cytometry particles, sulfonate-polystyrene particles, nitrocellulose surfaces, reinforced nitrocellulose membranes, nylon membranes, glass surfaces, activated glass surfaces, activated quartz surfaces, polyvinylidene difluoride (PVDF) membranes, polyacrylamide-based substrates, poly-vinyl chloride substrates, poly-methyl methacrylate substrates, poly(dimethyl siloxane) substrates, and photopolymers which contain photoreactive species (such as nitrenes, carbenes, and ketyl radicals) capable of forming covalent linkages. Other examples of solid substrates to which a molecule of the invention may be immobilized on are commonly used in molecular display systems, such as, e.g., cellular surfaces, phages, and virus particles.

### IX. Delivery Devices and Kits

In certain embodiments, the invention relates to a device comprising one or more compositions of matter of the invention, such as a pharmaceutical composition, for delivery to a subject. Thus, a delivery devices comprising one or more compositions of matter of the invention may be used to administer to a patient a composition of matter of the invention by various delivery methods, including: intravenous, subcutaneous, intramuscular or intraperitoneal injection; oral administration; transdermal administration; pulmonary or transmucosal administration; administration by implant, osmotic pump, cartridge or micro pump; or by other means recognized by a person of skill in the art.

Also within the scope of the present invention are kits comprising at least one composition of matter of the invention, and optionally, packaging and instructions for use. Kits may be useful for drug administration and/or diagnostic information gathering. A kit of the invention may optionally comprise at least one additional reagent (e.g., standards, markers and the like). Kits typically include a label indicating the intended use of the contents of the kit. The kit may further comprise reagents and other tools for detecting a cell type (e.g. tumor cell) in a sample or in a subject, or for diagnosing whether a patient belongs to a group that responds to a therapeutic strategy which makes use of a compound, composition or related method of the present invention as described herein.

### X. Methods for Using a Cell-Targeted Molecule of the Present Invention or a Composition Thereof

Generally, it is an object of the invention to provide pharmacologically active agents, as well as compositions comprising the same, that can be used in the prevention and/or treatment of diseases, disorders, and conditions, such as certain cancers, tumors, growth abnormalities, immune disorders, microbial infections, or further pathological conditions mentioned herein. Accordingly, the present invention provides methods of using the proteins and pharmaceutical compositions of the invention for the targeted killing of cells, for delivering additional exogenous materials into target cells, for labeling of the interiors of target cells, for collecting diagnostic information, and for treating diseases, disorders, and conditions as described herein.

In particular, it is an object of the invention to provide such pharmacologically active agents, compositions, and/or methods that have certain advantages compared to the agents, compositions, and/or methods that are currently known in the art. Accordingly, the present invention provides methods of using proteins of the invention characterized by specified polypeptide sequences and pharmaceutical compositions thereof. For example, any of the polypeptide sequences in SEQ ID NOs: 1-35 may be specifically utilized as a component of the cell-targeted molecule used in the following methods.

The present invention provides methods of killing a cell comprising the step of contacting the cell, either in vitro or in vivo, with a cell-targeted molecule or pharmaceutical composition of the present invention. The cell-targeted molecules and pharmaceutical compositions of the invention can be used to kill a specific cell type by contacting a cell or cells with one of the claimed compositions of matter. In certain embodiments, a cell-targeted molecule or pharmaceutical composition of the present invention can be used to kill specific cell types in a mixture of different cell types, such as mixtures comprising cancer cells, infected cells, and/or hematological cells. In certain embodiments, a cell-targeted molecule or pharmaceutical composition of the present invention can be used to kill cancer cells in a mixture of different cell types. In certain embodiments, a cell-targeted molecule or pharmaceutical composition of the present invention can be used to kill specific cell types in a mixture of different cell types, such as pre-transplantation tissues. In certain embodiments, the cell-targeted molecules or pharmaceutical composition of the present invention can be used to kill specific cell types in a mixture of cell types, such as pre-administration tissue material for therapeutic purposes. In certain embodiments, a cell-targeted molecule or pharmaceutical composition of the present invention can be used to selectively kill cells infected by viruses or microorganisms, or otherwise selectively kill cells expressing a particular extracellular target biomolecule, such as a cell surface biomolecule. The cell-targeted molecules and pharmaceutical compositions of the invention have varied applications, including, e.g., uses in depleting unwanted cell types from tissues either in vitro or in vivo, uses in modulating immune responses to treat graft-versus-host disease, uses as antiviral agents, uses as anti-parasitic agents, and uses in purging transplantation tissues of unwanted cell types.

In certain embodiments, a cell-targeted molecule or pharmaceutical composition of the present invention, alone or in combination with other compounds or pharmaceutical compositions, can show potent cell-kill activity when administered to a population of cells, in vitro or in vivo in a subject such as in a patient in need of treatment. By targeting the delivery of enzymatically active Shiga toxin regions using high-affinity immunoglobulin-type binding regions to cancer cell types, this potent cell-kill activity can be restricted to specifically and selectively kill certain cell types within an organism, such as certain cancer cells, neoplastic cells, malignant cells, non-malignant tumor cells, or infected cells.

The present invention provides a method of killing a cell in a patient, the method comprising the step of administering to the patient in need thereof at least one cell-targeted molecule of the present invention or a pharmaceutical composition thereof.

Certain embodiments of the cell-targeted molecules of the present invention or pharmaceutical compositions thereof can be used to kill a cancer and/or tumor cell in a patient by targeting an extracellular biomolecule found physically coupled with a cancer and/or tumor cell. The terms “cancer cell” or “cancerous cell” refers to various neoplastic cells which grow and divide in an abnormally accelerated fashion and will be clear to the skilled person. The term “tumor cell” includes both malignant and non-malignant cells (e.g. non-cancerous, benign tumor cells, non-cancerous “cancer” stem cells, tumor stem cells, pre-malignant cancer-initiating cells, tumor-initiating cells, or tumorigenic cells all of which can give rise to daughter cells which become malignant tumor and/or cancer cells but are unable to metastasize on their own (see e.g. Martinez-Climent J et al., Haematologica 95: 293-302 (2010)). Generally, cancers and/or tumors can be defined as diseases, disorders, or conditions that are amenable to treatment and/or prevention. The cancers and tumors (either malignant or non-malignant) which are comprised of cancer cells and/or tumor cells which may benefit from methods and compositions of the invention will be clear to the skilled person. Neoplastic cells are often associated with one or more of the following: unregulated growth, lack of differentiation, local tissue invasion, angiogenesis, and metastasis.

The present invention may be used to kill cancer stem cells, which commonly are slow dividing and resistant to cancer therapies like chemotherapy and radiation. For example, acute myeloid leukemias (AMLs) may be treated with the present invention by killing AML stem cells and/or dormant AML progenitor cells (see e.g. Shlush L et al., Blood 120: 603-12 (2012)). Cancer stem cells often overexpress cell surface targets, such as CD44 and CD200, which can be used to target therapeutic molecules of the present invention (see e.g. Kawasaki B et al., Biochem Biophys Res Commun 364:778-82 (2007); Reim F et al., CancerRes 69: 8058-66 (2009)).

Certain embodiments of the cell-targeted molecules of the invention or pharmaceutical compositions thereof can be used to kill an immune cell (whether healthy or malignant) in a patient by targeting an extracellular biomolecule found physically coupled with an immune cell.

Certain embodiments of the cell-targeted molecules of the invention or pharmaceutical compositions thereof can be used to kill an infected cell in a patient by targeting an extracellular biomolecule found physically coupled with an infected cell.

It is within the scope of the present invention to utilize the cell-targeted molecule of the invention or pharmaceutical composition thereof for the purposes of purging patient cell populations (e.g. bone marrow) of infected malignant, neoplastic, or otherwise unwanted B-cells and/or T-cells and then reinfusing the B-cell and/or T-cell depleted material into the patient (see e.g. van Heeckeren W et al., Br J Haematol 132: 42-55 (2006); Alpdogan O, van den Brink M, Semin Oncol 39: 629-42 (2012)).

It is within the scope of the present invention to utilize the cell-targeted molecule of the invention or pharmaceutical composition thereof for the purposes of ex vivo depletion of B-cells and/or T cells from isolated cell populations removed from a patient. In one non-limiting example, the cell-targeted molecule of the invention may be used in a method for prophylaxis of organ and/or tissue transplant rejection wherein the donor organ or tissue is perfused prior to transplant with a cytotoxic cell-targeted molecule of the invention or a pharmaceutical composition thereof in order to purge the organ of unwanted donor B-cells and/or T-cells (see e.g. Alpdogan O, van den Brink M, Semin Oncol 39: 629-42 (2012)).

It is also within the scope of the present invention to utilize the cell-targeted molecule of the invention or pharmaceutical composition thereof for the purposes of depleting B-cells, NK cells, and/or T-cells from a donor cell population as a prophylaxis against graft-versus-host disease, and induction of tolerance, in a patient to undergo a bone marrow and/or stem cell transplant (see e.g. Sarantopoulos S et al., Biol Blood Marrow Transplant 21: 16-23 (2015)).

Certain embodiments of the cell-targeted molecule of the invention or pharmaceutical compositions thereof can be used to kill an infected cell in a patient by targeting an extracellular biomolecule found physically coupled with an infected cell.

Certain embodiments of the cell-targeted molecule of the invention or pharmaceutical compositions thereof can be used to kill a cell(s) of a multicellular parasite. In certain further embodiments, the cell killing occurs while the multicellular parasite is present in a host organism or subject. In certain further embodiments, the cell-targeted molecule of the invention may be used to kill a helminth, such as, e.g., a plathelminth, nemathelminth, cestode, mongenean, nematode, and/or trematode.

Additionally, the present invention provides a method of treating a disease, disorder, or condition in a patient comprising the step of administering to a patient in need thereof a therapeutically effective amount of at least one of the cell-targeted molecules of the present invention or a pharmaceutical composition thereof. Contemplated diseases, disorders, and conditions that can be treated using this method include cancers, malignant tumors, non-malignant tumors, growth abnormalities, immune disorders, and microbial infections. Administration of a “therapeutically effective dosage” of a compound of the invention can result in a decrease in severity of disease symptoms, an increase in frequency and duration of disease symptom-free periods, or a prevention of impairment or disability due to the disease affliction.

The therapeutically effective amount of a cell-targeted molecule of the present invention or composition thereof will depend on the route of administration, the type of mammal being treated, and the physical characteristics of the specific patient under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained by the present invention, and may be confirmed in properly designed clinical trials. An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

An acceptable route of administration may refer to any administration pathway known in the art, including but not limited to aerosol, enteral, nasal, ophthalmic, oral, parenteral, rectal, vaginal, or transdermal (e.g. topical administration of a cream, gel or ointment, or by means of a transdermal patch). “Parenteral administration” is typically associated with injection at or in communication with the intended site of action, including intratumoral injection, infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal administration.

For administration of a pharmaceutical composition of the invention, the dosage range will generally be from about 0.0001 to 100 milligrams per kilogram (mg/kg), and more, usually 0.01 to 5 mg/kg, of the subject's body weight. Exemplary dosages may be 0.25 mg/kg body weight, 1 mg/kg body weight, 3 mg/kg body weight, 5 mg/kg body weight or 10 mg/kg body weight or within the range of 1-10 mg/kg. An exemplary treatment regime is a once or twice daily administration, or a once or twice weekly administration, once every two weeks, once every three weeks, once every four weeks, once a month, once every two or three months or once every three to 6 months. Dosages may be selected and readjusted by the skilled health care professional as required to maximize therapeutic benefit for a particular patient.

Pharmaceutical compositions of the invention will typically be administered to the same patient on multiple occasions. Intervals between single dosages can be, for example, one to four days, weekly, monthly, every two or three months, every six months, or yearly. Intervals between administrations can also be irregular, based on regulating blood levels of the active compound or based on other markers, indications, or signs present in a particular subject or patient. Dosage regimens for a compound of the invention include intravenous administration of 1 mg/kg body weight or 3 mg/kg body weight with the pharmaceutical composition administered every two to four weeks for six dosages, then every three months at 3 mg/kg body weight or 1 mg/kg body weight.

A pharmaceutical composition of the present invention may be administered via one or more routes of administration, using one or more of a variety of methods known in the art. As will be appreciated by the skilled worker, the route and/or mode of administration will vary depending upon the desired results. Routes of administration for cell-targeted molecules of the present invention or compositions thereof include, e.g. intravenous, intramuscular, intradermal, intraperitoneal, subcutaneous, spinal, or other parenteral routes of administration, for example by injection or infusion at or in communication with the intended site of action (e.g. intratumoral injection). In other embodiments, a cell-targeted molecule or pharmaceutical composition of the invention may be administered by a non-parenteral route, such as a topical, epidermal or mucosal route of administration, for example, intranasally, orally, vaginally, rectally, sublingually, or topically.

Cell-targeted molecules or pharmaceutical compositions of the present invention may be administered with one or more of a variety of medical devices known in the art. For example, in one embodiment, a pharmaceutical composition of the invention may be administered with a needleless hypodermic injection device. Examples of well-known implants and modules useful in the present invention are in the art, including e.g., implantable micro-infusion pumps for controlled rate delivery; devices for administering through the skin; infusion pumps for delivery at a precise infusion rate; variable flow implantable infusion devices for continuous drug delivery; and osmotic drug delivery systems. These and other such implants, delivery systems, and modules are known to those skilled in the art.

A cell-targeted molecule or pharmaceutical composition of the present invention may be administered alone or in combination with one or more other therapeutic or diagnostic agents. A combination therapy may include a cell-targeted molecule of the invention or pharmaceutical composition thereof combined with at least one other therapeutic agent selected based on the particular patient, disease or condition to be treated. Examples of other such agents include, inter alia, a cytotoxic, anti-cancer or chemotherapeutic agent, an anti-inflammatory or anti-proliferative agent, an antimicrobial or antiviral agent, growth factors, cytokines, an analgesic, a therapeutically active small molecule or polypeptide, a single chain antibody, a classical antibody or fragment thereof, or a nucleic acid molecule which modulates one or more signaling pathways, and similar modulating therapeutics which may complement or otherwise be beneficial in a therapeutic or prophylactic treatment regimen.

Treatment of a patient with a cell-targeted molecule or pharmaceutical composition of the invention preferably leads to cell death of targeted cells and/or the inhibition of growth of targeted cells. As such, cell-targeted molecules of the invention, and pharmaceutical compositions comprising them, will be useful in methods for treating a variety of pathological disorders in which killing or depleting target cells may be beneficial, such as, inter alia, cancers, tumors, growth abnormalities, immune disorders, and infected cells. The present invention provides methods for suppressing cell proliferation, and treating cell disorders, including neoplasia, overactive B-cells, and overactive T-cells.

In certain embodiments, cell-targeted molecules and pharmaceutical compositions of the invention may be used to treat or prevent cancers, tumors (malignant and non-malignant), growth abnormalities, immune disorders, and microbial infections. In a further aspect, the above ex vivo method can be combined with the above in vivo method to provide methods of treating or preventing rejection in bone marrow transplant recipients, and for achieving immunological tolerance.

In certain embodiments, the present invention provides methods for treating malignancies or neoplasms and other blood cell associated cancers in a mammalian subject, such as a human, the method comprising the step of administering to a subject in need thereof a therapeutically effective amount of a cell-targeted molecule or pharmaceutical composition of the invention.

The cell-targeted molecules and pharmaceutical compositions of the invention have varied applications, including, e.g., uses in removing unwanted B-cells and/or T-cells, uses in modulating immune responses to treat graft-versus-host diseases, uses as antiviral agents, uses as antimicrobial agents, and uses in purging transplantation tissues of unwanted cell types. The cell-targeted molecules and pharmaceutical compositions of the present invention are commonly anti-neoplastic agents—meaning they are capable of treating and/or preventing the development, maturation, or spread of neoplastic or malignant cells by inhibiting the growth and/or causing the death of cancer or tumor cells.

In certain embodiments, a cell-targeted molecule or pharmaceutical composition of the present invention is used to treat a B-cell-, plasma cell-, T-cell, or antibody-mediated disease or disorder, such as for example leukemia, lymphoma, myeloma, amyloidosis, ankylosing spondylitis, asthma, Crohn's disease, diabetes, graft rejection, graft-versus-host disease, Hashimoto's thyroiditis, hemolytic uremic syndrome, Human Immunodeficiency Virus-related (HIV-related) diseases, lupus erythematosus, multiple sclerosis, polyarteritis nodosa, polyarthritis, psoriasis, psoriatic arthritis, rheumatoid arthritis, scleroderma, septic shock, Sjorgren's syndrome, ulcerative colitis, and vasculitis.

In another aspect, certain embodiments of the cell-targeted molecules and pharmaceutical compositions of the present invention are antimicrobial agents—meaning they are capable of treating and/or preventing the acquisition, development, or consequences of microbiological pathogenic infections, such as caused by viruses, bacteria, fungi, prions, or protozoans.

It is within the scope of the present invention to provide a prophylaxis or treatment for diseases or conditions mediated by B-cells and/or T-cells, the prophylaxis or treatment involving administering the cell-targeted molecule or the invention, or a pharmaceutical composition thereof, to a patient in need thereof for the purpose of killing B-cells and/or T-cells in the patient. This usage is compatible with preparing or conditioning a patient for bone marrow transplantation, stem cell transplantation, tissue transplantation, or organ transplantation, regardless of the source of the transplanted material, e.g. human or non-human sources.

It is within the scope of the present invention to provide a bone marrow recipient for prophylaxis or treatment of host-versus-graft disease via the targeted cell-killing of host B-cells, NK cells, and/or T-cells using a cell-targeted molecule or pharmaceutical composition of the present invention (see e.g. Sarantopoulos S et al., Biol Blood Marrow Transplant 21: 16-23 (2015)).

The cell-targeted molecules and pharmaceutical compositions of the present invention may be utilized in a method of treating cancer comprising administering to a patient, in need thereof, a therapeutically effective amount of the cell-targeted molecule or a pharmaceutical composition of the present invention. In certain embodiments of the methods of the present invention, the cancer being treated is selected from the group consisting of: bone cancer (such as multiple myeloma or Ewing's sarcoma), breast cancer, central/peripheral nervous system cancer (such as brain cancer, neurofibromatosis, or glioblastoma), gastrointestinal cancer (such as stomach cancer or colorectal cancer), germ cell cancer (such as ovarian cancers and testicular cancers, glandular cancer (such as pancreatic cancer, parathyroid cancer, pheochromocytoma, salivary gland cancer, or thyroid cancer), head-neck cancer (such as nasopharyngeal cancer, oral cancer, or pharyngeal cancer), hematological cancers (such as leukemia, lymphoma, or myeloma), kidney-urinary tract cancer (such as renal cancer and bladder cancer), liver cancer, lung/pleura cancer (such as mesothelioma, small cell lung carcinoma, or non-small cell lung carcinoma), prostate cancer, sarcoma (such as angiosarcoma, fibrosarcoma, Kaposi's sarcoma, or synovial sarcoma), skin cancer (such as basal cell carcinoma, squamous cell carcinoma, or melanoma), and uterine cancer.

The cell-targeted molecules and pharmaceutical compositions of the present invention may be utilized in a method of treating an immune disorder comprising administering to a patient, in need thereof, a therapeutically effective amount of the cell-targeted molecule or a pharmaceutical composition of the present invention. In certain embodiments of the methods of the present invention, the immune disorder is related to an inflammation associated with a disease selected from the group consisting of: amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriasis, psoriatic arthritis, rheumatoid arthritis, scleroderma, septic shock, Sjorgren's syndrome, ulcerative colitis, and vasculitis.

Among certain embodiments of the present invention is using the cell-targeted molecule of the invention as a component of a pharmaceutical composition or medicament for the treatment or prevention of a cancer, tumor, growth abnormality, immune disorder, and/or microbial infection. For example, immune disorders presenting on the skin of a patient may be treated with such a medicament in efforts to reduce inflammation. In another example, skin tumors may be treated with such a medicament in efforts to reduce tumor size or eliminate the tumor completely.

Certain cell-targeted molecules of the invention may be used in molecular neurosurgery applications such as immunolesioning and neuronal tracing (see, Wiley R, Lappi D, Adv Drug Deliv Rev 55: 1043-54 (2003), for review). For example, the targeting domain may be selected or derived from various ligands, such as neurotransmitters and neuropeptides, which target specific neuronal cell types by binding neuronal surface receptors, such as a neuronal circuit specific G-protein coupled receptor. Similarly, the targeting domain may be selected from or derived from antibodies that bind neuronal surface receptors. Because Shiga toxins robustly direct their own retrograde axonal transport, certain cytotoxic cell-targeted molecules of the invention may be used to kill a neuron(s) which expresses the extracellular target at a site of cytotoxic cell-targeted molecule injection distant from the cell body (see Llewellyn-Smith I et al., J Neurosci Methods 103: 83-90 (2000)). These neuronal cell type specific targeting cytotoxic cell-targeted molecules have uses in neuroscience research, such as for elucidating mechanisms of sensations (see e.g. Mishra S, Hoon M, Science 340: 968-71 (2013)), and creating model systems of neurodegenerative diseases, such as Parkinson's and Alzheimer's (see e.g. Hamlin A et al., PLoS One e53472 (2013)).

Among certain embodiments of the present invention is a method of using a cell-targeted molecule, pharmaceutical composition, and/or diagnostic composition of the invention to detect the presence of a cell type for the purpose of information gathering regarding diseases, conditions and/or disorders. The method comprises contacting a cell with a diagnostically sufficient amount of a cell-targeted molecule of the invention to detect the cell-targeted molecule by an assay or diagnostic technique. The term “diagnostically sufficient amount” refers to an amount that provides adequate detection and accurate measurement for information gathering purposes by the particular assay or diagnostic technique utilized. Generally, the diagnostically sufficient amount for whole organism in vivo diagnostic use will be a non-cumulative dose of between 0.1 mg to 100 mg of the detection-promoting-agent-linked cell-targeted molecule per kg of subject per subject. Typically, the amount of cell-targeted molecule of the invention used in these information gathering methods will be as low as possible provided that it is still a diagnostically sufficient amount. For example, for in vivo detection in an organism, the amount of cell-targeted molecule of the invention administered to a subject will be as low as feasibly possible.

The cell-type specific targeting of cell-targeted molecules of the present invention combined with detection promoting agents provides a way to detect and image cells physically coupled with an extracellular target biomolecule of a binding region of the cell-targeted molecules of the invention. Imaging of cells using the cell-targeted molecules of the invention may be performed in vitro or in vivo by any suitable technique known in the art. For example, the method of using a cell-targeted molecule, pharmaceutical composition, or diagnostic composition of the invention to detect the presence of a cell type for the purpose of information gathering may be performed on cells in vivo within a patient, including on cells in situ, e.g. at a disease locus, on cells in vitro, and/or in an ex vivo setting on cells and tissues removed from an organism, e.g. a biopsy material.

Diagnostic information may be collected using various methods known in the art, including whole body imaging of an organism or using ex vivo samples taken from an organism. The term sample used herein refers to any number of things, but not limited to, fluids such as blood, urine, serum, lymph, saliva, anal secretions, vaginal secretions, and semen, and tissues obtained by biopsy procedures. For example, various detection promoting agents may be utilized for non-invasive in vivo tumor imaging by techniques such as magnetic resonance imaging (MRI), optical methods (such as direct, fluorescent, and bioluminescent imaging), positron emission tomography (PET), single-photon emission computed tomography (SPECT), ultrasound, x-ray computed tomography, and combinations of the aforementioned (see, Kaur S et al., Cancer Lett 315: 97-111 (2012), for review).

The method of using a cell-targeted molecule, pharmaceutical composition, or diagnostic composition of the invention to detect the presence of a target biomolecule positive cell type for the purpose of information gathering may be performed on cells in vivo within a patient, on cells in situ, e.g. at a disease locus, on cells in vitro, and/or in an ex vivo setting on cells and tissues removed from an organism, e.g. a biopsy material. The detection of specific cells, cell types, and cell populations using a composition of the invention may be used for diagnosis and imaging of cells, such as, e.g., tumor, cancer, immune, and infected cells. For example, cell-targeted molecules and diagnostic compositions of the invention may be employed to image or visualize a site of possible accumulation of target biomolecule expressing cells in an organism. These methods may be used to identify sites of tumor development or residual tumor cells after a therapeutic intervention.

Certain embodiments of the method used to detect the presence of a cell type may be used to gather information regarding diseases, disorders, and conditions, such as, for example bone cancer (such as multiple myeloma or Ewing's sarcoma), breast cancer, central/peripheral nervous system cancer (such as brain cancer, neurofibromatosis, or glioblastoma), gastrointestinal cancer (such as stomach cancer or colorectal cancer), germ cell cancer (such as ovarian cancers and testicular cancers, glandular cancer (such as pancreatic cancer, parathyroid cancer, pheochromocytoma, salivary gland cancer, or thyroid cancer), head-neck cancer (such as nasopharyngeal cancer, oral cancer, or pharyngeal cancer), hematological cancers (such as leukemia, lymphoma, or myeloma), kidney-urinary tract cancer (such as renal cancer and bladder cancer), liver cancer, lung/pleura cancer (such as mesothelioma, small cell lung carcinoma, or non-small cell lung carcinoma), prostate cancer, sarcoma (such as angiosarcoma, fibrosarcoma, Kaposi's sarcoma, or synovial sarcoma), skin cancer (such as basal cell carcinoma, squamous cell carcinoma, or melanoma), uterine cancer, AIDS, amyloidosis, ankylosing spondylitis, asthma, autism, cardiogenesis, Crohn's disease, diabetes, erythematosus, gastritis, graft rejection, graft-versus-host disease, Grave's disease, Hashimoto's thyroiditis, hemolytic uremic syndrome, HIV-related diseases, lupus erythematosus, lymphoproliferative disorders, multiple sclerosis, myasthenia gravis, neuroinflammation, polyarteritis nodosa, polyarthritis, psoriasis, psoriatic arthritis, rheumatoid arthritis, scleroderma, septic shock, Sjorgren's syndrome, systemic lupus erythematosus, ulcerative colitis, vasculitis, cell proliferation, inflammation, leukocyte activation, leukocyte adhesion, leukocyte chemotaxis, leukocyte maturation, leukocyte migration, neuronal differentiation, acute lymphoblastic leukemia (ALL), T acute lymphocytic leukemia/lymphoma (ALL), acute myelogenous leukemia, acute myeloid leukemia (AML), B-cell chronic lymphocytic leukemia (B-CLL), B-cell prolymphocytic lymphoma, Burkitt's lymphoma (BL), chronic lymphocytic leukemia (CLL), chronic myelogenous leukemia (CML-BP), chronic myeloid leukemia (CML), diffuse large B-cell lymphoma, follicular lymphoma, hairy cell leukemia (HCL), Hodgkin's Lymphoma (HL), intravascular large B-cell lymphoma, lymphomatoid granulomatosis, lymphoplasmacytic lymphoma, MALT lymphoma, mantle cell lymphoma, multiple myeloma (MM), natural killer cell leukemia, nodal marginal B-cell lymphoma, Non-Hodgkin's lymphoma (NHL), plasma cell leukemia, plasmacytoma, primary effusion lymphoma, pro-lymphocytic leukemia, promyelocytic leukemia, small lymphocytic lymphoma, splenic marginal zone lymphoma, T-cell lymphoma (TCL), heavy chain disease, monoclonal gammopathy, monoclonal immunoglobulin deposition disease, myelodusplastic syndromes (MDS), smoldering multiple myeloma, and Waldenstrom macroglobulinemia.

Among certain embodiment of the present invention is a method of using a cell-targeted molecule, pharmaceutical composition, and/or diagnostic composition of the invention to label or detect the interiors of a cell type, such as, e.g., neoplastic and/or immune cell types (see e.g., Koyama Y et al., Clin Cancer Res 13: 2936-45 (2007); Ogawa M et al., Cancer Res 69: 1268-72 (2009); Yang L et al., Small 5: 235-43 (2009)). Based on the ability of the cell-targeted molecules and pharmaceutical compositions of the invention to enter specific cell types and route within cells via retrograde intracellular transport, the interior compartments of specific cell types may be labeled for detection. This can be performed in vivo on cells in situ within an organism, e.g. a patient, or in vitro on cells and tissues removed from an organism, e.g. biopsy material.

Diagnostic compositions of the invention may be used to characterize a disease, disorder, or condition as potentially treatable by a related pharmaceutical composition of the invention. Certain compositions of matter of the invention may be used to determine whether a patient belongs to a group that responds to a therapeutic strategy which makes use of a compound, composition or related method of the invention as described herein or is well suited for using a delivery device of the invention.

Diagnostic compositions of the invention may be used after a disease, e.g. a cancer, is detected in order to better characterize it, such as to monitor distant metastases, heterogeneity, and stage of cancer progression. The phenotypic assessment of disease disorder or infection can help prognostic and prediction during therapeutic decision making. In disease reoccurrence, certain methods of the invention may be used to determine if local or systemic problem.

Diagnostic compositions of the invention may be used to assess responses to therapeutic(s) regardless of the type of therapeutic, e.g. small molecule drug, biological drug, or cell-based therapy. For example, certain embodiments of the diagnostics of the invention may be used to measure changes in tumor size, changes in antigen positive cell populations including number and distribution, and/or monitor a different marker than the antigen targeted by a therapy already being administered to a patient (see e.g. Smith-Jones P et al., Nat Biotechnol 22: 701-6 (2004); Evans M et al., Proc Natl Acad Sci USA 108: 9578-82 (2011)).

In certain embodiments, the cell-targeted molecules of the invention or pharmaceutical and/or diagnostic compositions thereof are used for both diagnosis and treatment, or for diagnosis alone.

The present invention provides various proteins, each protein comprising 1) an immunoglobulin-type binding region for cell targeting and 2) a Shiga toxin effector region for cellular internalization, intracellular routing, and/or cell killing. The linking of cell targeting immunoglobulin-type binding regions with Shiga-toxin-Subunit-A-derived regions enables the engineering of cell-type specific targeting of the potent Shiga toxin cytotoxicity. The linking of immunoglobulin-type binding regions with Shiga-toxin-Subunit-A-derived polypeptide regions enabled the engineering of cell-type specific targeting of Shiga toxin cytotoxicity, and cytotoxicity was highest when these two regions were combined such that the immunoglobulin-type binding regions were not proximal relative to the Shiga toxin regions to the amino-terminals of the proteins.

In certain embodiments, the protein of the present invention comprises 1) an immunoglobulin-type binding region for cell targeting and 2) a cytotoxic Shiga toxin effector region. In certain embodiments, the protein of the present invention comprises a Shiga toxin effector region derived from one or more A Subunits of members of the Shiga toxin family linked to an immunoglobulin-type binding region which can bind specifically to at least one extracellular target biomolecule in physical association with a cell, such as a target biomolecule expressed on the surface of a cell.

In certain embodiments of the proteins of the present invention, the specific order or orientation of the Shiga toxin effector region and immunoglobulin-type binding region is fixed such that the immunoglobulin-type binding region is located within the protein more proximal to the carboxy-terminus of the Shiga toxin effector region than to the amino-terminus of the Shiga toxin effector region.

For certain embodiments of the proteins of the present invention, the term “immunoglobulin-type binding region” refers to a polypeptide region capable of binding one or more target biomolecules, such as an antigen or epitope.

In certain embodiments, the protein of the present invention comprises (a) an immunoglobulin-type binding region comprising one or more polypeptides and capable of specifically binding at least one extracellular target biomolecule and (b) a Shiga toxin effector region comprising a polypeptide derived from the amino acid sequence of the A Subunit of at least one member of the Shiga toxin family; wherein said immunoglobulin-type binding region and said Shiga toxin effector region are physically arranged or oriented within the cytotoxic protein such that the immunoglobulin-type binding region is not located proximally to the amino-terminus of the Shiga toxin effector region.

In certain embodiments, the protein of the present invention comprises (a) an immunoglobulin-type binding region comprising one or more polypeptides and capable of specifically binding at least one extracellular target biomolecule and (b) a Shiga toxin effector region comprising a polypeptide derived from the amino acid sequence of the A Subunit of at least one member of the Shiga toxin family; wherein said immunoglobulin-type binding region and said Shiga toxin effector region are physically arranged or oriented within the cytotoxic protein such that the immunoglobulin-type binding region is not located proximally to the amino-terminus of the protein relative to the Shiga toxin effector region.

In certain further embodiments, the protein of the present invention comprises (a) an immunoglobulin-type binding region comprising one or more polypeptides and capable of specifically binding at least one extracellular target biomolecule and (b) a Shiga toxin effector region comprising a polypeptide derived from the amino acid sequence of the A Subunit of at least one member of the Shiga toxin family; wherein said immunoglobulin-type binding region and said Shiga toxin effector region are physically arranged or oriented within the cytotoxic protein such that the Shiga toxin effector region is located proximally to the amino terminus of the protein.

In certain embodiments, the binding region of the protein of the present invention comprises a polypeptide region capable of binding specifically to an extracellular target biomolecule, preferably which is physically-coupled to the surface of a cell type of interest, such as a cancer cell, tumor cell, plasma cell, infected cell, or host cell harboring an intracellular pathogen

In certain embodiments, the binding region of a protein of the present invention comprises an immunoglobulin-type binding region comprising one or more polypeptides capable of selectively and specifically binding an extracellular target biomolecule.

In certain embodiments of the proteins of the present invention, the immunoglobulin-type binding region comprises a polypeptide selected from the group consisting of: single-domain antibody (sdAb) fragment, nanobody, heavy-chain antibody domain derived from a camelid (V_{H}H fragment), heavy-chain antibody domain derived from a cartilaginous fish, immunoglobulin new antigen receptor (IgNAR), V_{NAR} fragment, single-chain variable fragment (scFv), antibody variable fragment (Fv), a complementary determining region 3 (CDR3) fragment, constrained FR3-CDR3-FR4 (FR3-CDR3-FR4) polypeptide, Fd fragment, small modular immunopharmaceutical (SMIP) domain, antigen-binding fragment (Fab), fibronectin-derived 10^{th} fibronectin type III domain (10Fn3) (e.g. monobody), tenascin type III domain (e.g. TNfn3), ankyrin repeat motif domain (ARD), low-density-lipoprotein-receptor-derived A-domain (A domain of LDLR or LDLR-A), lipocalin (anticalin), Kunitz domain, Protein-A-derived Z domain, gamma-B crystalline-derived domain, ubiquitin-derived domain, Sac7d-derived polypeptide (affitin), Fyn-derived SH2 domain, miniprotein, C-type lectin-like domain scaffold, engineered antibody mimic, and any genetically manipulated counterparts of any of the foregoing which retain binding functionality.

For certain embodiments, whereby administration of the protein of the present invention to a cell physically coupled with the extracellular target biomolecule of the protein's binding region, the protein is capable of causing death of the cell. In certain further embodiments, administration of the protein of the invention to two different populations of cell types which differ with respect to the presence or level of an extracellular target biomolecule, the protein is capable of causing cell death to the cell-types physically coupled with an extracellular target biomolecule of the cytotoxic protein's binding region at a CD₅₀ at least three times or less than the CD₅₀ to cell types which are not physically coupled with an extracellular target biomolecule of the protein's binding region. For certain embodiments, whereby administration of the protein of the invention to a first population of cells whose members are physically coupled to extracellular target biomolecules of the protein's binding region, and a second population of cells whose members are not physically coupled to any extracellular target biomolecule of the binding region, the cytotoxic effect of the cell-targeted molecule to members of said first population of cells relative to members of said second population of cells is at least 3-fold greater. For certain embodiments, whereby administration of the protein of the invention to a first population of cells whose members are physically coupled to a significant amount of the extracellular target biomolecule of the protein's binding region, and a second population of cells whose members are not physically coupled to a significant amount of any extracellular target biomolecule of the binding region, the cytotoxic effect of the cell-targeted molecule to members of said first population of cells relative to members of said second population of cells is at least 3-fold greater. For certain embodiments, whereby administration of the protein of the invention to a first population of target biomolecule positive cells, and a second population of cells whose members do not express a significant amount of a target biomolecule of the protein's binding region at a cellular surface, the cytotoxic effect of the protein to members of the first population of cells relative to members of the second population of cells is at least 3-fold greater.

In certain embodiments, the immunoglobulin-type binding region is designed or selected by its ability to bind an extracellular target biomolecule selected from the group consisting of: CD20, CD22, CD40, CD74, CD79, CD25, CD30, HER2/neu/ErbB2, EGFR, EpCAM, EphB2, prostate-specific membrane antigen, Cripto, CDCP1, endoglin, fibroblast activated protein, Lewis-Y, CD19, CD21, CS1/SLAMF7, CD33, CD52, CD133, EpCAM, CEA, gpA33, Mucins, TAG-72, tyrosine-protein kinase transmembrane receptor (ROR1 or NTRKR1), carbonic anhydrase IX, folate binding protein, ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANK, FAP, Tenascin, CD64, mesothelin, BRCA1, MART-1/MelanA, gp100, tyrosinase, TRP-1, TRP-2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen, prostate specific antigen, prostate stem cell antigen, human aspartyl (asparaginyl) beta-hydroxylase, EphA2, HER3/ErbB-3, MUC1, MART-1/MelanA, gp100, tyrosinase associated antigen, HPV-E7, Epstein-Barr Virus antigens, Bcr-Abl, alpha-fetoprotein antigen, 17-A1, bladder tumor antigen, CD38, CD15, CD23, CD53, CD88, CD129, CD183, CD191, CD193, CD244, CD294, CD305, C3AR, FceRIa, galectin-9, mrp-14, programmed death-ligand 1 (PD-L1), siglec-8, siglec-10, CD49d, CD13, CD44, CD54, CD63, CD69, CD123, CD193, TLR4, FceRIa, IgE, CD107a, CD203c, CD14, CD15, CD33, CD64, CD68, CD80, CD86, CD105, CD115, F4/80, ILT-3, Galectin-3, CD11a-c, GITRL, MHC Class I molecule (optionally complexed with a peptide), MHC Class II molecule (optionally complexed with a peptide), CD284-TLR4, CD107-Mac3, CD195-CCR5, HLA-DR, CD16/32, CD282-TLR2, CD11c, CD123, and any immunogenic fragment of any of the foregoing.

In certain embodiments, the Shiga toxin effector region of the present invention comprises or consists essentially of a polypeptide derived from a Shiga toxin A Subunit dissociated from any form of its native Shiga toxin B Subunit. In addition, the proteins of the present invention do not comprise any polypeptide comprising or consisting essentially of a functional binding domain of a native Shiga toxin B subunit. Rather, the Shiga toxin A Subunit derived regions are functionally associated with heterologous immunoglobulin-type binding regions to effectuate cell targeting.

In certain embodiments, the proteins of the present invention comprise the Shiga toxin effector region derived from amino acids 75 to 251 of SEQ ID NO:1, SEQ ID NO:2, or SEQ ID NO:3. In certain further embodiments, the protein of the present invention comprises the Shiga toxin effector region derived from amino acids 1 to 241 of SEQ ID NO:1, SEQ ID NO:2, or SEQ ID NO:3. In certain further embodiments, the Shiga toxin effector region is derived from amino acids 1 to 251 of SEQ ID NO:1, SEQ ID NO:2, or SEQ ID NO:3. In certain further embodiments, the Shiga toxin effector region is derived from amino acids 1 to 261 of SEQ ID NO:1, SEQ ID NO:2, or SEQ ID NO:3.

In certain embodiments, the proteins of the present invention comprise a carboxy-terminal endoplasmic reticulum retention/retrieval signal motif. In certain further embodiments, the proteins of the present invention comprise a carboxy-terminal endoplasmic reticulum retention/retrieval signal motif selected from the group consisting of: KDEL, HDEF, HDEL, RDEF, RDEL, WDEL, YDEL, HEEF, HEEL, KEEL, REEL, KAEL, KCEL, KFEL, KGEL, KHEL, KLEL, KNEL, KQEL, KREL, KSEL, KVEL, KWEL, KYEL, KEDL, KIEL, DKEL, FDEL, KDEF, KKEL, HADL, HAEL, HIEL, HNEL, HTEL, KTEL, HVEL, NDEL, QDEL, REDL, RNEL, RTDL, RTEL, SDEL, TDEL, SKEL, STEL, and EDEL.

In certain further embodiments, the protein of the present invention comprises the binding region comprising or consisting essentially of amino acids 269-508 of any one of SEQ ID NOs: 4-19.

In certain embodiments, the protein of the present invention comprises or consists essentially of the polypeptide shown in any one SEQ ID NOs: 4-31.

In certain embodiments, the proteins of the present invention comprise a Shiga toxin effector region which comprises a mutation relative to a naturally occurring A Subunit of a member of the Shiga toxin family that changes the enzymatic activity of the Shiga toxin effector region. In certain further embodiments, the mutation is selected from at least one amino acid residue deletion, insertion, or substitution that reduces or eliminates cytotoxicity of the Shiga toxin region. In certain further embodiments, the protein comprises a mutation which reduces or eliminates catalytic activity but retains other Shiga toxin effector functions, such as, e.g., promoting cellular internalization and/or directing intracellular routing. In certain embodiments, the mutation is selected from at least one amino acid residue substitution, such as, e.g., A231E, R75A, Y77S, Y114S, E167D, R170A, R176K and/or W203A in SEQ ID NO:1, SEQ ID NO:2, or SEQ ID NO:3.

The present invention also provides pharmaceutical compositions comprising a protein of the present invention and at least one pharmaceutically acceptable excipient or carrier; and the use of such a protein or a composition comprising it in the methods of the invention as further described herein. In certain embodiments of the present invention are pharmaceutical compositions comprising any protein of the present invention and at least one pharmaceutically acceptable excipient or carrier.

Among certain embodiments of the present invention is a diagnostic composition comprising a protein of the invention further comprising a detection promoting agent for the collection of information, such as diagnostically useful information about a cell type, tissue, organ, disease, disorder, condition, and/or patient.

Beyond the proteins of the present invention and compositions thereof, polynucleotides capable of encoding a protein of the invention are within the scope of the present invention, as well as expression vectors which comprise a polynucleotide of the invention and host cells comprising an expression vector of the invention. Host cells comprising an expression vector may be used, e.g., in methods for producing a protein of the invention or a polypeptide component or fragment thereof by recombinant expression.

The invention also includes a system for conferring improved cytotoxicity to a protein which comprises (a) an immunoglobulin-type binding region comprising one or more polypeptides and capable of specifically binding at least one extracellular target biomolecule and (b) a Shiga toxin effector region comprising a polypeptide derived from the amino acid sequence of the A Subunit of at least one member of the Shiga toxin family; the system comprising the step of arranging said immunoglobulin-type binding region proximally to the carboxy-terminus of said Shiga toxin effector region within the protein. The invention also includes a system for conferring improved cytotoxicity to a protein which comprises (a) an immunoglobulin-type binding region comprising one or more polypeptides and capable of specifically binding at least one extracellular target biomolecule and (b) a Shiga toxin effector region comprising a polypeptide derived from the amino acid sequence of the A Subunit of at least one member of the Shiga toxin family; the system comprising the step of arranging said immunoglobulin-type binding region proximally to the carboxy-terminus of the protein relative to said Shiga toxin effector region. The invention also includes a system for conferring improved cytotoxicity to a protein which comprises (a) an immunoglobulin-type binding region comprising one or more polypeptides and capable of specifically binding at least one extracellular target biomolecule and (b) a Shiga toxin effector region comprising a polypeptide derived from the amino acid sequence of the A Subunit of at least one member of the Shiga toxin family; the system comprising the step of arranging said Shiga toxin effector region proximally to the amino-terminus of the protein.

Additionally, the present invention provides methods of killing cell(s) comprising the step of contacting a cell(s) with a protein of the invention or a pharmaceutical composition comprising a protein of the invention. In certain embodiments, the step of contacting the cell(s) occurs in vitro. In certain other embodiments, the step of contacting the cell(s) occurs or in vivo. In further embodiments of the cell killing methods, the method is capable of selectively killing cell(s) and/or cell types preferentially over other cell(s) and/or cell types when contacting a mixture of cells which differ with respect to the extracellular presence and/or expression level of an extracellular target biomolecule of the binding region of the protein.

The present invention further provides methods of treating diseases, disorders, and/or conditions in patients comprising the step of administering to a patient in need thereof a therapeutically effective amount of a protein or a pharmaceutical composition of the invention. In certain embodiments, the disease, disorder, or condition to be treated using a method of the invention is selected from: a cancer, tumor, growth abnormality, immune disorder, or microbial infection. In certain embodiments of these methods, the cancer to be treated is selected from the group consisting of: bone cancer, breast cancer, central/peripheral nervous system cancer, gastrointestinal cancer, germ cell cancer, glandular cancer, head-neck cancer, hematological cancer, kidney-urinary tract cancer, liver cancer, lung/pleura cancer, prostate cancer, sarcoma, skin cancer, and uterine cancer. In certain embodiments of these methods, the immune disorder to be treated is an immune disorder associated with a disease selected from the group consisting of: amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriatic arthritis, rheumatoid arthritis, scleroderma, septic shock, Sjorgren's syndrome, ulcerative colitis, and vasculitis.

The use of any composition of the present invention for the treatment or prevention of a cancer, tumor, growth abnormality, and/or immune disorder is within the scope of the present invention. Among certain embodiments of the present invention is a protein of the present invention and/or a pharmaceutical composition thereof for the treatment or prevention of a cancer, tumor, growth abnormality, immune disorder, and/or microbial infection. Among certain embodiments of the present invention is the use of a protein of the invention and/or pharmaceutical composition thereof in the manufacture of a medicament for the treatment or prevention of a cancer, tumor, growth abnormality, immune disorder, or microbial infection.

Certain embodiments of the proteins of the invention may be utilized for the delivery of additional exogenous material into a cell physically coupled with an extracellular target biomolecule of the protein of the invention. Additionally, the present invention provides a method for delivering exogenous material to the inside of a cell(s) comprising contacting the cell(s), either in vitro or in vivo, with a protein, pharmaceutical composition, and/or diagnostic composition of the present invention. The present invention further provides a method for delivering exogenous material to the inside of a cell(s) in a patient, the method comprising the step of administering to the patient a protein of the present invention (with or without cytotoxic activity), wherein the target cell(s) is physically coupled with an extracellular target biomolecule of the protein.

The use of any composition of the present invention for the diagnosis, prognosis, and/or characterization of a disease, disorder, and/or condition is within the scope of the present invention. Among certain embodiments of the present invention is a method of using a protein of the invention comprising a detection promoting agent and/or composition of the invention (e.g. a diagnostic composition) for the collection of information useful in the diagnosis, prognosis, or characterization of a disease, disorder, or condition. Among certain embodiments of the present invention is the method of detecting a cell using a protein and/or diagnostic composition of the invention comprising the steps of contacting a cell with the protein and/or diagnostic composition and detecting the presence of said cell-targeted molecule and/or diagnostic composition. In certain embodiments, the step of contacting the cell(s) occurs in vitro. In certain embodiments, the step of contacting the cell(s) occurs in vivo. In certain embodiments, the step of detecting the cell(s) occurs in vitro. In certain embodiments, the step of detecting the cell(s) occurs in vivo. In certain further embodiments, the method involves the detection of the location of the protein in an organism using one or more imaging procedures after the administration of the protein to said organism. For example, proteins of the invention which incorporate detection promoting agents as described herein may be used to characterize diseases as potentially treatable by a related pharmaceutical composition of the invention. For example, certain compounds (e.g. proteins) of the invention, compositions (e.g. pharmaceutical compositions and diagnostic compositions) of the invention, and methods of the invention may be used to determine if a patient belongs to a group that responds to a pharmaceutical composition of the invention.

Certain embodiments of the present invention are described below, numbered 1-33 (see also WO 2015/138452 A1): (1) A protein comprising a) an immunoglobulin-type binding region comprising one or more polypeptides and capable of specifically binding at least one extracellular target biomolecule, and b) a Shiga toxin effector region comprising a polypeptide, with an amino-terminus, derived from the amino acid sequence of the A Subunit of at least one member of the Shiga toxin family; wherein the immunoglobulin-type binding region and the Shiga toxin effector region are physically oriented within the protein such that the immunoglobulin-type binding region is not located proximally to the amino-terminus of the Shiga toxin effector region. (2) The protein of embodiment 1, wherein the immunoglobulin-type binding region comprises the polypeptide selected from the group consisting of: single-domain antibody fragment, single-chain variable fragment, antibody variable fragment, complementary determining region 3 fragment, constrained FR3-CDR3-FR4 polypeptide, Fd fragment, antigen-binding fragment, fibronectin-derived 10^{th} fibronectin type III domain, tenascin type III domain, ankyrin repeat motif domain, low-density-lipoprotein-receptor-derived A-domain, lipocalin, Kunitz domain, Protein-A-derived Z domain, gamma-B crystalline-derived domain, ubiquitin-derived domain, Sac7d-derived polypeptide, Fyn-derived SH2 domain, miniprotein, C-type lectin-like domain scaffold, engineered antibody mimic, and any genetically manipulated counterparts of any of the foregoing which retain binding functionality. (3) The protein of any one of embodiments 1-2, whereby administration of the protein to a cell physically coupled with an extracellular target biomolecule of the protein's immunoglobulin-type binding region, the protein is capable of causing death of the cell. (4) The protein of embodiment 3, whereby upon administration of the protein to a first population of cells whose members are physically coupled to the extracellular target biomolecule of the immunoglobulin-type binding region of the protein, and a second population of cells whose members are not physically coupled to the extracellular target biomolecule of the immunoglobulin-type binding region, the cytotoxic effect of the protein to members of said first population of cells relative to members of said second population of cells is at least 3-fold greater. (5) The protein of any one of embodiments 1-4, wherein the immunoglobulin-type binding region is capable of binding to an extracellular target biomolecule selected from the group consisting of: CD20, CD22, CD40, CD74, CD79, CD25, CD30, HER2/neu/ErbB2, EGFR, EpCAM, EphB2, prostate-specific membrane antigen, Cripto, CDCP1, endoglin, fibroblast activated protein, Lewis-Y, CD19, CD21, CS1/SLAMF7, CD33, CD52, CD133, EpCAM, CEA, gpA33, Mucins, TAG-72, tyrosine-protein kinase transmembrane receptor, carbonic anhydrase IX, folate binding protein, ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANK, FAP, Tenascin, CD64, mesothelin, BRCA1, MART-1/MelanA, gp100, tyrosinase, TRP-1, TRP-2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen, prostate specific antigen, prostate stem cell antigen, human aspartyl (asparaginyl) beta-hydroxylase, EphA2, HER3/ErbB-3, MUC1, MART-1/MelanA, gp100, tyrosinase associated antigen, HPV-E7, Epstein-Barr Virus antigens, Bcr-Abl, alpha-fetoprotein antigen, 17-A1, bladder tumor antigen, CD38, CD15, CD23, CD53, CD88, CD129, CD183, CD191, CD193, CD244, CD294, CD305, C3AR, FceRIa, galectin-9, mrp-14, PD-L1, siglec-8, siglec-10, CD49d, CD13, CD44, CD54, CD63, CD69, CD123, CD193, TLR4, FceRIa, IgE, CD107a, CD203c, CD14, CD15, CD33, CD64, CD68, CD80, CD86, CD105, CD115, F4/80, ILT-3, Galectin-3, CD11a-c, GITRL, MHC class I molecule, MHC Class II molecule, CD284-TLR4, CD107-Mac3, CD195-CCR5, HLA-DR, CD16/32, CD282-TLR2, CD11c, CD123, and any immunogenic fragment of any of the foregoing. (6) The protein of any one of embodiments 1-5, wherein the Shiga toxin effector region comprises or consists essentially of amino acids 75 to 251 of SEQ ID NO:1, SEQ ID NO:2, or SEQ ID NO:3. (7) The protein of any one of embodiments 1-5, wherein the Shiga toxin effector region comprises or consists essentially of amino acids 1 to 241 of SEQ ID NO:1, SEQ ID NO:2, or SEQ ID NO:3. (8) The protein of embodiment 7, wherein the Shiga toxin effector region comprises or consists essentially of amino acids 1 to 251 of SEQ ID NO:1, SEQ ID NO:2, or SEQ ID NO:3. (9) The protein of embodiment 8, wherein the Shiga toxin effector region comprises or consists essentially of amino acids 1 to 261 of SEQ ID NO:1, SEQ ID NO:2, or SEQ ID NO:3. (10) The protein of embodiment 5, wherein the immunoglobulin-type binding region comprises or consists essentially of amino acids 269-508 of any one of SEQ ID NOs: 4, 8, 12, 16. (11) The protein of any one of embodiments 6-9, wherein the immunoglobulin-type binding region comprises or consists essentially of amino acids 269-508 of any one of SEQ ID NOs: 4, 8, 12, 16. (12) The protein of embodiment 5, which comprises or consists essentially of the polypeptide of shown in any one of SEQ ID NOs: 4-31. (13) The protein of any one of embodiments 1-12, wherein the Shiga toxin effector region comprises a mutation relative to a naturally occurring A Subunit of a member of the Shiga toxin family which changes the enzymatic activity of the Shiga toxin effector region, the mutation selected from at least one amino acid residue deletion, insertion, or substitution. (14) The protein of embodiment 13, wherein said mutation reduces or eliminates cytotoxicity of the Shiga toxin effector region. (15) A pharmaceutical composition comprising the protein of any one of embodiments 1-14 and at least one pharmaceutically acceptable excipient or carrier. (16) A diagnostic composition comprising the protein of any one of embodiments 1-14 and a detection promoting agent. (17) A polynucleotide capable of encoding the protein of any one of embodiments 1-14 or a complement thereof, or a fragment of any of the foregoing. (18) An expression vector comprising the polynucleotide of embodiment 17. (19) A host cell comprising any one of the polynucleotides or expression vectors of embodiments 17-18. (20) A system for conferring improved cytotoxicity to a protein which comprises a) an immunoglobulin-type binding region comprising one or more polypeptides and capable of specifically binding at least one extracellular target biomolecule, and b) a Shiga toxin effector region comprising a polypeptide, with a carboxy-terminus, derived from the amino acid sequence of the A Subunit of at least one member of the Shiga toxin family; the system comprising the step of arranging the immunoglobulin-type binding region proximally to the carboxy-terminus of the Shiga toxin effector region within the protein. (21) A method of killing a cell comprising the step of contacting the cell with the protein of any one of embodiments 1-14 or the pharmaceutical composition of embodiment 15. (22) The method of embodiment 20, wherein the contacting occurs in vitro. (23) The method of embodiment 20, wherein the contacting occurs in vivo. (24) A method of treating a disease, disorder, or condition in a patient comprising the step of administering to a patient in need thereof a therapeutically effective amount of the protein of any one of embodiments 1-14 or the pharmaceutical composition of embodiment 15. (25) The method of embodiment 24, wherein the disease, disorder, or condition is selected from the group consisting of: cancer, tumor, immune disorder, and microbial infection. (26) The method of embodiment 25, wherein the cancer selected from the group consisting of: bone cancer, breast cancer, central/peripheral nervous system cancer, gastrointestinal cancer, germ cell cancer, glandular cancer, head-neck cancer, hematological cancer, kidney-urinary tract cancer, liver cancer, lung/pleura cancer, prostate cancer, sarcoma, skin cancer, and uterine cancer. (27) The method of embodiment 25, wherein the immune disorder is associated with a disease selected from the group consisting of: amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriasis, psoriatic arthritis, rheumatoid arthritis, scleroderma, septic shock, Sjorgren's syndrome, ulcerative colitis, and vasculitis. (28) A composition of matter of any one of embodiments 1-19 for the treatment or prevention of a cancer, a tumor, immune disorder, or microbial infection. (29) Use of a composition of matter of any one of embodiments 1-19 in the manufacture of a medicament for the treatment or prevention of a cancer, tumor, immune disorder, or microbial infection. (30) A method of collecting information regarding the presence of a cell physically coupled with an extracellular target of the immunoglobulin-type binding region of the protein of the invention comprising the steps of contacting a cell with the diagnostic composition of embodiment 16 and detecting the presence of the diagnostic agent. (31) The method of embodiment 30, wherein the contacting occurs in vivo. (32) The method of embodiment 30, wherein the detecting occurs in vivo. (33) A kit comprising the composition of matter of any one of embodiments 1-19 and an additional reagent and/or pharmaceutical delivery device.

In certain embodiments of the proteins of the present invention, a linker may be used which comprises one or more protease sensitive sites to provide for cleavage by a protease present within a target cell. In certain embodiments of the proteins of the invention, a linker may be used which is not cleavable to reduce unwanted toxicity after administration to a vertebrate organism (see e.g. Poison A et al., Cancer Res 69: 2358-64 (2009)).

In certain embodiments of the proteins of the invention, a cell-targeting binding region is linked to a Shiga toxin effector region using any number of means known to the skilled worker, including both covalent and noncovalent linkages (see e.g. Chen X et al., Adv Drug Deliv Rev 65: 1357-69 (2013); Behrens C, Liu B, MAbs 6: 46-53 (2014).

In certain embodiments of the proteins of the present invention, the protein comprises a binding region which is a scFv with a linker connecting a heavy chain variable (V_{H}) domain and a light chain variable (V_{L}) domain. There are numerous linkers known in the art suitable for this purpose, such as, e.g., the 15-residue (Gly4Ser)₃ peptide. Suitable scFv linkers which may be used in forming non-covalent multivalent structures include GGS, GGGS (Gly3Ser or G3S), GGGGS (Gly4Ser or G4S), GGGGSGGG, GGSGGGG, GSTSGGGSGGGSGGGGSS, and GSTSGSGKPGSSEGSTKG (Plückthun A, Pack P, Immunotechnology 3: 83-105 (1997); Atwell J et al., Protein Eng 12: 597-604 (1999); Wu A et al., Protein Eng 14: 1025-33 (2001); Yazaki P et al., J Immunol Methods 253: 195-208 (2001); Carmichael J et al., J Mol Biol 326: 341-51 (2003); Arndt M et al., FEBS Lett 578: 257-61 (2004); Bie C et al., World J Hepatol 2: 185-91 (2010)).

Among certain embodiments, the proteins of the present invention comprise the Shiga toxin effector region comprising or consisting essentially of amino acids 75 to 251 of SLT-1A (SEQ ID NO:1), StxA (SEQ ID NO:2), and/or SLT-2A (SEQ ID NO:3). Further embodiments are proteins in which the Shiga toxin effector region comprises or consists essentially of amino acids 1 to 241 of SLT-1A (SEQ ID NO:1), StxA (SEQ ID NO:2), and/or SLT-2A (SEQ ID NO:3). Further embodiments are proteins in which the Shiga toxin effector region comprises or consists essentially of amino acids 1 to 251 of SLT-1A (SEQ ID NO:1), StxA (SEQ ID NO:2), and/or SLT-2A (SEQ ID NO:3). Further embodiments are proteins in which the Shiga toxin effector region comprises or consists essentially of amino acids 1 to 261 of SLT-1A (SEQ ID NO:1), StxA (SEQ ID NO:2), and/or SLT-2A (SEQ ID NO:3).

In certain embodiments, the proteins comprise the immunoglobulin-type binding region comprising or consisting essentially of amino acids 269-508 of SEQ ID NO:4, which exhibits high affinity binding specifically to human CD38. Further embodiments are the proteins comprising or consisting essentially of any one of the polypeptides shown in SEQ ID NOs: 4-7.

In certain embodiments, the proteins of the present invention comprise the immunoglobulin-type binding region comprising or consisting essentially of amino acids 269-512 of SEQ ID NO:8, which exhibits high affinity binding specifically to human HER2. Further embodiments are the proteins comprising or consisting essentially of any one of the polypeptides shown in SEQ ID NOs: 8-11.

In certain embodiments, the proteins comprise the immunoglobulin-type binding region comprising or consisting essentially of amino acids 269-516 of SEQ ID NO:12, which exhibits high affinity binding specifically to human CD19. Further embodiments are the proteins comprising or consisting essentially of any one of the polypeptides shown in SEQ ID NOs: 12-15.

In certain embodiments, the proteins comprise the immunoglobulin-type binding region comprising or consisting essentially of amino acids 269-518 of SEQ ID NO:16, which exhibits high affinity binding specifically to human CD74. Further embodiments are the proteins comprising or consisting essentially of any one of the polypeptides shown in SEQ ID NOs: 16-19.

Among certain embodiments of the proteins of the present invention, the binding region is a single domain immunoglobulin-derived region V_{H}H which exhibits high affinity binding specifically to HER2, such as derived from a single-domain variable region of the camelid antibody (V_{H}H) protein 5F7, as described in U.S. Patent Application Publication 2011/0059090. In certain further embodiments, the proteins comprise the immunoglobulin-type binding region comprising or consisting essentially of amino acids 268-385 of SEQ ID NO:20, which exhibits high affinity binding specifically to human HER2. Further embodiments are the proteins comprising or consisting essentially of any one of the polypeptides shown in SEQ ID NOs: 20-29.

In certain embodiments, the proteins comprise the immunoglobulin-type binding region comprising or consisting essentially of amino acids 269-365 of SEQ ID NO:16, which exhibits high affinity binding specifically to human CD20. Further embodiments are the proteins comprising or consisting essentially of any one of the polypeptides shown in SEQ ID NOs: 30-31.

In certain embodiments, the proteins of the invention are capable of binding extracellular target biomolecules associated with the cell surface of particular cell types and entering those cells. Once internalized within a targeted cell type, certain embodiments of the proteins of the invention are capable of routing a cytotoxic Shiga toxin effector polypeptide fragment into the cytosol of the target cell. Once in the cytosol of a targeted cell type, certain embodiments of the cytotoxic proteins of the invention are capable of enzymatically inactivating ribosomes, interfering with cell homeostasis, and eventually killing the cell.

In certain embodiments of the cytotoxic proteins of the present invention, upon contacting a cell physically coupled with an extracellular target biomolecule of the immunoglobulin-type binding region of a cytotoxic protein of the invention, the cytotoxic protein is capable of causing death of the cell. Cell kill may be accomplished using a cytotoxic protein of the invention under varied conditions of target cells, such as an ex vivo manipulated target cell, a target cell cultured in vitro, a target cell within a tissue sample cultured in vitro, or a target cell in vivo.

In certain embodiments, administration of the cytotoxic protein of the present invention to a mixture of cell types, the cytotoxic protein is capable of selectively killing those cells which are physically coupled with a certain extracellular target biomolecule compared to cell types not physically coupled with any extracellular target biomolecule specifically bound by the binding region of that cytotoxic protein. Because members of the Shiga toxin family are adapted for killing eukaryotic cells, cytotoxic proteins designed using Shiga toxin effector regions can show potent cytotoxic activity. By targeting the delivery of enzymatically active Shiga toxin regions to specific cell types using high-affinity binding regions, this potent cell kill activity can be restricted to preferentially killing only those cell types desired to be targeted by their physical association with a target biomolecule specifically bound by chosen binding regions.

In certain embodiments, the cytotoxic protein of the present invention is capable of selectively or preferentially causing the death of a specific cell type within a mixture of two or more different cell types. This enables the targeting of cytotoxic activity to specific cell types with a high preferentiality, such as a 3-fold cytotoxic effect, over “bystander” cell types that do not express the target biomolecule. Alternatively, the expression of the target biomolecule of the binding region may be non-exclusive to one cell type if the target biomolecule is expressed in low enough amounts and/or physically coupled in low amounts with cell types that are not to be targeted. This enables the targeted cell-killing of specific cell types with a high preferentiality, such as a 3-fold cytotoxic effect, over “bystander” cell types that do not express significant amounts of the target biomolecule or are not physically coupled to significant amounts of the target biomolecule.

In certain further embodiments, administration of the cytotoxic protein of the invention to two populations of cell types which differ with respect to the presence and/or polypeptide sequence of an extracellular target biomolecule, the cytotoxic protein is capable of causing cell death as defined by the half-maximal cytotoxic concentration (CD₅₀) on a population of target cells, whose members express an extracellular target biomolecule of the binding region of the cytotoxic protein, e.g., at a dose at least three-times lower than the CD₅₀ dose of the same cytotoxic protein to a population of cells whose members do not express an extracellular target biomolecule of the binding region of the cytotoxic protein.

In certain embodiments, the cytotoxic activity of a protein of the present invention toward populations of cell types physically coupled with an extracellular target biomolecule is at least 3-fold higher than the cytotoxic activity toward populations of cell types not physically coupled with any extracellular target biomolecule bound specifically by that protein of the invention. According to the present invention, selective cytotoxicity may be quantified in terms of the ratio (a/b) of (a) cytotoxicity towards a population of cells of a specific cell type physically coupled with a target biomolecule of the binding region to (b) cytotoxicity towards a population of cells of a cell type not physically coupled with a target biomolecule of the binding region. In certain embodiments, the cytotoxicity ratio is indicative of selective cytotoxicity which is at least 3-fold, 5-fold, 10-fold, 15-fold, 20-fold, 25-fold, 30-fold, 40-fold, 50-fold, 75-fold, 100-fold, 250-fold, 500-fold, 750-fold, 1000-fold, or higher for populations of cells or cell types physically coupled with a target biomolecule of the binding region compared to populations of cells or cell types not physically coupled with a target biomolecule of the binding region.

In certain embodiments, the proteins of the present invention are capable of selectively or preferentially causing the death of a specific cell type within a mixture of two or more different cell types. This enables the targeted cytotoxic activity to specific cell types with a high preferentiality, such as a 3-fold cytotoxic effect, over “bystander” cell types that do not express extracellular target bound specifically by the binding region of that cytotoxic protein of the invention. Alternatively, the expression of an extracellular target biomolecule may be non-exclusive to one cell type if the extracellular target biomolecule is expressed in low enough amounts by cell types that are not to be targeted. This enables the preferential cell-killing of only those cell type(s) expressing the highest amounts of extracellular target biomolecules, such as a 3-fold cytotoxic effect, over “bystander” cell types that do not express significant amounts of the target biomolecule bound specifically by that cytotoxic protein and/or are not physically coupled to significant amounts of extracellularly exposed target biomolecule bound specifically by that cytotoxic protein.

In certain embodiments, the cytotoxic activity of a protein of the present invention toward populations of cell types physically coupled with a certain extracellular target biomolecule is at least 3-fold higher than the cytotoxic activity toward populations of cell types not physically coupled with significant amounts of an extracellular target biomolecule bound specifically by the binding region of that particular protein of the invention. According to the present invention, selective cytotoxicity may be quantified in terms of the ratio (a/b) of (a) cytotoxicity towards a population of cells physically coupled with a significant amount of an extracellular target biomolecule bound by the binding region of the cytotoxic protein of the invention to (b) cytotoxicity towards a population of cells of a cell type not physically coupled with a significant amount of any extracellular target biomolecule bound specifically by the binding region of that particular cytotoxic protein of the invention. In certain embodiments, the cytotoxicity ratio is indicative of selective cytotoxicity which is at least 3-fold, 5-fold, 10-fold, 15-fold, 20-fold, 25-fold, 30-fold, 40-fold, 50-fold, 75-fold, 100-fold, 250-fold, 500-fold, 750-fold, 1000-fold or higher for populations of cells or cell types expressing an extracellular target biomolecule or physically coupled with an extracellular target biomolecule bound by the binding region of the cytotoxic protein of the invention compared to populations of cells or cell types which do not express an extracellular target biomolecule or that are not physically coupled with significant amounts of an extracellular target biomolecule bound specifically the binding region of that particular cytotoxic protein of the invention. For example, administration of certain cytotoxic proteins of the present invention to two different populations of cells which differ with respect to the presence and/or polypeptide sequence of an extracellular target biomolecule, the cytotoxic protein of the invention is capable of causing cell death to the cell-types physically coupled with an extracellular target biomolecule bound by the cytotoxic protein's binding region, e.g., at a CD₅₀ at least three times or less than the CD₅₀ of binding to cell types that are not physically coupled with an extracellular target biomolecule bound by the cytotoxic protein's binding region or to cell types that are physically coupled only with forms of that extracellular target biomolecule which comprise sequence variations or mutations which disrupt binding specificity by the binding region of that cytotoxic protein.

In certain embodiments of the cytotoxic proteins of the present invention, administration of the cytotoxic protein to two different populations of cell types, the cytotoxic protein is capable of causing cell death as defined by the half-maximal cytotoxic concentration (CD50) on a first cell population, whose members express a certain target biomolecule at a cellular surface, at a dose at least three-times lower than the CD₅₀ dose of the same cytotoxic protein to a second population of cells whose members do not express that target biomolecule, do not express a significant amount of that target biomolecule, or are not exposing a significant amount of that target biomolecule bound by the cytotoxic protein's binding region.

In addition to direct cell killing, proteins of the present invention optionally may be used for delivery of additional exogenous materials into the interiors of target cells. In certain embodiments of the proteins of the present invention for delivery of additional exogenous material, the additional exogenous material is a cytotoxic agent, such as, e.g., a small molecule chemotherapeutic agent, cytotoxic antibiotic, alkylating agent, antimetabolite, topoisomerase inhibitor, and/or tubulin inhibitor. Non-limiting examples of cytotoxic agents include aziridines, cisplatins, tetrazines, procarbazine, hexamethylmelamine, vinca alkaloids, taxanes, camptothecins, etoposide, doxorubicin, mitoxantrone, teniposide, novobiocin, aclarubicin, anthracyclines, actinomycin, bleomycin, plicamycin, mitomycin, daunorubicin, epirubicin, idarubicin, dolastatins, maytansines, docetaxel, adriamycin, calicheamicin, auristatins, pyrrolobenzodiazepine, carboplatin, 5-fluorouracil (5-FU), capecitabine, mitomycin C, paclitaxel, 1,3-Bis(2-chloroethyl)-1-nitrosourea (BCNU), rifampicin, cisplatin, methotrexate, and gemcitabine.

Certain proteins of the invention have uses in the in vitro and/or in vivo detection of specific cells, cell types, and/or cell populations. In certain embodiments, the cytotoxic proteins described herein are used for both diagnosis and treatment, or for diagnosis alone. When the same cytotoxic protein is used for both diagnosis and treatment, the cytotoxic protein variant which incorporates a detection promoting agent for diagnosis may be rendered non-toxic by catalytic inactivation of a Shiga toxin effector region via one or more amino acid substitutions, including exemplary substitutions described herein. Catalytically inactive forms of the cytotoxic proteins of the invention that are conjugated to detection promoting agents optionally may be used for diagnostic functions, such as for companion diagnostics used in conjunction with a therapeutic regimen comprising the same or a related binding region.

The ability to conjugate detection promoting agents known in the art to various proteins of the invention provides useful compositions for the detection of cancer, tumor, immune, and infected cells. These diagnostic embodiments of the proteins of the invention may be used for information gathering via various imaging techniques and assays known in the art. For example, diagnostic embodiments of the proteins of the invention may be used for information gathering via imaging of intracellular organelles (e.g. endocytotic, Golgi, endoplasmic reticulum, and cytosolic compartments) of individual cancer cells, immune cells, or infected cells in a patient or biopsy sample.

Various types of information may be gathered using the diagnostic embodiments of the proteins of the invention whether for diagnostic uses or other uses. This information may be useful, for example, in diagnosing neoplastic cell types, determining therapeutic susceptibilities of a patient's disease, assaying the progression of antineoplastic therapies over time, assaying the progression of immunomodulatory therapies over time, assaying the progression of antimicrobial therapies over time, evaluating the presence of infected cells in transplantation materials, evaluating the presence of unwanted cell types in transplantation materials, and/or evaluating the presence of residual tumor cells after surgical excision of a tumor mass.

In certain of the above embodiments, the protein of the present invention is a variant in which there are one or more conservative amino acid substitutions introduced into the polypeptide region(s). In certain embodiments, a protein of the present invention may comprise functional fragments or variants of a polypeptide region of the invention that have, at most, 20, 15, 10, 9, 8, 7, 6, 5, 4, 3, 2, or 1 amino acid substitution(s) compared to a polypeptide sequence recited herein, as long as the substituted polypeptide region retains measurable biological activity alone or as a component of a protein of the invention. Variants of proteins of the invention are within the scope of the invention as a result of changing a polypeptide of the protein of the invention by altering one or more amino acids or deleting or inserting one or more amino acids, such as within the immunoglobulin-type binding region or the Shiga toxin effector region, in order to achieve desired properties, such as changed cytotoxicity, changed cytostatic effects, changed immunogenicity, and/or changed serum half-life. A polypeptide of a protein of the invention may further be with or without a signal sequence.

In certain embodiments, a protein of the present invention shares at least 85%, 90%, 95%, 96%, 97%, 98%, 99% or more amino acid sequence identity to any one of the amino acid sequences of a protein recited herein, as long as it retains measurable biological activity, such as cytotoxicity, extracellular target biomolecule binding, enzymatic catalysis, or subcellular routing. The immunoglobulin-type binding region may differ from the amino acid sequences of a protein recited herein, as long as it retains binding functionality to its extracellular target biomolecule. Binding functionality will most likely be retained if the amino acid sequences of the CDRs or ABRs are identical. For example, a protein is within the claim scope that comprises or consists essentially of 85% amino acid identity to a protein recited herein which for the purposes of determining the degree of amino acid identity, the amino acid residues that form the CDRs or ABRs are disregarded. Binding functionality can be determined by the skilled worker using standard techniques.

In certain embodiments, the proteins of the invention may optionally be purified in homo-multimeric forms (i.e. a protein complex of two or more identical proteins) or in hetero-multimeric forms (i.e. a protein complex of two or more non-identical proteins).

In certain embodiments, the pharmaceutical composition of the invention may comprise homo-multimeric and/or hetero-multimeric forms of the proteins of the invention.

Beyond the proteins of the present invention, the polynucleotides which encode such proteins, or functional portions thereof, are within the scope of the present invention.

In one aspect, the invention provides polynucleotides which encode a protein of the invention, or a fragment or derivative thereof. The polynucleotides may include, e.g., nucleic acid sequence encoding a polypeptide at least 50%, 55%, 60%, 65%, 70%, 75%, 80%, 85%, 90%, 95%, 99% or more, identical to a polypeptide comprising one of the amino acid sequences of the protein of the invention. The invention also includes polynucleotides comprising nucleotide sequences that hybridize under stringent conditions to a polynucleotide which encodes a protein of the invention, or a fragment or derivative thereof, or the antisense or complement of any such sequence.

In certain embodiments, a protein or pharmaceutical composition of the present invention can be used to kill specific cell types in a mixture of different cell types, such as mixtures comprising cancer cells, infected cells, and/or hematological cells. In certain embodiments, a protein or pharmaceutical composition of the present invention can be used to kill cancer cells in a mixture of different cell types. In certain embodiments, a protein or pharmaceutical composition of the present invention can be used to kill specific cell types in a mixture of different cell types, such as pre-transplantation tissues. In certain embodiments, a protein or pharmaceutical composition of the present invention can be used to kill specific cell types in a mixture of cell types, such as pre-administration tissue material for therapeutic purposes. In certain embodiments, a protein or pharmaceutical composition of the present invention can be used to selectively kill cells infected by viruses or microorganisms, or otherwise selectively kill cells expressing a particular extracellular target biomolecule, such as a cell surface biomolecule. The proteins and pharmaceutical compositions of the invention have varied applications, including, e.g., uses in depleting unwanted cell types from tissues either in vitro or in vivo, uses in modulating immune responses to treat graft-versus-host disease, uses as antiviral agents, uses as anti-parasitic agents, and uses in purging transplantation tissues of unwanted cell types.

In certain embodiments, a protein or pharmaceutical composition of the present invention, alone or in combination with other compounds or pharmaceutical compositions, can show potent cell-kill activity when administered to a population of cells, in vitro or in vivo in a subject such as in a patient in need of treatment. By targeting the delivery of enzymatically active Shiga toxin regions using high-affinity immunoglobulin-type binding regions to cancer cell types, this potent cell-kill activity can be restricted to specifically and selectively kill certain cell types within an organism, such as certain cancer cells, neoplastic cells, malignant cells, non-malignant tumor cells, or infected cells.

Certain embodiments of the proteins of the invention or pharmaceutical compositions thereof can be used to kill a cancer and/or tumor cell in a patient by targeting an extracellular biomolecule found physically coupled with a cancer and/or tumor cell. The terms “cancer cell” or “cancerous cell” refers to various neoplastic cells which grow and divide in an abnormally accelerated fashion and will be clear to the skilled person. The term “tumor cell” includes both malignant and non-malignant cells (e.g. non-cancerous, benign tumor cells, non-cancerous “cancer” stem cells, tumor stem cells, pre-malignant cancer-initiating cells, tumor-initiating cells, or tumorigenic cells all of which can give rise to daughter cells which become malignant tumor and/or cancer cells but are unable to metastasize on their own (see e.g. Martinez-Climent J et al., Haematologica 95: 293-302 (2010)). Generally, cancers and/or tumors can be defined as diseases, disorders, or conditions that are amenable to treatment and/or prevention. The cancers and tumors (either malignant or non-malignant) which are comprised of cancer cells and/or tumor cells which may benefit from methods and compositions of the invention will be clear to the skilled person. Neoplastic cells are often associated with one or more of the following: unregulated growth, lack of differentiation, local tissue invasion, angiogenesis, and metastasis.

Certain embodiments of the proteins of the invention or pharmaceutical compositions thereof can be used to kill an immune cell (whether healthy or malignant) in a patient by targeting an extracellular biomolecule found physically coupled with an immune cell.

Certain embodiments of the proteins of the invention or pharmaceutical compositions thereof can be used to kill an infected cell in a patient by targeting an extracellular biomolecule found physically coupled with an infected cell.

It is within the scope of the present invention to utilize the protein of the invention or pharmaceutical composition thereof for the purposes of purging patient cell populations (e.g. bone marrow) of infected malignant, neoplastic, or otherwise unwanted B-cells and/or T-cells and then reinfusing the B-cell and/or T-cell depleted material into the patient (see e.g. van Heeckeren W et al., Br J Haematol 132: 42-55 (2006); Alpdogan O, van den Brink M, Semin Oncol 39: 629-42 (2012)).

It is within the scope of the present invention to utilize the protein of the invention or pharmaceutical composition thereof for the purposes of ex vivo depletion of B-cells and/or T cells from isolated cell populations removed from a patient. In one non-limiting example, the protein of the invention may be used in a method for prophylaxis of organ and/or tissue transplant rejection wherein the donor organ or tissue is perfused prior to transplant with a cytotoxic protein of the invention or a pharmaceutical composition thereof in order to purge the organ of unwanted donor B-cells and/or T-cells (see e.g. Alpdogan O, van den Brink M, Semin Oncol 39: 629-42 (2012)).

It is also within the scope of the present invention to utilize the protein of the invention or pharmaceutical composition thereof for the purposes of depleting B-cells and/or T-cells from a donor cell population as a prophylaxis against graft-versus-host disease, and induction of tolerance, in a patient to undergo a bone marrow and or stem cell transplant.

Certain embodiments of the protein of the invention or pharmaceutical compositions thereof can be used to kill an infected cell in a patient by targeting an extracellular biomolecule found physically coupled with an infected cell.

Certain embodiments of the protein of the invention or pharmaceutical compositions thereof can be used to kill a cell(s) of a multicellular parasite. In certain further embodiments, the cell killing occurs while the multicellular parasite is present in a host organism or subject. In certain further embodiments, the protein of the invention may be used to kill a helminth, such as, e.g., a plathelminth, nemathelminth, cestode, mongenean, nematode, and/or trematode.

Additionally, the present invention provides a method of treating a disease, disorder, or condition in a patient comprising the step of administering to a patient in need thereof a therapeutically effective amount of at least one of the proteins of the present invention or a pharmaceutical composition thereof. Contemplated diseases, disorders, and conditions that can be treated using this method include cancers, malignant tumors, non-malignant tumors, growth abnormalities, immune disorders, and microbial infections. Administration of a “therapeutically effective dosage” of a compound of the invention can result in a decrease in severity of disease symptoms, an increase in frequency and duration of disease symptom-free periods, or a prevention of impairment or disability due to the disease affliction.

Routes of administration for proteins of the present invention or compositions thereof include, e.g. intravenous, intramuscular, intradermal, intraperitoneal, subcutaneous, spinal, or other parenteral routes of administration, for example by injection or infusion at or in communication with the intended site of action (e.g. intratumoral injection). In other embodiments, a protein or pharmaceutical composition of the invention may be administered by a non-parenteral route, such as a topical, epidermal or mucosal route of administration, for example, intranasally, orally, vaginally, rectally, sublingually, or topically.

Proteins or pharmaceutical compositions of the invention may be administered with one or more of a variety of medical devices known in the art. For example, in one embodiment, a pharmaceutical composition of the invention may be administered with a needleless hypodermic injection device. Examples of well-known implants and modules useful in the present invention are in the art, including e.g., implantable micro-infusion pumps for controlled rate delivery; devices for administering through the skin; infusion pumps for delivery at a precise infusion rate; variable flow implantable infusion devices for continuous drug delivery; and osmotic drug delivery systems. These and other such implants, delivery systems, and modules are known to those skilled in the art.

A protein or pharmaceutical composition of the present invention may be administered alone or in combination with one or more other therapeutic or diagnostic agents. A combination therapy may include a protein of the invention or pharmaceutical composition thereof combined with at least one other therapeutic agent selected based on the particular patient, disease or condition to be treated. Examples of other such agents include, inter alia, a cytotoxic, anti-cancer or chemotherapeutic agent, an anti-inflammatory or anti-proliferative agent, an antimicrobial or antiviral agent, growth factors, cytokines, an analgesic, a therapeutically active small molecule or polypeptide, a single chain antibody, a classical antibody or fragment thereof, or a nucleic acid molecule which modulates one or more signaling pathways, and similar modulating therapeutics which may complement or otherwise be beneficial in a therapeutic or prophylactic treatment regimen.

Treatment of a patient with a protein or pharmaceutical composition of the invention preferably leads to cell death of targeted cells and/or the inhibition of growth of targeted cells. As such, proteins of the invention, and pharmaceutical compositions comprising them, will be useful in methods for treating a variety of pathological disorders in which killing or depleting target cells may be beneficial, such as, inter alia, cancers, tumors, growth abnormalities, immune disorders, and infected cells. The present invention provides methods for suppressing cell proliferation, and treating cell disorders, including neoplasia, overactive B-cells, and overactive T-cells.

In certain embodiments, proteins and pharmaceutical compositions of the invention may be used to treat or prevent cancers, tumors (malignant and non-malignant), growth abnormalities, immune disorders, and microbial infections. In a further aspect, the above ex vivo method can be combined with the above in vivo method to provide methods of treating or preventing rejection in bone marrow transplant recipients, and for achieving immunological tolerance.

In certain embodiments, the present invention provides methods for treating malignancies or neoplasms and other blood cell associated cancers in a mammalian subject, such as a human, the method comprising the step of administering to a subject in need thereof a therapeutically effective amount of a protein or pharmaceutical composition of the invention.

The proteins and pharmaceutical compositions of the invention have varied applications, including, e.g., uses in removing unwanted B-cells and/or T-cells, uses in modulating immune responses to treat graft-versus-host diseases, uses as antiviral agents, uses as antimicrobial agents, and uses in purging transplantation tissues of unwanted cell types. The proteins and pharmaceutical compositions of the present invention are commonly anti-neoplastic agents—meaning they are capable of treating and/or preventing the development, maturation, or spread of neoplastic or malignant cells by inhibiting the growth and/or causing the death of cancer or tumor cells.

In certain embodiments, a protein or pharmaceutical composition of the present invention is used to treat a B-cell-, plasma cell-, T-cell, or antibody-mediated disease or disorder, such as for example leukemia, lymphoma, myeloma, amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriasis, psoriatic arthritis, rheumatoid arthritis, scleroderma, septic shock, Sjorgren's syndrome, ulcerative colitis, and vasculitis.

In another aspect, certain embodiments of the proteins and pharmaceutical compositions of the present invention are antimicrobial agents—meaning they are capable of treating and/or preventing the acquisition, development, or consequences of microbiological pathogenic infections, such as caused by viruses, bacteria, fungi, prions, or protozoans.

It is within the scope of the present invention to provide a prophylaxis or treatment for diseases or conditions mediated by B-cells and/or T-cells, the prophylaxis or treatment involving administering the protein or the invention, or a pharmaceutical composition thereof, to a patient in need thereof for the purpose of killing B-cells and/or T-cells in the patient. This usage is compatible with preparing or conditioning a patient for bone marrow transplantation, stem cell transplantation, tissue transplantation, or organ transplantation, regardless of the source of the transplanted material, e.g. human or non-human sources.

The proteins and pharmaceutical compositions of the present invention may be utilized in a method of treating an immune disorder comprising administering to a patient, in need thereof, a therapeutically effective amount of the protein or a pharmaceutical composition of the present invention. In certain embodiments of the methods of the present invention, the immune disorder is related to an inflammation associated with a disease selected from the group consisting of: amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriasis, psoriatic arthritis, rheumatoid arthritis, scleroderma, septic shock, Sjorgren's syndrome, ulcerative colitis, and vasculitis.

Among certain embodiments of the present invention is using the protein of the invention as a component of a pharmaceutical composition or medicament for the treatment or prevention of a cancer, tumor, growth abnormality, immune disorder, and/or microbial infection. For example, immune disorders presenting on the skin of a patient may be treated with such a medicament in efforts to reduce inflammation. In another example, skin tumors may be treated with such a medicament in efforts to reduce tumor size or eliminate the tumor completely.

Certain proteins of the invention may be used in molecular neurosurgery applications such as immunolesioning and neuronal tracing (see, Wiley R, Lappi D, Adv Drug Deliv Rev 55: 1043-54 (2003), for review). For example, the targeting domain may be selected or derived from various ligands, such as neurotransmitters and neuropeptides, which target specific neuronal cell types by binding neuronal surface receptors, such as a neuronal circuit specific G-protein coupled receptor. Similarly, the targeting domain may be selected from or derived from antibodies that bind neuronal surface receptors. Because Shiga toxins robustly direct their own retrograde axonal transport, certain cytotoxic proteins of the invention may be used to kill a neuron(s) which expresses the extracellular target at a site of cytotoxic protein injection distant from the cell body (see Llewellyn-Smith I et al., J Neurosci Methods 103: 83-90 (2000)). These neuronal cell type specific targeting cytotoxic proteins have uses in neuroscience research, such as for elucidating mechanisms of sensations (see e.g. Mishra S, Hoon M, Science 340: 968-71 (2013)), and creating model systems of neurodegenerative diseases, such as Parkinson's and Alzheimer's (see e g Hamlin A et al., PLoS One e53472 (2013)).

Among certain embodiments of the present invention is a method of using a protein, pharmaceutical composition, and/or diagnostic composition of the invention to detect the presence of a cell type for the purpose of information gathering regarding diseases, conditions and/or disorders. The method comprises contacting a cell with a diagnostically sufficient amount of a protein of the invention to detect the protein by an assay or diagnostic technique. The term “diagnostically sufficient amount” refers to an amount that provides adequate detection and accurate measurement for information gathering purposes by the particular assay or diagnostic technique utilized. Generally, the diagnostically sufficient amount for whole organism in vivo diagnostic use will be a non-cumulative dose of between 0.1 mg to 100 mg of the detection promoting agent linked protein per kg of subject per subject. Typically, the amount of protein of the invention used in these information gathering methods will be as low as possible provided that it is still a diagnostically sufficient amount. For example, for in vivo detection in an organism, the amount of protein of the invention administered to a subject will be as low as feasibly possible.

The cell-type specific targeting of proteins of the invention combined with detection promoting agents provides a way to detect and image cells physically coupled with an extracellular target biomolecule of a binding region of the proteins of the invention. Imaging of cells using the proteins of the invention may be performed in vitro or in vivo by any suitable technique known in the art. For example, the method of using a protein, pharmaceutical composition, or diagnostic composition of the invention to detect the presence of a cell type for the purpose of information gathering may be performed on cells in vivo within a patient, including on cells in situ, e.g. at a disease locus, on cells in vitro, and/or in an ex vivo setting on cells and tissues removed from an organism, e.g. a biopsy material.

The method of using a protein, pharmaceutical composition, or diagnostic composition of the invention to detect the presence of a target biomolecule positive cell type for the purpose of information gathering may be performed on cells in vivo within a patient, on cells in situ, e.g. at a disease locus, on cells in vitro, and/or in an ex vivo setting on cells and tissues removed from an organism, e.g. a biopsy material. The detection of specific cells, cell types, and cell populations using a composition of the invention may be used for diagnosis and imaging of cells, such as, e.g., tumor, cancer, immune, and infected cells. For example, proteins and diagnostic compositions of the invention may be employed to image or visualize a site of possible accumulation of target biomolecule expressing cells in an organism. These methods may be used to identify sites of tumor development or residual tumor cells after a therapeutic intervention.

Among certain embodiment of the present invention is a method of using a protein, pharmaceutical composition, and/or diagnostic composition to label or detect the interiors of neoplastic cells and/or immune cell types (see e.g., Koyama Y et al., Clin Cancer Res 13: 2936-45 (2007); Ogawa M et al., Cancer Res 69: 1268-72 (2009); Yang L et al., Small 5: 235-43 (2009)). Based on the ability of the proteins and pharmaceutical compositions of the invention to enter specific cell types and route within cells via retrograde intracellular transport, the interior compartments of specific cell types are labeled for detection. This can be performed on cells in situ within a patient or on cells and tissues removed from an organism, e.g. biopsy material.

In certain embodiments, the proteins of the invention or pharmaceutical and/or diagnostic compositions thereof are used for both diagnosis and treatment, or for diagnosis alone.

Below, certain embodiments of the present invention are described by referring to biological sequence information in Table C, below.

In certain embodiments, the cell-targeted molecule of the present invention comprises 1) a binding region comprising an immunoglobulin-type binding region comprising one or more polypeptides and capable of specifically binding at least one extracellular target biomolecule, and 2) a Shiga toxin effector polypeptide comprising a Shiga toxin A1 fragment region having a carboxy terminus and a disrupted furin-cleavage motif at the carboxy terminus of the A1 fragment region. In certain further embodiments of the cell-targeted molecule of the invention, the binding region is associated with the carboxy terminus of the Shiga toxin effector polypeptide. In further certain embodiments of the cell-targeted molecule of the invention, the binding region is fused to the Shiga toxin effector polypeptide. In further certain embodiments of the cell-targeted molecule of the invention, the binding region is fused to the Shiga toxin effector polypeptide to form a single, continuous polypeptide.

In certain embodiments of the cell-targeted molecule of the invention, the binding region is fused to the carboxy terminus of the Shiga toxin effector polypeptide, whether directly or indirectly.

In certain embodiments of the cell-targeted molecule of the invention, the Shiga toxin effector polypeptide is linked to the binding region by at least one covalent bond which is not a disulfide bond.

In certain further embodiments of the cell-targeted molecule of the invention, the immunoglobulin-type binding region is selected from the group consisting of: single-domain antibody (sdAb) fragment, nanobody, heavy-chain antibody domain derived from a camelid (V_{H}H fragment), heavy-chain antibody domain derived from a cartilaginous fish, immunoglobulin new antigen receptor (IgNAR), V_{NAR} fragment, single-chain variable fragment (scFv), antibody variable fragment (Fv), a complementary determining region 3 (CDR3) fragment, constrained FR3-CDR3-FR4 (FR3-CDR3-FR4) polypeptide, Fd fragment, antigen-binding fragment (Fab), fibronectin-derived 10^{th} fibronectin type III domain (10Fn3), tenascin type III domain, ankyrin repeat motif domain, low-density-lipoprotein-receptor-derived A-domain (LDLR-A), lipocalin (anticalin), Kunitz domain, Protein-A-derived Z domain, gamma-B crystalline-derived domain, ubiquitin-derived domain, Sac7d-derived polypeptide (affitin), Fyn-derived SH2 domain, miniprotein, C-type lectin-like domain scaffold, engineered antibody mimic, and any genetically manipulated counterparts of any of the foregoing which retain binding functionality. In certain further embodiments of the cell-targeted molecule of the invention, the binding region is capable of binding to the extracellular target biomolecule selected from the group consisting of: CD20, CD22, CD40, CD79, CD25, CD30, HER2/neu/ErbB2, EGFR, EpCAMs (e.g. EGP-2, EGP-40), EphB2, prostate-specific membrane antigen, Cripto, endoglin, fibroblast activated protein, Lewis-Y, CD19, CD21, CS1/SLAMF7, CD33, CD52, CD133, CEA, gpA33, Mucins, TAG-72, carbonic anhydrase IX, folate binding protein, ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANKL, FAP, Tenascin, CD64, mesothelin, BRCA1, MART-1/MelanA, gp100, tyrosinase, human tyrosinase related protein 1 (TYRP1), human tyrosinase related protein 2, TRP-1, TRP-2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen, prostate specific antigen, prostate stem cell antigen, human aspartyl (asparaginyl) beta-hydroxylase, EphA2, HER3/ErbB-3, MUC1, MART-1/MelanA, gp100, tyrosinase associated antigen, HPV-E7, Epstein-Barr Virus antigens, Bcr-Abl, alpha-fetoprotein antigen, 17-A1, bladder tumor antigen, CD38, CD15, CD23, CD52, CD133, CD53, CD88, CD129, CD183, CD191, CD193, CD244, CD294, CD305, C3AR, FceRIa, galectin-9, mrp-14, siglec-8, siglec-10, CD49d, CD13, CD44, CD54, CD63, CD69, CD123, CD193, TLR4, FceRIa, IgE, CD107a, CD203c, CD14, CD15, CD33, CD64, CD68, CD80, CD86, CD105, CD115, F4/80, ILT-3, Galectin-3, CD11a-c, GITRL, MHC Class II, CD284-TLR4, CD107-Mac3, CD195-CCR5, HLA-DR, CD16/32, CD282-TLR2, CD11c, CD123, and any immunogenic fragment of any of the foregoing.

For certain embodiments, administration of the cell-targeted molecule of the invention to a cell physically coupled with an extracellular target biomolecule of the binding region, the cell-targeted molecule is capable of causing death of the cell. For certain further embodiments, administration of the cell-targeted molecule of the invention to two different populations of cell types which differ with respect to the presence or level of an extracellular target biomolecule, the cell-targeted molecule is capable of causing cell death to the cell-types physically coupled with an extracellular target biomolecule of the cell-targeted molecule's binding region at a CD₅₀ at least three times or less than the CD₅₀ to cell types which are not physically coupled with an extracellular target biomolecule of the cell-targeted molecule's binding region. For certain embodiments, administration of the cell-targeted molecule of the invention to a first population of cells whose members are physically coupled to extracellular target biomolecules of the cell-targeted molecule's binding region, and a second population of cells whose members are not physically coupled to any extracellular target biomolecule of the binding region, the cytotoxic effect of the cell-targeted molecule to members of said first population of cells relative to members of said second population of cells is at least 3-fold greater. For certain embodiments, administration of the cell-targeted molecule of the invention to a first population of cells whose members are physically coupled to a significant amount of the extracellular target biomolecule of the cell-targeted molecule's binding region, and a second population of cells whose members are not physically coupled to a significant amount of any extracellular target biomolecule of the binding region, the cytotoxic effect of the cell-targeted molecule to members of said first population of cells relative to members of said second population of cells is at least 3-fold greater. For certain embodiments, administration of the cell-targeted molecule of the invention to a first population of target biomolecule positive cells, and a second population of cells whose members do not express a significant amount of a target biomolecule of the cell-targeted molecule's binding region at a cellular surface, the cytotoxic effect of the cell-targeted molecule to members of the first population of cells relative to members of the second population of cells is at least 3-fold greater. In certain further embodiments of the cell-targeted molecule of the invention, the Shiga toxin effector polypeptide comprises or consists essentially of the polypeptide shown in any one of ID NOs: 4-49 of Table C, below. In certain further embodiments, the cell-targeted molecule of the invention comprises or consists essentially of the polypeptide shown in any one of ID NOs: 50-61 of Table C, below. In certain further embodiments, the cell-targeted molecule of the invention further comprises a carboxy-terminal endoplasmic reticulum retention/retrieval signal motif of a member of the KDEL family. In certain further embodiments, the cell-targeted molecule of the invention comprises the carboxy-terminal endoplasmic reticulum retention/retrieval signal motif selected from the group consisting of: KDEL, HDEF, HDEL, RDEF, RDEL, WDEL, YDEL, HEEF, HEEL, KEEL, REEL, KAEL, KCEL, KFEL, KGEL, KHEL, KLEL, KNEL, KQEL, KREL, KSEL, KVEL, KWEL, KYEL, KEDL, KIEL, DKEL, FDEL, KDEF, KKEL, HADL, HAEL, HIEL, HNEL, HTEL, KTEL, HVEL, NDEL, QDEL, REDL, RNEL, RTDL, RTEL, SDEL, TDEL, and SKEL. In certain further embodiments of the cell-targeted molecule of the invention, the Shiga toxin effector polypeptide comprises a mutation relative to a naturally occurring A Subunit of a member of the Shiga toxin family which reduces or eliminates the enzymatic activity of the Shiga toxin effector polypeptide but does not reduce the subcellular routing to the cytosol, of at least a part of the Shiga toxin effector polypeptide, below the subcellular routing level of a wild-type, Shiga toxin effector polypeptide.

In certain embodiments, the cell-targeted molecule of the present invention comprises 1) a Shiga toxin effector polypeptide comprising a Shiga toxin A1 fragment region having a carboxy terminus and a disrupted furin-cleavage motif, at the carboxy terminus of the A1 fragment region, comprising one or more mutations in the minimal, furin-cleavage motif relative to a wild-type, Shiga toxin A Subunit; and 2) a binding region capable of specifically binding at least one extracellular target biomolecule and associated with the carboxy terminus of the Shiga toxin effector polypeptide. In these embodiments of the cell-targeted molecules of the invention, a mutation in the minimal, furin-cleavage motif is an amino acid deletion, insertion, and/or substitution of at least one amino acid residue in the R/Y-x-x-R furin cleavage motif. In certain embodiments of the cell-targeted molecule of the invention, the Shiga toxin effector polypeptide is linked to the binding region by at least one covalent bond which is not a disulfide bond. In certain embodiments of the cell-targeted molecule of the invention, the binding region is fused to the carboxy terminus of the Shiga toxin effector polypeptide, whether directly or indirectly. In certain embodiments of the cell-targeted molecule of the invention, the binding region is fused to the carboxy terminus of the Shiga toxin effector polypeptide to form a single, continuous polypeptide. In certain embodiments, the binding region sterically covers the carboxy terminus of the A1 fragment region. In certain further embodiments of the cell-targeted molecule of the invention, the immunoglobulin-type binding region is selected from the group consisting of: single-domain antibody (sdAb) fragment, nanobody, heavy-chain antibody domain derived from a camelid (V_{H}H fragment), heavy-chain antibody domain derived from a cartilaginous fish, immunoglobulin new antigen receptor (IgNAR), V_{NAR} fragment, single-chain variable fragment (scFv), antibody variable fragment (Fv), a complementary determining region 3 (CDR3) fragment, constrained FR3-CDR3-1R4 (FR3-CDR3-FR4) polypeptide, Fd fragment, antigen-binding fragment (Fab), fibronectin-derived 10^{th} fibronectin type III domain (10Fn3), tenascin type III domain, ankyrin repeat motif domain, low-density-lipoprotein-receptor-derived A-domain (LDLR-A), lipocalin (anticalin), Kunitz domain, Protein-A-derived Z domain, gamma-B crystalline-derived domain, ubiquitin-derived domain, Sac7d-derived polypeptide (affitin), Fyn-derived SH2 domain, miniprotein, C-type lectin-like domain scaffold, engineered antibody mimic, and any genetically manipulated counterparts of any of the foregoing which retain binding functionality. In certain further embodiments of the cell-targeted molecule of the invention, the binding region is capable of binding to the extracellular target biomolecule selected from the group consisting of: CD20, CD22, CD40, CD79, CD25, CD30, HER2/neu/ErbB2, EGFR, EpCAMs (e.g. EGP-2, EGP-40), EphB2, prostate-specific membrane antigen, Cripto, endoglin, fibroblast activated protein, Lewis-Y, CD19, CD21, CS1/SLAMF7, CD33, CD52, CD133, CEA, gpA33, Mucins, TAG-72, carbonic anhydrase IX, folate binding protein, ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANKL, FAP, Tenascin, CD64, mesothelin, BRCA1, MART-1/MelanA, gp100, tyrosinase, human tyrosinase related protein 1 (TYRP1), human tyrosinase related protein 2, TRP-1, TRP-2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen, prostate specific antigen, prostate stem cell antigen, human aspartyl (asparaginyl) beta-hydroxylase, EphA2, HER3/ErbB-3, MUC1, MART-1/MelanA, gp100, tyrosinase associated antigen, HPV-E7, Epstein-Barr Virus antigens, Bcr-Abl, alpha-fetoprotein antigen, 17-A1, bladder tumor antigen, CD38, CD15, CD23, CD52, CD133, CD53, CD88, CD129, CD183, CD191, CD193, CD244, CD294, CD305, C3AR, FceRIa, galectin-9, mrp-14, siglec-8, siglec-10, CD49d, CD13, CD44, CD54, CD63, CD69, CD123, CD193, TLR4, FceRIa, IgE, CD107a, CD203c, CD14, CD15, CD33, CD64, CD68, CD80, CD86, CD105, CD115, F4/80, ILT-3, Galectin-3, CD11a-c, GITRL, MHC Class II, CD284-TLR4, CD107-Mac3, CD195-CCR5, HLA-DR, CD16/32, CD282-TLR2, CD11c, CD123, and any immunogenic fragment of any of the foregoing. For certain further embodiments of the cell-targeted molecule of the invention, administration of the cell-targeted molecule to a cell physically coupled with an extracellular target biomolecule of the binding region, the cell-targeted molecule is capable of causing death of the cell. For certain further embodiments, administration of the cell-targeted molecule of the invention to two different populations of cell types which differ with respect to the presence or level of an extracellular target biomolecule, the cell-targeted molecule is capable of causing cell death to the cell-types physically coupled with an extracellular target biomolecule of the cell-targeted molecule's binding region at a CD₅₀ at least three times or less than the CD₅₀ to cell types which are not physically coupled with an extracellular target biomolecule of the cell-targeted molecule's binding region. For certain embodiments, administration of the cell-targeted molecule of the invention to a first population of cells whose members are physically coupled to extracellular target biomolecules of the cell-targeted molecule's binding region, and a second population of cells whose members are not physically coupled to any extracellular target biomolecule of the binding region, the cytotoxic effect of the cell-targeted molecule to members of said first population of cells relative to members of said second population of cells is at least 3-fold greater. For certain embodiments, administration of the cell-targeted molecule of the invention to a first population of cells whose members are physically coupled to a significant amount of the extracellular target biomolecule of the cell-targeted molecule's binding region, and a second population of cells whose members are not physically coupled to a significant amount of any extracellular target biomolecule of the binding region, the cytotoxic effect of the cell-targeted molecule to members of said first population of cells relative to members of said second population of cells is at least 3-fold greater. For certain embodiments, administration of the cell-targeted molecule of the invention to a first population of target biomolecule positive cells, and a second population of cells whose members do not express a significant amount of a target biomolecule of the cell-targeted molecule's binding region at a cellular surface, the cytotoxic effect of the cell-targeted molecule to members of the first population of cells relative to members of the second population of cells is at least 3-fold greater. In certain further embodiments of the cell-targeted molecule of the invention, the Shiga toxin effector polypeptide comprises or consists essentially of the polypeptide shown in any one of ID NOs: 4-36 of Table C, below. In certain further embodiments, the cell-targeted molecule of the invention comprises or consists essentially of the polypeptide shown in any one of ID NOs: 50-61 of Table C, below. In certain further embodiments, the cell-targeted molecule of the invention further comprises a carboxy-terminal endoplasmic reticulum retention/retrieval signal motif of a member of the KDEL family. In certain further embodiments, the cell-targeted molecule of the invention comprises the carboxy-terminal endoplasmic reticulum retention/retrieval signal motif selected from the group consisting of: KDEL, HDEF, HDEL, RDEF, RDEL, WDEL, YDEL, HEEF, HEEL, KEEL, REEL, KAEL, KCEL, KFEL, KGEL, KHEL, KLEL, KNEL, KQEL, KREL, KSEL, KVEL, KWEL, KYEL, KEDL, KIEL, DKEL, FDEL, KDEF, KKEL, HADL, HAEL, HIEL, HNEL, HTEL, KTEL, HVEL, NDEL, QDEL, REDL, RNEL, RTDL, RTEL, SDEL, TDEL, and SKEL. In certain further embodiments of the cell-targeted molecule of the invention, the Shiga toxin effector polypeptide comprises a mutation relative to a naturally occurring A Subunit of a member of the Shiga toxin family which reduces or eliminates the enzymatic activity of the Shiga toxin effector polypeptide but does not reduce the subcellular routing to the cytosol, of at least a part of the Shiga toxin effector polypeptide, below the subcellular routing level of a wild-type, Shiga toxin effector polypeptide.

In certain embodiments, the cytotoxic molecule of the present invention comprises a Shiga toxin effector polypeptide comprising 1) a Shiga toxin A1 fragment region having a carboxy terminus and 2) a disrupted furin-cleavage motif at the carboxy terminus of the A1 fragment region; wherein the cytotoxic molecule is capable, when a component of a first cell-targeted molecule comprising a binding region capable of specifically binding at least one extracellular target biomolecule and a molecular moiety associated with the carboxy terminus of the Shiga toxin effector polypeptide, of exhibiting cytotoxicity equivalent to cytotoxicity of a second cell-targeted molecule consisting of the cell-targeted molecule except for the Shiga toxin effector polypeptide consists of a with a wild-type, Shiga toxin A1 polypeptide. This means the second cell-targeted molecule comprises the same binding region and the same molecular moiety as the first cell-targeted molecule of the invention but instead of comprising the same Shiga toxin effector polypeptide, the second cell-targeted molecule comprises a wild-type, Shiga toxin effector polypeptide comprising a Shiga toxin A1 fragment region (e.g. amino acids 1-251 of SEQ ID NO:1 or SEQ ID NO:2, or amino acids 1-250 of SEQ ID NO:3) having a carboxy terminus and a wild-type furin-cleavage site at the carboxy terminus of the A1 fragment region of the wild-type, Shiga toxin effector polypeptide; wherein the molecular moiety associated with the carboxy terminus of the wild-type, Shiga toxin A1 polypeptide with the same association as in the first cell-targeted molecule. In certain further embodiments, the molecular moiety comprises at least one amino acid residue fused to the carboxy terminus of the Shiga toxin effector polypeptide, either directly or indirectly. In certain embodiments, the molecular moiety sterically covers the carboxy terminus of the A1 fragment region. In certain embodiments, the molecular moiety comprises a peptide and/or polypeptide derived from the Shiga toxin A2 fragment of a naturally occurring Shiga toxin. In certain embodiments, the Shiga toxin effector polypeptide is linked to the molecular moiety by at least one covalent bond which is not a disulfide bond. In certain further embodiments, the molecular moiety comprises a polypeptide fused to the carboxy terminus of the Shiga toxin effector polypeptide to form a single, continuous polypeptide. In certain further embodiments of the cytotoxic molecule of the invention, the disrupted furin-cleavage motif comprises at least one mutation relative to a wild-type, Shiga toxin A Subunit, the mutation altering at least one amino acid residue in the region natively positioned 1) at 248-251 of the A Subunit of Shiga-like toxin 1 (SEQ ID NO: 1) or Shiga toxin (SEQ ID NO: 2), or 2) at 247-250 of the A Subunit of Shiga-like toxin 2 (SEQ ID NO:3). In certain further embodiments of the cytotoxic molecule of the invention, the mutation is an amino acid residue substitution of an arginine residue with a non-positively charged, amino acid residue. In certain further embodiments of the cytotoxic molecule of the invention, the Shiga toxin effector polypeptide comprises or consists essentially of the polypeptide shown in any one of ID NOs: 4-49 of Table C, below. In certain further embodiments of the cytotoxic molecule of the invention, the first cell-targeted molecule is capable of exhibiting improved, in vivo tolerability compared to the second cell-targeted molecule. In certain further embodiments of the cytotoxic molecule of the invention, the Shiga toxin effector polypeptide comprises a mutation relative to a naturally occurring A Subunit of a member of the Shiga toxin family which reduces or eliminates the enzymatic activity of the Shiga toxin effector polypeptide but does not reduce the subcellular routing to the cytosol, of at least a part of the Shiga toxin effector polypeptide, below the subcellular routing level of a wild-type, Shiga toxin effector polypeptide.

In certain embodiments of the cytotoxic molecule of the present invention, the Shiga toxin effector polypeptide does not consist of the polypeptide shown in SEQ ID NO:2 further comprising both of the following amino acid residue substitutions: R248H and R251H. In certain embodiments of the cytotoxic molecule, the Shiga toxin effector polypeptide does not comprise both of the following amino acid residue substitutions: R248H and R251H. In certain embodiments of the cytotoxic molecule, the Shiga toxin effector polypeptide does not consist of the polypeptide shown in SEQ ID NO:1 further comprising both of the following amino acid residue substitutions: R248G and R251G. In certain embodiments of the cytotoxic molecule, the Shiga toxin effector polypeptide does not comprise both of the following amino acid residue substitutions: R248G and R251G. In certain embodiments of the cytotoxic molecule, the Shiga toxin effector polypeptide does not consist of the polypeptide shown in SEQ ID NO:1 further comprising all of the following amino acid residue substitutions: A246G, S247A, A253G, and S254A. In certain embodiments of the cytotoxic molecule, the Shiga toxin effector polypeptide does not comprise all of the following amino acid residue substitutions: A246G, S247A, A253G, and S254A. In certain embodiments of the cytotoxic molecule, the Shiga toxin effector polypeptide does not consist of the polypeptide shown in SEQ ID NO:1 further comprising all of the following amino acid residue substitutions: A246G, S247A, R248G, R251G, A253G, and S254A. In certain embodiments of the cytotoxic molecule, the Shiga toxin effector polypeptide does not comprise all of the following amino acid residue substitutions: A246G, S247A, R248G, R251G, A253G, and S254A. In certain embodiments of the cytotoxic molecule, the Shiga toxin effector polypeptide does not consist of the polypeptide shown in SEQ ID NO:2 further comprising the deletion of the region natively positioned at 247-252. In certain embodiments of the cytotoxic molecule, the Shiga toxin effector polypeptide does not comprise a Shiga toxin effector polypeptide comprising the deletion of the region natively positioned at 247-252. In certain embodiments of the cytotoxic molecule, the Shiga toxin effector polypeptide does not consist of the polypeptide shown in SEQ ID NO:2 further comprising both of the following deletions: 245-247 and 253-255. In certain embodiments of the cytotoxic molecule, the Shiga toxin effector polypeptide does not comprise both of the following deletions: 245-247 and 253-255.

In certain embodiments, the cytotoxic, cell-targeted molecule of the present invention comprises 1) a binding region capable of specifically binding at least one extracellular target biomolecule, 2) a Shiga toxin effector polypeptide comprising a Shiga toxin A1 fragment region having a carboxy terminus and a disrupted furin-cleavage motif at the carboxy terminus of the A1 fragment region, and 3) a molecular moiety associated with the carboxy terminus of the Shiga toxin effector polypeptide; and wherein the cytotoxic, cell-targeted molecule is capable of exhibiting cytotoxicity equivalent to cytotoxicity of a second cell-targeted molecule consisting of the cell-targeted molecule except for the Shiga toxin effector polypeptide consists of a wild-type, Shiga toxin A1 polypeptide. This means the second cell-targeted molecule comprises the same binding region and the same molecular moiety as the cytotoxic, cell-targeted molecule of the invention but instead of comprising the same Shiga toxin effector polypeptide, the second cell-targeted molecule comprises a wild-type, Shiga toxin effector polypeptide comprising a Shiga toxin A1 fragment region having a carboxy terminus and a wild-type furin-cleavage site at the carboxy terminus of the A1 fragment region of the wild-type, Shiga toxin effector polypeptide; wherein the molecular moiety associated with the carboxy terminus of the wild-type, Shiga toxin A1 polypeptide with the same association as in the first cell-targeted molecule. In certain embodiments of the cytotoxic, cell-targeted molecule of the invention, the molecular moiety sterically covers the carboxy terminus of the A1 fragment region. In certain embodiments of the cytotoxic, cell-targeted molecule of the invention, the binding region sterically covers the carboxy terminus of the A1 fragment region. In certain further embodiments of the cytotoxic, cell-targeted molecule of the invention, the molecular moiety comprises the binding region. In certain embodiments of the cytotoxic, cell-targeted molecule of the invention, the molecular moiety comprises a peptide and/or polypeptide derived from the Shiga toxin A2 fragment of a naturally occurring Shiga toxin. In certain further embodiments of the cytotoxic, cell-targeted molecule of the invention, the binding region comprises a polypeptide comprising an immunoglobulin-type binding region. In certain further embodiments of the cytotoxic, cell-targeted molecule of the invention, the immunoglobulin-type binding region is selected from the group consisting of: single-domain antibody (sdAb) fragment, nanobody, heavy-chain antibody domain derived from a camelid (V_{H}H fragment), heavy-chain antibody domain derived from a cartilaginous fish, immunoglobulin new antigen receptor (IgNAR), V_{NAR} fragment, single-chain variable fragment (scFv), antibody variable fragment (Fv), a complementary determining region 3 (CDR3) fragment, constrained FR3-CDR3-FR4 (FR3-CDR3-FR4) polypeptide, Fd fragment, antigen-binding fragment (Fab), fibronectin-derived 10^{th} fibronectin type III domain (10Fn3), tenascin type III domain, ankyrin repeat motif domain, low-density-lipoprotein-receptor-derived A-domain (LDLR-A), lipocalin (anticalin), Kunitz domain, Protein-A-derived Z domain, gamma-B crystalline-derived domain, ubiquitin-derived domain, Sac7d-derived polypeptide (affitin), Fyn-derived SH2 domain, miniprotein, C-type lectin-like domain scaffold, engineered antibody mimic, and any genetically manipulated counterparts of any of the foregoing which retain binding functionality. In certain embodiments of the cytotoxic, cell-targeted molecule of the invention, the Shiga toxin effector polypeptide is linked to the molecular moiety by at least one covalent bond which is not a disulfide bond. In certain further embodiments, the molecular moiety comprises at least one amino acid residue fused to the carboxy terminus of the Shiga toxin effector polypeptide. In certain further embodiments of the cytotoxic, cell-targeted molecule of the invention, the molecular moiety comprises a polypeptide fused to the carboxy terminus of the Shiga toxin effector polypeptide to form a single, continuous polypeptide. For certain embodiments, administration of the cytotoxic, cell-targeted molecule of the invention to two different populations of cell types which differ with respect to the presence or level of an extracellular target biomolecule, the cell-targeted molecule is capable of causing cell death to the cell-types physically coupled with an extracellular target biomolecule of the cell-targeted molecule's binding region at a CD₅₀ at least three times or less than the CD₅₀ to cell types which are not physically coupled with an extracellular target biomolecule of the cell-targeted molecule's binding region. For certain embodiments, administration of the cytotoxic, cell-targeted molecule of the invention to a first population of cells whose members are physically coupled to extracellular target biomolecules of the cell-targeted molecule's binding region, and a second population of cells whose members are not physically coupled to any extracellular target biomolecule of the binding region, the cytotoxic effect of the cell-targeted molecule to members of said first population of cells relative to members of said second population of cells is at least 3-fold greater. For certain embodiments, administration of the cytotoxic, cell-targeted molecule of the invention to a first population of cells whose members are physically coupled to a significant amount of the extracellular target biomolecule of the cell-targeted molecule's binding region, and a second population of cells whose members are not physically coupled to a significant amount of any extracellular target biomolecule of the binding region, the cytotoxic effect of the cell-targeted molecule to members of said first population of cells relative to members of said second population of cells is at least 3-fold greater. For certain embodiments, administration of the cytotoxic, cell-targeted molecule of the invention to a first population of target biomolecule positive cells, and a second population of cells whose members do not express a significant amount of a target biomolecule of the cell-targeted molecule's binding region at a cellular surface, the cytotoxic effect of the cell-targeted molecule to members of the first population of cells relative to members of the second population of cells is at least 3-fold greater. In certain further embodiments of the cytotoxic, cell-targeted molecule of the invention, the binding region is capable of binding to the extracellular target biomolecule selected from the group consisting of: CD20, CD22, CD40, CD79, CD25, CD30, HER2/neu/ErbB2, EGFR, EpCAMs (e.g. EGP-2, EGP-40), EphB2, prostate-specific membrane antigen, Cripto, endoglin, fibroblast activated protein, Lewis-Y, CD19, CD21, CS1/SLAMF7, CD33, CD52, CD133, CEA, gpA33, Mucins, TAG-72, carbonic anhydrase IX, folate binding protein, ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANKL, FAP, Tenascin, CD64, mesothelin, BRCA1, MART-1/MelanA, gp100, tyrosinase, human tyrosinase related protein 1 (TYRP1), human tyrosinase related protein 2, TRP-1, TRP-2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen, prostate specific antigen, prostate stem cell antigen, human aspartyl (asparaginyl) beta-hydroxylase, EphA2, HER3/ErbB-3, MUC1, MART-1/MelanA, gp100, tyrosinase associated antigen, HPV-E7, Epstein-Barr Virus antigens, Bcr-Abl, alpha-fetoprotein antigen, 17-A1, bladder tumor antigen, CD38, CD15, CD23, CD52, CD133, CD53, CD88, CD129, CD183, CD191, CD193, CD244, CD294, CD305, C3AR, FceRIa, galectin-9, mrp-14, siglec-8, siglec-10, CD49d, CD13, CD44, CD54, CD63, CD69, CD123, CD193, TLR4, FceRIa, IgE, CD107a, CD203c, CD14, CD15, CD33, CD64, CD68, CD80, CD86, CD105, CD115, F4/80, ILT-3, Galectin-3, CD11a-c, GITRL, MHC Class II, CD284-TLR4, CD107-Mac3, CD195-CCR5, HLA-DR, CD16/32, CD282-TLR2, CD11c, CD123, and any immunogenic fragment of any of the foregoing. In certain further embodiments of the cytotoxic, cell-targeted molecule of the invention, the Shiga toxin effector polypeptide comprises or consists essentially of the polypeptide shown in any one of ID NOs: 4-49 of Table C, below. In certain further embodiments, the cytotoxic, cell-targeted molecule of the invention comprises or consists essentially of the polypeptide shown in any one of ID NOs: 50-61 of Table C, below. In certain further embodiments, the cell-targeted molecule of the invention further comprises a carboxy-terminal endoplasmic reticulum retention/retrieval signal motif of a member of the KDEL family. In certain further embodiments, the cell-targeted molecule of the invention comprises the carboxy-terminal endoplasmic reticulum retention/retrieval signal motif selected from the group consisting of: KDEL, HDEF, HDEL, RDEF, RDEL, WDEL, YDEL, HEEF, HEEL, KEEL, REEL, KAEL, KCEL, KFEL, KGEL, KHEL, KLEL, KNEL, KQEL, KREL, KSEL, KVEL, KWEL, KYEL, KEDL, KIEL, DKEL, FDEL, KDEF, KKEL, HADL, HAEL, HIEL, HNEL, HTEL, KTEL, HVEL, NDEL, QDEL, REDL, RNEL, RTDL, RTEL, SDEL, TDEL, and SKEL. In certain further embodiments of the cytotoxic, cell-targeted molecule of the invention, the cell-targeted molecule is capable of exhibiting improved, in vivo tolerability compared to the second cell-targeted molecule. In certain further embodiments of the cell-targeted molecule of the invention, the Shiga toxin effector polypeptide comprises a mutation relative to a naturally occurring A Subunit of a member of the Shiga toxin family which reduces or eliminates the enzymatic activity of the Shiga toxin effector polypeptide but does not reduce the subcellular routing to the cytosol of at least a part of the Shiga toxin effector polypeptide below the subcellular routing level of a wild-type, Shiga toxin effector polypeptide. In certain embodiments, the cell-targeted molecule of the present invention does not comprise a carboxy-terminal, binding region comprising a fragment of an immune cell surface receptor. In certain embodiments of the cell-targeted molecule of the present invention, the binding region does not comprise a fragment of a human, immune cell surface co-receptor. In certain further embodiments of the cell-targeted molecule of the present invention, the binding region does not comprise a fragment of human CD4, a type-I transmembrane glycoprotein, such as, the HIV envelope glycoprotein GP120-binding region of the immune cell-surface, co-receptor, type-I transmembrane glycoprotein CD4 (cluster of differentiation 4) of H. sapiens. In certain embodiments, the cell-targeted molecules of the present invention do not comprise a Shiga toxin effector polypeptide comprising amino acids 1-247 of SEQ ID NO:2, 45-247 of SEQ ID NO:2, and/or 75-247 of SEQ ID NO:2 fused to a carboxy-terminal, binding region comprising a fragment of human CD4 corresponding to amino acid residues 19-183.

In certain embodiments, the cytotoxic, cell targeted molecule of the present invention comprises 1) a binding region capable of specifically binding at least one extracellular target biomolecule; 2) a Shiga toxin effector polypeptide comprising a Shiga toxin A1 fragment region having a carboxy terminus and a disrupted furin-cleavage motif at the carboxy terminus of the A1 fragment region; and 3) a molecular moiety associated with the carboxy terminus of the Shiga toxin effector polypeptide; and wherein the cytotoxic, cell-targeted molecule is capable of exhibiting improved, in vivo tolerability compared to in vivo tolerability of a second cell-targeted molecule consisting of the cell-targeted molecule except for the Shiga toxin effector polypeptide consists of a wild-type, Shiga toxin A1 polypeptide. This means the second cell-targeted molecule comprises the same binding region and the same molecular moiety as the cytotoxic, cell-targeted molecule of the invention but instead of comprising the same Shiga toxin effector polypeptide, the second cell-targeted molecule comprises a wild-type, Shiga toxin effector polypeptide comprising a Shiga toxin A1 fragment region having a carboxy terminus and a wild-type furin-cleavage site at the carboxy terminus of the A1 fragment region of the wild-type, Shiga toxin effector polypeptide; wherein the molecular moiety associated with the carboxy terminus of the wild-type, Shiga toxin A1 polypeptide with the same association as in the first cell-targeted molecule. In certain embodiments of the cytotoxic, cell targeted molecule of the invention, the Shiga toxin effector polypeptide is not cytotoxic and the molecular moiety is toxic. In certain embodiments of the cytotoxic, cell-targeted molecule of the invention, the molecular moiety sterically covers the carboxy terminus of the A1 fragment region. In certain embodiments of the cytotoxic, cell-targeted molecule of the invention, the binding region sterically covers the carboxy terminus of the A1 fragment region. In certain embodiments of the cytotoxic, cell targeted molecule of the invention, the molecular moiety comprises the binding region. In certain embodiments of the cytotoxic, cell-targeted molecule of the invention, the Shiga toxin effector polypeptide is linked to the molecular moiety by at least one covalent bond which is not a disulfide bond. In certain further embodiments, the molecular moiety comprises at least one amino acid residue fused to the carboxy terminus of the Shiga toxin effector polypeptide. In certain further embodiments of the cytotoxic, cell-targeted molecule of the invention, the molecular moiety comprises a polypeptide fused to the carboxy terminus of the Shiga toxin effector polypeptide to form a single, continuous polypeptide. In certain embodiments of the cytotoxic, cell-targeted molecule of the invention, the molecular moiety comprises a peptide and/or polypeptide derived from the Shiga toxin A2 fragment of a naturally occurring Shiga toxin. For certain further embodiments, administration of the cytotoxic, cell-targeted molecule of the invention to two different populations of cell types which differ with respect to the presence or level of an extracellular target biomolecule, the cell-targeted molecule is capable of causing cell death to the cell-types physically coupled with an extracellular target biomolecule of the cell-targeted molecule's binding region at a CD₅₀ at least three times or less than the CD₅₀ to cell types which are not physically coupled with an extracellular target biomolecule of the cell-targeted molecule's binding region. For certain embodiments, administration of the cytotoxic, cell-targeted molecule of the invention to a first population of cells whose members are physically coupled to extracellular target biomolecules of the cell-targeted molecule's binding region, and a second population of cells whose members are not physically coupled to any extracellular target biomolecule of the binding region, the cytotoxic effect of the cell-targeted molecule to members of said first population of cells relative to members of said second population of cells is at least 3-fold greater. For certain embodiments, administration of the cytotoxic, cell-targeted molecule of the invention to a first population of cells whose members are physically coupled to a significant amount of the extracellular target biomolecule of the cell-targeted molecule's binding region, and a second population of cells whose members are not physically coupled to a significant amount of any extracellular target biomolecule of the binding region, the cytotoxic effect of the cell-targeted molecule to members of said first population of cells relative to members of said second population of cells is at least 3-fold greater. For certain embodiments, administration of the cytotoxic, cell-targeted molecule of the invention to a first population of target biomolecule positive cells, and a second population of cells whose members do not express a significant amount of a target biomolecule of the cell-targeted molecule's binding region at a cellular surface, the cytotoxic effect of the cell-targeted molecule to members of the first population of cells relative to members of the second population of cells is at least 3-fold greater. In certain embodiments of the cytotoxic, cell targeted molecule of the invention, the binding region comprises a polypeptide comprising an immunoglobulin-type binding region. In certain further embodiments of the cytotoxic, cell-targeted molecule of the invention, the immunoglobulin-type binding region is selected from the group consisting of: single-domain antibody (sdAb) fragment, nanobody, heavy-chain antibody domain derived from a camelid (V_{H}H fragment), heavy-chain antibody domain derived from a cartilaginous fish, immunoglobulin new antigen receptor (IgNAR), V_{NAR} fragment, single-chain variable fragment (scFv), antibody variable fragment (Fv), a complementary determining region 3 (CDR3) fragment, constrained FR3-CDR3-FR4 (FR3-CDR3-FR4) polypeptide, Fd fragment, antigen-binding fragment (Fab), fibronectin-derived 10^{th} fibronectin type III domain (10Fn3), tenascin type III domain, ankyrin repeat motif domain, low-density-lipoprotein-receptor-derived A-domain (LDLR-A), lipocalin (anticalin), Kunitz domain, Protein-A-derived Z domain, gamma-B crystalline-derived domain, ubiquitin-derived domain, Sac7d-derived polypeptide (affitin), Fyn-derived SH2 domain, miniprotein, C-type lectin-like domain scaffold, engineered antibody mimic, and any genetically manipulated counterparts of any of the foregoing which retain binding functionality. In certain further embodiments of the cytotoxic, cell-targeted molecule of the invention, the Shiga toxin effector polypeptide comprises or consists essentially of the polypeptide shown in any one of ID NOs: 4-49 of Table C, below. In certain further embodiments of the cytotoxic, cell-targeted molecule of the invention, the molecular moiety comprises at least one amino acid residue fused to the carboxy terminus of the Shiga toxin effector polypeptide, either directly or indirectly. In certain further embodiments, the cytotoxic, cell-targeted molecule of the invention comprises or consists essentially of the polypeptide shown in any one of ID NOs: 50-61 of Table C, below. In certain further embodiments, the cytotoxic, cell-targeted molecule of the invention further comprises a carboxy-terminal endoplasmic reticulum retention/retrieval signal motif of a member of the KDEL family. In certain further embodiments, the cytotoxic, cell-targeted molecule of the invention comprises the carboxy-terminal endoplasmic reticulum retention/retrieval signal motif selected from the group consisting of: KDEL, HDEF, HDEL, RDEF, RDEL, WDEL, YDEL, HEEF, HEEL, KEEL, REEL, KAEL, KCEL, KFEL, KGEL, KHEL, KLEL, KNEL, KQEL, KREL, KSEL, KVEL, KWEL, KYEL, KEDL, KIEL, DKEL, FUEL, KDEF, KKEL, HADL, HAEL, HIEL, HNEL, HTEL, KTEL, HVEL, NDEL, QDEL, REDL, RNEL, RTDL, RTEL, SDEL, TDEL, and SKEL. In certain further embodiments of the cytotoxic, cell-targeted molecule of the invention, the Shiga toxin effector polypeptide comprises a mutation relative to a naturally occurring A Subunit of a member of the Shiga toxin family which reduces or eliminates the enzymatic activity of the Shiga toxin effector polypeptide but does not reduce the subcellular routing to the cytosol of at least a part of the Shiga toxin effector polypeptide below the subcellular routing level of a wild-type, Shiga toxin effector polypeptide.

In certain embodiments, the cell-targeted molecule of the present invention, whether cytotoxic or non-cytotoxic, does not comprise a naturally occurring Shiga toxin B Subunit. In certain embodiments, the cell-targeted molecule of the invention does not comprise any polypeptide comprising or consisting essentially of a functional binding domain of a native, Shiga toxin B subunit. Rather, in certain embodiments of the cell-targeted molecules of the invention, the Shiga toxin A Subunit derived regions are functionally associated with heterologous binding regions to effectuate cell targeting.

In certain embodiments, the cell-targeted molecule of the present invention, whether cytotoxic or non-cytotoxic, does not comprise any Shiga toxin A2 fragment of a member of the Shiga toxin family or functional fragment thereof. In certain embodiments, the cell-targeted molecule of the invention does not comprise, carboxy-terminal of the disrupted furin-cleavage motif, any amino acid sequence from a native, wild-type, Shiga toxin A2 fragment.

In certain embodiments, the cytotoxic molecule of the present invention does not comprise any Shiga toxin A2 fragment of a member of the Shiga toxin family or functional fragment thereof. In certain embodiments, the cytotoxic molecule of the present invention does not comprise, carboxy-terminal of the disrupted furin-cleavage motif, any amino acid sequence from a native, wild-type, Shiga toxin A2 fragment.

In certain embodiments of the cytotoxic, cell-targeted molecule of the present invention, the Shiga toxin effector polypeptide does not consist of the polypeptide shown in SEQ ID NO:2 further comprising both of the following amino acid residue substitutions: R248H and R251H. In certain embodiments of the cytotoxic, cell-targeted molecule, the Shiga toxin effector polypeptide does not comprise both of the following amino acid residue substitutions: R248H and R251H. In certain embodiments of the cytotoxic, cell-targeted molecule, the Shiga toxin effector polypeptide does not consist of the polypeptide shown in SEQ ID NO:1 further comprising both of the following amino acid residue substitutions: R248G and R251G. In certain embodiments of the cytotoxic, cell-targeted molecule, the Shiga toxin effector polypeptide does not comprise both of the following amino acid residue substitutions: R248G and R251G. In certain embodiments of the cytotoxic, cell-targeted molecule, the Shiga toxin effector polypeptide does not consist of the polypeptide shown in SEQ ID NO:1 further comprising all of the following amino acid residue substitutions: A246G, S247A, A253G, and S254A. In certain embodiments of the cytotoxic, cell-targeted molecule, the Shiga toxin effector polypeptide does not comprise all of the following amino acid residue substitutions: A246G, S247A, A253G, and S254A. In certain embodiments of the cytotoxic, cell-targeted molecule, the Shiga toxin effector polypeptide does not consist of the polypeptide shown in SEQ ID NO:1 further comprising all of the following amino acid residue substitutions: A246G, S247A, R248G, R251G, A253G, and S254A. In certain embodiments of the cytotoxic, cell-targeted molecule, the Shiga toxin effector polypeptide does not comprise all of the following amino acid residue substitutions: A246G, S247A, R248G, R251G, A253G, and S254A. In certain embodiments of the cytotoxic, cell-targeted molecule, the Shiga toxin effector polypeptide does not consist of the polypeptide shown in SEQ ID NO:2 further comprising the deletion of the region natively positioned at 247-252. In certain embodiments of the cytotoxic, cell-targeted molecule, the Shiga toxin effector polypeptide does not comprise a Shiga toxin effector polypeptide comprising the deletion of the region natively positioned at 247-252. In certain embodiments of the cytotoxic, cell-targeted molecule, the Shiga toxin effector polypeptide does not consist of the polypeptide shown in SEQ ID NO:2 further comprising both of the following deletions: 245-247 and 253-255. In certain embodiments of the cytotoxic, cell-targeted molecule, the Shiga toxin effector polypeptide does not comprise both of the following deletions: 245-247 and 253-255.

The present invention also provides pharmaceutical compositions comprising a molecule of the invention and at least one pharmaceutically acceptable excipient or carrier; and the use of such a molecule or a composition comprising it in making such pharmaceutical compositions and in methods of the invention as further described herein. In certain embodiments of the present invention are pharmaceutical compositions comprising any cytotoxic molecule of the present invention and at least one pharmaceutically acceptable excipient or carrier.

Beyond the molecules of the present invention, polynucleotides capable of encoding any of the foregoing, e.g., a polypeptide comprising a protease-cleavage resistant, Shiga toxin effector polypeptide or protein of a molecule of the present invention, are within the scope of the present invention, as well as expression vectors which comprise a polynucleotide of the invention and host cells comprising an expression vector of the invention. Host cells comprising an expression vector may be used, e.g., in methods for producing a molecule of the invention (e.g. polypeptide or protein), or a polypeptide component or fragment thereof, by recombinant expression.

The present invention also encompasses any composition of matter of the present invention which is immobilized on a solid substrate. Such arrangements of the compositions of matter of the present invention may be utilized, e.g., in methods of screening molecules as described herein.

Beyond the compositions of matter of the present invention, the present invention is directed to a variety of methods, such as, e.g., methods which use a composition of matter of the invention and/or methods which create a composition of matter of the invention.

In certain embodiments of the present invention is a method for improving the in vivo tolerability and/or in vitro stability of a molecule comprising 1) a Shiga toxin effector polypeptide comprising a Shiga toxin A1 fragment region having a carboxy terminus and a furin-cleavage site proximal to the carboxy terminus of the A1 fragment region, and 2) a heterologous, molecular moiety associated with the carboxy terminus of the Shiga toxin effector polypeptide and comprising a binding region capable of specifically binding at least one extracellular target biomolecule; the method comprising the step of disrupting a furin-cleavage motif comprising the furin-cleavage site. In certain embodiments of this method, the disrupting step involves creating a mutation, truncation, and/or amino acid functional group modification which reduces the protease-cleavage sensitivity of the carboxy terminus of the Shiga toxin effector polypeptide. In certain embodiments of this method, the heterologous, molecular moiety sterically covers the carboxy terminus of the A1 fragment region. The present invention also encompasses any molecule created using this method which is capable of exhibiting improved in vivo tolerability as compared to a parental molecule comprising an undisrupted furin-cleavage motif proximal to the carboxy terminus of the A1 fragment region.

In certain embodiments of the present invention is a method for improving the in vivo tolerability and/or in vitro stability of a molecule comprising 1) a Shiga toxin effector polypeptide comprising a Shiga toxin A1 fragment region having a carboxy terminus and a furin-cleavage site proximal to the carboxy terminus of the A1 fragment region, and 2) a heterologous, molecular moiety associated with the carboxy terminus of the Shiga toxin effector polypeptide and which is toxic; the method comprising the step of disrupting a furin-cleavage motif comprising the furin-cleavage site. In certain embodiments of this method, the disrupting step involves creating a mutation, truncation, and/or amino acid functional group modification which reduces the protease-cleavage sensitivity of the carboxy terminus of the Shiga toxin effector polypeptide. In certain embodiments of this method, the heterologous, molecular moiety sterically covers the carboxy terminus of the A1 fragment region. The present invention also encompasses any molecule created using this method which is capable of exhibiting improved in vivo tolerability as compared to a parental molecule comprising an undisrupted furin-cleavage motif proximal to the carboxy terminus of the A1 fragment region.

Among certain embodiments of the present invention is a method of killing a cell comprising the step of contacting the cell with any of the above cell-targeted molecules of the present invention or the above pharmaceutical composition of the present invention. In certain embodiments, the step of contacting the cell(s) occurs in vitro. In certain other embodiments, the step of contacting the cell(s) occurs or in vivo. In further embodiments of the cell killing methods, the method is capable of selectively killing cell(s) and/or cell types preferentially over other cell(s) and/or cell types when contacting a mixture of cells which differ with respect to the extracellular presence and/or expression level of an extracellular target biomolecule of the binding region of the protein.

The present invention further provides methods of treating diseases, disorders, and/or conditions in patients comprising the step of administering to a patient in need thereof a therapeutically effective amount of a molecule or a pharmaceutical composition of the invention. In certain embodiments, the disease, disorder, or condition to be treated using this method of the invention is selected from: a cancer, tumor, growth abnormality, immune disorder, or microbial infection. In certain embodiments of this method, the cancer to be treated is selected from the group consisting of: bone cancer, breast cancer, central/peripheral nervous system cancer, gastrointestinal cancer, germ cell cancer, glandular cancer, head-neck cancer, hematological cancer, kidney-urinary tract cancer, liver cancer, lung/pleura cancer, prostate cancer, sarcoma, skin cancer, and uterine cancer. In certain embodiments of this method, the immune disorder to be treated is an immune disorder associated with a disease selected from the group consisting of: amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis, psoriasis, psoriatic arthritis, rheumatoid arthritis, scleroderma, septic shock, Sjorgren's syndrome, ulcerative colitis, and vasculitis.

Among certain embodiments of the present invention is a composition comprising a molecule of the invention (e.g. polypeptide or protein), compound comprising a molecule of the invention, or a composition of the invention (e.g. pharmaceutical composition) for the treatment or prevention of a cancer, immune disorder, or microbial infection. Among certain embodiments of the present invention is the use of a compound (e.g. protein) or composition of the invention in the manufacture of a medicament for the treatment or prevention of a cancer, tumor, immune disorder, or microbial infection.

Certain embodiments of the molecules of the present invention may be used to deliver one or more additional exogenous materials into a cell physically coupled with an extracellular target biomolecule of the molecule of the present invention. Additionally, the present invention provides a method for delivering exogenous material to the inside of a cell(s) comprising contacting the cell(s), either in vitro or in vivo, with a molecule, pharmaceutical composition, and/or diagnostic composition of the present invention. The present invention further provides a method for delivering exogenous material to the inside of a cell(s) in a patient in need thereof, the method comprising the step of administering to the patient a molecule of the present invention, wherein the target cell(s) is physically coupled with an extracellular target biomolecule of the molecule of the present invention.

The use of any composition of the present invention (e.g. a cell-targeted molecule, a pharmaceutical composition, or diagnostic composition) for the diagnosis, prognosis, and/or characterization of a disease, disorder, and/or condition is within the scope of the present invention. Among certain embodiments of the present invention is the use of one or more compositions of matter of the invention (e.g. a pharmaceutical composition) in the treatment or prevention of a cancer, tumor, or immune disorder. Among certain embodiments of the present invention is the use of one or more compositions of matter of the invention (e.g. a pharmaceutical composition) in the manufacture of a medicament for the treatment or prevention of a cancer, tumor, or immune disorder.

Among certain embodiments of the present invention is a diagnostic composition comprising a molecule of the invention (e.g. molecule, cell-targeted molecule, polypeptide or protein) and a detection promoting agent for the collection of information, such as diagnostically useful information about a cell type, tissue, organ, disease, disorder, condition, and/or patient.

Among certain embodiments of the present invention is the method of detecting a cell using a molecule and/or diagnostic composition of the invention comprising the steps of contacting a cell with said molecule and/or diagnostic composition and detecting the presence of said molecule and/or diagnostic composition. In certain embodiments, the step of contacting the cell(s) occurs in vitro. In certain embodiments, the step of contacting the cell(s) occurs or in vivo. In certain embodiments, the step of detecting the cell(s) occurs in vitro. In certain embodiments, the step of detecting the cell(s) occurs or in vivo.

For example, a diagnostic composition of the invention may be used to detect a cell in vivo by administering to a mammalian subject a composition comprising molecule of the present invention which comprises a detection promoting agent and detecting the presence of the molecule of the present invention either in vitro or in vivo. The information collected may regard the presence of a cell physically coupled with an extracellular target of the binding region of the molecule of the present invention and may be useful in the diagnosis, prognosis, characterization, and/or treatment of a disease, disorder, or condition. Certain compounds (e.g. polypeptides and proteins), compositions (e.g. pharmaceutical compositions), and methods of the invention may be used to determine if a patient belongs to a group that responds to a pharmaceutical composition of the invention.

Certain embodiments of the present invention are described below, numbered 101-168 and referring to Table C, below, for biological sequences: (101) A cell-targeted molecule comprising i) a binding region comprising an immunoglobulin-type binding region comprising one or more polypeptides and capable of specifically binding at least one extracellular target biomolecule, and ii) a Shiga toxin effector polypeptide comprising a Shiga toxin A1 fragment region having a carboxy terminus and a disrupted furin-cleavage motif at the carboxy terminus of the A1 fragment region. (102) The cell-targeted molecule of embodiment 101, wherein the binding region is associated with the carboxy terminus of the Shiga toxin effector polypeptide. (103) The cell-targeted molecule of embodiment 101 or 102, wherein the binding region and the Shiga toxin effector polypeptide are fused. (104) The cell-targeted molecule of embodiment 101 or 102, wherein the immunoglobulin-type binding region is selected from the group consisting of: single-domain antibody fragment, single-chain variable fragment, antibody variable fragment, complementary determining region 3 fragment, constrained FR3-CDR3-FR4 polypeptide, Fd fragment, antigen-binding fragment, fibronectin-derived 10^{th} fibronectin type III domain, tenascin type III domain, ankyrin repeat motif domain, low-density-lipoprotein-receptor-derived A-domain, lipocalin, Kunitz domain, Protein-A-derived Z domain, gamma-B crystalline-derived domain, ubiquitin-derived domain, Sac7d-derived polypeptide, Fyn-derived SH2 domain, miniprotein, C-type lectin-like domain scaffold, engineered antibody mimic, and any genetically manipulated counterparts of any of the foregoing which retain binding functionality. (105) The cell-targeted molecule of embodiment 104, wherein the binding region is capable of binding to the extracellular target biomolecule selected from the group consisting of: CD20, CD22, CD40, CD79, CD25, CD30, HER2/neu/ErbB2, EGFR, EpCAM, EphB2, prostate-specific membrane antigen, Cripto, endoglin, fibroblast activated protein, Lewis-Y, CD19, CD21, CS1/SLAMF7, CD33, CD52, CD133, EpCAM, CEA, gpA33, Mucins, TAG-72, carbonic anhydrase IX, folate binding protein, ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANKL, FAP, Tenascin, CD64, mesothelin, BRCA1, MART-1/MelanA, gp100, tyrosinase, human tyrosinase related protein 1, tyrosinase related protein 2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen, prostate specific antigen, prostate stem cell antigen, human aspartyl (asparaginyl) beta-hydroxylase, EphA2, HER3/ErbB-3, MUC1, MART-1/MelanA, gp100, tyrosinase associated antigen, HPV-E7, Epstein-Barr Virus antigens, Bcr-Abl, alpha-fetoprotein antigen, 17-A1, bladder tumor antigen, CD38, CD15, CD23, CD52, CD53, CD88, CD129, CD183, CD191, CD193, CD244, CD294, CD305, C3AR, FceRIa, galectin-9, mrp-14, siglec-8, siglec-10, CD49d, CD13, CD44, CD54, CD63, CD69, CD123, CD193, TLR4, FceRIa, IgE, CD107a, CD203c, CD14, CD15, CD33, CD64, CD68, CD80, CD86, CD105, CD115, F4/80, ILT-3, Galectin-3, CD11a-c, GITRL, MHC Class II, CD284-TLR4, CD107-Mac3, CD195-CCR5, HLA-DR, CD16/32, CD282-TLR2, CD11c, CD123, and any immunogenic fragment of any of the foregoing. (106) The cell-targeted molecule of embodiment 104, whereby administration of the cell-targeted molecule to a cell physically coupled with an extracellular target biomolecule of the binding region, the cell-targeted molecule is capable of causing death of the cell. (107) The cell-targeted molecule of embodiment 106, whereby administration of the cell-targeted molecule to a first population of cells whose members are physically coupled to extracellular target biomolecules of the binding region, and a second population of cells whose members are not physically coupled to any extracellular target biomolecule of the binding region, the cytotoxic effect of the cell-targeted molecule to members of said first population of cells relative to members of said second population of cells is at least 3-fold greater. (108) The cell-targeted molecule of embodiment 107, wherein the Shiga toxin effector polypeptide comprises or consists essentially of the polypeptide shown in any one of ID NOs: 4-49. (109) The cell-targeted molecule of embodiment 108, which comprises or consists essentially of the polypeptide shown in any one of ID NOs: 50-61. (110) The cell-targeted molecule of any one of embodiments 101-109, further comprising a carboxy-terminal endoplasmic reticulum retention/retrieval signal motif of a member of the KDEL family (111) The cell-targeted molecule of embodiment 110, comprising the carboxy-terminal endoplasmic reticulum retention/retrieval signal motif selected from the group consisting of: KDEL, HDEF, HDEL, RDEF, RDEL, WDEL, YDEL, HEEF, HEEL, KEEL, REEL, KAEL, KCEL, KFEL, KGEL, KHEL, KLEL, KNEL, KQEL, KREL, KSEL, KVEL, KWEL, KYEL, KEDL, MEL, DKEL, FDEL, KDEF, KKEL, HADL, HAEL, HIEL, HNEL, HTEL, KTEL, HVEL, NDEL, QDEL, REDL, RNEL, RTDL, RTEL, SDEL, TDEL, and SKEL. (112) A cell-targeted molecule comprising i) a Shiga toxin effector polypeptide comprising a Shiga toxin A1 fragment region having a carboxy terminus and a disrupted furin-cleavage motif, at the carboxy terminus of the A1 fragment region, comprising one or more mutations in the minimal, furin-cleavage motif relative to a wild-type, Shiga toxin A Subunit; and ii) a binding region capable of specifically binding at least one extracellular target biomolecule and associated with the carboxy terminus of the Shiga toxin effector polypeptide. (113) The cell-targeted molecule of embodiment 112, wherein the Shiga toxin effector polypeptide and the binding region are fused. (114) The cell-targeted molecule of embodiment 113, whereby administration of the cell-targeted molecule to a cell physically coupled with an extracellular target biomolecule of the binding region, the cell-targeted molecule is capable of causing death of the cell. (115) The cell-targeted molecule of embodiment 114, whereby administration of the cell-targeted molecule to a first population of cells whose members are physically coupled to extracellular target biomolecules of the binding region, and a second population of cells whose members are not physically coupled to any extracellular target biomolecule of the binding region, the cytotoxic effect of the cell-targeted molecule to members of said first population of cells relative to members of said second population of cells is at least 3-fold greater. (116) The cell-targeted molecule of embodiment 115, wherein the Shiga toxin effector polypeptide comprises or consists essentially of the polypeptide shown in any one of ID NOs: 4-36. (117) The cell-targeted molecule of embodiment 116, which comprises or consists essentially of the polypeptide shown in any one of ID NOs: 50-61. (118) A cytotoxic molecule comprising a Shiga toxin effector polypeptide comprising i) a Shiga toxin A1 fragment region having a carboxy terminus and ii) a disrupted furin-cleavage motif at the carboxy terminus of the A1 fragment region; and capable, when a component of a first cell-targeted molecule comprising a binding region capable of specifically binding at least one extracellular target biomolecule and a molecular moiety associated with the carboxy terminus of the Shiga toxin effector polypeptide, of exhibiting cytotoxicity equivalent to cytotoxicity of a second cell-targeted molecule consisting of the cell-targeted molecule except for the Shiga toxin effector polypeptide consists of a wild-type, Shiga toxin A1 polypeptide. (119) The cytotoxic molecule of embodiment 118, wherein the molecular moiety comprises at least one amino acid residue and the Shiga toxin effector polypeptide is fused to at least one amino acid residue of the molecular moiety. (120) The cytotoxic molecule of embodiment 119, wherein the disrupted furin-cleavage motif comprises one or more mutations relative to a wild-type, Shiga toxin A Subunit, the mutation altering at least one amino acid residue in the region natively positioned at 248-251 of the A Subunit of Shiga-like toxin 1 (SEQ ID NO: 1) or Shiga toxin (SEQ ID NO: 2), or at 247-250 of the A Subunit of Shiga-like toxin 2 (SEQ ID NO:3). (121) The cytotoxic molecule of embodiment 120, wherein at least one of the mutations is a substitution of an arginine residue with a non-positively charged, amino acid residue. (122) The cytotoxic molecule of any one of embodiments 118-120, wherein the Shiga toxin effector polypeptide comprises or consists essentially of the polypeptide shown in any one of ID NOs: 4-49. (123) The cytotoxic molecule of any one of embodiments 118-122, further comprising a mutation relative to a naturally occurring A Subunit of a member of the Shiga toxin family which reduces or eliminates the enzymatic activity of the Shiga toxin effector polypeptide. (124) The cytotoxic molecule of any one of embodiments 118-123, wherein the first cell-targeted molecule is capable of exhibiting improved, in vivo tolerability compared to the second cell-targeted molecule. (125) A cytotoxic, cell-targeted molecule comprising i) a binding region capable of specifically binding at least one extracellular target biomolecule, ii) a Shiga toxin effector polypeptide comprising a Shiga toxin A1 fragment region having a carboxy terminus and a disrupted furin-cleavage motif at the carboxy terminus of the A1 fragment region, and iii) a molecular moiety associated with the carboxy terminus of the Shiga toxin effector polypeptide; and wherein the cytotoxic, cell-targeted molecule is capable of exhibiting cytotoxicity equivalent to cytotoxicity of a second cell-targeted molecule consisting of the cell-targeted molecule except for the Shiga toxin effector polypeptide consists of a wild-type, Shiga toxin A1 polypeptide. (126) The cytotoxic, cell-targeted molecule of embodiment 125, wherein the molecular moiety comprises the binding region. (127) The cytotoxic, cell-targeted molecule of embodiment 126, wherein the binding region comprises a polypeptide comprising an immunoglobulin-type binding region. (128) The cytotoxic, cell-targeted molecule of embodiment 127, wherein the immunoglobulin-type binding region is selected from the group consisting of: single-domain antibody fragment, single-chain variable fragment, antibody variable fragment, complementary determining region 3 fragment, constrained FR3-CDR3-FR4 polypeptide, Fd fragment, antigen-binding fragment, fibronectin-derived 10^{th} fibronectin type III domain, tenascin type III domain, ankyrin repeat motif domain, low-density-lipoprotein-receptor-derived A-domain, lipocalin, Kunitz domain, Protein-A-derived Z domain, gamma-B crystalline-derived domain, ubiquitin-derived domain, Sac7d-derived polypeptide, Fyn-derived SH2 domain, miniprotein, C-type lectin-like domain scaffold, engineered antibody mimic, and any genetically manipulated counterparts of any of the foregoing which retain binding functionality. (129) The cytotoxic, cell-targeted molecule of any one of embodiments 125-128, wherein the molecular moiety comprises at least one amino acid residue and the Shiga toxin effector polypeptide is fused to at least one amino acid residue of the molecular moiety. (130) The cytotoxic, cell-targeted molecule of any one of embodiments 125-129, whereby administration of the cytotoxic, cell-targeted molecule to a first population of cells whose members are physically coupled to extracellular target biomolecules of the binding region, and a second population of cells whose members are not physically coupled to any extracellular target biomolecule of the binding region, the cytotoxic effect of the cytotoxic, cell-targeted molecule to members of said first population of cells relative to members of said second population of cells is at least 3-fold greater. (131) The cytotoxic, cell-targeted molecule of embodiment 130, wherein the binding region is capable of binding to an extracellular target biomolecule selected from the group consisting of: CD20, CD22, CD40, CD79, CD25, CD30, HER2/neu/ErbB2, EGFR, EpCAM, EphB2, prostate-specific membrane antigen, Cripto, endoglin, fibroblast activated protein, Lewis-Y, CD19, CD21, CS1/SLAMF7, CD33, CD52, CD133, EpCAM, CEA, gpA33, Mucins, TAG-72, carbonic anhydrase IX, folate binding protein, ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANKL, FAP, Tenascin, CD64, mesothelin, BRCA1, MART-1/MelanA, gp100, tyrosinase, human tyrosinase related protein 1, human tyrosinase related protein 2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen, prostate specific antigen, prostate stem cell antigen, human aspartyl (asparaginyl) beta-hydroxylase, EphA2, HER3/ErbB-3, MUC1, MART-1/MelanA, gp100, tyrosinase associated antigen, HPV-E7, Epstein-Barr Virus antigens, Bcr-Abl, alpha-fetoprotein antigen, 17-A1, bladder tumor antigen, CD38, CD15, CD23, CD52, CD53, CD88, CD129, CD183, CD191, CD193, CD244, CD294, CD305, C3AR, FceRIa, galectin-9, mrp-14, siglec-8, siglec-10, CD49d, CD13, CD44, CD54, CD63, CD69, CD123, CD193, TLR4, FceRIa, IgE, CD107a, CD203c, CD14, CD15, CD33, CD64, CD68, CD80, CD86, CD105, CD115, F4/80, ILT-3, Galectin-3, CD11a-c, GITRL, MHC Class II, CD284-TLR4, CD107-Mac3, CD195-CCR5, HLA-DR, CD16/32, CD282-TLR2, CD11c, CD123, and any immunogenic fragment of any of the foregoing. (132) The cytotoxic, cell-targeted molecule of embodiment 131, wherein the Shiga toxin effector polypeptide comprises or consists essentially of the polypeptide shown in any one of ID NOs: 4-49. (133) The cytotoxic, cell-targeted molecule of embodiment 132, which comprises or consists essentially of the polypeptide shown in any one of ID NOs: 51-60. (134) The cytotoxic, cell-targeted molecule of any one of embodiments 125-133, capable of exhibiting improved, in vivo tolerability compared to the second cell-targeted molecule. (135) A cytotoxic, cell-targeted molecule comprising i) a binding region capable of specifically binding at least one extracellular target biomolecule; ii) a Shiga toxin effector polypeptide comprising a Shiga toxin A1 fragment region having a carboxy terminus and a disrupted furin-cleavage motif at the carboxy terminus of the A1 fragment region; and iii) a molecular moiety associated with the carboxy terminus of the Shiga toxin effector polypeptide; and wherein the cell-targeted molecule is capable of exhibiting improved, in vivo tolerability compared to in vivo tolerability of a second cell-targeted molecule consisting of the cell-targeted molecule except for the Shiga toxin effector polypeptide consists of a wild-type, Shiga toxin A1 polypeptide. (136) The cytotoxic, cell-targeted molecule of embodiment 135, wherein the Shiga toxin effector polypeptide is not cytotoxic and the molecular moiety is toxic. (137) The cytotoxic, cell-targeted molecule of embodiment 135, wherein the molecular moiety comprises the binding region. (138) The cytotoxic, cell-targeted molecule of any one of embodiments 135-137, whereby administration of the cytotoxic, cell-targeted molecule to a first population of cells whose members are physically coupled to extracellular target biomolecules of the binding region, and a second population of cells whose members are not physically coupled to any extracellular target biomolecule of the binding region, the cytotoxic effect of the cytotoxic, cell-targeted molecule to members of said first population of cells relative to members of said second population of cells is at least 3-fold greater. (139) The cytotoxic, cell-targeted molecule of embodiment 137 or 138, wherein the binding region comprises a polypeptide comprising an immunoglobulin-type binding region. (140) The cytotoxic, cell-targeted molecule of embodiment 139, wherein the immunoglobulin-type binding region is selected from the group consisting of: single-domain antibody fragment, single-chain variable fragment, antibody variable fragment, complementary determining region 3 fragment, constrained FR3-CDR3-FR4 polypeptide, Fd fragment, antigen-binding fragment, fibronectin-derived 10^{th} fibronectin type III domain, tenascin type III domain, ankyrin repeat motif domain, low-density-lipoprotein-receptor-derived A-domain, lipocalin, Kunitz domain, Protein-A-derived Z domain, gamma-B crystalline-derived domain, ubiquitin-derived domain, Sac7d-derived polypeptide, Fyn-derived SH2 domain, miniprotein, C-type lectin-like domain scaffold, engineered antibody mimic, and any genetically manipulated counterparts of any of the foregoing which retain binding functionality. (141) The cytotoxic, cell-targeted molecule of embodiment 140, wherein the Shiga toxin effector polypeptide comprises or consists essentially of the polypeptide shown in any one of ID NOs: 4-49. (142) The cytotoxic, cell-targeted molecule of any one of embodiments 135-140, wherein the molecular moiety comprises at least one amino acid residue and the Shiga toxin effector polypeptide is fused to at least one amino acid residue of the molecular moiety. (143) The cytotoxic, cell-targeted molecule of embodiment 142, which comprises or consists essentially of the polypeptide shown in any one of ID NOs: 51-60. (144) A pharmaceutical composition comprising the composition of matter of any one of embodiments 101-143 and at least one pharmaceutically acceptable excipient or carrier. (145) A polynucleotide capable of encoding the polypeptide or protein of the molecule of any one of embodiments 101-143, or a complement thereof, or a fragment of any of the foregoing. (146) An expression vector comprising the polynucleotide of embodiment 145. (147) A host cell comprising any one of the polynucleotides or expression vectors of embodiments 145-146. (148) A diagnostic composition comprising composition of matter of any one of embodiments 101-143 and a detection promoting agent. (149) The composition of matter of any of one of embodiments 101-148, which is immobilized on a solid substrate. (150) A method for improving in vivo tolerability of a molecule comprising i) a Shiga toxin effector polypeptide comprising a Shiga toxin A1 fragment region having a carboxy terminus and a furin-cleavage site proximal to the carboxy terminus of the A1 fragment region, and ii) a heterologous, molecular moiety associated with the carboxy terminus of the Shiga toxin effector polypeptide and comprising a binding region capable of specifically binding at least one extracellular target biomolecule; the method comprising the step of disrupting a furin-cleavage motif comprising the furin-cleavage site. (151) A method for improving in vivo tolerability of a molecule comprising i) a Shiga toxin effector polypeptide comprising a Shiga toxin A1 fragment region having a carboxy terminus and a furin-cleavage site proximal to the carboxy terminus of the A1 fragment region, and ii) a heterologous, molecular moiety associated with the carboxy terminus of the Shiga toxin effector polypeptide and which is toxic; the method comprising the step of disrupting a furin-cleavage motif comprising the furin-cleavage site. (152) A molecule produced by the method of embodiment 150 or embodiment 151. (153) A method of killing a cell comprising the step of contacting the cell with the molecule of any one of embodiments 101-143 or the pharmaceutical composition of embodiment 144. (154) The method of embodiment 153, wherein the contacting occurs in vitro. (155) The method of embodiment 153, wherein the contacting occurs in vivo. (156) A method of treating a disease, disorder, or condition in a patient comprising the step of administering to a patient in need thereof a therapeutically effective amount of the molecule of any one of embodiments 101-143 or the pharmaceutical composition of embodiment 144. (157) The method of embodiment 156, wherein the disease, disorder, or condition is selected from the group consisting of: cancer, tumor, immune disorder, and microbial infection. (158) The method of embodiment 157, wherein the cancer selected from the group consisting of: bone cancer, breast cancer, central/peripheral nervous system cancer, gastrointestinal cancer, germ cell cancer, glandular cancer, head-neck cancer, hematological cancer, kidney-urinary tract cancer, liver cancer, lung/pleura cancer, prostate cancer, sarcoma, skin cancer, and uterine cancer. (159) The method of embodiment 157, wherein the immune disorder associated is with a disease selected from the group consisting of: amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriatic arthritis, rheumatoid arthritis, scleroderma, septic shock, Sjorgren's syndrome, ulcerative colitis, and vasculitis. (160) A composition comprising the molecule of any one of embodiments 101-148 for the treatment or prevention of a cancer, tumor, immune disorder, or microbial infection. (161) Use of the composition of matter of any one of embodiments 101-148 in the manufacture of a medicament for the treatment or prevention of a cancer, tumor, immune disorder, or microbial infection. (162) Use of the composition of matter of any one of embodiments 101-148 in the diagnosis, prognosis, or characterization of a disease, disorder, or condition. (163) A method of detecting a cell, the method comprising the steps of: contacting the cell with the diagnostic composition of embodiment 148 and detecting the presence of the diagnostic composition. (164) The method of embodiment 163, wherein the contacting occurs in vitro. (165) The method of embodiment 163, wherein the contacting occurs in vivo. (166) The method of embodiment 163, wherein the detecting occurs in vitro. (167) The method of embodiment 163, wherein the detecting occurs in vivo. (168) A kit comprising the composition of matter of any one of embodiments 101-148 and an additional reagent and/or pharmaceutical delivery device.

TABLE C
Biological sequences referred to in embodiments 101-168

| Number | Text Description | Sequence |
|---|---|---|
| NO: 1 | Shiga-like toxin 1 Subunit | KEFTLDFSTAKTYVDSLNVIRSAIGTPL |
| | A (SLT-1A) | QTISSGGTSLLMIDSGSGDNLFAVDVR |
| | | GIDPEEGRFNNLRLIVERNNLYVTGFV |
| | | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASRVARMASDEFPSMCPADGRVRGI |
| | | THNKILWDSSTLGAILMRRTISS |
| NO: 2 | Shiga toxin Subunit A | KEFTLDFSTAKTYVDSLNVIRSAIGTPL |
| | | QTISSGGTSLLMIDSGTGDNLFAVDVR |
| | | GIDPEEGRFNNLRLIVERNNLYVTGFV |
| | | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASRVARMASDEFPSMCPADGRVRGI |
| | | THNKILWDSSTLGAILMRRTISS |
| NO: 3 | Shiga-like toxin 2 Subunit | DEFTVDFSSQKSYVDSLNSIRSAISTPLG |
| | A (SLT-2A) | NISQGGVSVSVINHVLGGNYISLNVRG |
| | | LDPYSERFNHLRLIMERNNLYVAGFIN |
| | | IETNIFYRFSDFSHISVPDVITVSMTTDS |
| | | SYSSLQRIADLERTGMQIGRHSLVGSY |
| | | LDLMEFRGRSMTRASSRAMLRFVTVIA |
| | | EALRFRQIQRGFRPALSEASPLYTMTA |
| | | QDVDLTLNWGRISNVLPEYRGEEGVRI |
| | | GRISFNSLSAILGSVAVILNCHSTGSYSV |
| | | RSVSQKQKTECQIVGDRAAIKVNNVL |
| | | WEANTIAALLNRKPQDLTEPNQ |
| NO: 4 | Protease-cleavage resistant, | NLYVTGFVNRTNNVFYRFADFSHVTFP |
| | Shiga toxin effector | GTTAVTLSGDSSYTTLQRVAGISRTGM |
| | polypeptide SLT-1A-FR | QINRHSLTTSYLDLMSHSGTSLTQSVA |
| | variant 1 | RAMLRFVTVTAEALRFRQIQRGFRTTL |
| | | DDLSGRSYVMTAEDVDLTLNWGRLSS |
| | | VLPDYHGQDSVRVGRISFGSINAILGSV |
| | | ALILNCHHHASRVAA |
| NO: 5 | Protease-cleavage resistant, | NLYVAGFINTETNIFYRFSDFSHISVPD |
| | Shiga toxin effector | VITVSMTTDSSYSSLQRIADLERTGMQI |
| | polypeptide SLT-1A-FR | GRHSLVGSYLDLMEFRGRSMTRASSR |
| | variant 2 | AMLRFVTVIAEALRFRQIQRGFRPALSE |
| | | ASPLYTMTAQDVDLTLNWGRISNVLPE |
| | | YRGEEGVRIGRISFNSLSAILGSVAVILN |
| | | CHSTGSYSVA |
| NO: 6 | Protease-cleavage resistant, | NLYVTGFVNRTNNVFYRFADFSHVTFP |
| | Shiga toxin effector | GTTAVTLSGDSSYTTLQRVAGISRTGM |
| | polypeptide SLT-1A-FR | QINRHSLTTSYLDLMSHSGTSLTQSVA |
| | variant 3 | RAMLRFVTVTAEALRFRQIQRGFRTTL |
| | | DDLSGRSYVMTAEDVDLTLNWGRLSS |
| | | VLPDYHGQDSVRVGRISFGSINAILGSV |
| | | ALILNCHHHASAVAR |
| NO: 7 | Protease-cleavage resistant, | NLYVAGFINTETNIFYRFSDFSHISVPD |
| | Shiga toxin effector | VITVSMTTDSSYSSLQRIADLERTGMQI |
| | polypeptide SLT-1A-FR | GRHSLVGSYLDLMEFRGRSMTRASSR |
| | variant 4 | AMLRFVTVIAEALRFRQIQRGFRPALSE |
| | | ASPLYTMTAQDVDLTLNWGRISNVLPE |
| | | YRGEEGVRIGRISFNSLSAILGSVAVILN |
| | | CHSTGSASVR |
| NO: 8 | Protease-cleavage resistant, | NLYVTGFVNRTNNVFYRFADFSHVTFP |
| | Shiga toxin effector | GTTAVTLSGDSSYTTLQRVAGISRTGM |
| | polypeptide SLT-1A-FR | QINRHSLTTSYLDLMSHSGTSLTQSVA |
| | variant 5 | RAMLRFVTVTAEALRFRQIQRGFRTTL |
| | | DDLSGRSYVMTAEDVDLTLNWGRLSS |
| | | VLPDYHGQDSVRVGRISFGSINAILGSV |
| | | ALILNCHHHASAVAA |
| NO: 9 | Protease-cleavage resistant, | NLYVAGFINTETNIFYRFSDFSHISVPD |
| | Shiga toxin effector | VITVSMTTDSSYSSLQRIADLERTGMQI |
| | polypeptide SLT-1A-FR | GRHSLVGSYLDLMEFRGRSMTRASSR |
| | variant 6 | AMLRFVTVIAEALRFRQIQRGFRPALSE |
| | | ASPLYTMTAQDVDLTLNWGRISNVLPE |
| | | YRGEEGVRIGRISFNSLSAILGSVAVILN |
| | | CHSTGSASVA |
| NO: 10 | Protease-cleavage resistant, | NLYVTGFVNRTNNVFYRFADFSHVTFP |
| | Shiga toxin effector | GTTAVTLSGDSSYTTLQRVAGISRTGM |
| | polypeptide SLT-1A-FR | QINRHSLTTSYLDLMSHSGTSLTQSVA |
| | variant 7 | RAMLRFVTVTAEALRFRQIQRGFRTTL |
| | | DDLSGRSYVMTAEDVDLTLNWGRLSS |
| | | VLPDYHGQDSVRVGRISFGSINAILGSV |
| | | ALILNCHHHASRVAAMASDEFPSMC |
| NO: 11 | Protease-cleavage resistant, | NLYVAGFINTETNIFYRFSDFSHISVPD |
| | Shiga toxin effector | VITVSMTTDSSYSSLQRIADLERTGMQI |
| | polypeptide SLT-1A-FR | GRHSLVGSYLDLMEFRGRSMTRASSR |
| | variant 8 | AMLRFVTVIAEALRFRQIQRGFRPALSE |
| | | ASPLYTMTAQDVDLTLNWGRISNVLPE |
| | | YRGEEGVRIGRISFNSLSAILGSVAVILN |
| | | CHSTGSYSVASVSQKQKTEC |
| NO: 12 | Protease-cleavage resistant, | NLYVTGFVNRTNNVFYRFADFSHVTFP |
| | Shiga toxin effector | GTTAVTLSGDSSYTTLQRVAGISRTGM |
| | polypeptide SLT-1A-FR | QINRHSLTTSYLDLMSHSGTSLTQSVA |
| | variant 9 | RAMLRFVTVTAEALRFRQIQRGFRTTL |
| | | DDLSGRSYVMTAEDVDLTLNWGRLSS |
| | | VLPDYHGQDSVRVGRISFGSINAILGSV |
| | | ALILNCHHHASAVARMASDEFPSMC |
| NO: 13 | Protease-cleavage resistant, | NLYVAGFINTETNIFYRFSDFSHISVPD |
| | Shiga toxin effector | VITVSMTTDSSYSSLQRIADLERTGMQI |
| | polypeptide SLT-1A-FR | GRHSLVGSYLDLMEFRGRSMTRASSR |
| | variant 10 | AMLRFVTVIAEALRFRQIQRGFRPALSE |
| | | ASPLYTMTAQDVDLTLNWGRISNVLPE |
| | | YRGEEGVRIGRISFNSLSAILGSVAVILN |
| | | CHSTGSASVRSVSQKQKTEC |
| NO: 14 | Protease-cleavage resistant, | NLYVTGFVNRTNNVFYRFADFSHVTFP |
| | Shiga toxin effector | GTTAVTLSGDSSYTTLQRVAGISRTGM |
| | polypeptide SLT-1A-FR | QINRHSLTTSYLDLMSHSGTSLTQSVA |
| | variant 11 | RAMLRFVTVTAEALRFRQIQRGFRTTL |
| | | DDLSGRSYVMTAEDVDLTLNWGRLSS |
| | | VLPDYHGQDSVRVGRISFGSINAILGSV |
| | | ALILNCHHHASAVAAMASDEFPSMC |
| NO: 15 | Protease-cleavage resistant, | NLYVAGFINTETNIFYRFSDFSHISVPD |
| | Shiga toxin effector | VITVSMTTDSSYSSLQRIADLERTGMQI |
| | polypeptide SLT-1A-FR | GRHSLVGSYLDLMEFRGRSMTRASSR |
| | variant 12 | AMLRFVTVIAEALRFRQIQRGFRPALSE |
| | | ASPLYTMTAQDVDLTLNWGRISNVLPE |
| | | YRGEEGVRIGRISFNSLSAILGSVAVILN |
| | | CHSTGSASVASVSQKQKTEC |
| NO: 16 | Protease-cleavage resistant, | KEFTLDFSTAKTYVDSLNVIRSAIGTPL |
| | Shiga toxin effector | QTISSGGTSLLMIDSGSGDNLFAVDVR |
| | polypeptide SLT-1A-FR | GIDPEEGRFNNLRLIVERNNLYVTGFV |
| | variant 13 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASRVAA |
| NO: 17 | Protease-cleavage resistant, | KEFTLDFSTAKTYVDSLNVIRSAIGTPL |
| | Shiga toxin effector | QTISSGGTSLLMIDSGTGDNLFAVDVR |
| | polypeptide SLT-1A-FR | GIDPEEGRFNNLRLIVERNNLYVTGFV |
| | variant 14 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASRVAA |
| NO: 18 | Protease-cleavage resistant, | DEFTVDFSSQKSYVDSLNSIRSAISTPLG |
| | Shiga toxin effector | NISQGGVSVSVINHVLGGNYISLNVRG |
| | polypeptide SLT-1A-FR | LDPYSERFNHLRLIMERNNLYVAGFIN |
| | variant 15 | IETNIFYRFSDFSHISVPDVITVSMTTDS |
| | | SYSSLQRIADLERTGMQIGRHSLVGSY |
| | | LDLMEFRGRSMTRASSRAMLRFVTVIA |
| | | EALRFRQIQRGFRPALSEASPLYTMTA |
| | | QDVDLTLNWGRISNVLPEYRGEEGVRI |
| | | GRISFNSLSAILGSVAVILNCHSTGSYSV |
| | | R |
| NO: 19 | Protease-cleavage resistant, | KEFTLDFSTAKTYVDSLNVIRSAIGTPL |
| | Shiga toxin effector | QTISSGGTSLLMIDSGSGDNLFAVDVR |
| | polypeptide SLT-1A-FR | GIDPEEGRFNNLRLIVERNNLYVTGFV |
| | variant 16 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAR |
| NO: 20 | Protease-cleavage resistant, | KEFTLDFSTAKTYVDSLNVIRSAIGTPL |
| | Shiga toxin effector | QTISSGGTSLLMIDSGTGDNLFAVDVR |
| | polypeptide SLT-1A-FR | GIDPEEGRFNNLRLIVERNNLYVTGFV |
| | variant 17 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAR |
| NO: 21 | Protease-cleavage resistant, | DEFTVDFSSQKSYVDSLNSIRSAISTPLG |
| | Shiga toxin effector | NISQGGVSVSVINHVLGGNYISLNVRG |
| | polypeptide SLT-1A-FR | LDPYSERFNHLRLIMERNNLYVAGFIN |
| | variant 18 | IETNIFYRFSDFSHISVPDVITVSMTTDS |
| | | SYSSLQRIADLERTGMQIGRHSLVGSY |
| | | LDLMEFRGRSMTRASSRAMLRFVTVIA |
| | | EALRFRQIQRGFRPALSEASPLYTMTA |
| | | QDVDLTLNWGRISNVLPEYRGEEGVRI |
| | | GRISFNSLSAILGSVAVILNCHSTGSASV |
| | | A |
| NO: 22 | Protease-cleavage resistant, | KEFTLDFSTAKTYVDSLNVIRSAIGTPL |
| | Shiga toxin effector | QTISSGGTSLLMIDSGSGDNLFAVDVR |
| | polypeptide SLT-1A-FR | GIDPEEGRFNNLRLIVERNNLYVTGFV |
| | variant 19 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAA |
| NO: 23 | Protease-cleavage resistant, | KEFTLDFSTAKTYVDSLNVIRSAIGTPL |
| | Shiga toxin effector | QTISSGGTSLLMIDSGTGDNLFAVDVR |
| | polypeptide SLT-1A-FR | GIDPEEGRFNNLRLIVERNNLYVTGFV |
| | variant 20 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAA |
| NO: 24 | Protease-cleavage resistant, | DEFTVDFSSQKSYVDSLNSIRSAISTPLG |
| | Shiga toxin effector | NISQGGVSVSVINHVLGGNYISLNVRG |
| | polypeptide SLT-1A-FR | LDPYSERFNHLRLIMERNNLYVAGFIN |
| | variant 21 | IETNIFYRFSDFSHISVPDVITVSMTTDS |
| | | SYSSLQRIADLERTGMQIGRHSLVGSY |
| | | LDLMEFRGRSMTRASSRAMLRFVTVIA |
| | | EALRFRQIQRGFRPALSEASPLYTMTA |
| | | QDVDLTLNWGRISNVLPEYRGEEGVRI |
| | | GRISFNSLSAILGSVAVILNCHSTGSASV |
| | | A |
| NO: 25 | Protease-cleavage resistant, | KEFTLDFSTAKTYVDSLNVIRSAIGTPL |
| | Shiga toxin effector | QTISSGGTSLLMIDSGSGDNLFAVDVR |
| | polypeptide SLT-1A-FR | GIDPEEGRFNNLRLIVERNNLYVTGFV |
| | variant 22 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASRVAAMASDEFPSMC |
| NO: 26 | Protease-cleavage resistant, | KEFTLDFSTAKTYVDSLNVIRSAIGTPL |
| | Shiga toxin effector | QTISSGGTSLLMIDSGTGDNLFAVDVR |
| | polypeptide SLT-1A-FR | GIDPEEGRFNNLRLIVERNNLYVTGFV |
| | variant 23 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASRVAAMASDEFPSMC |
| NO: 27 | Protease-cleavage resistant, | DEFTVDFSSQKSYVDSLNSIRSAISTPLG |
| | Shiga toxin effector | NISQGGVSVSVINHVLGGNYISLNVRG |
| | polypeptide SLT-1A-FR | LDPYSERFNHLRLIMERNNLYVAGFIN |
| | variant 24 | IETNIFYRFSDFSHISVPDVITVSMTTDS |
| | | SYSSLQRIADLERTGMQIGRHSLVGSY |
| | | LDLMEFRGRSMTRASSRAMLRFVTVIA |
| | | EALRFRQIQRGFRPALSEASPLYTMTA |
| | | QDVDLTLNWGRISNVLPEYRGEEGVRI |
| | | GRISFNSLSAILGSVAVILNCHSTGSYSV |
| | | ASVSQKQKTEC |
| NO: 28 | Protease-cleavage resistant, | KEFTLDFSTAKTYVDSLNVIRSAIGTPL |
| | Shiga toxin effector | QTISSGGTSLLMIDSGSGDNLFAVDVR |
| | polypeptide SLT-1A-FR | GIDPEEGRFNNLRLIVERNNLYVTGFV |
| | variant 25 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVARMASDEFPSMC |
| NO: 29 | Protease-cleavage resistant, | KEFTLDFSTAKTYVDSLNVIRSAIGTPL |
| | Shiga toxin effector | QTISSGGTSLLMIDSGTGDNLFAVDVR |
| | polypeptide SLT-1A-FR | GIDPEEGRFNNLRLIVERNNLYVTGFV |
| | variant 26 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVARMASDEFPSMC |
| NO: 30 | Protease-cleavage resistant, | DEFTVDFSSQKSYVDSLNSIRSAISTPLG |
| | Shiga toxin effector | NISQGGVSVSVINHVLGGNYISLNVRG |
| | polypeptide SLT-1A-FR | LDPYSERFNHLRLIMERNNLYVAGFIN |
| | variant 27 | TETNIFYRFSDFSHISVPDVITVSMTTDS |
| | | SYSSLQRIADLERTGMQIGRHSLVGSY |
| | | LDLMEFRGRSMTRASSRAMLRFVTVIA |
| | | EALRFRQIQRGFRPALSEASPLYTMTA |
| | | QDVDLTLNWGRISNVLPEYRGEEGVRI |
| | | GRISFNSLSAILGSVAVILNCHSTGSASV |
| | | RSVSQKQKTEC |
| NO: 31 | Protease-cleavage resistant, | KEFTLDFSTAKTYVDSLNVIRSAIGTPL |
| | Shiga toxin effector | QTISSGGTSLLMIDSGSGDNLFAVDVR |
| | polypeptide SLT-1A-FR | GIDPEEGRFNNLRLIVERNNLYVTGFV |
| | variant 28 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAAMASDEFPSMC |
| NO: 32 | Protease-cleavage resistant, | KEFTLDFSTAKTYVDSLNVIRSAIGTPL |
| | Shiga toxin effector | QTISSGGTSLLMIDSGTGDNLFAVDVR |
| | polypeptide SLT-1A-FR | GIDPEEGRFNNLRLIVERNNLYVTGFV |
| | variant 29 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAAMASDEFPSMC |
| NO: 33 | Protease-cleavage resistant, | DEFTVDFSSQKSYVDSLNSIRSAISTPLG |
| | Shiga toxin effector | NISQGGVSVSVINHVLGGNYISLNVRG |
| | polypeptide SLT-1A-FR | LDPYSERFNHLRLIMERNNLYVAGFIN |
| | variant 30 | TETNIFYRFSDFSHISVPDVITVSMTTDS |
| | | SYSSLQRIADLERTGMQIGRHSLVGSY |
| | | LDLMEFRGRSMTRASSRAMLRFVTVIA |
| | | EALRFRQIQRGFRPALSEASPLYTMTA |
| | | QDVDLTLNWGRISNVLPEYRGEEGVRI |
| | | GRISFNSLSAILGSVAVILNCHSTGSASV |
| | | ASVSQKQKTEC |
| NO: 34 | Protease-cleavage resistant, | KEFTLDFSTAKTYVDSLNVIRSAIGTPL |
| | Shiga toxin effector | QTISSGGTSLLMIDSGSGDNLFAVDVR |
| | polypeptide SLT-1A-FR | GIDPEEGRFNNLRLIVERNNLYVTGFV |
| | variant 31 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASMASDEFPSMC |
| NO: 35 | Protease-cleavage resistant, | KEFTLDFSTAKTYVDSLNVIRSAIGTPL |
| | Shiga toxin effector | QTISSGGTSLLMIDSGTGDNLFAVDVR |
| | polypeptide SLT-1A-FR | GIDPEEGRFNNLRLIVERNNLYVTGFV |
| | variant 32 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASMASDEFPSMC |
| NO: 36 | Protease-cleavage resistant, | DEFTVDFSSQKSYVDSLNSIRSAISTPLG |
| | Shiga toxin effector | NISQGGVSVSVINHVLGGNYISLNVRG |
| | polypeptide SLT-1A-FR | LDPYSERFNHLRLIMERNNLYVAGFIN |
| | variant 33 | TETNIFYRFSDFSHISVPDVITVSMTTDS |
| | | SYSSLQRIADLERTGMQIGRHSLVGSY |
| | | LDLMEFRGRSMTRASSRAMLRFVTVIA |
| | | EALRFRQIQRGFRPALSEASPLYTMTA |
| | | QDVDLTLNWGRISNVLPEYRGEEGVRI |
| | | GRISFNSLSAILGSVAVILNCHSTGSSVS |
| | | QKQKTEC |
| NO: 37 | Protease-cleavage resistant, | KEFTLDFSTAKTYVDSLNVIRSAIGTPL |
| | Shiga toxin effector | QTISSGGTSLLMIDSGSGDNLFAVDVR |
| | polypeptide SLT-1A-FR | GIDPEEGRFNNLRLIVERNNLYVTGFV |
| | variant 34 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HAS |
| NO: 38 | Protease-cleavage resistant, | KEFTLDFSTAKTYVDSLNVIRSAIGTPL |
| | Shiga toxin effector | QTISSGGTSLLMIDSGTGDNLFAVDVR |
| | polypeptide SLT-1A-FR | GIDPEEGRFNNLRLIVERNNLYVTGFV |
| | variant 35 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HAS |
| NO: 39 | Protease-cleavage resistant, | DEFTVDFSSQKSYVDSLNSIRSAISTPLG |
| | Shiga toxin effector | NISQGGVSVSVINHVLGGNYISLNVRG |
| | polypeptide SLT-1A-FR | LDPYSERFNHLRLIMERNNLYVAGFIN |
| | variant 36 | TETNIFYRFSDFSHISVPDVITVSMTTDS |
| | | SYSSLQRIADLERTGMQIGRHSLVGSY |
| | | LDLMEFRGRSMTRASSRAMLRFVTVIA |
| | | EALRFRQIQRGFRPALSEASPLYTMTA |
| | | QDVDLTLNWGRISNVLPEYRGEEGVRI |
| | | GRISFNSLSAILGSVAVILNCHSTGS |
| NO: 40 | Protease-cleavage resistant, | KEFTLDFSTAKTYVDSLNVIRSAIGTPL |
| | Shiga toxin effector | QTISSGGTSLLMIDSGSGDNLFAVDVR |
| | polypeptide SLT-1A-FR | GIDPEEGRFNNLRLIVERNNLYVTGFV |
| | variant 37 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCH |
| NO: 41 | Protease-cleavage resistant, | KEFTLDFSTAKTYVDSLNVIRSAIGTPL |
| | Shiga toxin effector | QTISSGGTSLLMIDSGTGDNLFAVDVR |
| | polypeptide SLT-1A-FR | GIDPEEGRFNNLRLIVERNNLYVTGFV |
| | variant 38 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCH |
| NO: 42 | Protease-cleavage resistant, | DEFTVDFSSQKSYVDSLNSIRSAISTPLG |
| | Shiga toxin effector | NISQGGVSVSVINHVLGGNYISLNVRG |
| | polypeptide SLT-1A-FR | LDPYSERFNHLRLIMERNNLYVAGFIN |
| | variant 39 | TETNIFYRFSDFSHISVPDVITVSMTTDS |
| | | SYSSLQRIADLERTGMQIGRHSLVGSY |
| | | LDLMEFRGRSMTRASSRAMLRFVTVIA |
| | | EALRFRQIQRGFRPALSEASPLYTMTA |
| | | QDVDLTLNWGRISNVLPEYRGEEGVRI |
| | | GRISFNSLSAILGSVAVILNCH |
| NO: 43 | Protease-cleavage resistant, | KEFTLDFSTAKTYVDSLNVIRSAIGTPL |
| | Shiga toxin effector | QTISSGGTSLLMIDSGSGDNLFAVDVR |
| | polypeptide SLT-1A-FR | GIDPEEGRFNNLRLIVERNNLYVTGFV |
| | variant 40 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILN |
| NO: 44 | Protease-cleavage resistant, | KEFTLDFSTAKTYVDSLNVIRSAIGTPL |
| | Shiga toxin effector | QTISSGGTSLLMIDSGTGDNLFAVDVR |
| | polypeptide SLT-1A-FR | GIDPEEGRFNNLRLIVERNNLYVTGFV |
| | variant 41 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILN |
| NO: 45 | Protease-cleavage resistant, | DEFTVDFSSQKSYVDSLNSIRSAISTPLG |
| | Shiga toxin effector | NISQGGVSVSVINHVLGGNYISLNVRG |
| | polypeptide SLT-1A-FR | LDPYSERFNHLRLIMERNNLYVAGFIN |
| | variant 42 | TETNIFYRFSDFSHISVPDVITVSMTTDS |
| | | SYSSLQRIADLERTGMQIGRHSLVGSY |
| | | LDLMEFRGRSMTRASSRAMLRFVTVIA |
| | | EALRFRQIQRGFRPALSEASPLYTMTA |
| | | QDVDLTLNWGRISNVLPEYRGEEGVRI |
| | | GRISFNSLSAILGSVAVILN |
| NO: 46 | Protease-cleavage resistant, | KEFTLDFSTAKTYVDSLNVIRSAIGTPL |
| | Shiga toxin effector | QTISSGGTSLLMIDSGSGDNLFAVDVR |
| | polypeptide SLT-1A-FR | GIDPEEGRFNNLRLIVERNNLYVTGFV |
| | variant 43 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALIL |
| NO: 47 | Protease-cleavage resistant, | KEFTLDFSTAKTYVDSLNVIRSAIGTPL |
| | Shiga toxin effector | QTISSGGTSLLMIDSGTGDNLFAVDVR |
| | polypeptide SLT-1A-FR | GIDPEEGRFNNLRLIVERNNLYVTGFV |
| | variant 44 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALIL |
| NO: 48 | Protease-cleavage resistant, | DEFTVDFSSQKSYVDSLNSIRSAISTPLG |
| | Shiga toxin effector | NISQGGVSVSVINHVLGGNYISLNVRG |
| | polypeptide SLT-1A-FR | LDPYSERFNHLRLIMERNNLYVAGFIN |
| | variant 45 | TETNIFYRFSDFSHISVPDVITVSMTTDS |
| | | SYSSLQRIADLERTGMQIGRHSLVGSY |
| | | LDLMEFRGRSMTRASSRAMLRFVTVIA |
| | | EALRFRQIQRGFRPALSEASPLYTMTA |
| | | QDVDLTLNWGRISNVLPEYRGEEGVRI |
| | | GRISFNSLSAILGSVAVIL |
| NO: 49 | Protease-cleavage resistant, | KEFTLDFSTAKTYVDSLNVIRSAIGTPL |
| | Shiga toxin effector | QTISSGGTSLLMIDSGSGDNLFAVDVR |
| | polypeptide SLT-1A-FR | GIDPEEGRFNNLRLIVERNNLYVTGFV |
| | variant 46 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNPAD |
| | | GRVRGITHNKILWDSSTLGAILMRRTIS |
| | | S |
| NO: 50 | Cytotoxic, Cell-Targeted | MKEFTLDFSTAKTYVDSLNVIRSAIGTP |
| | Molecule: αCD20: : SLT- | LQTISSGGTSLLMIDSGSGDNLFAVDVR |
| | 1A-FR variant #1 | GIDPEEGRFNNLRLIVERNNLYVTGFV |
| | | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAAEFPKPSTPPGSSGGAPMQVQ |
| | | LQQPGAELVKPGASVKMSCKTSGYTF |
| | | TSYNVHWVKQTPGQGLEWIGAIYPGN |
| | | GDTSFNQKFKGKATLTADKSSSTVYM |
| | | QLSSLTSEDSAVYYCARSNYYGSSYV |
| | | WFFDVWGAGTTVTVSSGSTSGSGKPG |
| | | SGEGSQIVLSQSPTILSASPGEKVTMTC |
| | | RASSSVSYMDWYQQKPGSSPKPWIYA |
| | | TSNLASGVPARFSGSGSGTSYSLTISRV |
| | | EAEDAATYYCQQWISNPPTFGAGTKLE |
| | | LK |
| NO: 51 | Cytotoxic, Cell-Targeted | MKEFTLDFSTAKTYVDSLNVIRSAIGTP |
| | Molecule: αCD20: : SLT- | LQTISSGGTSLLMIDSGSGDNLFAVDVR |
| | 1A-FR variant #2 | GIDPEEGRFNNLRLIVERNNLYVTGFV |
| | | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAAGGGGSGGMQVQLVQSGAE |
| | | LVKPGASVKMSCKASGYTFTSYNMH |
| | | WVKQTPGQGLEWIGAIYPGNGDTSYN |
| | | QKFKGKATLTADKSSSTAYMQLSSLTS |
| | | EDSAVYYCARAQLRPNYWYFDVWGA |
| | | GTTVTVSSGGGGSGGGGSGGGGSGGG |
| | | GSGGGGSDIVLSQSPAILSASPGEKVTM |
| | | TCRASSSVSYMHWYQQKPGSSPKPWI |
| | | YATSNLASGVPARFSGSGSGTSYSLTIS |
| | | RVEAEDAATYYCQQWISNPPTFGAGT |
| | | KLELK |
| NO: 52 | Cytotoxic, Cell-Targeted | MKEFTLDFSTAKTYVDSLNVIRSAIGTP |
| | Molecule: αCD20: : SLT- | LQTISSGGTSLLMIDSGSGDNLFAVDVR |
| | 1A-FR variant #3 | GIDPEEGRFNNLRLIVERNNLYVTGFV |
| | | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAAEFPKPSTPPGSSGGAPMQVQ |
| | | LQQPGAELVKPGASVKMSCKASGYTF |
| | | TSYNMHWVKQTPGRGLEWIGAIYPGN |
| | | GDTSYNQKFKGKATLTADKSSSTAYM |
| | | QLSSLTSEDSAVYYCARSTYYGGDWY |
| | | FNVWGAGTTVTVSAGSTSGSGKPGSG |
| | | EGSTKGQIVLSQSPAILSASPGEKVTMT |
| | | CRASSSVSYIHWFQQKPGSSPKPWIYA |
| | | TSNLASGVPVRFSGSGSGTSYSLTISRV |
| | | EAEDAATYYCQQWTSNPPTFGGGTKL |
| | | EIK |
| NO: 53 | Cytotoxic, Cell-Targeted | MKEFTLDFSTAKTYVDSLNVIRSAIGTP |
| | Molecule: αCD20: : SLT- | LQTISSGGTSLLMIDSGSGDNLFAVDVR |
| | 1A-FR variant #4 | GIDPEEGRFNNLRLIVERNNLYVTGFV |
| | | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAAEFPKPSTPPGSSGGAPGILGF |
| | | VFTLMQVQLQQPGAELVKPGASVKMS |
| | | CKTSGYTFTSYNVHWVKQTPGQGLEW |
| | | IGAIYPGNGDTSFNQKFKGKATLTADK |
| | | SSSTVYMQLSSLTSEDSAVYYCARSNY |
| | | YGSSYVWFFDVWGAGTTVTVSSGSTS |
| | | GSGKPGSGEGSQIVLSQSPTILSASPGE |
| | | KVTMTCRASSSVSYMDWYQQKPGSSP |
| | | KPWIYATSNLASGVPARFSGSGSGTSY |
| | | SLTISRVEAEDAATYYCQQWISNPPTFG |
| | | AGTKLELK |
| NO: 54 | Cytotoxic, Cell-Targeted | MKEFTLDFSTAKTYVDSLNVIRSAIGTP |
| | Molecule: HER2- | LQTISSGGTSLLMIDSGSGDNLFAVDVR |
| | V_{H}H: : SLT-1A-FR | GIDPEEGRFNNLRLIVERNNLYVTGFV |
| | | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAAHHSEDPSSKAPKAPEVQLVE |
| | | SGGGLVQAGGSLRLSCAASGITFSINT |
| | | MGWYRQAPGKQRELVALISSIGDTYY |
| | | ADSVKGRFTISRDNAKNTVYLQMNSL |
| | | KPEDTAVYYCKRFRTAAQGTDYWGQ |
| | | GTQVTVSS |
| NO: 55 | Cytotoxic, Cell-Targeted | MKEFTLDFSTAKTYVDSLNVIRSAIGTP |
| | Molecule: αCD20- | LQTISSGGTSLLMIDSGTGDNLFAVDV |
| | FN3: : StxA-FR: : KDEL | RGIDPEEGRFNNLRLIVERNNLYVTGF |
| | | VNRTNNVFYRFADFSHVTFPGTTAVTL |
| | | SGDSSYTTLQRVAGISRTGMQINRHSL |
| | | TTSYLDLMSHSGTSLTQSVARAMLRFV |
| | | TVTAEALRFRQIQRGFRTTLDDLSGRS |
| | | YVMTAEDVDLTLNWGRLSSVLPDYHG |
| | | QDSVRVGRISFGSINAILGSVALILNCH |
| | | HHASAVAAEFPKPSTPPGSSGGAPASV |
| | | SDVPRDLEVVAATPTSLLISWCRQRCA |
| | | DSYRITYGETGGNSPVQEFTVPGSWKT |
| | | ATISGLKPGVDYTITVYVVTHYYGWD |
| | | RYSHPISINYRTGSKDEL |
| NO: 56 | Cytotoxic, Cell-Targeted | MKEFTLDFSTAKTYVDSLNVIRSAIGTP |
| | Molecule: IL-2: : SLT-1A- | LQTISSGGTSLLMIDSGSGDNLFAVDVR |
| | FR: : KDEL variant 1 | GIDPEEGRFNNLRLIVERNNLYVTGFV |
| | | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAAEFPKPSTPPGSSGGAPAPTSS |
| | | STKKTQLQLEHLLLDLQMILNGINNYK |
| | | NPKLTRMLTFKFYMPKKATELKHLQC |
| | | LEEELKPLEEVLNLAQSKNFHLRPRDLI |
| | | SNINVIVLELKGSETTFMCEYADETATI |
| | | VEFLNRWITFCQSIISTLTKDEL |
| NO: 57 | Cytotoxic, Cell-Targeted | MKEFTLDFSTAKTYVDSLNVIRSAIGTP |
| | Molecule: | LQTISSGGTSLLMIDSGSGDNLFAVDVR |
| | αCD38scFv: : SLT-1A-FR | GIDPEEGRFNNLRLIVERNNLYVTGFV |
| | variant 1 | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAAEFPKPSTPPGSSGGAPDIELT |
| | | QSPSSFSVSLGDRVTITCKASEDIYNRL |
| | | AWYQQKPGNAPRLLISGATSLETGVPS |
| | | RFSGSGSGKDYTLSITSLQTEDVATYY |
| | | CQQYWSTPTFGGGTKLEIKGSTSGSGK |
| | | PGSGEGSKVQLQESGPSLVQPSQRLSIT |
| | | CTVSGFSLISYGVHWVRQSPGKGLEWL |
| | | GVIWRGGSTDYNAAFMSRLSITKDNSK |
| | | SQVFFKMNSLQADDTAIYFCAKTLITT |
| | | GYAMDYWGQGTTVTVSS |
| NO: 58 | Cytotoxic, Cell-Targeted | MKEFTLDFSTAKTYVDSLNVIRSAIGTP |
| | Molecule: SLT-1A- | LQTISSGGTSLLMIDSGSGDNLFAVDVR |
| | FR: : αHER2scFv variant 1 | GIDPEEGRFNNLRLIVERNNLYVTGFV |
| | | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAAEFPKPSTPPGSSGGAPDIQM |
| | | TQSPSSLSASVGDRVTITCRASQDVNT |
| | | AVAWYQQKPGKAPKWYSASFLYSG |
| | | VPSRFSGSRSGTDFTLTISSLQPEDFATY |
| | | YCQQHYTTPPTFGQGTKVEIKRTGSTS |
| | | GSGKPGSGEGSEVQLVESGGGLVQPG |
| | | GSLRLSCAASGFNIKDTYIHWVRQAPG |
| | | KGLEWVARIYPTNGYTRYADSVKGRF |
| | | TISADTSKNTAYLQMNSLRAEDTAVY |
| | | YCSRWGGDGFYAMDVWGQGTLVTVS |
| | | S |
| NO: 59 | Cytotoxic, Cell-Targeted | MKEFTLDFSTAKTYVDSLNVIRSAIGTP |
| | Molecule: SLT-1A- | LQTISSGGTSLLMIDSGSGDNLFAVDVR |
| | FR: : αCD19scFv variant 1 | GIDPEEGRFNNLRLIVERNNLYVTGFV |
| | | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAAEFPKPSTPPGSSGGAPDIVM |
| | | TQAAPSIPVTPGESVSISCRSSKSLLNSN |
| | | GNTYLYWFLQRPGQSPQLLIYRMSNLA |
| | | SGVPDRFSGSGSGTAFTLRISRVEAEDV |
| | | GVYYCMQHLEYPFTFGAGTKLELKGS |
| | | TSGSGKPGSGEGSEVQLQQSGPELIKPG |
| | | ASVKMSCKASGYTFTSYVMHWVKQK |
| | | PGQGLEWIGYINPYNDGTKYNEKFKG |
| | | KATLTSDKSSSTAYMELSSLTSEDSAV |
| | | YYCARGTYYYGSRVFDYWGQGTTLT |
| | | VSS |
| NO: 60 | Cytotoxic, Cell-Targeted | MKEFTLDFSTAKTYVDSLNVIRSAIGTP |
| | Molecule: SLT-1A- | LQTISSGGTSLLMIDSGSGDNLFAVDVR |
| | FR: : αCD74scFv variant 1 | GIDPEEGRFNNLRLIVERNNLYVTGFV |
| | | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAAEFPKPSTPPGSSGGAPDIQLT |
| | | QSPLSLPVTLGQPASISCRSSQSLVHRN |
| | | GNTYLHWFQQRPGQSPRLLIYTVSNRF |
| | | SGVPDRFSGSGSGTDFTLKISRVEAEDV |
| | | GVYFCSQSSHVPPTFGAGTRLEIKGSTS |
| | | GSGKPGSGEGSTKGQVQLQQSGSELK |
| | | KPGASVKVSCKASGYTFTNYGVNWIK |
| | | QAPGQGLQWMGWINPNTGEPTFDDDF |
| | | KGRFAFSLDTSVSTAYLQISSLKADDT |
| | | AVYFCSRSRGKNEAWFAYWGQGTLV |
| | | TVSS |
| NO: 61 | Cytotoxic, Cell-Targeted | MKEFTLDFSTAKTYVDSLNVIRSAIGTP |
| | Molecule: SLT-1A- | LQTISSGGTSLLMIDSGSGDNLFAVDVR |
| | FR: : HER2 binding region | GIDPEEGRFNNLRLIVERNNLYVTGFV |
| | | NRTNNVFYRFADFSHVTFPGTTAVTLS |
| | | GDSSYTTLQRVAGISRTGMQINRHSLT |
| | | TSYLDLMSHSGTSLTQSVARAMLRFVT |
| | | VTAEALRFRQIQRGFRTTLDDLSGRSY |
| | | VMTAEDVDLTLNWGRLSSVLPDYHGQ |
| | | DSVRVGRISFGSINAILGSVALILNCHH |
| | | HASAVAAEFPKPSTPPGSSGGAPRGSH |
| | | HHHHHGSDLGKKLLEAARAGQDDEV |
| | | RILMANGADVNAKDEYGLTPLYLATA |
| | | HGHLEIVEVLLKNGADVNAVDAIGFTP |
| | | LHLAAFIGHLEIAEVLLKHGADVNAQD |
| | | KFGKTAFDISIGNGNEDLAEILQKLN |

In certain embodiments, the cell-targeted molecules of the present invention are capable of binding extracellular target biomolecules associated with the cell surface of particular cell types and entering those cells. Once internalized within a targeted cell type, certain embodiments of the cell-targeted molecules of the invention are capable of routing a cytotoxic Shiga toxin effector polypeptide fragment into the cytosol of the target cell. Once in the cytosol of a targeted cell type, certain embodiments of the cell-targeted molecules of the invention are capable of enzymatically inactivating ribosomes and eventually killing the cell. This system is modular in that any number of diverse cell-targeting binding regions, such as, e.g., immunoglobulin-type polypeptides, can be used to target this potent cytotoxicity to various, diverse cell types while providing the improvement of reduced protease-cleavage sensitivity.

In certain embodiments of the molecules of the present invention, the disrupted furin-cleavage motif exhibits a reduction in in vitro furin cleavage of 30%, 40%, 50%, 60%, 70%, 80%, 90%, 95%, 97%, 98% or greater compared to a reference molecule comprising a wild-type, Shiga toxin A1 fragment fused at its carboxy terminus to a polypeptide, such as, e.g., the reference molecule SLT-1A-WT::scFv-1 described in the Examples.

In certain embodiments of the molecules of the present invention, the disrupted furin-cleavage motif comprises a disruption in terms of existence, position, or functional group of one or both of the consensus amino acid residues P1 and P4, such as, e.g., the amino acid residues in positions 1 and 4 of the minimal furin-cleavage motif R/Y-x-x-R.

In certain embodiments, the disrupted furin-cleavage motif comprises a disruption in the spacing between the consensus amino acid residues P4 and P1 in terms of the number of intervening amino acid residues being other than two, and, thus, changing either P4 and/or P1 into a different position and eliminating the P4 and/or P1 designations.

In certain embodiments of the molecules of the present invention, the disrupted furin-cleavage motif comprises the deletion of nine, ten, eleven, or more of the carboxy-terminal amino acid residues within the furin-cleavage motif. In these embodiments, the disrupted furin-cleavage motif will not comprise a furin-cleavage site or a minimal furin-cleavage motif. In other words, certain embodiments lack a furin-cleavage site at the carboxy terminus of the A1 fragment region.

In certain embodiments, a molecule of the present invention comprises a Shiga toxin effector polypeptide comprising a disrupted furin-cleavage motif comprising a mutation in the surface-exposed, protease sensitive loop conserved among Shiga toxin A Subunits. For example, in StxA and SLT-1A, this protease-sensitive loop is natively positioned from position 242 to position 261, and in SLT-2A, this loop is natively positioned from position 241 to position 260. Based on polypeptide sequence homology, the skilled worker can identify this conserved, protease-sensitive loop in other Shiga toxin A Subunits. In certain further embodiments, a molecule of the present invention comprises a Shiga toxin effector polypeptide comprising a disrupted furin-cleavage motif comprising a mutation in this protease-sensitive loop of Shiga toxin A Subunits, the mutation which reduce the surface accessibility of certain amino acid residues within the loop such that furin-cleavage sensitivity is reduced.

In certain embodiments, a molecule of the present invention comprises the disrupted furin-cleavage motif comprising the amino acid residue substitution of one or both of the arginine residues in the minimal, cleavage site consensus motif with A, G, or H. In certain further embodiments, the disrupted furin-cleavage motif comprises a deletion of the region natively positioned at 247-252 in StxA (SEQ ID NO:2) and SLT-1A (SEQ ID NO:3), or the region natively positioned at 246-251 in SLT-2A (SEQ ID NO:3); a deletion of the region natively positioned at 244-246 in StxA (SEQ ID NO:2) and SLT-1A (SEQ ID NO:3), or the region natively positioned at 243-245 in SLT-2A (SEQ ID NO:3); or a deletion of the region natively positioned at 253-259 in StxA (SEQ ID NO:2) and SLT-1A (SEQ ID NO:3), or the region natively positioned at 252-258 in SLT-2A (SEQ ID NO:3). Certain further embodiments comprise the disrupted furin-cleavage motif comprising a combination of any of the aforementioned mutations, where possible.

Certain molecules of the present invention comprise a molecular moiety associated with the carboxy terminus of the Shiga toxin effector polypeptide. In certain further embodiments, the association comprises a covalent bond linking the carboxy terminus of the Shiga toxin effector polypeptide, either directly or indirectly, with the molecular moiety. In certain further embodiments, the association comprises the peptide bond which fuses the carboxy terminus of the Shiga toxin effector polypeptide with one or more amino acid residues of the molecular moiety. In certain further embodiments, the Shiga toxin effector polypeptide and the molecular moiety are fused to form a single, continuous polypeptide such that the Shiga toxin effector polypeptide is physically located within the continuous polypeptide amino-terminal to the molecular moiety.

In certain embodiments, a molecule of the invention may comprise the molecular moiety comprising a peptide. In certain embodiments, a molecule of the invention may comprise the molecular moiety having a mass of 1.5 kDa or greater. In certain embodiments, a molecule of the invention may comprise the molecular moiety that has a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the molecule retains the appropriate Shiga toxin biological activity noted herein.

In certain embodiments, the molecular moiety has a mass of about 4.5 kDa or another equivalent mass of a Shiga toxin A2 fragment. It was unexpected that a moiety of this size can remain attached to the carboxy terminus of the Shiga toxin A1 fragment without disrupting the efficiency of sub-cellular routing and ribosome inactivation within intoxicated cells.

In certain embodiments, the molecular moiety has a mass of about 7.6 kDa or another equivalent mass of a Shiga toxin B Subunit. It was unexpected that a moiety of this size can remain attached to the carboxy terminus of the Shiga toxin A1 fragment without disrupting the efficiency of sub-cellular routing and ribosome inactivation within intoxicated cells.

In certain embodiments, the molecular moiety has a mass of about 6-10 kDa or greater and comprises a binding region comprising an antibody mimetic or alternative antibody format, such as, e.g., engineered Armadillo repeat polypeptides (ArmRPs), engineered, fibronectin-derived, 10^{th} fibronectin type III (10Fn3) domain (monobodies, AdNectins™, or AdNexins™); engineered, ankyrin repeat motif containing polypeptide (DARPins™); engineered, low-density-lipoprotein-receptor-derived, A domain (LDLR-A) (Avimers™); engineered, Protein-A-derived, Z domain (Affibodies™); engineered, gamma-B crystalline-derived scaffold or engineered, ubiquitin-derived scaffold (Affilins); and Sac7d-derived polypeptides (Nanoffitins® or affitins).

In certain embodiments, the molecular moiety has a mass of about 11 kDa or more and comprises a binding region comprising an immunoglobulin domain(s) and which specifically binds an extracellular target biomolecule with high affinity, such as, e.g., a V_{H}H or nanobody. In certain further embodiments, the molecular moiety has a mass of about 24 kDa or more and comprises a binding region comprising an immunoglobulin domain and which specifically binds an extracellular target biomolecule with high affinity, such as, e.g., a scFv.

In certain embodiments, the molecular moiety has a mass of about 12 kDa or another equivalent mass of a Shiga toxin A2 fragment and B Subunit complex. It was unexpected that a moiety of this size can remain attached to the carboxy terminus of the Shiga toxin A1 fragment without disrupting the efficiency of sub-cellular routing and ribosome inactivation within intoxicated cells.

In certain embodiments, the molecular moiety has a mass of about 28 kDa. It was unexpected that a moiety of this size can remain attached to the carboxy terminus of the Shiga toxin A1 fragment without disrupting the efficiency of sub-cellular routing and ribosome inactivation within intoxicated cells; however, the Examples herein demonstrate that a furin-cleavage resistant molecule comprising a Shiga toxin A1 fragment fused to a 28 kDa molecular moiety did not exhibit any apparent disruption in sub-cellular routing, ribosome inhibition, or cytotoxicity.

In certain embodiments, the relatively large, molecular moiety has a mass of about 39 kDa or another equivalent mass of a Shiga toxin B Subunit pentamer. It was unexpected that a moiety of this size can remain attached to the carboxy terminus of the Shiga toxin A1 fragment without disrupting the efficiency of sub-cellular routing and ribosome inactivation within intoxicated cells.

In certain embodiments, the relatively large, molecular moiety has a mass of about 43.2 kDa or another equivalent mass of a Shiga toxin A2 fragment and B Subunit pentamer complex. It was unexpected that a moiety of this size can remain attached to the carboxy terminus of the Shiga toxin A1 fragment without disrupting the efficiency of sub-cellular routing and ribosome inactivation within intoxicated cells.

In certain embodiments, the molecular moiety is branched. In certain embodiments, the molecule moiety is non-proteinaceous. In certain embodiments, the molecular moiety is a cytotoxic agent or detection promoting agent, such as agents described herein.

In certain embodiments, the molecular moiety sterically covers the carboxy-terminus of the Shiga toxin A1 fragment polypeptide of the Shiga toxin effector polypeptide of the invention. For purposes of the present invention, “sterically cover” or “sterically covering” refers to a moiety covalently attached directly to the carboxy terminal region of the Shiga toxin A1 fragment polypeptide of the Shiga toxin effector polypeptide of the invention. In certain embodiments, the molecular moiety sterically covers the carboxy terminal region of the Shiga toxin A1 fragment polypeptide of the Shiga toxin effector polypeptide of the invention such that the hydrophobic region within the carboxy-terminal region of the Shiga toxin A1 fragment polypeptide of the Shiga toxin effector polypeptide of the invention remain buried and is not surface exposed in the endoplasmic reticulum, thereby keeping the carboxy terminus of the A1 fragment region covered and preventing cellular recognition of the carboxy terminus of the A1 fragment-derived region, such as, e.g. recognition by the ERAD machinery.

In certain embodiments, the molecular moiety comprises a polypeptide which is more polar and hydrophilic than the carboxy-terminal region of a Shiga toxin A1 fragment such that the hydrophobic region within the carboxy-terminal region of the Shiga toxin A1 fragment polypeptide of the Shiga toxin effector polypeptide of the invention remain buried and is not surface exposed in the endoplasmic reticulum, thereby keeping the carboxy terminus of the A1 fragment region covered and preventing cellular recognition of the carboxy terminus of the A1 fragment-derived region, such as, e.g. recognition by the ERAD machinery.

In certain embodiments, the molecular moiety comprises a binding region capable of specifically binding at least one target biomolecule which is membrane bound in the endoplasmic reticulum membrane.

In certain embodiments, the molecular moiety comprises a binding region capable of specifically binding at least one extracellular target biomolecule.

In certain embodiments of the molecules of the invention, the Shiga toxin effector polypeptide, molecular moiety, and optional, endoplasmic reticulum retention/retrieval signal motif may be directly linked to each other and/or suitably linked to each other via one or more intervening polypeptide sequences, such as with one or more linkers well known in the art and/or described herein. In the above embodiments of the cell-targeted molecules of the invention, the Shiga toxin effector polypeptide regions, binding regions, and other components present in certain embodiments (e.g. molecular moiety and/or endoplasmic reticulum retention/retrieval signal motif) may be directly linked to each other and/or suitably linked to each other via one or more intervening polypeptide sequences, such as with one or more linkers well known in the art and/or described herein.

In certain embodiments, the molecules of the present invention (e.g. cell-targeted molecules of the invention) exhibit increased stability and/or improved, in vivo tolerability as compared to furin-cleavage sensitive analogs. The increased stability can be exhibited in vitro and/or in vivo.

Certain embodiments of the molecules of the invention will have longer half-lives as compared to furin-cleavage sensitive variants, especially with regard to the continued association of the Shiga toxin effector component and one or more other components. For example, certain embodiments of the molecules of the invention will have longer half-lives with regard to the continued association of the Shiga toxin effector component and another component, e.g. a cell-targeting moiety, as compared to a furin-cleavage sensitive variant wherein the furin-cleavage sensitive site(s) lies between those two components.

In certain embodiments of the molecules of the present invention for delivery of additional exogenous material, the additional exogenous material is a cytotoxic agent, such as, e.g., a small molecule chemotherapeutic agent, cytotoxic antibiotic, alkylating agent, antimetabolite, topoisomerase inhibitor, and/or tubulin inhibitor. Non-limiting examples of cytotoxic agents include aziridines, cisplatins, tetrazines, procarbazine, hexamethylmelamine, vinca alkaloids, taxanes, camptothecins, etoposide, doxorubicin, mitoxantrone, teniposide, novobiocin, aclarubicin, anthracyclines, actinomycin, bleomycin, plicamycin, mitomycin, daunorubicin, epirubicin, idarubicin, dolastatins, maytansines, docetaxel, adriamycin, calicheamicin, auristatins, pyrrolobenzodiazepine, carboplatin, 5-fluorouracil (5-FU), capecitabine, mitomycin C, paclitaxel, 1,3-Bis(2-chloroethyl)-1-nitrosourea (BCNU), rifampicin, cisplatin, methotrexate, and gemcitabine.

In certain embodiments, the molecular moiety of a molecule of the present invention comprises or consists essentially of an additional exogenous material.

In certain embodiments of the molecules of the present invention, one or more amino acid residues may be mutated, inserted, or deleted in order to increase the enzymatic activity of the protease-cleavage resistant, Shiga toxin effector polypeptide region as long as the disrupted furin-cleavage motif remains disrupted. For example, mutating residue-position alanine-231 in Stx1A to glutamate increased its enzymatic activity in vitro (Suhan M, Hovde C, Infect Immun 66: 5252-9 (1998)), but will not restore furin-cleavage sensitivity.

Certain embodiments of the present invention include a molecule of the present invention (e.g. a protease-cleavage resistant, cytotoxic molecule or cell-targeted molecule) or any effector fragment thereof, immobilized on a solid substrate. Solid substrates contemplated herein include, but are not limited to, microbeads, nanoparticles, polymers, matrix materials, microarrays, microtiter plates, or any solid surface known in the art (see e.g. U.S. Pat. No. 7,771,955). In accordance with these embodiments, a molecule of the present invention may be covalently or non-covalently linked to a solid substrate, such as, e.g., a bead, particle, or plate, using techniques known to the skilled worker. Immobilized molecules of the invention may be used for screening applications using techniques known in the art (see e.g. Bradbury A et al., Nat Biotechnol 29: 245-54 (2011); Sutton C, Br J Pharmacol 166: 457-75 (2012); Diamante L et al., Protein Eng Des Sel 26: 713-24 (2013); Houlihan G et al., J Immunol Methods 405: 47-56 (2014)).

Non-limiting examples of solid substrates to which a molecule of the invention may be immobilized on include: microbeads, nanoparticles, polymers, nanopolymers, nanotubes, magnetic beads, paramagnetic beads, superparamagnetic beads, streptavidin coated beads, reverse-phase magnetic beads, carboxy terminated beads, hydrazine terminated beads, silica (sodium silica) beads and iminodiacetic acid (IDA)-modified beads, aldehyde-modified beads, epoxy-activated beads, diaminodipropylamine (DADPA)-modified beads (beads with primary amine surface group), biodegradable polymeric beads, polystyrene substrates, amino-polystyrene particles, carboxyl-polystyrene particles, epoxy-polystyrene particles, dimethylamino-polystyrene particles, hydroxy-polystyrene particles, colored particles, flow cytometry particles, sulfonate-polystyrene particles, nitrocellulose surfaces, reinforced nitrocellulose membranes, nylon membranes, glass surfaces, activated glass surfaces, activated quartz surfaces, polyvinylidene difluoride (PVDF) membranes, polyacrylamide-based substrates, poly-vinyl chloride substrates, poly-methyl methacrylate substrates, poly(dimethyl siloxane) substrates, and photopolymers which contain photoreactive species (such as nitrenes, carbenes, and ketyl radicals) capable of forming covalent linkages. Other examples of solid substrates to which a molecule of the invention may be immobilized on are commonly used in molecular display systems, such as, e.g., cellular surfaces, phages, and virus particles.

Certain embodiments of the cytotoxic molecule of the invention, or pharmaceutical compositions thereof, can be used to kill an immune cell (whether healthy or malignant) in a patient by targeting an extracellular biomolecule found physically coupled with an immune cell.

Certain embodiments of the cytotoxic molecule of the invention, or pharmaceutical compositions thereof, can be used to kill an infected cell in a patient by targeting an extracellular biomolecule found physically coupled with an infected cell.

It is within the scope of the present invention to utilize the cytotoxic molecule of the invention, or pharmaceutical compositions thereof, for the purposes of ex vivo depletion of B-cells and/or T-cells from isolated cell populations removed from a patient. In one non-limiting example, the cytotoxic molecule can be used in a method for prophylaxis of organ transplant rejection wherein the donor organ is perfused prior to transplant with the cytotoxic molecule of the invention or a pharmaceutical composition thereof in order to purge the organ of unwanted donor B-cells and/or T-cells.

It is also within the scope of the present invention to utilize the cytotoxic molecule of the invention, or pharmaceutical composition thereof, for the purposes of purging patient cell populations (e.g. bone marrow) of malignant, neoplastic, or otherwise unwanted B-cells and/or T-cells and then reinfusing the B-cell and/or T-cell depleted material into the patient.

It is also within the scope of the present invention to utilize the cytotoxic molecule of the invention, or pharmaceutical composition thereof, for the purposes of depleting B-cells, NK cells, and/or T-cells from a donor cell population as a prophylaxis against graft-versus-host disease, and induction of tolerance, in a patient to undergo a bone marrow and or stem cell transplant (see e.g. Sarantopoulos S et al., Biol Blood Marrow Transplant 21: 16-23 (2015)).

Certain embodiments of the cytotoxic molecule of the invention, or pharmaceutical compositions thereof, can be used to kill an infected cell in a patient by targeting an extracellular biomolecule found physically coupled with an infected cell.

Additionally, the present invention provides a method of treating a disease, disorder, or condition in a patient comprising the step of administering to a patient in need thereof a therapeutically effective amount of at least one of the cytotoxic molecule of the invention, or a pharmaceutical composition thereof. Contemplated diseases, disorders, and conditions that can be treated using this method include cancers, malignant tumors, non-malignant tumors, growth abnormalities, immune disorders, and microbial infections. Administration of a “therapeutically effective dosage” of a molecule or composition of the invention may result in a decrease in severity of disease symptoms, an increase in frequency and duration of disease symptom-free periods, or a prevention of impairment or disability due to the disease affliction.

The therapeutically effective amount of a molecule or composition of the present invention will depend on the route of administration, the type of mammal being treated, and the physical characteristics of the specific patient under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained by the present invention, and may be confirmed in properly designed clinical trials. An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

In certain embodiments, molecules and pharmaceutical compositions of the invention can be used to treat or prevent cancers, tumors (malignant and non-malignant), growth abnormalities, immune disorders, and microbial infections. In a further aspect, the above ex vivo method can be combined with the above in vivo method to provide methods of treating or preventing rejection in bone marrow transplant recipients, and for achieving immunological tolerance.

In certain embodiments, the present invention provides methods for treating malignancies or neoplasms and other blood cell associated cancers in a mammalian subject, such as a human, the method comprising the step of administering to a subject in need thereof a therapeutically effective amount of a cytotoxic molecule or pharmaceutical composition of the invention.

The molecules and pharmaceutical compositions of the invention have varied applications, including, e.g., uses in removing unwanted B-cells and/or T-cells, uses in modulating immune responses to treat graft-versus-host disease, uses as antiviral agents, uses as antimicrobial agents, and uses in purging transplantation tissues of unwanted cell types. The molecules and pharmaceutical compositions of the present invention are commonly anti-neoplastic agents—meaning they are capable of treating and/or preventing the development, maturation, or spread of neoplastic or malignant cells by inhibiting the growth and/or causing the death of cancer or tumor cells.

In another aspect, certain embodiments of the molecules and pharmaceutical compositions of the present invention are antimicrobial agents—meaning they are capable of treating and/or preventing the acquisition, development, or consequences of microbiological pathogenic infections, such as caused by viruses, bacteria, fungi, prions, or protozoans.

It is within the scope of the present invention to provide a prophylaxis or treatment for diseases or conditions mediated by B-cells and/or by T-cells, the prophylaxis or treatment involving administering the cytotoxic molecule of the invention, or a pharmaceutical composition thereof, to a patient for the purpose of killing B-cells and/or T-cells in the patient. This usage is compatible with preparing or conditioning a patient for bone marrow transplantation, stem cell transplantation, tissue transplantation, or organ transplantation, regardless of the source of the transplanted material, e.g. human or non-human sources.

The molecules, cell-targeted molecules, and pharmaceutical compositions of the present invention may be utilized in a method of treating cancer comprising administering to a patient, in need thereof, a therapeutically effective amount of a molecule, cell-targeted molecule, or pharmaceutical composition of the present invention. In certain embodiments of the methods of the present invention, the cancer being treated is selected from the group consisting of: bone cancer (such as multiple myeloma or Ewing's sarcoma), breast cancer, central/peripheral nervous system cancer (such as brain cancer, neurofibromatosis, or glioblastoma), gastrointestinal cancer (such as stomach cancer or colorectal cancer), germ cell cancer (such as ovarian cancers and testicular cancers, glandular cancer (such as pancreatic cancer, parathyroid cancer, pheochromocytoma, salivary gland cancer, or thyroid cancer), head-neck cancer (such as nasopharyngeal cancer, oral cancer, or pharyngeal cancer), hematological cancers (such as leukemia, lymphoma, or myeloma), kidney-urinary tract cancer (such as renal cancer and bladder cancer), liver cancer, lung/pleura cancer (such as mesothelioma, small cell lung carcinoma, or non-small cell lung carcinoma), prostate cancer, sarcoma (such as angiosarcoma, fibrosarcoma, Kaposi's sarcoma, or synovial sarcoma), skin cancer (such as basal cell carcinoma, squamous cell carcinoma, or melanoma), and uterine cancer.

The molecules and pharmaceutical compositions of the present invention may be utilized in a method of treating an immune disorder comprising administering to a patient, in need thereof, a therapeutically effective amount of the cytotoxic molecule or a pharmaceutical composition of the present invention. In certain embodiments of the methods of the present invention, the immune disorder is related to an inflammation associated with a disease selected from the group consisting of: amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriatic arthritis, rheumatoid arthritis, scleroderma, septic shock, Sjorgren's syndrome, ulcerative colitis, and vasculitis.

Among certain embodiments of the present invention is using the molecule of the invention as a component of a pharmaceutical composition or medicament for the treatment or prevention of a cancer, tumor, growth abnormality, immune disorder, and/or microbial infection. For example, immune disorders presenting on the skin of a patient may be treated with such a medicament in efforts to reduce inflammation. In another example, skin tumors may be treated with such a medicament in efforts to reduce tumor size or eliminate the tumor completely.

Certain cytotoxic molecules, pharmaceutical compositions, and diagnostic compositions of the invention may be used in molecular neurosurgery applications such as immunolesioning and neuronal tracing (see, Wiley R, Lappi D, Adv Drug Deliv Rev 55: 1043-54 (2003), for review). For example, the targeting domain may be selected or derived from various ligands, such as neurotransmitters and neuropeptides, which target specific neuronal cell types by binding neuronal surface receptors, such as a neuronal circuit specific G-protein coupled receptor. Similarly, the targeting domain may be selected from or derived from antibodies that bind neuronal surface receptors. Because Shiga toxin effector polypeptides can robustly direct their own retrograde axonal transport, certain cytotoxic molecules of the invention may be used to kill a neuron(s) which expresses the extracellular target at a site of cytotoxic molecule injection distant from the cell body (see Llewellyn-Smith I et al., J Neurosci Methods 103: 83-90 (2000)). These neuronal cell type specific targeting cytotoxic molecules of the invention have uses in neuroscience research, such as for elucidating mechanisms of sensations (see e.g. Mishra S, Hoon M, Science 340: 968-71 (2013), and creating model systems of neurodegenerative diseases, such as Parkinson's and Alzheimer's (see e.g. Hamlin A et al., PLoS One e53472 (2013)).

Among certain embodiments of the present invention is a method of using a molecule (e.g. cytotoxic molecule or cell-targeted molecule), polypeptide, protein, pharmaceutical composition, and/or diagnostic composition of the invention to detect the presence of a cell type for the purpose of information gathering regarding diseases, conditions and/or disorders. The method comprises contacting a cell with a diagnostically sufficient amount of a cytotoxic molecule to detect the cytotoxic molecule by an assay or diagnostic technique. The phrase “diagnostically sufficient amount” refers to an amount that provides adequate detection and accurate measurement for information gathering purposes by the particular assay or diagnostic technique utilized. Generally, the diagnostically sufficient amount for whole organism in vivo diagnostic use will be a non-cumulative dose of between 0.1 mg to 100 mg of the detection promoting agent linked cell-targeted molecule per kg of subject per subject. Typically, the amount of molecule of the invention (e.g. cell-targeted molecule) used in these information gathering methods will be as low as possible provided that it is still a diagnostically sufficient amount. For example, for in vivo detection in an organism, the amount of cytotoxic molecule, cell-targeted molecule, pharmaceutical composition, or diagnostic composition of the invention administered to a subject will be as low as feasibly possible.

Among certain embodiments of the present invention is a method of using a molecule or pharmaceutical composition of the invention as a diagnostic composition to label or detect the interiors of cancer, tumor, and/or immune cell types (see e.g., Koyama Y et al., Clin Cancer Res 13: 2936-45 (2007); Ogawa M et al., Cancer Res 69: 1268-72 (2009); Yang L et al., Small 5: 235-43 (2009)). Based on the ability of certain molecules, cell-targeted molecules, and pharmaceutical compositions of the invention to enter specific cell types and route within cells via retrograde intracellular transport, the interior compartments of specific cell types are labeled for detection. This method may be performed in vivo within a patient, including on cells in situ, e.g. at a disease locus, and/or in vitro on cells removed from an organism, e.g. biopsy material.

The present invention is further illustrated by the following non-limiting examples of selectively cytotoxic, cell-targeted proteins, each protein comprising 1) a Shiga toxin effector region derived from an A Subunit of a member of the Shiga toxin family and 2) an immunoglobulin-type binding region capable of binding an extracellular target biomolecule physically coupled to a specific cell type(s); and wherein the binding region is linked to the protein carboxy-terminal to the Shiga toxin effector region.

### EXAMPLES

The following examples demonstrate certain embodiments of the present invention. However, it is to be understood that these examples are for illustration purposes only and do not intend, nor should any be construed, to be wholly definitive as to conditions and scope of this invention. The examples were carried out using standard techniques, which are well known and routine to those of skill in the art, except where otherwise described in detail.

The following examples demonstrate the more optimized ability of exemplary, cell-targeted, fusion proteins of the present invention to potently kill cells physically coupled with an extracellular target biomolecule of the immunoglobulin-type binding region. The exemplary proteins of the invention bound to target biomolecules expressed by targeted cell types and entered the targeted cells. The internalized, exemplary proteins effectively routed their Shiga toxin effector region polypeptides to the cytosol to inactivate ribosomes and subsequently caused the apoptotic death of the targeted cells with a cytotoxic potency approximately 4-fold to 9-fold or higher as compared to the cytotoxic potency of related fusion proteins engineered with their polypeptide components (i.e. binding region and Shiga toxin effector region) oriented in the reverse amino-carboxy orientation.

In addition, the examples below describe the unexpected discovery that disruption of a conserved furin-cleavage motif at the carboxy terminus of the Shiga toxin A1 fragment did not diminish the cytotoxicity of Shiga-toxin-A-Subunit-derived, cell-targeted molecules despite the uncleavable, Shiga toxin A1 fragment being covalently linked at its carboxy terminus to binding region moieties of a relatively large size, e.g. greater than 28 kDa in size. This was surprising because the Shiga toxin intoxication process was thought to require liberation of the Shiga toxin A1 fragment from all other large molecular moieties, such as, e.g., the Shiga toxin A2 fragment and pentamer of Shiga toxin B Subunits. This was also surprising because the Shiga toxin intoxication process was thought to require liberation of the Shiga toxin A1 fragment from the Shiga holotoxin's cell-targeting, binding regions (i.e. its Shiga toxin B Subunits). Furthermore, this was surprising because the optimal Shiga toxin intoxication process was thought to require the liberation of the carboxy-terminus of the Shiga toxin A1 fragment from all other large molecular moieties in order to present a hydrophobic domain in its carboxy-terminus for recognition by the ERAD system and provide for the efficient retrotranslocation of the A1 fragment from the endoplasmic reticulum to the cytosol where the A1 fragment catalytically inactivates the intoxicated cell's ribosomes.

As demonstrated in the examples below, the cytotoxicity of exemplary cell-targeted molecules comprising a furin-cleavage resistant, Shiga toxin effector region to target cells was equivalent to the cytotoxicity of cell-targeted molecules comprising furin-cleavage sensitive, Shiga toxin effector regions. Similarly, the selective cytotoxicity of exemplary cell-targeted molecules comprising a furin-cleavage resistant, Shiga toxin effector polypeptide to selectively kill cells physically coupled with an extracellular target biomolecule of their binding regions was equivalent to the cytotoxicity of cell-targeted molecules comprising a furin-cleavage sensitive, Shiga toxin effector polypeptide. Certain exemplary, cytotoxic, cell-targeted molecules of the present invention effectively 1) entered target cells; 2) routed their furin-cleavage resistant, Shiga toxin effector polypeptide to the cytosol; 3) inactivated ribosomes; and 4) killed target cells with a cytotoxic potency equivalent to cell-targeted molecules comprising a furin-cleavage sensitive, wild-type, Shiga toxin effector region. Further, after administration to mammals, certain exemplary cell-targeted molecules exhibited improved in vivo, non-specific, toxicity as compared to cell-targeted molecules comprising a furin-cleavage sensitive, Shiga toxin effector region.

Two exemplary, cytotoxic, cell-targeted proteins comprised a Shiga toxin A Subunit fragment recombined with single-chain, variable fragment, binding regions capable of binding CD38 with high affinity. These exemplary proteins were capable of selectively killing cells that express CD38 on their surface, and one of them comprised a disrupted furin-cleavage motif. An exemplary cell-targeted protein targeting CD38 exhibited better optimized cytotoxic potency than its reverse orientation variant. Two, other, exemplary, cytotoxic, cell-targeted proteins comprised a Shiga toxin A Subunit fragment recombined with single-chain, variable fragment, binding regions capable of binding HER2 with high affinity. These exemplary proteins were capable of selectively killing cells that express HER2 on their surface, and one of them comprised a disrupted furin-cleavage motif. An exemplary cell-targeted protein targeting HER2 exhibited better optimized cytotoxic potency than its reverse orientation variant. One, exemplary, cytotoxic, cell-targeted protein comprised a Shiga toxin A Subunit fragment recombined with a single-chain, variable fragment, binding region capable of binding CD19 with high affinity. This exemplary protein was capable of selectively killing cells that express CD19 on their surface with better optimized cytotoxic potency than its reverse orientation variant. A second, exemplary, cytotoxic, cell-targeted protein comprised a Shiga toxin A Subunit fragment recombined with a single-chain, variable fragment, binding region capable of binding CD74 with high affinity. This second, exemplary protein was capable of killing cells that express CD74 on their surface with better optimized cytotoxic potency than its reverse orientation variant. Other exemplary cell-targeted molecules include those with binding regions targeting Epstein-Barr virus antigens, Leishmania antigens, HIV envelope glycoproteins and capsid protein antigens, cytomegalovirus antigens, H. capsulatum antigens, helminth intestinal proteins, neurotensin receptors, epidermal growth factor receptors, B-lymphocyte antigens, the immune cell receptor CCR5, and MHC molecules complexed with peptide-epitopes.

The linking of immunoglobulin-type binding regions with Shiga toxin A Subunit effector polypeptides, such that the Shiga toxin effector polypeptides were more proximal to an amino-terminus of a polypeptide component of the cell-targeted molecule as compared to the position of an immunoglobulin-type binding region, enabled the engineering of cell-type specific targeting of Shiga toxin cytotoxicity with better optimized, cytotoxic potency, such as, e.g., 4-fold, 5-fold, 6-fold, 9-fold, or greater improvement over cell-targeted molecules without amino-terminus proximal Shiga toxin effector polypeptide regions. In certain further embodiments, the cell-targeted molecules of the present invention have a disrupted furin-cleavage motif between the Shiga toxin effector region and the immunoglobulin-type binding region to reduce the likelihood of premature separation of Shiga toxin effector polypeptides from immunoglobulin-type binding regions.

### Example 1

### A CD38-Targeted, Cytotoxic Protein Derived from the A Subunit of Shiga-Like Toxin-1 (SLT-1A::αCD38scFv)

The cytotoxic protein of this example SLT-1A::αCD38scFv comprises a Shiga toxin A Subunit fragment recombined with a single-chain, variable fragment, binding region capable of binding CD38 with high affinity such that the Shiga toxin effector region is more proximal to the amino-terminus of the cytotoxic protein than the CD38 binding region.

### Construction, Production, and Purification of the Cytotoxic Protein SLT-1A::αCD38scFv

First, a Shiga toxin effector region and an immunoglobulin-type binding region were designed or selected. In this example, the Shiga toxin effector region was derived from the A subunit of Shiga-like Toxin 1 (SLT-1A). A polynucleotide was obtained that encoded amino acids 1-251 of SLT-1A (Cheung M et al., Mol Cancer 9: 28 (2010). An immunoglobulin-type binding region αCD38scFv was derived from the monoclonal antibody anti-CD38 HB7 (Peng et al., Blood 101: 2557-62 (2003); see also GenBank Accession BD376144, National Center for Biotechnology Information, U.S.) such that a single-chain variable fragment (scFv) is created with the two immunoglobulin variable regions (V_{L} and V_{H}) separated by a linker known in the art.

Second, the binding region and Shiga toxin effector region were linked together to form a fusion protein. In this example, a polynucleotide encoding the Shiga toxin effector region from SLT-1A (amino acids 1-251 of SEQ ID NO:1) was cloned in frame with a polynucleotide encoding a linker, such as a “murine hinge” derived from a murine IgG3 molecule (or other linkers known to the skilled person) and in frame with a polynucleotide encoding the immunoglobulin-type binding region αCD38scFv comprising amino acids 269-508 of SEQ ID NO:4. In certain experiments, the full-length coding sequence of the cytotoxic protein of this example began with a polynucleotide encoding a Strep-tag® to facilitate detection and purification. The polynucleotide sequence encoding the cytotoxic protein SLT-1A::αCD38scFv of this example was codon optimized for efficient expression in E. coli using services from DNA 2.0, Inc. (Menlo Park, Calif., U.S.).

Third, a fusion protein was produced by expressing the polynucleotide encoding the cytotoxic protein SLT-1A::αCD38scFv (SEQ ID NO:4). Expression of the SLT-1A::αCD38scFv cytotoxic protein was accomplished using both bacterial and cell-free, protein translation systems.

In this example of SLT-1A::αCD38scFv production by an E. coli expression system, the polynucleotide “insert” sequence encoding SLT-1A::αCD38scFv was cloned into the pTxb1 vector (New England Biolabs, Ipswich, Mass., U.S.) using standard procedures to produce a polynucleotide sequence encoding the cytotoxic protein SLT-1A::αCD38scFv ligated in frame to polynucleotide sequences encoding the amino-terminal intein of the vector. The plasmid insert polynucleotide sequence was verified by Sanger sequencing (Functional Biosciences, Madison, Wis., U.S.) and transformed into T7 Shuffle® cells (New England Biolabs, Ipswich, Mass., U.S.). The SLT-1A::αCD38scFv protein was produced and purified according to the IMPACT™ (Intein Mediated Purification with an Affinity Chitin-binding Tag) system manual (New England Biolabs, Ipswich, Mass., U.S.). Purification was accomplished using standard techniques known in the art, such as using immobilized targets of the Strep-tag® or the immunoglobulin-type binding region.

In this example of SLT-1A::αCD38scFv production by a cell-free, protein translation system, the polynucleotide “insert” sequence encoding SLT-1A::αCD38scFv was cloned into the pIVEX2.3 vector with a stop codon directly after the coding region using the In-Fusion® HD Cloning Kit (Clonetech, Mountain View, Calif., U.S.) according to the manufacturer's instructions. The plasmid insert polynucleotide sequence was verified by Sanger sequencing (Functional Biosciences, Madison, Wis., U.S.). SLT-1A::αCD38scFv protein was produced using the rapid translation system 5 Prime™ RTS 100 E. coli Disulfide Kit (5 Prime, Gaithersburg, Md., U.S.) according to the manufacturer's instructions. Purification was accomplished using standard techniques known in the art, such as using immobilized targets of the Strep-tag® or the immunoglobulin-type binding region.

Determining the Maximum Specific Binding (Bₘₐₓ) and Equilibrium Binding Constant (K_{D}) of SLT-1A::αCD38scFv Binding Target Cell Types

The binding characteristics of the SLT-1A::αCD38scFv protein produced as described above were determined by a fluorescence-based, flow-cytometry assay. Samples containing CD38 positive (CD38+) cells and CD38 negative (CD38−) cells were suspended in phosphate buffered saline (1×PBS) (Hyclone Brand, Fisher Scientific, Waltham, Mass., U.S.) containing one percent bovine serum albumin (BSA) (Calbiochem, San Diego, Calif., U.S.), hereinafter referred to as “1×PBS+1% BSA” and incubated for one hour at 4 degrees Celsius (° C.) with 100 μL of various dilutions of the SLT-1A::αCD38scFv protein to be assayed. The highest concentrations of SLT-1A::αCD38scFv protein was selected to lead to saturation of the binding reaction. After the one-hour incubation, cell samples were washed twice with 1×PBS+1% BSA. The cell samples were incubated for 1 hour at 4° C. with 100 μL of 1×PBS+1% BSA containing 0.3 μg of anti-Strep-tag® mAb-FITC (# A01736-100, Genscript, Piscataway, N.J., U.S.).

The cell samples were next washed twice with 1×PBS+1% BSA, resuspended in 200 μL of 1×PBS and subjected to fluorescence-based, flow cytometry. The mean fluorescence intensity (MFI) data for all the samples was obtained by gating the data using a FITC-only sample as a negative control. Graphs were plotted of MFI versus “concentration of cells” using Prism software (GraphPad Software, San Diego, Calif., U.S.). Using the Prism software function of one-site binding [Y=Bₘₐₓ*X/(K_{D}+X)] under the heading binding-saturation, the Bₘₐₓ and K_{D} were calculated using baseline corrected data. Abs values were corrected for background by subtracting the Abs values measured for wells containing only PBS. Bₘₐₓ is the maximum specific binding reported in MFI. K_{D} is the equilibrium binding constant, reported in nM.

The Bₘₐₓ for SLT-1A::αCD38scFv binding to CD38+ cells was measured to be about 100,000 MFI with a K_{D} of about 13 nM (Table 1). This result was similar to the Bₘₐₓ for the reverse orientation protein αCD38scFv::SLT-1A binding to CD38+ cells which was measured to be about 110,000 MFI with a K_{D} of about 17 nM (Table 1). Neither protein bound to CD38− cells. This shows that the “orientation of engineering” effect is probably not related to a perturbation of the immunoglobulin-derived domain's target cell binding properties.

TABLE 1
Orientation of Engineering Had No Significant Effect
on Binding Characteristics: Representative values for Bₘₐₓ
and K_{D} for SLT-1A::αCD38scFv as compared to the
reverse orientation αCD38scFv::SLT-1A

| | target | Target Positive Cells | |
|---|---|---|---|
| Cytotoxic Protein | biomolecule | Bₘₐₓ (MFI) | K_{D} (nM) |
| SLT-1A::αCD38scFv | CD38 | 104,000 | 13.4 |
| αCD38scFv::SLT-1A | CD38 | 108,000 | 17.0 |

### Determining the Half-Maximal Inhibitory Concentration (IC₅₀) of SLT-1A::αCD38scFv to Eukaryotic Ribosomes In Vitro

The ribosome inactivation capabilities of SLT-1A::αCD38scFv was determined in a cell-free, in vitro protein translation assay using the TNT® Quick Coupled Transcription/Translation Kit (L1170 Promega, Madison, Wis., U.S.). The kit includes Luciferase T7 Control DNA and TNT® Quick Master Mix. The ribosome activity reaction was prepared according to the manufacturer's instructions to create “TNT” reaction mixtures.

A series of 10-fold dilutions of SLT-1A::αCD38scFv to be tested was prepared in appropriate buffer and a series of identical TNT reaction mixture components was created for each dilution of SLT-1A::αCD38scFv. Each sample in the dilution series of SLT-1A::αCD38scFv protein was combined with each of the TNT reaction mixtures along with the Luciferase T7 Control DNA. The test samples were incubated for 1.5 hours at 30° C. After the incubation, Luciferase Assay Reagent (E1483 Promega, Madison, Wis., U.S.) was added to all test samples and the amount of luciferase protein translation was measured by luminescence according to the manufacturer instructions. The level of translational inhibition was determined by non-linear regression analysis of log-transformed concentrations of total protein versus relative luminescence units. Using statistical software (GraphPad Prism, San Diego, Calif., U.S.), the half maximal inhibitory concentration (IC₅₀) value was calculated for each sample. Then, the data were normalized by calculating the “percent of SLT-1A-only control protein” using the Prism software function of log(inhibitor) vs. response (three parameters) [Y=Bottom+((Top−Bottom)/(1+10̂(X−Log IC50)))] under the heading dose-response-inhibition. The IC₅₀ for experimental proteins and SLT-1A-only control protein were calculated. The percent of SLT-1A-only control protein was calculated by [(IC50 of SLT-1A control protein/IC50 of experimental protein)×100].

The inhibitory effect of SLT-1A::αCD38scFv on cell-free protein synthesis was strong. Dose dependence experiments determined that the IC₅₀ SLT-1A::αCD38scFv on protein synthesis in this cell-free assay was about 14 picomolar (pM) or 109 percent of the SLT-1A-only positive control (Table 2). This result was not substantially different from the IC₅₀ for the reverse orientation protein αCD38scFv::SLT-1A, which was measured to be about 15 pM or equivalent of the SLT-1A-only positive control (Table 2). This shows that the “orientation of engineering” effect is probably not related to any significant perturbation of Shiga toxin A Subunit enzymatic activity.

TABLE 2
Orientation of Engineering Had No Significant Effect on
Ribosome Inactivation: Representative half-maximal inhibitory
concentration (IC₅₀) for SLT-1A::αCD38scFv as compared
to the reverse orientation αCD38scFv::SLT-1A

| | | IC₅₀ of SLT-1A | Percent of IC₅₀ |
|---|---|---|---|
| | | only positive | of SLT-1A |
| Cytotoxic Protein | IC₅₀ (pM) | control (pM) | control protein |
| SLT-1A::αCD38scFv | 13.7 | 15.0 | 109.0% |
| αCD38scFv::SLT-1A | 14.8 | 15.0 | 99.0% |

### Determining the Selective Cytotoxicity and Half-Maximal Cytotoxic Concentration (CD₅₀) of SLT-1A::αCD38scFv Using a Cell-Kill Assay

The cytotoxicity characteristics of SLT-1A::αCD38scFv was determined by the following cell-kill assay. This assay determines the capacity of a cytotoxic protein to kill cells expressing the target biomolecule of the cytotoxic protein's immunoglobulin-type binding region as compared to cells that do not express the target biomolecule. Cells were plated (2×10³ cells per well) in 20 μL cell culture medium in 384-well plates. The SLT-1A::αCD38scFv protein was diluted either 5-fold or 10-fold in a 1×PBS and 5 μL of the dilutions were added to the cells. Control wells containing only cell culture medium were used for baseline correction. The cell samples were incubated with SLT-1A::αCD38scFv, or just buffer, for 3 days at 37° C. and an atmosphere of 5 percent (%) carbon dioxide (CO₂). The total cell survival or percent viability was determined using a luminescent readout using the CellTiter-Glo® Luminescent Cell Viability Assay (G7573 Promega Madison, Wis., U.S.) according to the manufacturer's instructions. The Percent Viability of experimental wells was calculated using the following equation: (Test RLU−Average Media RLU)/(Average Cells RLU−Average Media RLU)*100. Log polypeptide concentration versus Percent Viability was plotted in Prism (GraphPad Prism, San Diego, Calif., U.S.) and log (inhibitor) vs. normalized response (variable slope) analysis was used to determine the half-maximal cytotoxic concentration (CD₅₀) value for SLT-1A::αCD38scFv.

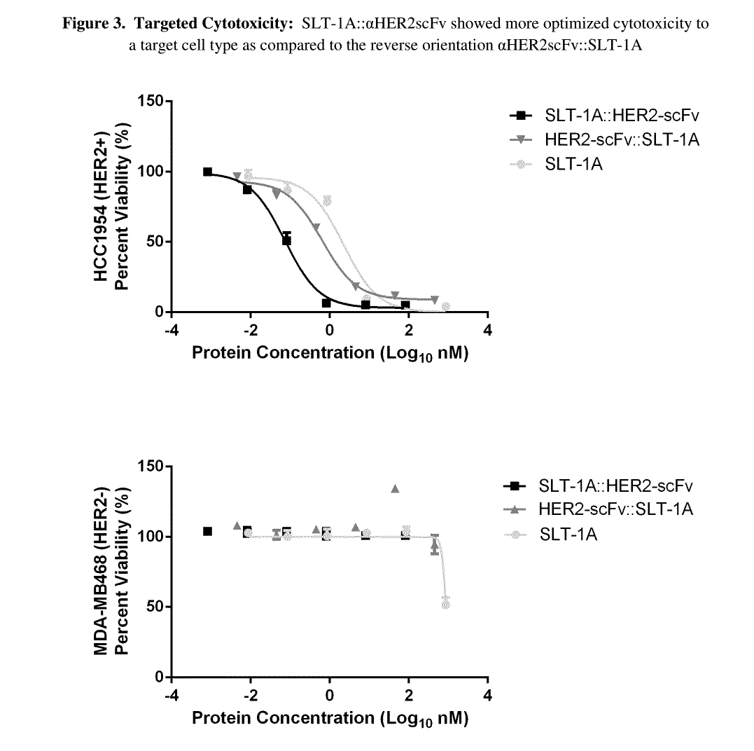

Dose dependence experiments determined that the CD₅₀ of the SLT-1A::αCD38scFv protein was about 0.2-0.7 nM for CD38+ cells depending on the cell line as compared to 470 nM for a CD38− cell line, which was similar to the CD₅₀ for the SLT-1A-only negative control (Table 3; FIG. 3). The CD₅₀ of the SLT-1A::αCD38scFv was about 700-3000 fold greater (less cytotoxic) for cells not physically coupled with the extracellular target biomolecule CD38 as compared to cells which were physically coupled with the extracellular target biomolecule CD38, e.g. cell lines which express CD38 on their cell surface (Table 3; FIG. 3). The CD₅₀ for the same protein domains recombined in the reverse orientation, αCD38scFv::SLT-1A, was measured to be about 0.8-3.2 nM (Table 3; FIG. 3).

TABLE 3
Orientation of Engineering Effect on Cytotoxicity: Representative
half-maximal cytotoxic concentrations (CD₅₀) of SLT-1A::αCD38scFv
as compared to the reverse orientation αCD38scFv::SLT-1A

| | | CD₅₀ (nM) | | |
|---|---|---|---|---|
| | | | | SLT-1A only |
| | CD38 | SLT-1A:: | αCD38scFv:: | negative |
| Cell Line | status | αCD38scFv | SLT-1A | control |
| Daudi | positive | 0.28 | 1.08 | 750 |
| Raji | positive | 0.68 | 3.21 | 1,100 |
| ST486 | positive | 0.21 | 0.75 | 940 |
| BC-1 | positive | 0.18 | 1.08 | 510 |
| U226 | negative | 470.00 | 674.00 | 490 |

These results showed that the better optimized orientation of engineering, where the immunoglobulin-type region was not located proximally to the amino-terminus of the cytotoxic protein relative to the Shiga toxin effector region, conferred a cytotoxic potency toward various CD38+ cell types of about 4-fold to 6-fold higher in CD₅₀ values compared to the less optimized, cytotoxic protein variants constructed using the suboptimal, reverse orientation of engineering (Table 3). These results exemplify the “orientation of engineering” effect on both cytotoxicity and selective cytotoxicity. The differences in cell-kill for the cytotoxic proteins shown in this example were not predictable based on the in vitro results for either ribosome inactivation or target cell-binding characteristics.

### Determining Cell Internalization of SLT-1A::αCD38scFv and its Reverse Orientation αCD38scFv::SLT-1A Using Immunofluorescence

The ability of cytotoxic proteins to enter target cells was investigated using standard immunocytochemical techniques known in the art. Briefly, 0.8×10⁶ cells of each cell type (Raji (CD38+), Ramos (CD38+), Daudi (CD38+), BC-1 (CD38+), and U266 (CD38−)) were harvested and suspended in 50 μL of cell culture medium containing a cocktail of protease inhibitors (e.g. P1860 Sigma-Aldrich Co., St. Louis, Mo., U.S.) and human Fc receptor protein to reduce non-specific immunofluorescent staining. Next, 100 nM of the cytotoxic protein to be analyzed was added to the cells, and the cells were incubated at 37° C. for 1 hour to allow for intoxication to progress. Then, cells were “fixed” and “permeabilized” using the Cytofix/Cytoperm™ Kit (BD Biosciences San Diego, Calif., U.S.) according to the manufacturer's instructions. The Shiga toxin effector region was “stained” using a mouse monoclonal antibody (mouse IgG anti-Shiga toxin 1 Subunit A, BEI NR-867 BEI Resources, Manassas, Va., U.S.). The mouse monoclonal antibody localization was then detected with the Alexa Fluor® 555 Monoclonal Antibody Labeling Kit (Life Technologies, Carlsbad Calif., U.S.) according to manufacturer's instructions.

In this assay, cell surface binding and cell internalization was observed in CD38+ cells for both SLT-1A::αCD38scFv and the reverse orientation protein αCD38scFv::SLT-1A. No cell internalization was observed for either protein to CD38− cells. This showed the differences in cytotoxicity (Table 3; FIG. 3) between these two variants, which differ only in the relative order of their immunoglobulin-type binding region and Shiga toxin effector region, is probably not related to any significant change in target cell binding and/or cell entry.

### Determining the In Vivo Effects of the Cytotoxic Protein αCD38scFv::SLT-1A Using Animal Models

Animal models are used to determine the in vivo effects of the cytotoxic protein αCD38scFv::SLT-1A on CD38+ neoplastic and/or immune cells. Various mice strains are used to test the effect of the cytotoxic protein after intravenous administration on xenograft tumors in mice resulting from the injection into those mice of human neoplastic and/or human immune cells which express CD38 on their cell surfaces.

### Example 2

### A HER2-Targeted, Cytotoxic Protein Derived from the A Subunit of Shiga-Like Toxin-1 (SLT-1A::αHER2scFv)

The cytotoxic protein of this example SLT-1A::αHER2scFv comprises a Shiga toxin A Subunit fragment recombined with a single-chain, variable fragment, binding region capable of binding HER2 with high affinity such that the Shiga toxin effector region is more proximal to the amino-terminus of the cytotoxic protein than the HER2 binding region.

Construction, Production, and Purification of the Cytotoxic Protein SLT-1A::αHER-2scFv

In this example, the Shiga toxin effector region was derived from the A subunit of Shiga-like Toxin 1 (SLT-1A). A polynucleotide was obtained that encoded amino acids 1-251 of SLT-1A (Cheung M et al., Mol Cancer 9: 28 (2010)). An immunoglobulin-type binding region αHER2scFv was derived from trastuzumab (marketed as Herceptin®, Genentech, South San Francisco, Calif.) monoclonal antibody as described (Zhao Y et al., J Immunol 183: 5563-74 (2009)) such that a single-chain variable fragment (scFv) is created with the two immunoglobulin variable regions (V_{L} and V_{H}) separated by a linker.

In this example, the immunoglobulin-type binding region and Shiga toxin effector region were linked together to form a fusion protein. In this example, a polynucleotide encoding the Shiga toxin effector region derived from SLT-1A (amino acids 1-251 of SEQ ID NO:1) was cloned in frame with a polynucleotide encoding a linker, such as a “murine hinge” derived from a murine IgG3 molecule or other linker known to the skilled worker, and in frame with a polynucleotide encoding the immunoglobulin-type binding region αHER2scFv comprising amino acids 269-512 of SEQ ID NO:8. In certain experiments, the full-length coding sequence of the cytotoxic protein of this example began with a polynucleotide encoding a Strep-tag® to facilitate detection and purification. The polynucleotide sequence encoding the cytotoxic protein SLT-1A::αHER2scFv of this example was codon optimized for efficient expression in E. coli using services from DNA 2.0, Inc. (Menlo Park, Calif., U.S.).

A fusion protein was produced by expressing the polynucleotide encoding the cytotoxic protein SLT-1A::αHER2scFv (SEQ ID NO:8). Expression of the SLT-1A::αHER2scFv cytotoxic protein was accomplished using both bacterial and cell-free, protein translation systems.

In this example of SLT-1A::αHER2scFv production by an E. coli expression system, the polynucleotide “insert” sequence encoding SLT-1A::αHER2scFv was cloned into the pTxb1 vector (New England Biolabs, Ipswich, Mass., U.S.) using standard procedures to produce a polynucleotide sequence encoding the cytotoxic protein SLT-1A::αHER2scFv ligated in frame to polynucleotide sequences encoding the amino-terminal intein of the vector. The plasmid insert polynucleotide sequence was verified by Sanger sequencing (Functional Biosciences, Madison, Wis., U.S.) and transformed into T7 Shuffle® cells (New England Biolabs, Ipswich, Mass., U.S.). The SLT-1A::αHER2scFv protein was produced and purified according to the IMPACT™ (Intein Mediated Purification with an Affinity Chitin-binding Tag) system manual (New England Biolabs, Ipswich, Mass., U.S.). Purification was accomplished using standard techniques known in the art, such as using immobilized targets of the Strep-tag® or the immunoglobulin-type binding region.

In this example of SLT-1A::αHER2scFv production by a cell-free, protein translation system, the polynucleotide “insert” sequence encoding SLT-1A::αHER2scFv was cloned into the pIVEX2.3 vector with a stop codon directly after the coding region using the In-Fusion® HD Cloning Kit (Clonetech, Mountain View, Calif., U.S.) according to the manufacturer's instructions. The plasmid insert polynucleotide sequence was verified by Sanger sequencing (Functional Biosciences, Madison, Wis., U.S.). SLT-1A::αHER2scFv protein was produced using the rapid translation system 5 Prime™ RTS 100 E. coli Disulfide Kit (5 Prime, Gaithersburg, Md., U.S.) according to the manufacturer's instructions. Purification was accomplished using standard techniques known in the art, such as using immobilized targets of the Strep-tag® or the immunoglobulin-type binding region.

Determining the Maximum Specific Binding (Bₘₐₓ) and Equilibrium Binding Constant (K_{D}) of SLT-1A::αHER2scFv Binding Target Cell Types

The binding characteristics of the SLT-1A::αHER2scFv protein produced as described above were determined by a fluorescence-based, flow-cytometry assay. Samples containing HER2 positive (HER2+) cells and HER2 negative (HER2−) cells were suspended in 1×PBS+1% BSA and incubated for 1 hour at 4° C. with 100 μL of various dilutions of the SLT-1A::αHER2scFv protein to be assayed. The highest concentrations of SLT-1A::αHER2scFv protein was selected to lead to saturation of the binding reaction. After the one hour incubation, cell samples were washed twice with 1×PBS+1% BSA. The cell samples were incubated for 1 hour at 4° C. with 100 μL of 1×PBS+1% BSA containing 0.3 μg of anti-Strep-tag® mAb-FITC (# A01736-100, Genscript, Piscataway, N.J., U.S.).

The cell samples were next washed twice with 1×PBS+1% BSA, resuspended in 200 μL of 1×PBS and subjected to fluorescence-based, flow cytometry. The mean fluorescence intensity (MFI) data for all the samples was obtained by gating the data using a FITC-only sample as a negative control. Graphs were plotted of MFI versus “concentration of cells” using Prism software (GraphPad Software, San Diego, Calif., U.S.). Using the Prism software function of one-site binding [Y=Bₘₐₓ*X/(K_{D}+X)] under the heading binding-saturation, the Bₘₐₓ and K_{D} were calculated using baseline corrected data. Abs values were corrected for background by subtracting the Abs values measured for wells containing only PBS. Bₘₐₓ is the maximum specific binding reported in MFI. K_{D} is the equilibrium binding constant, reported in nanomolar (nM).

The Bₘₐₓ for SLT-1A::αHER2scFv binding to HER2+ cells was measured to be about 230,000 MFI with a K_{D} of about 110 nM (Table 4). These binding characteristics results were relatively similar to the Bₘₐₓ for the reverse orientation protein αHER2scFv::SLT-1A binding to HER2+ cells which was measured to be about 140,000 MFI with a K_{D} of about 180 nM (Table 4). Neither protein was observed to have measurable binding to HER2− negative cells in this assay. This shows that the “orientation of engineering” effect is probably not related to a perturbation of the immunoglobulin-derived domain's target cell binding properties.

TABLE 4
Orientation of Engineering Had No Effect on Binding Characteristics:
Representative values for Bₘₐₓ and K_{D} for SLT-1A::αHER2scFv
as compared to its reverse orientation αHER2scFv::SLT-1A

| | target | Target Positive Cells | |
|---|---|---|---|
| Cytotoxic Protein | biomolecule | Bₘₐₓ (MFI) | K_{D} (nM) |
| SLT-1A::αHER2scFv | HER2 | 231,000 | 110 |
| αHER2scFv::SLT-1A | HER2 | 141,000 | 182 |

### Determining the Half-Maximal Inhibitory Concentration (IC₅₀) of SLT-1A::αHER2scFv to Eukaryotic Ribosomes In Vitro

The ribosome inactivation capabilities of SLT-1A::αHER2scFv was determined in a cell-free, in vitro protein translation assay using the TNT® Quick Coupled Transcription/Translation Kit (L1170 Promega, Madison, Wis., U.S.). The kit includes Luciferase T7 Control DNA and TNT® Quick Master Mix. The ribosome activity reaction was prepared according to the manufacturer's instructions to create “TNT” reaction mixtures.

A series of 10-fold dilutions of SLT-1A::αHER2scFv to be tested were prepared in appropriate buffer and a series of identical TNT reaction mixture components were created for each dilution of SLT-1A::αHER2scFv. Each sample in the dilution series of SLT-1A::αHER2scFv protein was combined with each of the TNT reaction mixtures along with the Luciferase T7 Control DNA. The test samples were incubated for 1.5 hours at 30° C. After the incubation, Luciferase Assay Reagent (E1483 Promega, Madison, Wis., U.S.) was added to all test samples and the amount of luciferase protein translation was measured by luminescence according to the manufacturer instructions. The level of translational inhibition was determined by non-linear regression analysis of log-transformed concentrations of total protein versus relative luminescence units. Using statistical software (GraphPad Prism, San Diego, Calif., U.S.), the half maximal inhibitory concentration (IC₅₀) value was calculated for each sample. Then, the data were normalized by calculating the “percent of SLT-1A-only control protein” using the Prism software function of log(inhibitor) vs. response (three parameters) [Y=Bottom+((Top−Bottom)/(1+10̂(X−Log IC50)))] under the heading dose-response-inhibition. The IC₅₀ for experimental proteins and SLT-1A-only control protein were calculated. The percent of SLT-1A-only control protein was calculated by [(IC50 of SLT-1A control protein/IC50 of experimental protein)×100].

The inhibitory effect of SLT-1A::αHER2scFv on cell-free protein synthesis was strong. Dose dependence experiments determined that the IC₅₀ of SLT-1A::αHER2scFv on protein synthesis in this cell-free assay was about 110 pM or within 19 percent of the SLT-1A-only positive control (Table 5). This result was not substantially different from the IC₅₀ for the reverse orientation protein αHER2scFv::SLT-1A which was measured to be 108 percent of the SLT-1A-only positive control (Table 5). This shows that the “orientation of engineering” effect is probably not related to any significant perturbation of Shiga toxin A Subunit enzymatic activity.

TABLE 5
Orientation of Engineering Had No Effect on Ribosome Inactivation:
Representative relative half-maximal inhibitory concentrations
for (IC₅₀) for SLT-1A::αHER2scFv as compared to the
reverse orientation αHER2scFv::SLT-1A

| | Percent of IC₅₀ of |
|---|---|
| Cytotoxic Protein | SLT-1A control protein |
| SLT-1A::αHER2scFv | 119% |
| αHER2scFv::SLT-1A | 108% |

### Determining the Selective Cytotoxicity and Half-Maximal Cytotoxic Concentration (CD₅₀) of SLT-1A::αHER2scFv Using a Cell-Kill Assay

The cytotoxicity characteristics of SLT-1A::αHER2scFv was determined by the following cell-kill assay. This assay determines the capacity of a cytotoxic protein to kill cells expressing the target biomolecule of the cytotoxic protein's immunoglobulin-type binding region as compared to cells that do not express the target biomolecule. Cells were plated (2×10³ cells per well) in 20 μL cell culture medium in 384-well plates. The SLT-1A::αHER2scFv protein was diluted either 5-fold or 10-fold in a 1×PBS and 5 μL of the dilutions were added to the cells. Control wells containing only cell culture medium were used for baseline correction. The cell samples were incubated with SLT-1A::αHER2scFv, or just buffer, for 3 days at 37° C. and an atmosphere 5% CO₂. The total cell survival or percent viability was determined using a luminescent readout using the CellTiter-Glo® Luminescent Cell Viability Assay (G7573 Promega Madison, Wis., U.S.) according to the manufacturer's instructions. The Percent Viability of experimental wells was calculated using the following equation: (Test RLU−Average Media RLU)/(Average Cells RLU−Average Media RLU)*100. Log polypeptide concentration versus Percent Viability was plotted in Prism (GraphPad Prism, San Diego, Calif., U.S.) and log (inhibitor) vs. normalized response (variable slope) analysis was used to determine the half-maximal cytotoxic concentration (CD₅₀) value for SLT-1A::αHER2scFv.

Dose dependence experiments determined that the CD₅₀ of the SLT-1A::αHER2scFv protein was about 0.07 nM for HER2+ cells (Table 6; FIG. 3). The CD₅₀ for the same protein domains recombined in the reverse orientation, αHER2scFv::SLT-1A, was measured to be about 0.64 nM (Table 6; FIG. 3). The CD₅₀ results for the HER2− cell line MDA-MB468 were not calculable because proper curves could not be fitted to the data when 50% cell death did not occur within in any sample in the concentration series tested.

TABLE 6
Orientation of Engineering Affects Cytotoxicity: Representative
half-maximal cytotoxic concentration (CD₅₀) for SLT-1A::αHER2scFv
as compared to the reverse orientation αHER2scFv::SLT-1A

| | | CD₅₀ (nM) | | |
|---|---|---|---|---|
| | | | | SLT-1A only |
| | HER2 | SLT-1A:: | αHER2scFv:: | negative |
| Cell Line | status | αHER2scFv | SLT-1A | control |
| HCC-1954 | positive | 0.070 | 0.640 | 2.00 |
| MDA-MB-468 | negative | NC | NC | NC |

* “NC” denotes not calculable

The results in Table 6 and FIG. 3 exemplify the impact that the “orientation of engineering” has on cytotoxicity. This showed that a more optimized cytotoxic potency of about 9-fold was obtained by the orientation where the immunoglobulin-type region was not located proximally to the amino-terminus of the cytotoxic protein relative to the Shiga toxin effector region. The differences in cell-kill for these cytotoxic proteins were not predictable based on the in vitro results for either ribosome inactivation or target cell-binding characteristics.

### Determining Cell Internalization of SLT-1A::αCDHER2scFv and its Reverse Orientation αHER2scFv::SLT-1A Using Immunofluorescence

The ability of cytotoxic proteins to enter target cells was investigated using standard immunocytochemical techniques known in the art. Briefly, 0.8×10⁶ cells of each cell type (SKBR3 (HER2+) and MDA-MB-231 (HER2−)) were harvested and suspended in 50 μL of cell culture medium containing a cocktail of protease inhibitors (e.g. P1860 Sigma-Aldrich Co., St. Louis, Mo., U.S.) and human Fc receptor protein to reduce non-specific immunofluorescent staining. Next, 100 nM of the cytotoxic protein to be analyzed was added to the cells, and the cells were incubated at 37° C. for 1 hour to allow for intoxication to progress. Then, cells were “fixed” and “permeabilized” using the Cytofix/Cytoperm™ Kit (BD Biosciences San Diego, Calif., U.S.) according to the manufacturer's instructions. The Shiga toxin effector region was “stained” using a mouse monoclonal antibody (mouse IgG anti-Shiga toxin 1 Subunit A, BEI NR-867 BEI Resources, Manassas, Va., U.S.). The mouse monoclonal antibody localization was then detected with the Alexa Fluor® 555 Monoclonal Antibody Labeling Kit (Life Technologies, Carlsbad, Calif., U.S.) according to manufacturer's instructions.

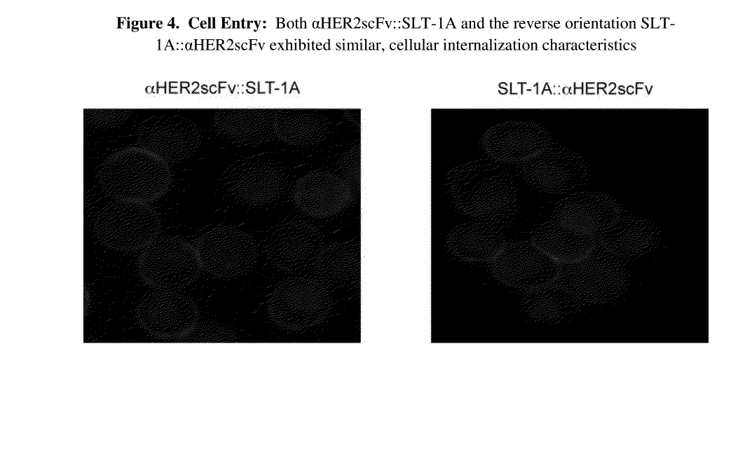

In this assay, cell surface binding and cell internalization was observed in HER2+ cells for both SLT-1A::αHER2scFv and the reverse orientation protein αHER2scFv::SLT-1A (FIG. 4). No cell internalization was observed for either protein to HER2− cells. This shows the differences in cytotoxicity (Table 6; FIG. 3) between these two cytotoxic protein variants, which differ only in the relative order of their immunoglobulin-type binding region and Shiga toxin effector region, were probably not related to any significant change in target cell binding and/or cell entry.

### Determining the In Vivo Effects of the Cytotoxic Protein αHER2scFv::SLT-1A Using Animal Models

Animal models are used to determine the in vivo effects of the cytotoxic protein αHER2scFv::SLT-1A on HER2+ neoplastic cells. Various mice strains are used to test the effect of the cytotoxic protein after intravenous administration on xenograft tumors in mice resulting from the injection into those mice of human neoplastic cells which express HER2 on their cell surfaces.

### Example 3

### A CD19-Targeted, Cytotoxic Protein Derived from the A Subunit of Shiga-Like Toxin-1 (SLT-1A::αCD19scFv)

The cytotoxic protein of this example SLT-1A::αCD19scFv comprises a Shiga toxin A Subunit fragment recombined with a single-chain, variable fragment, binding region capable of binding CD19 with high affinity such that the Shiga toxin effector region is more proximal to the amino-terminus of the cytotoxic protein than the CD19 binding region.

### Construction, Production, and Purification of the Cytotoxic Protein SLT-1A::αCD19scFv

In this example, the Shiga toxin effector region was derived from the A subunit of Shiga-like Toxin 1 (SLT-1A). A polynucleotide was obtained that encoded amino acids 1-251 of SLT-1A (Cheung M et al., Mol Cancer 9: 28 (2010). An immunoglobulin-type binding region αCD19scFv was derived from the humanized, monoclonal antibody anti-CD19 4G7 (Peipp M et al., J Immunol Methods 285: 265-80 (2004) and references therein) such that a single-chain variable fragment (scFv) is created with the two immunoglobulin variable regions (V_{L} and V_{H}) separated by a linker known in the art.

The binding region and Shiga toxin effector region were linked together to form a fusion protein. In this example, a polynucleotide encoding the Shiga toxin effector region from SLT-1A (amino acids 1-251 of SEQ ID NO:1) was cloned in frame with a polynucleotide encoding a linker, such as a “murine hinge” derived from a murine IgG3 molecule (and other linkers known to the skilled worker) and in frame with a polynucleotide encoding the immunoglobulin-type binding region αCD19scFv comprising amino acids 269-516 of SEQ ID NO:12. The polynucleotide sequence encoding the cytotoxic protein SLT-1A::αCD19scFv of this example was codon optimized for efficient expression in E. coli using services from DNA 2.0, Inc. (Menlo Park, Calif., U.S.).

A fusion protein was produced by expressing the polynucleotide encoding the cytotoxic protein SLT-1A::αCD19scFv (SEQ ID NO:12). Expression of the SLT-1A::αCD19scFv cytotoxic protein was accomplished using a bacterial system known in the art.

In this example of SLT-1A::αCD19scFv production by an E. coli expression system, the polynucleotide “insert” sequence encoding SLT-1A::αCD19scFv was cloned into the pTxb1 vector (New England Biolabs, Ipswich, Mass., U.S.) using standard procedures to produce a polynucleotide sequence encoding the cytotoxic protein SLT-1A::αCD19scFv ligated in frame to polynucleotide sequences encoding the amino-terminal intein of the vector. The plasmid insert polynucleotide sequence was verified by Sanger sequencing (Functional Biosciences, Madison, Wis., U.S.) and transformed into T7 Shuffle® cells (New England Biolabs, Ipswich, Mass., U.S.). The SLT-1A::αCD19scFv protein was produced and purified according to the IMPACT™ (Intein Mediated Purification with an Affinity Chitin-binding Tag) system manual (New England Biolabs, Ipswich, Mass., U.S.). Purification was accomplished using standard techniques known in the art, such as affinity chromatography.

### Determining the Half-Maximal Inhibitory Concentration (IC₅₀) of SLT-1A::αCD19scFv to Eukaryotic Ribosomes In Vitro

The ribosome inactivation capabilities of SLT-1A::αCD19scFv was determined in a cell-free, in vitro protein translation assay using the TNT® Quick Coupled Transcription/Translation Kit (L1170 Promega, Madison, Wis., U.S.). The kit includes Luciferase T7 Control DNA and TNT® Quick Master Mix. The ribosome activity reaction was prepared according to the manufacturer's instructions to create “TNT” reaction mixtures.

A series of 10-fold dilutions of SLT-1A::αCD19scFv to be tested was prepared in appropriate buffer and a series of identical TNT reaction mixture components was created for each dilution of SLT-1A::αCD19scFv. Each sample in the dilution series of SLT-1A::αCD19scFv protein was combined with each of the TNT reaction mixtures along with the Luciferase T7 Control DNA. The test samples were incubated for 1.5 hours at 30° C. After the incubation, Luciferase Assay Reagent (E1483 Promega, Madison, Wis., U.S.) was added to all test samples and the amount of luciferase protein translation was measured by luminescence according to the manufacturer instructions. The level of translational inhibition was determined by non-linear regression analysis of log-transformed concentrations of total protein versus relative luminescence units. Using statistical software (GraphPad Prism, San Diego, Calif., U.S.), the half maximal inhibitory concentration (IC₅₀) value was calculated for each sample.

The inhibitory effect of SLT-1A::αCD19scFv on cell-free protein synthesis was strong. Dose dependence experiments determined that the IC₅₀ SLT-1A::αCD19scFv on protein synthesis in this cell-free assay was about 5.2 pM (Table 7). This result was not substantially different from the IC₅₀ for the reverse orientation protein αCD19scFv::SLT-1A, which was measured to be about 3.2 pM or equivalent of the SLT-1A-only positive control (Table 7). This shows that the “orientation of engineering” effect is probably not related to any significant perturbation of Shiga toxin A Subunit enzymatic activity.

TABLE 7
Orientation of Engineering Had No Effect on Ribosome
Inactivation: Representative half-maximal inhibitory concentrations
(IC₅₀) for SLT-1A::αCD19scFv as compared to the reverse
orientation αCD19scFv::SLT-1A

| | | IC₅₀ of SLT-1A only |
|---|---|---|
| Cytotoxic Protein | IC₅₀ (pM) | positive control (pM) |
| SLT-1A::αCD19scFv | 5.2 | 7.9 |
| αCD19scFv::SLT-1A | 3.2 | 7.9 |

### Determining the Selective Cytotoxicity and Half-Maximal Cytotoxic Concentration (CD₅₀) of SLT-1A::αCD19scFv Using a Cell-Kill Assay

The cytotoxicity characteristics of SLT-1A::αCD19scFv was determined by the following cell-kill assay. This assay determines the capacity of a cytotoxic protein to kill cells expressing the target biomolecule of its immunoglobulin-type binding region as compared to cells that do not express the target biomolecule. Cells were plated (2×10³ cells per well) in 20 μL of cell culture medium in 384-well plates. The SLT-1A::αCD19scFv protein was diluted 10-fold in buffer and 5 μL of the dilutions were added to the cells. Control wells containing only cell culture medium were used for baseline correction. The cell samples were incubated with SLT-1A::αCD19scFv, or just buffer, for 3 days at 37° C. and an atmosphere 5% CO₂. The total cell survival or percent viability was determined using a luminescent readout using the CellTiter-Glo® Luminescent Cell Viability Assay (G7573 Promega Madison, Wis., U.S.) according to the manufacturer's instructions. The Percent Viability of experimental wells was calculated using the following equation: (Test RLU−Average Media RLU)/(Average Cells RLU−Average Media RLU)*100. Log polypeptide concentration versus Percent Viability was plotted in Prism (GraphPad Prism, San Diego, Calif., U.S.) and log (inhibitor) vs. response (three parameter) analysis was used to determine the half-maximal cytotoxic concentration (CD₅₀) value for SLT-1A::αCD19scFv.

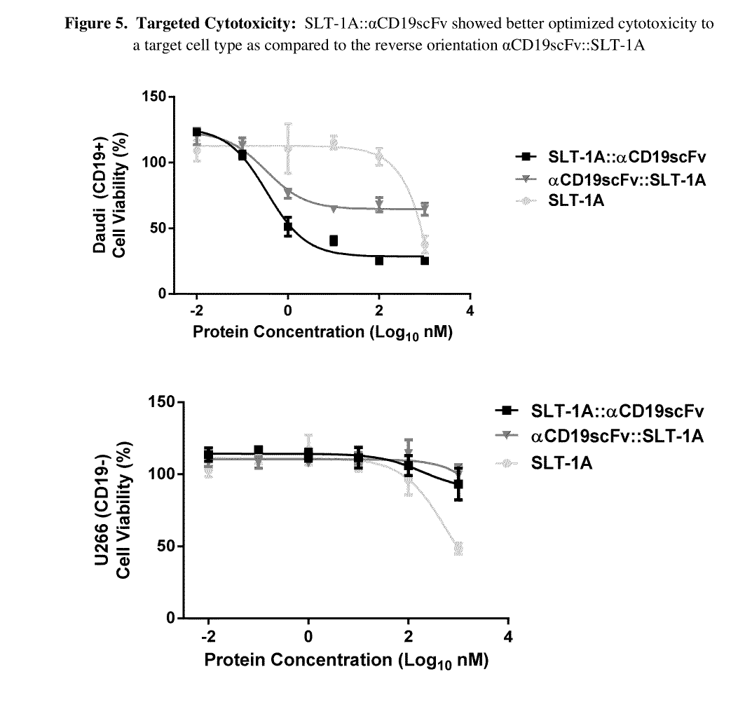

Dose dependence experiments determined that the CD₅₀ of the SLT-1A::αCD19scFv protein was about 0.28 nM for CD19+ Daudi cells (Table 8; FIG. 5). The CD₅₀ for the SLT-1A-only negative control and the same protein domains recombined in the reverse orientation, αCD19scFv::SLT-1A, could not be accurately measured based on the shape of the curve. The CD₅₀ of the SLT-1A::αCD19scFv for CD19 negative U266 cells could not be calculated due to the shape of the curve; these cells are not physically coupled with the extracellular target biomolecule CD19.

TABLE 8
Orientation of Engineering Affected Cytotoxicity: Representative
half-maximal cytotoxic concentrations (CD₅₀) of SLT-1A::αCD19scFv
as compared to the reverse orientation αCD19scFv::SLT-1A

| | | CD₅₀ (nM) | | |
|---|---|---|---|---|
| | | | | SLT-1A only |
| | CD19 | SLT-1A:: | αCD19scFv:: | negative |
| Cell Line | status | αCD19scFv | SLT-1A | control |
| Daudi | positive | 0.28 | NC | NC |
| U266 | negative | NC | NC | NC |

* “NC” denotes not calculable

These results showed that a specific orientation of protein engineering, where the immunoglobulin-type region was not located proximally to the amino-terminus of the cytotoxic protein relative to the Shiga toxin effector region, conferred a better optimized cytotoxic potency toward CD19+ cells (Table 8; FIG. 5). The better optimized orientation was qualitatively better than the other orientation over the concentration range tested. The CD19-targeted protein SLT-1A::αCD19scFv exhibited a CD₅₀ value of 0.28 nM to CD19+ cells (Table 8), which was comparable to the CD₅₀ value of the exemplary CD38-targeted protein in Example 1 toward a CD38+ cell type (see Table 3, 0.28 nM to Daudi cells). The differences in cell-kill for the cytotoxic proteins of this Example were not predictable based on the in vitro results for ribosome inactivation and are not expected to be predictable based on target cell-binding characteristics or cellular internalization given the results in Examples 1 and 2.

### Determining the In Vivo Effects of the Cytotoxic Protein SLT-1A::αCD19scFv Using Animal Models

Animal models are used to determine the in vivo effects of the cytotoxic protein SLT-1A::αCD19scFv on neoplastic and/or immune cells. Various mice strains are used to test the effect of the cytotoxic protein after intravenous administration on xenograft tumors in mice resulting from the injection into those mice of human neoplastic and/or human immune cells which express CD19 on their cell surfaces.

### Example 4

### A CD74-Targeted, Cytotoxic Protein Derived from the A Subunit of Shiga-Like Toxin-1 (SLT-1A::αCD74scFv)

The cytotoxic protein of this example SLT-1A::αCD74scFv comprises a Shiga toxin A Subunit fragment recombined with a single-chain, variable fragment, binding region capable of binding CD74 with high affinity such that the Shiga toxin effector region is more proximal to the amino-terminus of the cytotoxic protein than the CD74 binding region.

### Construction, Production, and Purification of the Cytotoxic Protein SLT-1A::αCD74scFv

In this example, the Shiga toxin effector region was derived from the A subunit of Shiga-like Toxin 1 (SLT-1A). A polynucleotide was obtained that encoded amino acids 1-251 of SLT-1A (Cheung M et al., Mol Cancer 9: 28 (2010). An immunoglobulin-type binding region αCD74scFv was derived from the humanized monoclonal antibody anti-CD74, Milatuzumab (Sapra P et al., Clin Cancer Res 11: 5257-64 (2005) and references therein) such that a single-chain variable fragment (scFv) is created with the two immunoglobulin variable regions (V_{L} and V_{H}) separated by a linker known in the art.

The binding region and Shiga toxin effector region were linked together to form a fusion protein. In this example, a polynucleotide encoding the Shiga toxin effector region from SLT-1A (amino acids 1-251 of SEQ ID NO:1) was cloned in frame with a polynucleotide encoding a linker, such as a “murine hinge” derived from a murine IgG3 molecule (and other linkers known to the skilled worker) and in frame with a polynucleotide encoding the immunoglobulin-type binding region αCD74scFv comprising amino acids 269-518 of SEQ ID NO:16. The polynucleotide sequence encoding the cytotoxic protein SLT-1A::αCD74scFv of this example was codon optimized for efficient expression in E. coli using services from DNA 2.0, Inc. (Menlo Park, Calif., U.S.).

A fusion protein was produced by expressing the polynucleotide encoding the cytotoxic protein SLT-1A::αCD74scFv (SEQ ID NO:16). Expression of the SLT-1A::αCD74scFv cytotoxic protein was accomplished using a bacterial system known in the art.

In this example of SLT-1A::αCD74scFv production by an E. coli expression system, the polynucleotide “insert” sequence encoding SLT-1A::αCD74scFv was cloned into the pTxb1 vector (New England Biolabs, Ipswich, Mass., U.S.) using standard procedures to produce a polynucleotide sequence encoding the cytotoxic protein SLT-1A::αCD74scFv ligated in frame to polynucleotide sequences encoding the amino-terminal intein of the vector. The plasmid insert polynucleotide sequence was verified by Sanger sequencing (Functional Biosciences, Madison, Wis., U.S.) and transformed into T7 Shuffle® cells (New England Biolabs, Ipswich, Mass., U.S.). The SLT-1A::αCD74scFv protein was produced and purified according to the IMPACT™ (Intein Mediated Purification with an Affinity Chitin-binding Tag) system manual (New England Biolabs, Ipswich, Mass., U.S.). Purification was accomplished using standard techniques known in the art, such as affinity chromatography.

### Determining the Half-Maximal Inhibitory Concentration (IC₅₀) of SLT-1A::αCD74scFv to Eukaryotic Ribosomes In Vitro

The ribosome inactivation capabilities of SLT-1A::αCD74scFv was determined in a cell-free, in vitro protein translation assay using the TNT® Quick Coupled Transcription/Translation Kit (L1170 Promega, Madison, Wis., U.S.). The kit includes Luciferase T7 Control DNA and TNT® Quick Master Mix. The ribosome activity reaction was prepared according to the manufacturer's instructions to create “TNT” reaction mixtures.

A series of 10-fold dilutions of SLT-1A::αCD74scFv to be tested was prepared in appropriate buffer and a series of identical TNT reaction mixture components was created for each dilution of SLT-1A::αCD74scFv. Each sample in the dilution series of SLT-1A::αCD74scFv protein was combined with each of the TNT reaction mixtures along with the Luciferase T7 Control DNA. The test samples were incubated for 1.5 hours at 30° C. After the incubation, Luciferase Assay Reagent (E1483 Promega, Madison, Wis., U.S.) was added to all test samples and the amount of luciferase protein translation was measured by luminescence according to the manufacturer instructions. The level of translational inhibition was determined by non-linear regression analysis of log-transformed concentrations of total protein versus relative luminescence units. Using statistical software (GraphPad Prism, San Diego, Calif., U.S.), the half maximal inhibitory concentration (IC₅₀) value was calculated for each sample.

The inhibitory effect of SLT-1A::αCD74scFv on cell-free protein synthesis was strong. Dose dependence experiments determined that the IC₅₀ SLT-1A::αCD74scFv on protein synthesis in this cell-free assay was about 8.7 pM (Table 9). This result was not substantially different from the IC₅₀ for the reverse orientation protein αCD74scFv::SLT-1A, which was measured to be about 3.6 pM or equivalent of the SLT-1A-only positive control (Table 9). This shows that “orientation of engineering” effects are probably not related to any significant perturbation of Shiga toxin A Subunit enzymatic activity.

TABLE 9
Orientation of Engineering Had No Effect on Ribosome
Inactivation: Representative half-maximal inhibitory concentrations
(IC₅₀) for SLT-1A::αCD74scFv as compared to the reverse
orientation αCD74scFv::SLT-1A

| | | IC₅₀ of SLT-1A only |
|---|---|---|
| Cytotoxic Protein | IC₅₀ (pM) | positive control (pM) |
| SLT-1A::αCD74scFv | 8.7 | 7.9 |
| αCD74scFv::SLT-1A | 3.6 | 7.9 |

### Determining the Selective Cytotoxicity and Half-Maximal Cytotoxic Concentration (CD₅₀) of SLT-1A::αCD74scFv Using a Cell-Kill Assay

The cytotoxicity characteristics of SLT-1A::αCD74scFv was determined by the following cell-kill assay. This assay determines the capacity of a cytotoxic protein to kill cells expressing the target biomolecule of its immunoglobulin-type binding region as compared to the SLT-1A protein that does not possess the binding region. Cells were plated (2×10³ cells per well) in 20 μL of cell culture medium in 384-well plates. The SLT-1A::αCD74scFv protein was diluted 10-fold in buffer and 5 μL of the dilutions were added to the cells. Control wells containing only cell culture medium were used for baseline correction. The cell samples were incubated with SLT-1A::αCD74scFv, or just buffer, for 3 days at 37° C. and an atmosphere 5% CO₂. The total cell survival or percent viability was determined using a luminescent readout using the CellTiter-Glo® Luminescent Cell Viability Assay (G7573 Promega Madison, Wis., U.S.) according to the manufacturer's instructions. The Percent Viability of experimental wells was calculated using the following equation: (Test RLU−Average Media RLU)/(Average Cells RLU−Average Media RLU)*100. Log polypeptide concentration versus Percent Viability was plotted in Prism (GraphPad Prism, San Diego, Calif., U.S.) and log (inhibitor) vs. response (three parameter) analysis was used to determine the half-maximal cytotoxic concentration (CD₅₀) value for SLT-1A::αCD74scFv.

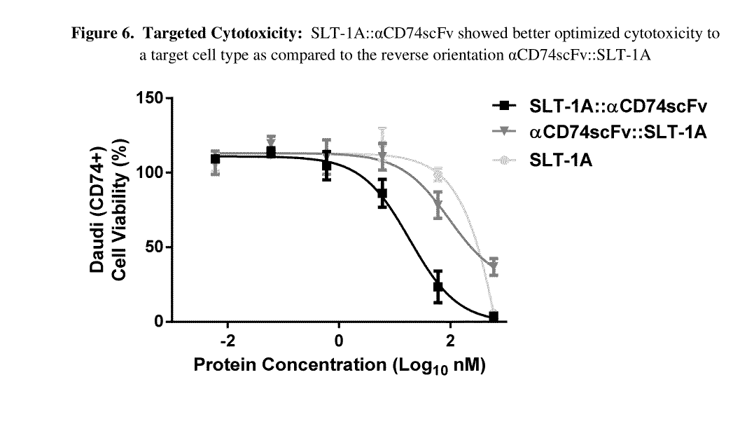

Dose dependence experiments determined that the CD₅₀ of the SLT-1A::αCD74scFv protein was about 18.7 nM for CD74+ Daudi cells (Table 10; FIG. 6). The CD₅₀ for the SLT-1A-only negative control was 2026 nM and the same protein domains recombined in the reverse orientation, αCD74scFv::SLT-1A, was 95.3 nM (Table 10; FIG. 6).

TABLE 10
Orientation of Engineering Affected Cytotoxicity: Representative
half-maximal cytotoxic concentrations (CD₅₀) of SLT-1A::αCD74scFv
as compared to the reverse orientation αCD74scFv::SLT-1A

| | | CD₅₀ (nM) | | |
|---|---|---|---|---|
| | | | | SLT-1A only |
| | CD74 | SLT-1A:: | αCD74scFv:: | negative |
| Cell Line | status | αCD74scFv | SLT-1A | control |
| Daudi | positive | 18.7 | 95.3 | 2026 |

These results showed that one particular orientation of cytotoxic protein engineering, where the immunoglobulin-type region was not located proximally to the amino-terminus of the cytotoxic protein relative to the Shiga toxin effector region, conferred a more optimized cytotoxic potency toward CD74+ cells of about 5-fold. The differences in cell-kill for these cytotoxic proteins were not predictable based on the in vitro results for protein synthesis inhibition and are not expected to be predictable based on target cell-binding characteristics or cellular internalization given the results in Examples 1 and 2.

### Determining the In Vivo Effects of the Cytotoxic Protein SLT-1A::αCD74scFv Using Animal Models

Animal models are used to determine the in vivo effects of the cytotoxic protein SLT-1A::αCD74scFv on CD74+ neoplastic and/or immune cells. Various mice strains are used to test the effect of the cytotoxic protein after intravenous administration on xenograft tumors in mice resulting from the injection into those mice of human neoplastic and/or human immune cells which express CD74 on their cell surfaces.

Summary: Linking the Binding Region Carboxy-Terminal to the Amino-Terminus of the Shiga Toxin Effector Region Resulted in a Cell-Targeted Molecules with More Optimized Cytotoxic Potencies

When different cell-targeted molecules derived from the Shiga toxin A Subunit SLT-1A were tested for cytotoxic potency, not all the cell-targeted molecules exhibited the expected cytotoxicity predicted by their shared functional characteristics related to 1) in vitro, Shiga toxin enzymatic activity; 2) in vitro, biomolecular target binding; 3) target-positive cell binding; and/or 4) cellular internalization into target-positive cells. Surprisingly, it was observed that cell-targeted, fusion proteins comprising heterologous binding regions linked amino-terminal to Shiga toxin effector regions did not result in potent cytotoxicity as compared to the same two polypeptide regions linked with the amino-terminus of the Shiga toxin effector region more proximal to an amino-terminus of a polypeptide component of the cell-targeted protein. In the examples above, more optimized cytotoxic potencies of approximately 4-fold to 9-fold were observed for different cell-targeted proteins engineered with heterologous binding regions linked carboxy-terminal of the Shiga toxin effector region as compared to related variants linked in the reverse orientation.

The examples above demonstrate that keeping the amino-terminus of Shiga toxin effector regions in close proximity to an amino-terminus of a component of a cell-targeted molecule is important for achieving better optimized cytotoxic potency. For example, when maximal cytotoxic potency is desirable for any cytotoxic cell-targeted molecule derived from a Shiga toxin A Subunit, a heterologous binding region should not be oriented amino-terminal of the Shiga toxin effector region within a single, linear polypeptide.

Intending to be unbound by theory, the exposure of the amino-terminus of the Shiga toxin effector region polypeptide may be important for achieving better optimized cytotoxicity (i.e. reduced CD₅₀ values). Thus, cell-targeted molecules derived from Shiga toxins which are engineered with a heterologous binding region that do not sterically cover the amino-terminus of the Shiga toxin A Subunit effector region have better optimized cytotoxic potency of at least 4-fold to 9-fold or greater compared to the same components arranged with the binding region oriented within the cell-targeted molecule amino-terminal to the Shiga toxin effector region and thereby sterically blocking structural and/or functional effector features in the amino-terminus of the Shiga toxin effector region. In addition, any Shiga toxin-derived cell-targeted molecule comprising an amino-terminal, Shiga toxin A Subunit effector region may exhibit more optimal cytotoxicity because the amino-terminus region of the Shiga toxin effector region polypeptide will be unencumbered by any other moiety and, thus, maximally available.

### Example 5

### Exemplary, Cell-Targeted Molecules Comprising Furin-Cleavage Resistant, Shiga Toxin Effector Regions (SLT-1A-FR::scFv-1, SLT-1A-FR::scFv-2, and SLT-1A-FR-2::scFv-2)

Furin-cleavage resistant, Shiga toxin A Subunit effector polypeptides were created and tested as a component of cell-targeted proteins, which each comprised a cell-targeting, immunoglobulin-type, binding region carboxy-terminal to the Shiga toxin effector polypeptide region. To engineer protease-cleavage resistance into Shiga toxin effector polypeptides, amino acid residue substitutions were introduced into the conserved, minimal, furin-cleavage motif R-x-x-R in a Shiga toxin effector polypeptide derived from the A subunit of Shiga-like Toxin 1 (SLT-1A) comprising amino acids 1-251 of SLT-1A. In one exemplary Shiga toxin effector polypeptide, two amino acid residue substitutions, R248A and R251A, were introduced. This furin-cleavage resistant R248A and R251A double mutant construct is referred to herein as “SLT-1A-FR” (for SLT-1A furin-cleavage resistant). A second furin-cleavage resistant mutant construct, referred to herein as “SLT-1A-FR-2,” was generated with the single residue substitution R248A. A third, furin-cleavage resistant mutant construct, referred to herein as “SLT-1A-FR-3,” comprises the single residue substitution R251A. The mutation of the minimal, furin protease, cleavage site R-x-x-R in the core of the furin consensus motif region 240-256 was predicted to disrupt the sensitivity of this region to proteolysis by furin and other proteases, such as, e.g., proprotein convertases and promiscuous proteases. The Shiga toxin effector polypeptide SLT-1A-FR comprising the R248A/R251A disruption of the furin-cleavage site was used to create exemplary, cytotoxic, cell-targeted molecules of the present invention.

The exemplary, cytotoxic proteins SLT-1A-FR::scFv-1 and SLT-1A-FR::scFv-2 were constructed such that each comprised a catalytic Shiga toxin A Subunit effector polypeptide region comprising a disrupted furin-cleavage site and a cell-targeting, immunoglobulin-type binding region. In SLT-1A-FR::scFv-1 and SLT-1A-FR::scFv-2, the Shiga toxin effector polypeptide was fused to carboxy-terminal, immunoglobulin-type binding regions via proteinaceous linkers, which together represented relatively large, carboxy-terminal moieties. SLT-1A-FR::scFv-1 and SLT-1A-FR::scFv-2 were produced in a bacterial system and purified by column chromatography. The immunoglobulin-type binding regions scFv-1 (αHER2scFv) and scFv-2 (αCD38scFv variant 2) are single-chain variable fragments which each bound with high-affinity to a certain cell-surface, target biomolecule physically coupled to the surface of certain human cancer cells as well as to certain human cancer cells.

### Testing the Furin Proteolysis Sensitivity of Exemplary Cell-Targeted Proteins Comprising SLT-1A-FR

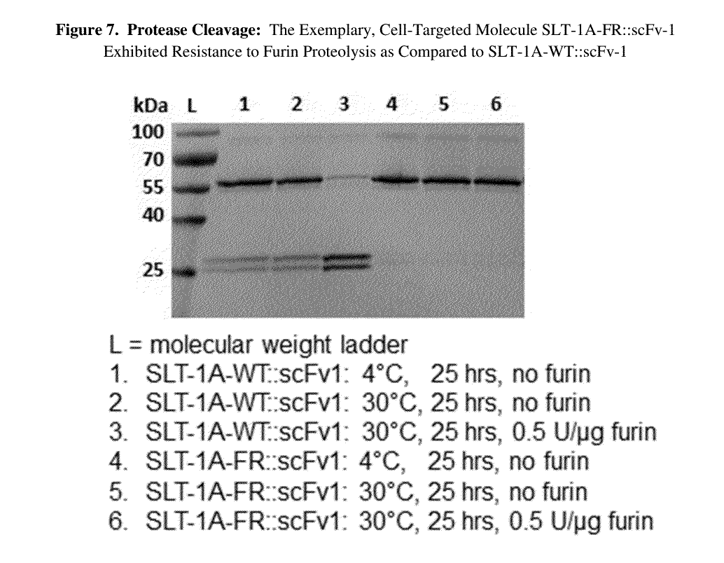

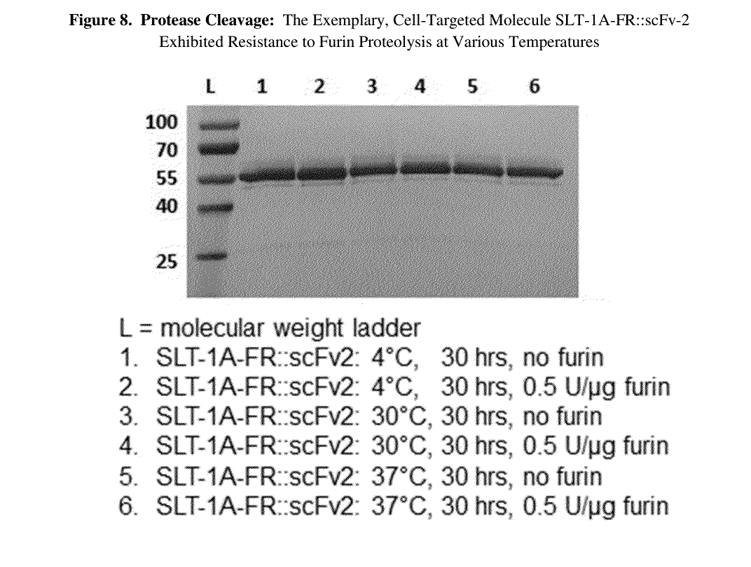

After mutating the protease-cleavage sensitive region 240-256 in the Shiga toxin effector polypeptide SLT-1A (1-251), the furin-cleavage sensitivity of different Shiga toxin effector polypeptides was tested in the molecular context of fusion proteins comprising carboxy-terminal, immunoglobulin-type binding regions. To assess the ability of furin to cleave SLT-1A-FR::scFv-1 and SLT-1A-FR::scFv-2, purified protein samples in phosphate buffered saline (PBS) were incubated with furin (New England Biolabs, Ipswich, Mass., U.S.) at 0.5 furin activity units (U) per microgram (μg) of sample protein in furin-cleavage buffer (100 millimolar (mM) HEPES (4-(2-hydroxyethyl)-1-piperazineethanesulfonic acid), pH 7, 1 mM CaCl₂) for 25-30 hours (hrs) at 30 or 37° C. Control samples were incubated without furin at 4, 30, or 37° C. in the same buffer. The various protein samples were electrophoresed on sodium dodecyl sulfate (SDS), polyacrylamide gels under denaturing conditions and stained with Coomassie (FIGS. 7 and 8).

FIGS. 7 and 8 show pictures of the gels with the lanes numbered and contain figure legends indicating which lane was loaded with which protein sample: either a cell-targeted protein comprising a wild-type, Shiga toxin effector polypeptide (SLT-1A-WT) or a furin-cleavage site disrupted, Shiga toxin effector polypeptide (SLT-1A-FR or SLT-1A-FR-2). The lanes marked “L” show the migration pattern of a protein molecular weight ladder along with the approximate size of individual ladder protein bands in kDa for use as an internal molecular weight reference that allows for the estimation of the sizes of proteins in the numbered lanes. The figure legends indicate the pre-treatment conditions of the protein samples with the temperature in degrees Celsius (° C.), duration, and whether any furin was added by denoting the amount of furin activity units per microgram (labeled “U/μg furin”) or “no furin” for zero units.

FIGS. 7 and 8 show that SLT-1A-FR::scFv-1 and SLT-1A-FR::scFv-2 were resistant to cleavage by human furin. The cell-targeted proteins tested in this assay were both about 55-57 kDa in size and comprised a Shiga toxin effector polypeptide of about 28 kDa (identical in size for both SLT-1A-WT and SLT-1A-FR) linked to a carboxy-terminal linker and binding region, which together were about 28-29 kDa in size. If furin cleavage had occurred in the surface exposed, extended loop 242-251 of SLT-1A, then the expected result would be two protein bands with near equal molecular weights of around 28 kDa each. If furin cleavage occurs precisely at the carboxy peptide bond of the arginine at position 251 of the WT scaffold in SLT-1A-WT::scFv-1 or SLT-1A-WT::scFv-2, then the two resulting protein bands should have the molecular weight of 27.5 kDa for SLT-1A (either WT or FR) and a second band of 28.8 kDa for SLT-1A-FR::scFv-1 or 27.6 kDa for SLT-1A-FR::scFv-2.

FIG. 7 shows that SLT-1A-FR::scFv-1 was not proteolyzed in vitro by human furin in this assay under the conditions tested. As expected, the control protein SLT-1A-WT::scFv-1, which comprised a wild-type Shiga toxin effector polypeptide, was cleaved by human furin (FIG. 7); however, SLT-1A-FR::scFv-1 was resistant (compare lanes 3 and 6 in FIG. 7).

SLT-1A-FR::scFv-2 was also resistant to furin cleavage in this assay at several different temperatures (FIG. 8). FIG. 8 shows that SLT-1A-FR::scFv-2 was not proteolyzed in vitro by human furin in this assay under the conditions tested, such as at temperatures ranging from 4° to 37° C.

In addition, a cell-targeted fusion protein SLT-1A-FR-2::scFv-2 was resistant to furin cleavage in this assay at 4° C.

Using this in vitro furin-cleavage assay, no furin proteolysis of cell-targeted fusion proteins was observed at any furin-cleavage site besides 248-251 in the Shiga toxin effector polypeptide region, such as, e.g., in the SLT-1A component at the furin-cleavage site natively positioned in the region from 220 to 223.

Thus, the mutation of the minimal, furin protease, cleavage site R-x-x-R in the core of the furin consensus motif region disrupted the sensitivity of this region to proteolysis by human furin in vitro.

### Testing the Ribosome Inhibitory Activity of Cell-Targeted Proteins Comprising SLT-1A-FR

The molecules of the present invention all comprise a catalytic domain derived from at least one Shiga toxin A Subunit. The enzymatic activity of the furin-cleavage resistant, Shiga toxin effector polypeptide SLT-1A-FR was tested using an in vitro ribosome inhibition assay. The ribosome inactivation activity of SLT-1A-FR was tested in the molecular context of a carboxy-terminal, cell-targeting, immunoglobulin-type binding regions using SLT-1A-FR::scFv-1 and SLT-1A-FR::scFv-2.

The ribosome inactivation capabilities of SLT-1A-FR::scFv-1 and SLT-1A-FR::scFv-2 were determined with a cell-free, in vitro, protein translation assay using the TNT® Quick Coupled Transcription/Translation Kit (L1170 Promega Madison, Wis., U.S.). The kit includes Luciferase T7 Control DNA (L4821 Promega Madison, Wis., U.S.) and TNT® Quick Master Mix. The ribosome activity reaction was prepared according to manufacturer's instructions. A series of 10-fold dilutions of the protein to be tested (cell-targeted proteins comprising either SLT-1A-WT or SLT-1A-FR) were prepared in an appropriate buffer and a series of identical TNT reaction mixture components were created for each dilution. Each sample in the dilution series was combined with each of the TNT reaction mixtures along with the Luciferase T7 Control DNA. The test samples were incubated for 1.5 hours at 30° C. After the incubation, Luciferase Assay Reagent (Catalog # E1483, Promega Corp., Madison, Wis., U.S.) was added to all test samples and the amount of luciferase protein translation was measured by luminescence according to manufacturer's instructions.

The level of translational inhibition was determined by non-linear regression analysis of log-transformed concentrations of total protein versus relative luminescence units. Using statistical software (GraphPad Prism, San Diego, Calif., U.S.), the half maximal inhibitory concentration (IC₅₀) value was calculated for each sample using the Prism software function of log(inhibitor) vs. response (three parameters) [Y=Bottom+((Top−Bottom)/(1+10̂(X−Log IC50)))] under the heading dose-response-inhibition.

The IC₅₀ for each cell-targeted protein comprising a furin-cleavage resistant Shiga toxin effector polypeptide (SLT-1A-FR) region and a wild-type (WT) control protein from one or more experiments was calculated and is shown in Table 11. The constructs comprising the furin-cleavage resistant SLT-1A-FR exhibited potent ribosome inhibition which was comparable to wild-type controls, such as a wild-type SLT-1 A1 fragment (SLT-1A1-WT) (Table 11). Both SLT-1A-FR::scFv-1 and SLT-1A-FR::scFv-2 exhibited ribosome inactivation activity in vitro comparable to a wild-type, Shiga toxin A1 fragment.

TABLE 11
The furin-cleavage resistant, Shiga toxin effector polypeptide
exhibited equivalent ribosome inhibition in vitro to a protease-
cleavage sensitive, Shiga toxin effector polypeptide

| Polypeptide | Ribosome Inhibition IC₅₀ (pM) |
|---|---|
| SLT-1A1-WT only | 158 |
| SLT-1A-WT::scFv-1 | 107 |
| SLT-1A-FR::scFv-1 | 131 |
| SLT-1A-WT::scFv-2 | 101 |
| SLT-1A-FR::scFv-2 | 187 |

Testing the Cytotoxicity of the Exemplary Proteins SLT-1A-FR::scFv-1 and SLT-1A-FR::scFv-2

The cytotoxicity of exemplary proteins of the invention, each comprising the protease-cleavage resistant, Shiga toxin effector polypeptide SLT-1A-FR, was determined using cell kill assays known to the skilled worker. Specific cytotoxicity was determined by comparing the cytotoxicity of exemplary proteins toward target expressing cells versus the cytotoxicity of an untargeted, wild-type, Shiga toxin effector control (SLT-1A-WT). Selective cytotoxicity was determined by comparing the cytotoxicity toward target expressing cells versus cells which did not express a target biomolecule of the protein's immunoglobulin-type binding region. Cells were selected that expressed a significant amount of an extracellular target biomolecule of scFv-1 or scFv-2 at least one cellular surface, i.e. cells that were binding-region target biomolecule positive (cell lines A, B, C, and D were HER2+, and cell lines E, F, and G were CD38+). Cells were selected that did not express a significant amount of any extracellular target biomolecules of scFv-1 at any cellular surface and/or any extracellular target biomolecules of scFv-2 at any cellular surface, i.e. cells that were target biomolecule negative for any target of one or both of the binding regions scFv-1 and scFv-2.

The cytotoxicity of nearly identical cell-targeted proteins comprising either a wild-type SLT-1A (SLT-1A-WT) or furin-cleavage resistant, Shiga toxin effector polypeptide (SLT-1A-FR) were directly compared to isolate any differences in cytotoxicity caused by the two point mutations which provided furin-cleavage resistance. It was expected that the cytotoxicity of proteins comprising a SLT-1A-FR whose carboxy terminus was covered by a relatively large moiety, would be reduced as compared to proteins comprising a SLT-1A-WT, which can be liberated from a carboxy-terminal moiety by proteolytic cleavage, particularly by the endoprotease furin (see Lea N et al., Microbiology 145: 999-1004 (1999)).

The cytotoxicities, specific cytotoxicities, and relative cytotoxicities for the exemplary proteins SLT-1A-FR::scFv-1 and SLT-1A-FR::scFv-2 were compared to proteins comprising wild-type, Shiga toxin effector polypeptides.

Certain human tumor cells (including cells of the cell lines A-G) were plated (2×10³ cells per well for adherent cells, plated the day prior to protein addition or 7.5×10³ cells per well for suspension cells, plated the same day as protein addition) in 20 microliters (μL) cell culture medium in 384-well plates. A series of 10-fold dilutions of the proteins to be tested was prepared in an appropriate buffer, and 5 μL of the dilutions or buffer control were added to the cells. Control wells containing only cell culture medium were used for baseline correction. The cell samples were incubated with the proteins or just buffer for 3 days at 37° C. and in an atmosphere of 5 percent (%) carbon dioxide (CO₂). In certain experiments, the cell samples were incubated with the proteins or just buffer for 1 hour or 2 hours. Then un-internalized proteins were washed away using buffer washes. The total cell survival or percent viability was determined using a luminescent readout using the CellTiter-Glo® Luminescent Cell Viability Assay (Catalog # G7573, Promega Corp., Madison, Wis., U.S.) according to the manufacturer's instructions.

The Percent Viability of experimental wells was calculated using the following equation: (Test RLU−Average Media RLU)/(Average Cells RLU−Average Media RLU)*100. Log polypeptide concentration versus Percent Viability was plotted in Prism (GraphPad Prism, San Diego, Calif., U.S.) and log (inhibitor) versus response (3 parameter) analysis were used to determine the half-maximal cytotoxic concentration (CD₅₀) value for the tested proteins. The CD₅₀ for each cell-targeted protein comprising a protease-cleavage resistant, Shiga toxin effector polypeptide or a wild-type control, Shiga toxin effector polypeptide were calculated.

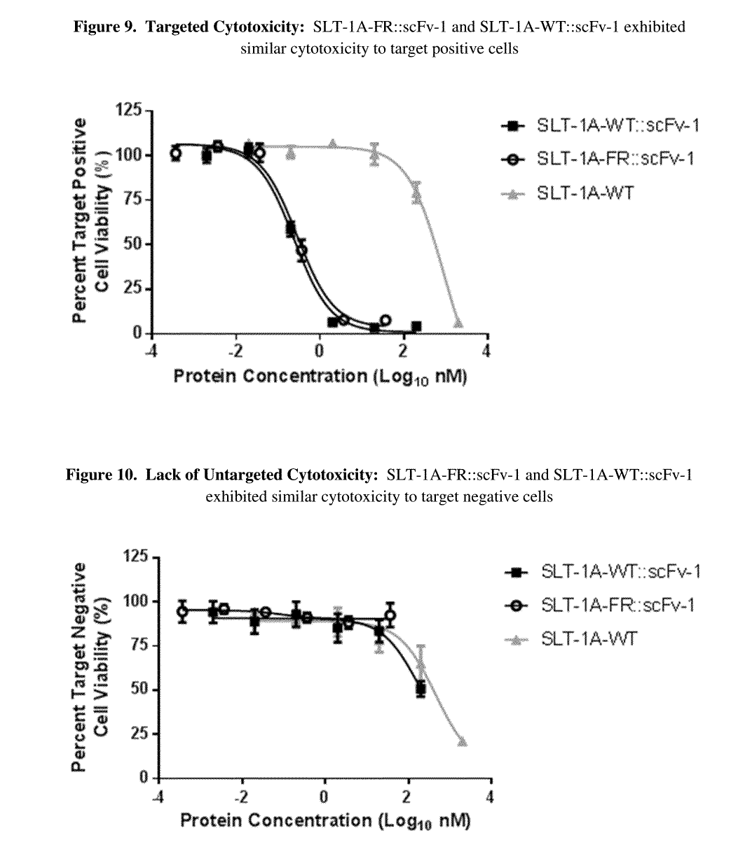

The cytotoxicity of SLT-1A-FR::scFv-1 and SLT-1A-FR::scFv-2 toward multiple, target-expressing, human tumor cell lines are shown in Table 12 as half-maximal cytotoxicity values (CD₅₀) in the cell-targeted protein concentration in nanomolar (nM). Surprisingly, cell-targeted proteins comprising SLT-1A-WT and SLT-1A-FR both displayed similar cytotoxicities (FIGS. 9-10; Table 12) instead of a noticeable reduction in cytotoxicity for cell-targeted proteins comprising SLT-1A1-R expected to be result whenever the Shiga toxin A1 fragment cannot efficiently be liberated from all other moieties, such as, e.g., a cell-targeting, binding region and a 4.5 kDa A2 fragment (see Garred Ø et al., Exp Cell Res 218: 39-49 (1995); Garred Ø et al., J Biol Chem 270: 10817-21 (1995); Lea N et al., Microbiology 145: 999-1004 (1999); Kurmanova A et al., Biochem Biophys Res Commun 357: 144-9 (2007)).

TABLE 12
The furin-cleavage resistant, Shiga toxin effector polypeptide exhibited equivalent
cytotoxicity to a furin-cleavable, wild-type, Shiga toxin effector polypeptide

| target positive | | | | | |
|---|---|---|---|---|---|
| cell line | Cytotoxicity CD₅₀ (nM) | | | | |
| binding | SLT-1A- | SLT-1A- | SLT-1A- | SLT-1A- | SLT-1A- |
| region target | WT only | WT::scFv-1 | FR::scFv-1 | WT::scFv-2 | FR::scFv-2 |
| A scFv-1 | 905.0 | 0.24 | 0.29 | N/T | N/T |
| B scFv-1 | 1800.0 | 0.47 | 0.29 | N/T | N/T |
| C scFv-1 | 37.0 | 0.22 | 0.41 | N/T | N/T |
| D scFv-1 | 4740.0 | 0.55 | 0.37 | N/T | N/T |
| E scFv-2 | 11050.0 | N/T | N/T | 0.920 | 1.30 |
| F scFv-2 | 32200.0 | N/T | N/T | 16.10 | 13.60 |
| G scFv-2 | 506.0 | N/T | N/T | 0.120 | 0.240 |

“N/T” indicates not tested

The CD₅₀ values for cell-targeted proteins comprising SLT-1A-FR were comparable to the CD₅₀ values for cell-targeted proteins comprising SLT-1A-WT (Table 12). Cell-targeted proteins comprising SLT-1A-WT and SLT-1A-FR both potently killed target expressing cells but did not kill comparable percentages of target negative cells at the same dosages (CD₅₀ values for target negative cells were uninformative for these data as an accurate curve could not be generated when there was not a sizeable decrease in cell viability at the highest tested concentrations). The results summarized in Table 12 show the comparable cytotoxicity of SLT-1A-FR and SLT-1A-WT as components of cell-targeted proteins. Both SLT-1A-FR::scFv-1 and SLT-1A-FR::scFv-2 were as cytotoxic as related proteins comprising furin-cleavage sensitive, wild-type, Shiga toxin A1 fragment regions. One example of the specific cytotoxicity of proteins comprising a protease-cleavage resistant, Shiga toxin effector polypeptide SLT-1A-FR is shown graphically: cytotoxicity was directed to target expressing, human tumor cells (FIG. 9) but not target negative, human tumor cells (FIG. 10).

Testing the In Vivo Tolerability of the Exemplary Cytotoxic Proteins SLT-1A-FR::scFv-1 and SLT-1A-FR::scFv-2 Using Laboratory Animals

The tolerability was tested for different dosages of the exemplary cytotoxic proteins SLT-1A-FR::scFv-1 and SLT-1A-FR::scFv-2, each of which comprises a furin-cleavage resistant, Shiga toxin A Subunit effector polypeptide region. Mice were used to determine the degree to which overt adverse effects were tolerated at various dosages of exemplary proteins. Therapeutic tolerability was determined using murine dose finding aimed at measuring maximum tolerated doses to inform a starting dose for in vivo efficacy studies. The tolerability studies were performed at Charles River Laboratories (Charles River Laboratories International, Inc., Morrisville, N.C., U.S.) in four separate studies described in Table 13. For each study, female C.B-17 SCID mice with severe combined immune deficiency (SCID) were sorted into groups with similar average body weight. Test agents or vehicle control were administered to mice at doses ranging from 0.25 to 5.00 milligrams per kilogram of body weight per injection (mg/kg/inj), and injections were administered three times a week for one or two weeks. Body weight and clinical signs were monitored throughout the study. The results of the in vivo tolerability studies using laboratory animals are summarized in Table 13. Table 13 denotes the number of mice per group, the administered sample, the injection dose, the cumulative dosage in mg per kg per mouse (mg/kg/mouse), the number of treatment-related deaths observed (Deaths), and the average day of death per group.

TABLE 13
Murine tolerability studies demonstrated the exemplary proteins SLT-1A-FR::scFv-1 and
SLT-1A-FR::scFv-2 were well tolerated in vivo at doses ranging from 0.25-2.50 mg/kg/inj

| | | | | Cumulative | Mice | | Average day |
|---|---|---|---|---|---|---|---|
| | | Dose | | dosage | per | | of death by |
| Study | Sample | (mg/kg/inj) | Doses | (mg/kg/mouse) | group | Deaths | group |
| #1 | vehicle control | 0.00 | 3 | 0.00 | 4 | 0 | all lived to |
| | | | | | | | study end |
| | SLT-1A-WT::scFv-1 | 1.25 | 3 | 3.75 | 4 | 4 | 8.5 |
| | SLT-1A-WT::scFv-1 | 2.50 | 3 | 7.50 | 4 | 4 | 7.3 |
| #2 | SLT-1A-WT::scFv-1 | 1.25 | 3 | 3.75 | 5 | 5 | 7.4 |
| | SLT-1A-WT::scFv-1 | 2.50 | 2 | 5.00 | 5 | 5 | 5.2 |
| | SLT-1A-WT::scFv-1 | 5.00 | 2 | 10.00 | 5 | 5 | 4.4 |
| #3 | vehicle control | 0.00 | 3 | 0.00 | 4 | 0 | all lived to |
| | | | | | | | study end |
| | SLT-1A-FR::scFv-1 | 0.25 | 3 | 0.75 | 4 | 0 | all lived to |
| | | | | | | | study end |
| | SLT-1A-FR::scFv-1 | 1.25 | 3 | 3.75 | 4 | 0 | all lived to |
| | | | | | | | study end |
| | SLT-1A-FR::scFv-1 | 2.50 | 3 | 7.5 | 4 | 0 | all lived to |
| | | | | | | | study end |
| #4 | vehicle control | 0.00 | 6 | 0.00 | 4 | 0 | all lived to |
| | | | | | | | study end |
| | SLT-1A-FR::scFv-2 | 0.25 | 6 | 1.50 | 4 | 0 | all lived to |
| | | | | | | | study end |
| | SLT-1A-FR::scFv-2 | 1.00 | 6 | 6.00 | 4 | 0 | all lived to |
| | | | | | | | study end |
| | SLT-1A-FR::scFv-2 | 2.00 | 6 | 12.00 | 4 | 0 | all lived to |
| | | | | | | | study end |

In study #1, the protein SLT-1A-WT::scFv-1 was tested along with a vehicle control. Mice were dosed on study days 1, 3 and 5 with vehicle control, 1.25, or 2.50 mg/kg/inj of SLT-1A-WT::scFv-1. In study 1, all the mice treated with SLT-1A-WT::scFv-1 had a treatment related death starting two days after the third dose. In study #1, mice from the group administered 1.25 mg/kg/inj of SLT-1A::WT-scFv-1 died on study days 7, 8, 9 and 10 (one mouse per day), and mice from the group administered 2.50 mg/kg/inj of SLT-1A-WT::scFv-1 died on day 7 (three mice) or day 8 (one mouse). All the mice in the group administered the vehicle control survived to the study end.

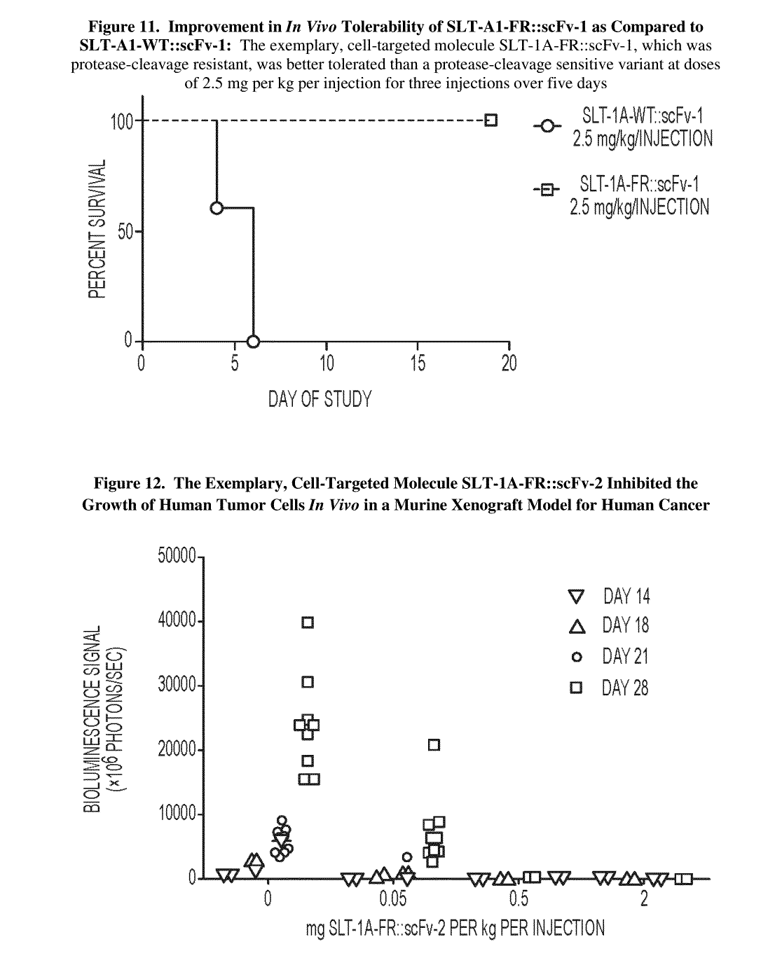

In study #2, mice were administered SLT-1A-WT::scFv-1 as in study #1 but with a higher maximum dose of 5.00 mg/kg/inj. In study #2, similar results occurred for mice administered SLT-1A-WT::scFv-1 as in study #1; however, in study #2, mice in the two highest dose groups only received two injections due to treatment-related deaths to mice in those groups. FIG. 11 shows a comparison of the survival of mice from a group administered 2.50 mg/kg/inj of SLT-1A-FR::scFv-1 (study #2) as compared to mice administered SLT-1A-WT::scFv-1 (study #1) using Kaplan-Meier plots.

In study 3, mice were administered with a vehicle control or the exemplary protein SLT-1A-FR::scFv-1 similar to the dosage regime for study #1 but with a lower dose of 0.25 mg/kg/inj. Mice were dosed on study days 1, 3, and 5 with 0.25, 1.25, or 2.50 mg/kg/inj of SLT-1A-FR::scFv-1. All mice administered SLT-1A-FR::scFv-1 survived to the end of study #3. A dose-dependent decrease in body weight was observed; however, a maximum tolerated dose was not observed for SLT-1A-FR::scFv-1 in study #3. The highest tested dosing group (administered 2.50 mg/kg/inj SLT-1A-FR::scFv-1) had a nadir of only 13.1% body weight loss. These results demonstrate that SLT-1A-FR::scFv-1 was well-tolerated in vivo at repeat doses ranging from 0.25-2.50 mg/kg/inj. These results also demonstrate that SLT-1A-FR::scFv-1 was better tolerated than SLT-1A-WT::scFv-1 under the conditions tested.

In study 4, mice were administered with a vehicle control or the exemplary protein SLT-1A-FR::scFv-2 at 0.25, 1.00, or 2.00 mg/kg/injection three times a week for two weeks (six total doses). All mice administered SLT-1A-FR::scFv-2 survived until the end of study #4 and no adverse clinical observations were noted during the course of the study. Study #4 was extended to day 32, and the average body weight was observed to be above 80% of the starting weight for all groups comprising mice administered SLT-1A-FR::scFv-2. These results demonstrate that SLT-1A-FR::scFv-2 was well-tolerated in vivo at repeat doses ranging from 0.25-2.00 mg/kg/inj.

Compared to the tolerability results for proteins comprising a protease-sensitive, wild-type, Shiga toxin effector polypeptide (studies #1 and #2), the exemplary proteins comprising furin-cleavage resistant, Shiga toxin effector polypeptides exhibited improved tolerability (studies #3 and #4) at dosages involving repeat doses ranging from 0.25 to 2.50 mg/kg/inj.

The improved in vivo tolerability observed for these two, exemplary, cytotoxic proteins of the invention suggests that much higher doses of the cytotoxic molecules of the invention may be safely administered to mammals as compared to parental molecules comprising a furin-cleavage sensitive, Shiga toxin effector polypeptide.

Despite retaining equivalent cytotoxicity to nearly identical proteins comprising wild-type Shiga toxin effector polypeptides, the exemplary proteins of the present invention which comprise furin-cleavage resistant, Shiga toxin effector polypeptides exhibited improved tolerability mammals, i.e. improved toxicity profiles due to a reduction in deleterious effects. The improved toxicity profiles might be due to a reduction in non-specific toxicity related to the generally improved protease resistance of the molecules. These results also suggest that disrupting the furin-cleavage motif might confer an increased stability for the entire cell-targeted fusion protein. In addition, a molecule's resistance to proteolysis might improve its pharmacokinetic profiles administration to an organism.

Testing the Targeted Cytotoxicity and Efficacy of the Exemplary Protein SLT-1A-FR::scFv-2 In Vivo Using Animal Models

A disseminated xenograft model for human tumors was used to determine the in vivo efficacy of the exemplary cytotoxic protein SLT-1A-FR::scFv-2 in human-tumor bearing mice. Human tumor cells that constitutively express luciferase and display cell-surface expression of the target of scFv-2 were used in this xenograft model.

On study day 0, CB.17 SCID mice with severe combined immune deficiency (SCID) were challenged intravenously with 2.5×10⁶ hTum-Luc tumor cells (Molecular Imaging, Ann Arbor, Mich., U.S.) in 200 microliters (μL) PBS. A confirmatory bioluminescent image (BLI) was taken 5 minutes after cell injection, and mice were divided into four groups of ten mice each (n=10 mice). On days 0 (1-hour post implant), 2, 4, 7, 9, and 11 following tumor cell challenge, the mice in the four groups received via intraperitoneal administration either vehicle control (0 mg/kg/inj) or SLT-1A-FR::scFv-2 at doses of 0.05, 0.50, or 2.00 mg/kg/inj. Bioluminescence was measured on days 14, 18, and 21 using a Caliper IVIS 50 optical imaging system (Perkin Elmer, Waltham, Mass., U.S.).

The exemplary cytotoxic protein SLT-1A-FR::scFv-2 reduced the human tumor burden in the mice at all dosage levels. The results of this study are reported in FIG. 12 and Table 14. In this study, the sample size (n) was 10 mice per group for all four groups. FIG. 12 shows the tumor burden as assayed by bioluminescence per individual mouse over time based on the human tumor cells' expression of the luciferase reporter. An individual mouse is represented by each symbol plotted on the graph, i.e. open triangle, filled triangle, open circle, or filled square. The Y-axis is the total bioluminescence signal of an individual mouse, which represents the tumor burden, in millions of photons per second (photons/sec), and the X-axis is the injection dose which ranged from 0 to 2 milligrams of SLT-1A-FR::scFv-2 per kilogram of body mass per injection. Table 14 reports the mean BLI and standard error of the mean (SEM) among mice in each group at different time points (study day 14, 18, 21, and 28).

TABLE 14
Murine xenograft study demonstrated that exemplary cytotoxic
molecule SLT-1A-FR::scFv-2 was efficacious in vivo

| | | | 0.05 mg SLT- | | 0.50 mg SLT- | | 2.00 mg SLT- | |
|---|---|---|---|---|---|---|---|---|
| | vehicle | | 1A-FR::scFv- | | 1A-FR::scFv- | | 1A-FR::scFv- | |
| | control | | 2/kg/inj | | 2/kg/inj | | 2/kg/inj | |
| | Mean | | Mean | | Mean | | Mean | |
| Day | BLI | SEM | BLI | SEM | BLI | SEM | BLI | SEM |
| 14 | 235 | 28 | 24 | 7 | 1.02 | 0.05 | 1.07 | 0.07 |
| 18 | 1900 | 201 | 251 | 80 | 1.36 | 0.18 | 0.90 | 0.04 |
| 21 | 5850 | 658 | 744 | 296 | 3.47 | 1.03 | 1.09 | 0.10 |
| 28 | 23700 | 2310 | 6960 | 1670 | 99.30 | 38.0 | 4.13 | 2.98 |

These results show SLT-1A-FR::scFv-2 was capable of significantly reducing the human tumor burden in SCID mice challenged with human tumor cells. All groups comprised of mice administered the exemplary protein SLT-1A-FR::scFv-2 showed significantly less total bioluminescence compared to the vehicle control (FIG. 12; Table 14). This effect was observed in mice administered dosages of SLT-1A-FR::scFv-2 ranging from 0.05 to 2.00 mg/kg/inj (FIG. 12; Table 14). The observed tumor-inhibition effect was dose dependent because mice administered 0.05 mg/kg/inj of SLT-1A-FR::scFv-2 showed some tumor growth as measured by BLI, while mice administered either 0.50 or 2.00 mg/kg/inj of SLT-1A-FR::scFv-2 did not display tumor growth (Table 14; FIG. 12).

These results demonstrate that the exemplary protein SLT-1A-FR::scFv-2 was 1) effective at inhibiting tumor growth in vivo in addition to exhibiting equivalent cytotoxicity to proteins comprising furin-cleavage sensitive, SLT-1A-WT; and 2) exhibited improved tolerability at higher doses compared to a nearly identical, protein comprising the furin-cleavage sensitive, SLT-1A-WT (these proteins differ by only two amino acid residues).

Summary: Protease-Cleavage Resistant, Cell-Targeted Molecules Retain Potent Cytotoxicity while Improving In Vivo Tolerability

The exemplary, cytotoxic, cell-targeted molecules of the present invention SLT-1A-FR::scFv-1, SLT-1A-FR::scFv-2, and SLT-1A-FR-2::scFv-2, which comprised mutations in the minimal, furin-cleavage motif R/Y-x-x-R, were not proteolyzed by human furin but exhibited specific cytotoxicities comparable to cell-targeted proteins comprising a wild-type, Shiga toxin effector region. The exemplary protein SLT-1A-FR::scFv-2 effectively inhibited human tumor growth in a mammalian model. In addition, SLT-1A-FR::scFv-1 and SLT-1A-FR::scFv-2 both exhibited improved tolerability as compared to parental molecules comprising a wild-type, Shiga toxin A Subunit effector region.

This example shows that disrupting a conserved, furin-cleavage event in the Shiga toxin A Subunit effector region of cell-targeted molecules did not impair the cytotoxicity of these cell-targeted molecules despite the presence of relatively large, molecule moieties (sized 28-29 kDa) linked carboxy-terminal to the Shiga toxin effector region. The exemplary, furin-cleavage resistant, cell-targeted molecules of this example exhibited cytotoxic potencies equivalent to related cell-targeted molecules comprising wild-type, Shiga toxin A1 fragments despite the presence of relatively large, carboxy-terminal, molecular moieties comprising immunoglobulin-type binding regions for cell-targeting. The relatively large, carboxy-terminal moieties of the cell-targeted molecules of this example, which physically covered the carboxy terminals of the Shiga toxin A1 fragment polypeptide regions of the exemplary, cell-targeted molecules, were expected to deleteriously function to tether Shiga toxin A1 fragment effector polypeptides to target biomolecules in the endoplasmic reticulum membrane and/or otherwise interfere with molecular mechanisms believed to be critical for the efficient intracellular routing of the A1 fragment effector polypeptide to the cytosol of an intoxicated cell. The potent cytotoxicities of the cell-targeted molecules of this example were surprising because Shiga toxins require proteolytic processing at this furin-cleavage site in the proper subcellular compartments for optimal cytotoxicity (see e.g. Garred Ø et al., Exp Cell Res 218: 39-49 (1995); Garred Ø et al., J Biol Chem 270: 10817-21 (1995); Lea N et al., Microbiology 145: 999-1004 (1999); Kurmanova A et al., Biochem Biophys Res Commun 357: 144-9 (2007)).

In addition, this example also shows that mutations disrupting the furin-cleavage of the protease-cleavage sensitive, surface-exposed loop in Shiga toxin A Subunit effector polypeptides enabled the engineering of cell-targeted molecules with improved in vivo tolerability while simultaneously retaining a Shiga toxin cytotoxicity as potent and efficient as cell-targeted molecules comprising wild-type, Shiga toxin A1 fragment regions.

The properties of SLT-1A-FR::scFv-1, SLT-1A-FR::scFv-2, and SLT-1A-FR-2::scFv-2, each which comprise Shiga toxin effector polypeptides comprising furin-cleavage disrupting mutations (R248A and/or R251A), suggest other disruptions of the furin-cleavage motif in the conserved, surface-exposed loop in Shiga toxin A Subunits may provide the same properties, such as, e.g., equivalent cytotoxicity to as molecules comprising wild-type, Shiga toxin A Subunit regions as well as improved in vivo tolerability.

Mutations similar to R248A and R251A in cell-targeted molecules comprising Shiga toxin A Subunit effector polypeptides can provide similar structure and function. For example, any mutation which perturbs the conserved, furin-cleavage, consensus motif S-R/Y-x-x-R in Shiga toxin A Subunits and/or Shiga toxin effector polypeptides derived therefrom will result in furin-cleavage resistance but not perturb cytotoxicity, e.g., such that significant Shiga toxin cytotoxic and/or wild-type level of Shiga toxin cytotoxicity is retained. In particular, amino acid residue substitutions of conserved, arginine residue(s) in the furin-cleavage site to any non-basic amino acid residue which lacks a positive charge, such as, e.g., A, G, P, S, T, D, E, Q, N, C, I, L, M, V, F, W, and Y, may be used to create a disrupted furin-cleavage motif of a Shiga toxin effector polypeptide of a cell-targeted molecule of the present invention. Similarly, truncations of the Shiga toxin A Subunit, internal deletions, and/or insertions within the furin-cleavage motif which perturb the minimal, furin-cleavage motif R/Y-x-x-R may be used to create Shiga toxin effector polypeptides with similar structure and function as to the Shiga toxin effector polypeptides shown in this example.

In summary, potently cytotoxic, cell-targeted molecules (e.g., fusion proteins) may be created by combining furin-cleavage resistant, Shiga toxin A Subunit derived polypeptides with cell-targeting binding region moieties of sizes greater than 28 kDa (such as, e.g., immunoglobulin-type binding regions like scFvs) which are linked carboxy-terminal to the carboxy-terminus of the Shiga toxin A1 fragment region of the Shiga toxin A Subunit derived effector region.

### Example 6

### A Cytotoxic, Cell-Targeted Molecule Derived from the A Subunit of Shiga-Like Toxin-1 and the Antibody αEpstein-Barr-Antigen

In this example, the Shiga toxin effector region is derived from the A subunit of Shiga-like Toxin 1 (SLT-1A). An immunoglobulin-type binding region αEpstein-Barr-antigen is derived from a monoclonal antibody against an Epstein Barr antigen (Fang C et al., J Immunol Methods 287: 21-30 (2004)), which comprises an immunoglobulin-type binding region capable of binding a human cell infected by the Epstein-Barr virus or a transformed cell expressing an Epstein-Barr antigen. The Epstein-Barr antigen is expressed on multiple cell types, such as cells infected by an Epstein-Barr virus and cancer cells (e.g. lymphoma and nasopharyngeal cancer cells). In addition, Epstein-Barr infection is associated with other diseases, e.g., multiple sclerosis.

### Construction, Production, and Purification of the Cytotoxic Protein SLT-1A::αEpsteinBarr

The immunoglobulin-type binding region αEpstein-Barr-antigen and Shiga toxin effector region are linked together to form a protein in which the immunoglobulin-type binding region is not located proximally to the protein's amino-terminus as compared to the Shiga toxin effector region. For example, a fusion protein is produced by expressing a polynucleotide encoding the αEpstein-Barr-antigen-binding protein SLT-1A::αEpsteinBarr. Expression of the SLT-1A::αEpsteinBarr cytotoxic protein is accomplished using either bacterial and/or cell-free, protein translation systems as described in the previous examples.

### Determining the In Vitro Characteristics of the Cytotoxic Protein SLT-1A::αEpsteinBarr

The binding characteristics of the cytotoxic protein of this example for Epstein-Barr antigen positive cells and Epstein-Barr antigen negative cells is determined by a fluorescence-based, flow-cytometry assay as described above in the previous examples. The Bₘₐₓ for SLT-1A::αEpsteinBarr to Epstein-Barr antigen positive cells is measured to be approximately 50,000-200,000 MFI with a K_{D} within the range of 0.01-100 nM, whereas there is no significant binding to Epstein-Barr antigen negative cells in this assay.

The ribosome inactivation abilities of the SLT-1A::αEpsteinBarr cytotoxic protein is determined in a cell-free, in vitro protein translation as described above in the previous examples. The inhibitory effect of the cytotoxic protein of this example on cell-free protein synthesis is significant. The IC₅₀ of SLT-1A::αEpsteinBarr on protein synthesis in this cell-free assay is approximately 0.1-100 pM.

### Determining the Cytotoxicity of the Cytotoxic Protein SLT-1A::αEpsteinBarr Using a Cell-Kill Assay

The cytotoxicity characteristics of SLT-1A::αEpsteinBarr are determined by the general cell-kill assay as described above in the previous examples using Epstein-Barr antigen positive cells. In addition, the selective cytotoxicity characteristics of SLT-1A::αEpsteinBarr are determined by the same general cell-kill assay using Epstein-Barr antigen negative cells as a comparison to the Epstein-Barr antigen positive cells. The CD₅₀ of the cytotoxic protein of this example is approximately 0.01-100 nM for Epstein-Barr antigen positive cells depending on the cell line. The CD₅₀ of the cytotoxic protein is approximately 10-10,000 fold greater (less cytotoxic) for cells not expressing the Epstein-Barr antigen on a cellular surface as compared to cells which do express the Epstein-Barr antigen on a cellular surface.

### Determining the In Vivo Effects of the Cytotoxic Protein SLT-1A::αEpsteinBarr Using Animal Models

Animal models are used to determine the in vivo effects of the cytotoxic protein SLT-1A::αEpsteinBarr on neoplastic cells. Various mice strains are used to test the effect of the cytotoxic protein after intravenous administration on xenograft tumors in mice resulting from the injection into those mice of human neoplastic cells which express Epstein-Barr viral antigens on their cell surfaces.

### Example 7

### A Cytotoxic, Cell-Targeted Molecule Derived from the A Subunit of Shiga-Like Toxin-1 and the Antibody αLeishmania-Antigen

In this example, the Shiga toxin effector region is derived from the A subunit of Shiga-like Toxin 1 (SLT-1A). An immunoglobulin-type binding region αLeishmania-antigen is derived from an antibody generated, using techniques known in the art, to a cell-surface Leishmania antigen present on human cells harboring an intracellular trypanosomatid protozoa (see Berman J, Dwyer D, Clin Exp Immunol 44: 342-348 (1981); Kenner J et al., J Cutan Pathol 26: 130-6 (1999); Silveira T et al., Int J Parasitol 31: 1451-8 (2001)).

### Construction, Production, and Purification of the Cytotoxic Protein SLT-1A::αLeishmania

The immunoglobulin-type binding region α-Leishmania-antigen and Shiga toxin effector region are linked together to form a protein in which the immunoglobulin-type binding region is not located proximally to the protein's amino-terminus as compared to the Shiga toxin effector region. For example, a fusion protein is produced by expressing a polynucleotide encoding the Leishmania-antigen-binding protein SLT-1A::αLeishmania. Expression of the SLT-1A::αLeishmania cytotoxic protein is accomplished using either bacterial and/or cell-free, protein translation systems as described in the previous examples.

### Determining the In Vitro Characteristics of the Cytotoxic Protein SLT-1A::αLeishmania

The binding characteristics of the cytotoxic protein of this example for Leishmania antigen positive cells and Leishmania antigen negative cells is determined by a fluorescence-based, flow-cytometry assay as described above in the previous examples. The Bₘₐₓ for SLT-1A::αLeishmania to Leishmania antigen positive cells is measured to be approximately 50,000-200,000 MFI with a K_{D} within the range of 0.01-100 nM, whereas there is no significant binding to Leishmania antigen negative cells in this assay.

The ribosome inactivation abilities of the SLT-1A::αLeishmania cytotoxic protein is determined in a cell-free, in vitro protein translation as described above in the previous examples. The inhibitory effect of the cytotoxic protein of this example on cell-free protein synthesis is significant. The IC₅₀ of SLT-1A::αLeishmania on protein synthesis in this cell-free assay is approximately 0.1-100 pM.

### Determining the Cytotoxicity of the Cytotoxic Protein SLT-1A::αLeishmania Using a Cell-Kill Assay

The cytotoxicity characteristics of SLT-1A::αLeishmania are determined by the general cell-kill assay as described above in the previous examples using Leishmania antigen positive cells. In addition, the selective cytotoxicity characteristics of SLT-1A::αLeishmania are determined by the same general cell-kill assay using Leishmania antigen negative cells as a comparison to the Leishmania antigen positive cells. The CD₅₀ of the cytotoxic protein of this example is approximately 0.01-100 nM for Leishmania antigen positive cells depending on the cell line. The CD₅₀ of the cytotoxic protein is approximately 10-10,000 fold greater (less cytotoxic) for cells not expressing the Leishmania antigen on a cellular surface as compared to cells which do express the Leishmania antigen on a cellular surface.

### Example 8

### A Cytotoxic, Cell-Targeted Molecule Derived from the A Subunit of Shiga-Like Toxin-1 and Immunoglobulin-Type Binding Region αNeurotensin-Receptor

In this example, the Shiga toxin effector region is derived from the A subunit of Shiga-like Toxin 1 (SLT-1A). An immunoglobulin-type binding region αNeurotensin-Receptor is derived from the DARPin™ (GenBank Accession: 2P2C_R) or a monoclonal antibody (Ovigne J et al., Neuropeptides 32: 247-56 (1998)) which binds the human neurotensin receptor. The neurotensin receptor is expressed by various cancer cells, such as breast cancer, colon cancer, lung cancer, melanoma, and pancreatic cancer cells.

### Construction, Production, and Purification of the Cytotoxic Protein SLT-1A::αNeurotensinR

The immunoglobulin-type binding region αNeurotensinR and Shiga toxin effector region are linked together to form a protein in which the immunoglobulin-type binding region is not located proximally to the protein's amino-terminus as compared to the Shiga toxin effector region. For example, a fusion protein is produced by expressing a polynucleotide encoding the neurotensin-receptor-binding protein SLT-1A::αNeurotensinR. Expression of the SLT-1A::αNeurotensinR cytotoxic protein is accomplished using either bacterial and/or cell-free, protein translation systems as described in the previous examples.

### Determining the In Vitro Characteristics of the Cytotoxic Protein SLT-1A::αNeurotensinR

The binding characteristics of the cytotoxic protein of this example for neurotensin receptor positive cells and neurotensin receptor negative cells is determined by a fluorescence-based, flow-cytometry assay as described above in the previous examples. The Bₘₐₓ for SLT-1A::αNeurotensinR to neurotensin receptor positive cells is measured to be approximately 50,000-200,000 MFI with a K_{D} within the range of 0.01-100 nM, whereas there is no significant binding to neurotensin receptor negative cells in this assay.

The ribosome inactivation abilities of the SLT-1A::αNeurotensinR cytotoxic protein is determined in a cell-free, in vitro protein translation as described above in the previous examples. The inhibitory effect of the cytotoxic protein of this example on cell-free protein synthesis is significant. The IC₅₀ of SLT-1A::αNeurotensinR on protein synthesis in this cell-free assay is approximately 0.1-100 pM.

### Determining the Cytotoxicity of the Cytotoxic Protein SLT-1A::αNeurotensinR Using a Cell-Kill Assay

The cytotoxicity characteristics of SLT-1A::αNeurotensinR are determined by the general cell-kill assay as described above in the previous examples using neurotensin receptor positive cells. In addition, the selective cytotoxicity characteristics of SLT-1A::αNeurotensinR are determined by the same general cell-kill assay using neurotensin receptor negative cells as a comparison to the neurotensin receptor positive cells. The CD₅₀ of the cytotoxic protein of this example is approximately 0.01-100 nM for neurotensin receptor positive cells depending on the cell line. The CD₅₀ of the cytotoxic protein is approximately 10-10,000 fold greater (less cytotoxic) for cells not expressing neurotensin receptor on a cellular surface as compared to cells which do express neurotensin receptor on a cellular surface.

### Determining the In Vivo Effects of the Cytotoxic Protein SLT-1A::αNeurotensinR Using Animal Models

Animal models are used to determine the in vivo effects of the cytotoxic protein SLT-1A::αNeurotensinR on neoplastic cells. Various mice strains are used to test the effect of the cytotoxic protein after intravenous administration on xenograft tumors in mice resulting from the injection into those mice of human neoplastic cells which express neurotensin receptors on their cell surfaces.

### Example 9

### A Cytotoxic, Cell-Targeted Molecule Derived from the A Subunit of Shiga-Like Toxin-1 and an Immunoglobulin-Type Binding Region αEGFR

In this example, the Shiga toxin effector region is derived from the A subunit of Shiga-like Toxin 1 (SLT-1A). An immunoglobulin-type binding region αEGFR is derived from the AdNectin™ (GenBank Accession: 3QWQ_B), the Affibody™ (GenBank Accession: 2KZI_A; U.S. Pat. No. 8,598,113), or an antibody, all of which bind one or more human epidermal growth factor receptors. The expression of epidermal growth factor receptors is associated with human cancer cells, such as, e.g., lung cancer cells, breast cancer cells, and colon cancer cells.

### Construction, Production, and Purification of the Cytotoxic Protein SLT-1A::αEGFR

The immunoglobulin-type binding region αEGFR and Shiga toxin effector region are linked together to form a protein in which the immunoglobulin-type binding region is not located proximally to the protein's amino-terminus as compared to the Shiga toxin effector region. For example, a fusion protein is produced by expressing a polynucleotide encoding the EGFR binding protein SLT-1A::αEGFR. Expression of the SLT-1A::αEGFR cytotoxic protein is accomplished using either bacterial and/or cell-free, protein translation systems as described in the previous examples.

### Determining the In Vitro Characteristics of the Cytotoxic Protein SLT-1A::αEGFR

The binding characteristics of the cytotoxic protein of this example for EGFR+ cells and EGFR− cells is determined by a fluorescence-based, flow-cytometry assay as described above in the previous examples. The Bₘₐₓ for SLT-1A::αEGFR to EGFR+ cells is measured to be approximately 50,000-200,000 MFI with a K_{D} within the range of 0.01-100 nM, whereas there is no significant binding to EGFR− cells in this assay.

The ribosome inactivation abilities of the SLT-1A::αEGFR cytotoxic protein is determined in a cell-free, in vitro protein translation as described above in the previous examples. The inhibitory effect of the cytotoxic protein of this example on cell-free protein synthesis is significant. The IC₅₀ of SLT-1A::αEGFR on protein synthesis in this cell-free assay is approximately 0.1-100 pM.

### Determining the Cytotoxicity of the Cytotoxic Protein SLT-1A::αEGFR Using a Cell-Kill Assay

The cytotoxicity characteristics of SLT-1A::αEGFR are determined by the general cell-kill assay as described above in the previous examples using EGFR+ cells. In addition, the selective cytotoxicity characteristics of SLT-1A::αEGFR are determined by the same general cell-kill assay using EGFR− cells as a comparison to the EGFR antigen positive cells. The CD₅₀ of the cytotoxic protein of this example is approximately 0.01-100 nM for EGFR+ cells depending on the cell line. The CD₅₀ of the cytotoxic protein is approximately 10-10,000 fold greater (less cytotoxic) for cells not expressing EGFR on a cellular surface as compared to cells which do express EGFR on a cellular surface.

### Determining the In Vivo Effects of the Cytotoxic Protein SLT-1A::αEGFR Using Animal Models

Animal models are used to determine the in vivo effects of the cytotoxic protein SLT-1A::αEGFR on neoplastic cells. Various mice strains are used to test the effect of the cytotoxic protein after intravenous administration on xenograft tumors in mice resulting from the injection into those mice of human neoplastic cells which express EGFR(s) on their cell surfaces.

### Example 10

### A Cytotoxic, Cell-Targeted Molecule Derived from the A Subunit of Shiga-Like Toxin-1 and the Antibody αCCR5

In this example, the Shiga toxin effector region is derived from the A subunit of Shiga-like Toxin 1 (SLT-1A). An immunoglobulin-type binding region αCCR5 is derived from a monoclonal antibody against human CCR5 (CD195) (Bernstone L et al., Hybridoma 31: 7-19 (2012)). CCR5 is predominantly expressed on T-cells, macrophages, dendritic cells, and microglia. In addition, CCR5 plays a role in the pathogenesis and spread of the Human Immunodeficiency Virus (HIV).

### Construction, Production, and Purification of the Cytotoxic Protein SLT-1A::αCCR5

The immunoglobulin-type binding region αCCR5 and Shiga toxin effector region are linked together to form a protein in which the immunoglobulin-type binding region is not located proximally to the protein's amino-terminus as compared to the Shiga toxin effector region. For example, a fusion protein is produced by expressing a polynucleotide encoding the αCCR5-binding protein SLT-1A::αCCR5. Expression of the SLT-1A::αCCR5 cytotoxic protein is accomplished using either bacterial and/or cell-free, protein translation systems as described in the previous examples.

### Determining the In Vitro Characteristics of the Cytotoxic Protein SLT-1A::αCCR5

The binding characteristics of the cytotoxic protein of this example for CCR5+ cells and CCR5− cells is determined by a fluorescence-based, flow-cytometry assay as described above in the previous examples. The Bₘₐₓ for SLT-1A::αCCR5 to CCR5+ positive cells is measured to be approximately 50,000-200,000 MFI with a K_{D} within the range of 0.01-100 nM, whereas there is no significant binding to CCR5− cells in this assay.

The ribosome inactivation abilities of the SLT-1A::αCCR5 cytotoxic protein is determined in a cell-free, in vitro protein translation as described above in the previous examples. The inhibitory effect of the cytotoxic protein of this example on cell-free protein synthesis is significant. The IC₅₀ of SLT-1A::αCCR5 on protein synthesis in this cell-free assay is approximately 0.1-100 pM.

### Determining the Cytotoxicity of the Cytotoxic Protein SLT-1A::αCCR5 Using a Cell-Kill Assay

The cytotoxicity characteristics of SLT-1A::αCCR5 are determined by the general cell-kill assay as described above in the previous examples using CCR5+ cells. In addition, the selective cytotoxicity characteristics of SLT-1A::αCCR5 are determined by the same general cell-kill assay using CCR5− cells as a comparison to the CCR5+ cells. The CD₅₀ of the cytotoxic protein of this example is approximately 0.01-100 nM for CCR5+ cells depending on the cell line. The CD₅₀ of the cytotoxic protein is approximately 10-10,000 fold greater (less cytotoxic) for cells not expressing CCR5 on a cellular surface as compared to cells which do express CCR5 on a cellular surface.

### Determining the In Vivo Effects of the Cytotoxic Protein SLT-1A::αCCR5 Using Animal Models

Animal models are used to determine the in vivo effects of the cytotoxic protein SLT-1A::αCCR5 on depleting T-cells from donor materials (see Tsirigotis P et al., Immunotherapy 4: 407-24 (2012)). Non-human primates are used to determine in vivo effects of SLT-1A::αCCR5. Graft-versus-host disease is analyzed in rhesus macaques after kidney transplantation when the donated organs are pretreated with SLT-1A::αCCR5 (see Weaver T et al., Nat Med 15: 746-9 (2009)). In vivo depletion of peripheral blood T lymphocytes in cynomolgus primates is observed after parenteral administration of different doses of SLT-1A::αCCR5. The use of SLT-1A::αCCR5 to block HIV infection is tested by giving an acute dose of SLT-1A::αCCR5 to non-human primates in order to severely deplete circulating T-cells upon exposure to a simian immunodeficiency virus (SIV) (see Sellier P et al., PLoS One 5: e10570 (2010)).

### Example 11

### A Cytotoxic, Cell-Targeted Molecule Derived from the A Subunit of Shiga Toxin and an Anti-Env Immunoglobulin Domain

In this example, the Shiga toxin effector region is derived from the A subunit of Shiga toxin (Stx-A). An immunoglobulin-type binding region αEnv is derived from existing antibodies that bind HIV envelope glycoprotein (Env), such as GP41, GP120, GP140, or GP160 (see e.g. Chen W et al., J Mol Bio 382: 779-89 (2008); Chen W et al., Expert Opin Biol Ther 13: 657-71 (2013); van den Kerkhof T et al., Retrovirology 10: 102 (2013) or from antibodies generated using standard techniques (see Prabakaran P et al., Front Microbiol 3: 277 (2012)). Envs are HIV surface proteins that are also displayed on the cell surfaces of HIV-infected cells during HIV replication. Although Envs are expressed in infected cells predominantly in endosomal compartments, sufficient amounts of Envs could be present on a cell surface to be targeted by a highly potent cytotoxic protein of the invention. In addition, Env-targeting cytotoxic proteins might bind HIV virions and enter newly infected cells during the fusion of virions with a host cell.

Because HIV displays a high rate of mutation, it is preferable to use an immunoglobulin domain that binds a functional constrained part of an Env, such as shown by broadly neutralizing antibodies that bind Envs from multiple strains of HIV (van den Kerkhof T et al., Retrovirology 10: 102 (2013)). Because the Envs present on an infected cell's surface are believed to present sterically restricted epitopes (Chen W et al., J Virol 88: 1125-39 (2014)), it is preferable to use binding regions smaller than 100 kDa and ideally smaller than 25 kDa, such as fragments of sdAbs like V_{H}H domains.

### Construction, Production, and Purification of the Cytotoxic Protein αEnv::SLT-1A

The immunoglobulin-type binding region αEnv and Shiga toxin effector region are linked together to form a cytotoxic protein. For example, a fusion protein is produced by expressing a polynucleotide encoding the αEnv-binding protein SLT-1A::αEnv. Expression of the SLT-1A::αEnv cytotoxic protein is accomplished using either bacterial and/or cell-free, protein translation systems as described in the previous examples.

### Determining the In Vitro Characteristics of the Cytotoxic Protein SLT-1A::αEnv

The binding characteristics of the cytotoxic protein of this example for Env+ cells and Env− cells is determined by a fluorescence-based, flow-cytometry assay as described above in the previous examples. The Bₘₐₓ for SLT-1A::αEnv to Env+ positive cells is measured to be approximately 50,000-200,000 MFI with a K_{D} within the range of 0.01-100 nM, whereas there is no significant binding to Env− cells in this assay.

The ribosome inactivation abilities of the SLT-1A::αEnv cytotoxic protein is determined in a cell-free, in vitro protein translation as described above in the previous examples. The inhibitory effect of the cytotoxic protein of this example on cell-free protein synthesis is significant. The IC₅₀ of SLT-1A::αEnv on protein synthesis in this cell-free assay is approximately 0.1-100 pM.

### Determining the Cytotoxicity of the Cytotoxic Protein SLT-1A::αEnv Using a Cell-Kill Assay

The cytotoxicity characteristics of SLT-1A::αEnv are determined by the general cell-kill assay as described above in the previous examples using Env+ cells. In addition, the selective cytotoxicity characteristics of SLT-1A::αEnv are determined by the same general cell-kill assay using Env− cells as a comparison to the Env+ cells. The CD₅₀ of the cytotoxic protein of this example is approximately 0.01-100 nM for Env+ cells depending on the cell line and/or the HIV strain used to infect the cells to make them Env+. The CD₅₀ of the cytotoxic protein is approximately 10-10,000 fold greater (less cytotoxic) for cells not expressing Env on a cellular surface as compared to cells which do express Env on a cellular surface.

### Determining the In Vivo Effects of the Cytotoxic Protein SLT-1A::αEnv Using Animal Models

The use of SLT-1A::αEnv to inhibit HIV infection is tested by administering SLT-1A::αEnv to simian immunodeficiency virus (SIV) infected non-human primates (see Sellier P et al., PLoS One 5: e10570 (2010)).

### Example 12

### A Cytotoxic, Cell-Targeted Molecule Derived from the A Subunit of Shiga-Like Toxin-1 and the Antibody αUL18

In this example, the Shiga toxin effector region is derived from the A subunit of Shiga-like Toxin 1 (SLT-1A). An immunoglobulin-type binding region αUL18 is derived from an antibody generated, using techniques known in the art, to the cell-surface cytomegalovirus protein UL18, which is present on human cells infected with cytomegalovirus (Yang Z, Bjorkman P, Proc Natl Acad Sci USA 105: 10095-100 (2008)). The human cytomegalovirus infection is associated with various cancers and inflammatory disorders.

### Construction, Production, and Purification of the Cytotoxic Protein SLT-1A::αUL18

The immunoglobulin-type binding region αUL18 and Shiga toxin effector region are linked together to form a protein in which the immunoglobulin-type binding region is not located proximally to the protein's amino-terminus as compared to the Shiga toxin effector region. For example, a fusion protein is produced by expressing a polynucleotide encoding the αUL18-binding protein SLT-1A::αUL18. Expression of the SLT-1A::αUL18 cytotoxic protein is accomplished using either bacterial and/or cell-free, protein translation systems as described in the previous examples.

### Determining the In Vitro Characteristics of the Cytotoxic Protein SLT-1A::αUL18

The binding characteristics of the cytotoxic protein of this example for cytomegalovirus protein UL18 positive cells and cytomegalovirus protein UL18 negative cells is determined by a fluorescence-based, flow-cytometry assay as described above in the previous examples. The Bₘₐₓ for SLT-1A::αUL18 to cytomegalovirus protein UL18 positive cells is measured to be approximately 50,000-200,000 MFI with a K_{D} within the range of 0.01-100 nM, whereas there is no significant binding to cytomegalovirus protein UL18 negative cells in this assay.

The ribosome inactivation abilities of the SLT-1A::αUL18 cytotoxic protein is determined in a cell-free, in vitro protein translation as described above in the previous examples. The inhibitory effect of the cytotoxic protein of this example on cell-free protein synthesis is significant. The IC₅₀ of SLT-1A::αUL18 on protein synthesis in this cell-free assay is approximately 0.1-100 pM.

### Determining the Cytotoxicity of the Cytotoxic Protein SLT-1A::αUL18 Using a Cell-Kill Assay

The cytotoxicity characteristics of SLT-1A::αUL18 are determined by the general cell-kill assay as described above in the previous examples using cytomegalovirus protein UL18 positive cells. In addition, the selective cytotoxicity characteristics of SLT-1A::αUL18 are determined by the same general cell-kill assay using cytomegalovirus protein UL18 negative cells as a comparison to the cytomegalovirus protein UL18 positive cells. The CD₅₀ of the cytotoxic protein of this example is approximately 0.01-100 nM for cytomegalovirus protein UL18 positive cells depending on the cell line. The CD₅₀ of the cytotoxic protein is approximately 10-10,000 fold greater (less cytotoxic) for cells not expressing the cytomegalovirus protein UL18 on a cellular surface as compared to cells which do express the cytomegalovirus protein UL18 on a cellular surface.

### Example 13

### A Cytotoxic, Cell-Targeted Molecule Derived from the A Subunit of Shiga-Like Toxin-1 and the Antibody αHelminth-Intestinal-Antigen

In this example, the Shiga toxin effector region is derived from the A subunit of Shiga-like Toxin 1 (SLT-1A). An immunoglobulin-type binding region αhelminth-intestinal-antigen is derived from an antibody generated, using techniques known in the art, to the helminth ortholog of a human transferrin receptor (see e.g. the nematode gene gcp-2.1 UniProt G8JYE4_CAEEL; Rosa B et al., Mol Cell Proteomics M114.046227 (2015)).

Construction, Production, and Purification of the Cytotoxic Protein SLT-1A::αHelminth-Intestinal-Antigen and/or SLT-1A-FR::αHelminth-Intestinal-Antigen

The immunoglobulin-type binding region αhelminth-intestinal-antigen and Shiga toxin effector region, which is optionally a protease-cleavage resistant Shiga toxin effector region, are linked together to form a protein in which the immunoglobulin-type binding region is not located proximally to the protein's amino-terminus as compared to the Shiga toxin effector region. For example, a fusion protein is produced by expressing a polynucleotide encoding an αhelminth-intestinal-antigen-binding protein fused to an amino-terminal SLT-1A or SLT-1A-FR (e.g., the Shiga toxin effector polypeptide comprising amino acids 1-251 of SEQ ID NO:1 or amino acids 2-252 of SEQ ID NO:32). Expression of the SLT-1A::αhelminth-intestinal-antigen-binding protein and/or SLT-1A-FR::αhelminth-intestinal-antigen-binding protein is accomplished using either bacterial and/or cell-free, protein translation systems as described in the previous examples.

Determining the In Vitro Characteristics of the Cytotoxic, Cell-Targeted Molecule SLT-1A::αHelminth-Intestinal-Antigen and/or SLT-1A-FR::αHelminth-Intestinal-Antigen

The binding characteristics of the cytotoxic, cell-targeted molecule of this example is determined by a molecular binding assay known in the art using a purified recombinant target protein. The K_{D} for SLT-1A::αhelminth-intestinal-antigen and/or SLT-1A-FR::αhelminth-intestinal-antigen to target protein is measured to be approximately 100 nM, whereas there is no significant binding to a negative control protein (e.g. purified, recombinant, helminth ortholog of human transferrin receptor) in this assay.

The ribosome inactivation abilities of the SLT-1A::αhelminth-intestinal-antigen and/or SLT-1A-FR::αhelminth-intestinal-antigen cytotoxic proteins is determined in a cell-free, in vitro protein translation as described above in the previous examples. The inhibitory effect of the cytotoxic, cell-targeted molecule of this example on cell-free protein synthesis is significant. The IC₅₀ of SLT-1A::αhelminth-intestinal-antigen and SLT-1A-FR::αhelminth-intestinal-antigen on protein synthesis in this cell-free assay is approximately 0.1-100 pM.

Determining the Toxicity of the Cytotoxic, Cell-Targeted Molecule SLT-1A::αHelminth-Intestinal-Antigen and/or SLT-1A-FR::αHelminth-Intestinal-Antigen Using Helminths

The toxicity of SLT-1A::αhelminth-intestinal-antigen and/or SLT-1A-FR::αhelminth-intestinal-antigen to helminths is determined using model helminths (see e.g. Iatsenko I et al., Toxins 2050-63 (2014)). The helminth can be administered purified SLT-1A::αhelminth-intestinal-antigen and/or SLT-1A-FR::αhelminth-intestinal-antigen by soaking or alternatively by feeding the helminth with bacteria expressing the SLT-1A::αhelminth-intestinal-antigen and/or SLT-1A-FR::αhelminth-intestinal-antigen fusion protein.

In addition, laboratory animals harboring helminths and/or displaying helminth-related diseases are administered SLT-1A::αhelminth-intestinal-antigen and/or SLT-1A-FR::αhelminth-intestinal-antigen and monitored for reduction or elimination of helminths and/or associated symptoms of parasitic helminth(s).

### Example 14

### A Cytotoxic, Cell-Targeted Molecule Comprising a Protease-Cleavage Resistant, Shiga Toxin Effector Polypeptide Fused to an Immunoglobulin-Type Binding Region Specific to HIV-1 Gag

In this example, the Shiga toxin effector polypeptide region is a protease-cleavage resistant, Shiga toxin effector polypeptide derived from the A subunit of Shiga-like Toxin 1 (SLT-1A), such as, e.g., the Shiga toxin effector polypeptide comprising amino acids 2-252 of SEQ ID NO:32. An immunoglobulin-type binding region αGag-antigen is derived from an immunoglobulin-type domain recognizing the HIV capsid protein HIV-1 Gag polyprotein (see e.g. Nagola S et al., Retrovirology 9: 17 (2012)), which comprises an artificial, Ankyrin domain repeat polypeptide capable of binding an extracellular part of Gag. Gag is the major structural protein involved in HIV virus assembly and oligomerizes into a lattice of as many as 700 to 5000 copies per virion to form a conical-shaped CA core (see e.g. Chen Y et al., Biophys J 96: 1961-9 (2009); Pornillos O et al., Nature 469: 424-7 (2011)).

### Construction, Production, and Purification of the Cytotoxic Protein “SLT-1A-FR::αGag”

The immunoglobulin-type binding region aGag and a protease-cleavage resistant, Shiga toxin effector polypeptide are fused to form a cytotoxic protein. For example, a fusion protein is produced by expressing a polynucleotide encoding the αGag-antigen-binding protein SLT-1A-FR::αGag. Expression of the SLT-1A-FR::αGag cytotoxic protein is accomplished using either bacterial and/or cell-free, protein translation systems as described in the previous examples.

### Determining the In Vitro Characteristics of the Cell-Targeted Molecule “SLT-1A-FR::αGag”

The binding characteristics of the cytotoxic protein of this example for purified, recombinant HIV-1 Gag is determined by an assay known in the art, such as an enzyme-linked immunosorbent assay. The K_{D} for SLT-1A-FR::αGag binding to Gag is measured to be approximately within the range of 0.01-100 nanomolar (nM).

The ribosome inactivation abilities of the SLT-1A-FR::αGag cytotoxic protein is determined in a cell-free, in vitro protein translation as described above in the previous examples. The inhibitory effect of the cytotoxic protein of this example on cell-free protein synthesis is significant. The IC₅₀ of SLT-1A-FR::αGag on protein synthesis in this cell-free assay is approximately 0.1-100 pM.

### Determining the Cytotoxicity of the Cell-Targeted Molecule “SLT-1A-FR::αGag” Using a Cell-Kill Assay

The cytotoxicity characteristics of SLT-1A-FR::αGag are determined by the general cell-kill assay as described above in the previous examples using HIV-infected human T-cells. In addition, the selective cytotoxicity characteristics of SLT-1A-FR::αGag are determined by the same general cell-kill assay using uninfected T-cells as a comparison to the infected T-cells. The CD₅₀ of the cytotoxic protein of this example is approximately 0.01-100 nM for infected T-cells with actively replicating virus. The CD₅₀ of the cytotoxic protein is approximately 10-10,000 fold greater (less cytotoxic) for uninfected T-cells.

### Determining the In Vivo Effects of the Cytotoxic, Cell-Targeted Molecule “SLT-1A-FR::αGag” Using Animal Models

The use of SLT-1A-FR::αGag to inhibit the progression of HIV infection is tested by administering SLT-1A-FR::αGag to simian immunodeficiency virus (SIV) infected non-human primates (see Sellier P et al., PLoS One 5: e10570 (2010)).

### Example 15

### A Cytotoxic, Cell-Targeted Molecule Comprising a Protease-Cleavage Resistant, Shiga Toxin Effector Region Fused to an Immunoglobulin-Type Binding Region Specific to CD20

In this example, the Shiga toxin effector polypeptide region is a protease-cleavage resistant, Shiga toxin effector polypeptide derived from the A subunit of Shiga-like Toxin 1 (SLT-1A), such as, e.g., the Shiga toxin effector polypeptide comprising amino acids 2-252 of SEQ ID NO:32. An immunoglobulin-type binding region αCD20-antigen is derived from an immunoglobulin-type domain recognizing human CD20 (see e.g. Haisma H et al., Blood 92: 184-90 (1999); Geng S et al., Cell Mol Immunol 3: 439-43 (2006); Olafesn T et al., Protein Eng Des Sel 23: 243-9 (2010)), which comprises an immunoglobulin-type binding region capable of binding an extracellular part of CD20. CD20 is expressed on multiple cancer cell types, such as B-cell lymphoma cells, hairy cell leukemia cells, B-cell chronic lymphocytic leukemia cells, and melanoma cells. In addition, CD20 is an attractive target for therapeutics to treat certain autoimmune diseases, disorders, and conditions involving overactive B-cells.

### Construction, Production, and Purification of the Cytotoxic, Cell-Targeted Molecule “SLT-1A-FR::αCD20”

The immunoglobulin-type binding region αCD20 and a protease-cleavage resistant, Shiga toxin effector polypeptide are linked together to form a cytotoxic, cell-targeted molecule. For example, a fusion protein is produced by expressing a polynucleotide encoding the αCD20-antigen-binding protein SLT-1A-FR::αCD20. Expression of the SLT-1A-FR::αCD20 cytotoxic molecule is accomplished using either bacterial and/or cell-free, protein translation systems as described in the previous examples.

### Determining the In Vitro Characteristics of the Cytotoxic, Cell-Targeted Molecule “SLT-1A-FR::αCD20”

The binding characteristics of the cytotoxic molecule of this example for CD20+ cells and CD20− cells is determined by a fluorescence-based, flow-cytometry assay known in the art. Using Prism software (GraphPad Software, San Diego, Calif., U.S.), the Bₘₐₓ and K_{D} are calculated using the Prism software function of one-site binding [Y=Bₘₐₓ*X/(K_{D}+X)] under the heading binding-saturation. Bₘₐₓ is the maximum specific binding reported in MFI. K_{D} is the equilibrium binding constant, reported in nM. The Bₘₐₓ for SLT-1A-FR::αCD20 to CD20+ cells is measured to be approximately 50,000-200,000 MFI with a K_{D} within the range of 0.01-100 nanomolar (nM), whereas there is no significant binding to CD20− cells in this assay.

The ribosome inactivation abilities of the SLT-1A-FR::αCD20 cytotoxic molecule is determined in a cell-free, in vitro protein translation as described above in the previous examples. The inhibitory effect of the cytotoxic molecule of this example on cell-free protein synthesis is significant. The IC₅₀ of SLT-1A-FR::αCD20 on protein synthesis in this cell-free assay is approximately 0.1-100 pM.

### Determining the Cytotoxicity of the Cytotoxic, Cell-Targeted Molecule “SLT-1A-FR::αCD20” Using a CD20+ Cell-Kill Assay

The cytotoxicity characteristics of SLT-1A-FR::αCD20 are determined by the general cell-kill assay as described above in the previous examples using CD20+ cells. In addition, the selective cytotoxicity characteristics of SLT-1A-FR::αCD20 are determined by the same general cell-kill assay using CD20− cells as a comparison to the CD20+ cells. The CD₅₀ of the cytotoxic molecule of this example is approximately 0.01-100 nM for CD20+ cells depending on the cell line. The CD₅₀ of the cytotoxic molecule is approximately 10-10,000 fold greater (less cytotoxic) for cells not expressing CD20 on a cellular surface as compared to cells which do express CD20 on a cellular surface.

### Determining the In Vivo Effects of the Cytotoxic, Cell-Targeted Molecule “SLT-1A-FR::αCD20” Using Animal Models

Animal models are used to determine the in vivo effects of the cytotoxic molecule SLT-1A-FR::αCD20 on neoplastic cells. Various mice strains are used to test the effect of the cytotoxic molecule after intravenous administration on xenograft tumors in mice resulting from the injection into those mice of human neoplastic cells which express CD20 on their cell surfaces.

### Example 16

### A Cytotoxic, Cell-Targeted Molecule Comprising a Protease-Cleavage Resistant, Shiga Toxin Effector Region Fused to an Immunoglobulin-Type Binding Region Specific to HER2

In this example, a protease-cleavage resistant, Shiga toxin effector polypeptide region was derived from the A subunit of Shiga-like Toxin 1 (SLT-1A). The immunoglobulin-type binding region is αHER2 V_{H}H derived from a single-domain variable region of the camelid antibody (V_{H}H) protein 5F7, as described in U.S. Patent Application Publication 2011/0059090.

Construction, Production, and Purification of the Cytotoxic, Cell-Targeted Molecule “SLT-1A-FR Linked with αHER2-V_{H}H”

The immunoglobulin-type binding region and a protease-cleavage resistant, Shiga toxin effector polypeptide are linked together to form a cytotoxic, cell-targeted molecule. For example, a polynucleotide encoding the Shiga toxin effector polypeptide comprising amino acids 2-252 of SEQ ID NO:32 is cloned in frame with a polynucleotide encoding a linker known in the art and in frame with a polynucleotide encoding the αHER2-V_{H}H variable region derived from protein 5F7. Expression of the “SLT-1A-FR linked with αHER2-V_{H}H” cytotoxic molecule is accomplished using either bacterial and/or cell-free, protein translation systems as described in the previous examples.

Determining the In Vitro Characteristics of the Cytotoxic, Cell-Targeted Molecule “SLT-1A-FR linked with αHER2-V_{H}H”

The binding characteristics of the cytotoxic molecule of this example for HER2+ cells and HER2− cells is determined by a fluorescence-based, flow-cytometry assay known in the art. The Bₘₐₓ for “SLT-1A-FR linked with αHER2-V_{H}H” variants to HER2+ cells is measured to be approximately 50,000-200,000 MFI with a K_{D} within the range of 0.01-100 nM, whereas there is no significant binding to HER2− cells in this assay.

The ribosome inactivation abilities of the “SLT-1A-FR linked with αHER2-V_{H}H” cytotoxic molecules are determined in a cell-free, in vitro protein translation as described above in the previous examples. The inhibitory effect of the cytotoxic molecule of this example on cell-free protein synthesis is significant. The IC₅₀ of “SLT-1A-FR linked with αHER2-V_{H}H” variants on protein synthesis in this cell-free assay is approximately 0.1-100 pM.

Determining the Cytotoxicity of the Cytotoxic, Cell-Targeted Molecule “SLT-1A-FR Linked with αHER2-V_{H}H” Using a HER2+ Cell-Kill Assay

The cytotoxicity characteristics of “SLT-1A-FR linked with αHER2-V_{H}H” variants are determined by the general cell-kill assay as described above in the previous examples using HER2+ cells. In addition, the selective cytotoxicity characteristics of “SLT-1A-FR linked with αHER2-V_{H}H” are determined by the same general cell-kill assay using HER2− cells as a comparison to the HER2+ cells. The CD₅₀ of the cytotoxic molecule of this example is approximately 0.01-100 nM for HER2+ cells depending on the cell line. The CD₅₀ of the cytotoxic molecule is approximately 10-10,000 fold greater (less cytotoxic) for cells not expressing HER2 on a cellular surface as compared to cells which do express HER2 on a cellular surface.

Determining the In Vivo Effects of the Cytotoxic, Cell-Targeted Molecule “SLT-1A-FR Linked with αHER2-V_{H}H” Using Animal Models

Animal models are used to determine the in vivo effects of the cytotoxic molecule “SLT-1A-FR linked with αHER2-V_{H}H” on neoplastic cells. Various mice strains are used to test the effect of the cytotoxic molecule after intravenous administration on xenograft tumors in mice resulting from the injection into those mice of human neoplastic cells which express HER2 on their cell surfaces.

### Example 17

### Cytotoxic, Cell-Targeted Molecules, Each Comprising a Protease-Cleavage Resistant, Shiga Toxin Effector Region Fused to an Immunoglobulin-Type Binding Region Specific to a Major Histo-Compatibility Molecule Complexed with a Peptide from an Infectious Agent

In this example, the Shiga toxin effector polypeptide region is a protease resistant Shiga toxin effector polypeptide derived from the A subunit of Shiga toxin (StxA), such as, e.g., the Shiga toxin effector polypeptide comprising amino acids 2-252 of SEQ ID NO:32. An immunoglobulin-type binding region which binds a human, major Histo-Compatibility (MHC) molecule complexed with a specific peptide is obtained or designed from an antibody and/or immunoglobulin-type library screened using standard techniques known to the skilled worker (see Tohidkia M et al., J Drug Target 20: 195-208 (2012); de Marco A, Crit Rev Biotechnol 33: 40-8 (2013); Wen F, Zhao H, Methods Mol Biol 1061: 245-64 (2013)).

For example, human cells infected with malaria can present on their cell surfaces MHC class I molecules complexed with antigens from the P. falciparum apical membrane antigen-1 (AMA1), such as, e.g., the HLA-A complexed with the peptide TLDEMRHFY (see e.g. Lal A et al., Infect Immun 64: 1054-9 (1996); Sedegah M et al., Malar J 9: 241 (2010); Schwenk R et al., Malar J 12:376 (2013)). Similarly, human cells infected with tuberculosis can present on their cell surfaces MHC class I molecules complexed with antigens from M. tuberculosis factors, such as, e.g., CFP10, PE/PPE, Rv0288, Rv1886c, Rv3875, and TB10.4, (Axelsson-Robertson R et al., Immunology 129: 496-505 (2010); Axelsson-Robertson R et al., Clin Vaccine Immunol 18: 125-34 (2011); Wang M et al., Immunology 132: 482-91 (2011); Axelsson-Robertson R et al., PLoS One 8: e58309 (2013)).

Construction, Production, and Purification of the Cytotoxic, Cell-Targeted Molecule “SLT-1A-FR Linked with αMHC-Peptide”

The immunoglobulin-type binding region αMHC-peptide and protease resistant Shiga toxin effector polypeptide are linked together to form a cytotoxic, cell-targeted molecule. For example, a fusion protein is produced by expressing a polynucleotide encoding the SLT-1A-FR::αMHC-peptide protein wherein the binding regions binds a specific human HLA subtype MHC molecule complexed with an antigenic peptide from M. tuberculosis or P. falciparum. Expression of the SLT-1A-FR::αMHC-peptide cytotoxic molecule is accomplished using either bacterial and/or cell-free, protein translation systems as described in the previous examples. Binding regions specific for other HLA types complexed to malarial antigens or mycobacterium antigens are designed and tested to provide better coverage of human subpopulations.

Determining the In Vitro Characteristics of the Cytotoxic, Cell-Targeted Molecule “SLT-1A-FR Linked with αMHC-Peptide”

The binding characteristics of the cytotoxic molecule of this example for infected human cells is determined by a fluorescence-based, flow-cytometry assay known in the art. The Bₘₐₓ for “SLT-1A-FR linked with αMHC-peptide” to antigen-presenting cells is measured to be approximately 50,000-200,000 MFI with a K_{D} within the range of 0.01-100 nM, whereas there is no significant binding to negative control cells in this assay.

The ribosome inactivation abilities of the “SLT-1A-FR linked with αMHC-peptide” cytotoxic molecule is determined in a cell-free, in vitro protein translation as described above in the previous examples. The inhibitory effect of the cytotoxic molecule of this example on cell-free protein synthesis is significant. The IC₅₀ of “SLT-1A-FR linked with αMHC-peptide” on protein synthesis in this cell-free assay is approximately 0.1-100 pM.

Determining the Cytotoxicity of the Cytotoxic, Cell-Targeted Molecule “SLT-1A-FR Linked with αMHC-Peptide” Using a Cell-Kill Assay

The cytotoxicity characteristics of “SLT-1A-FR linked with αMHC-peptide” are determined by the general cell-kill assay as described above in the previous examples using infected cells and/or antigen presenting cells positive for specific MHC molecule-peptide complexes. In addition, the selective cytotoxicity characteristics of “SLT-1A-FR linked with αMHC-peptide” are determined by the same general cell-kill assay. The CD₅₀ of the cytotoxic molecule of this example is approximately 0.01-100 nM for MHC-peptide+ cells depending on the cell line. The CD₅₀ of the cytotoxic molecule is approximately 10-10,000 fold greater (less cytotoxic) for cells not expressing the same MHC-peptide on a cellular surface as compared to cells which do present a specifically targeted, MHC-peptide on a cellular surface.

Determining the In Vivo Effects of the Cytotoxic, Cell-Targeted Molecule “SLT-1A-FR Linked with αMHC-Peptide” Using Animal Models

The use of “SLT-1A-FR linked with αMHC-peptide” to inhibit plasmodium or mycobacterium infections is tested by administering “SLT-1A-FR linked with αMHC-peptide” to animal models of malarial infections, mycobacterium, sporozoite infections, and liver stage Plasmodium parasitic infections. This type of MHC-peptide complex-targeted therapeutic may be particularly useful in mycobacterium or plasmodium infected individuals who are also immuno-compromised, such as, e.g., asplenia, T-cell deficient, and/or HIV-infected patients.

### Example 18

### A Cytotoxic, Cell-Targeted Molecule Derived from the A Subunit of Shiga Toxin and the Antibody ΔHistoplasma-Antigen

In this example, the Shiga toxin effector region is derived from the A subunit of Shiga Toxin (StxA). An immunoglobulin-type binding region αhistoplasma-antigen is derived from a known antibody or an antibody generated, using techniques known in the art, to a Histoplasma capsulatum surface antigen (see e.g., H. capsulatum H antigen Deepe G, Durose G, Infect Immun 63: 3151-7 (1995) and the mAb H1C (Lopes L et al., Clin Vaccine Immunol 17: 1155-8 (2010); H. capsulatum, cell surface, histone-like protein H2B (Nosanchuk J et al., J Clin Invest 112: 1164-1175 (2003)).

Construction, Production, and Purification of the Cytotoxic Protein StxA::αHistoplasma-Antigen and/or StxA-FR::αHistoplasma-Antigen

The immunoglobulin-type binding region αhelminth-intestinal-antigen and Shiga toxin effector region, which is optionally a protease-cleavage resistant Shiga toxin effector region, are linked together to form a protein in which the immunoglobulin-type binding region is not located proximally to the protein's amino-terminus as compared to the Shiga toxin effector region. For example, a fusion protein is produced by expressing a polynucleotide encoding a Histoplasma-surface-antigen-binding protein fused to an amino-terminal, Shiga toxin effector region derived from StxA (e.g. the polypeptide represented by amino acids 1-251 of SEQ ID NO:2 or protease-cleavage resistant variant thereof). Expression of the StxA::αHistoplasma-antigen-binding protein and/or StxA-FR::αHistoplasma-antigen-binding protein is accomplished using either bacterial and/or cell-free, protein translation systems as described in the previous examples.

Determining the In Vitro Characteristics of the Cytotoxic, Cell-Targeted Molecule “StxA::αHistoplasma-Antigen” and/or “StxA-FR::αHistoplasma-Antigen”

The binding characteristics of the cytotoxic, cell-targeted molecule of this example is determined by a molecular binding assay known in the art using a purified recombinant target protein. The K_{D} for StxA::αHistoplasma-antigen and/or StxA-FR::αHistoplasma-antigen to target protein (e.g. a purified, recombinant, H. capsulatum surface antigen) is measured to be approximately 100 nM, whereas there is no significant binding to a negative control protein in this assay.

The ribosome inactivation abilities of the StxA::αHistoplasma-antigen and/or StxA-FR::αHistoplasma-antigen cytotoxic proteins are determined in a cell-free, in vitro protein translation as described above in the previous examples. The inhibitory effect of the cytotoxic, cell-targeted molecule of this example on cell-free protein synthesis is significant. The IC₅₀ of StxA::αHistoplasma-antigen and/or StxA-FR::αHistoplasma-antigen on protein synthesis in this cell-free assay is approximately 0.1-100 pM.

Determining the Anti-Fungal Activity of the Cytotoxic, Cell-Targeted Molecule StxA::αHistoplasma-Antigen and/or StxA-FR::αHistoplasma-Antigen Using Fungi

The fungicidal activity of StxA::αHistoplasma-antigen and/or StxA-FR::αHistoplasma-antigen to fungal cells is determined. Purified, StxA::αHistoplasma-antigen and/or StxA-FR::αHistoplasma-antigen is directly administered to fungal cultures in order to measure fungicidal activity (see e.g. Li R et al., Antimicrob Agents Chemother 44: 1734-6 (2000)). In addition, laboratory animals infected with fungi (e.g. H. capsulatum) and/or displaying histoplasmosis, systemic mycoses, and/or other H. capsulatum-related diseases are administered StxA::αHistoplasma-antigen and/or StxA-FR::αHistoplasma-antigen and monitored for reduction or elimination of fungal pathogens and/or associated symptoms of fungal infections (see e.g. Kobayashi G et al., Antimicrob Agents Chemother 34: 524-8 (1990)).

### Example 19

### Cell-Targeted Molecules Targeting Various Cell Types and for Uses Involving Various Diseases, Disorders, and Conditions

In this example, the Shiga toxin effector region is derived from the A subunit of Shiga-like Toxin 1 (SLT-1A), Shiga toxin (StxA), and/or Shiga-like Toxin 2 (SLT-2A). An immunoglobulin-type binding region is derived from the protein chosen from column 1 of Table 15 and which binds the extracellular target biomolecule indicated in column 2 of Table 15. The exemplary cell-targeted molecules of this example are created with amino-terminus and/or amino-terminus proximal, Shiga toxin effector regions (such as, e.g., using protease-cleavage resistant, Shiga toxin effector polypeptides or wild-type, Shiga toxin polypeptides) using techniques known in the art and optionally linked with a detection promoting agent(s). The exemplary cell-targeted molecules of this example are created and tested as described in the previous examples using cells expressing the appropriate extracellular target biomolecules. The exemplary cell-targeted molecules of this example may be used, e.g., to diagnose and treat diseases, conditions, and/or disorders indicated in column 3 of Table 15.

TABLE 15
Various Immunoglobulin-Type Binding Regions for Cell Targeting
of Cell-Targeted Molecules of the Present Invention

| Source of | Extracellular | |
|---|---|---|
| binding region | target | Application(s) |
| alemtuzumab | CD52 | B-cell cancers, such as lymphoma and |
| | | leukemia, and B-cell related immune |
| | | disorders, such as autoimmune disorders |
| basiliximab | CD25 | T-cell disorders, such as prevention of organ |
| | | transplant rejections, and some B-cell lineage |
| | | cancers |
| brentuximab | CD30 | hematological cancers, B-cell related |
| | | immune disorders, and T-cell related |
| | | immune disorders |
| catumaxomab | EpCAM | various cancers, such as ovarian cancer, |
| | | malignant ascites, gastric cancer |
| cetuximab | EGFR | various cancers, such as colorectal cancer |
| | | and head and neck cancer |
| daclizumab | CD25 | B-cell lineage cancers and T-cell disorders, |
| | | such as rejection of organ transplants |
| daratumumab | CD38 | hematological cancers, B-cell related |
| | | immune disorders, and T-cell related |
| | | immune disorders |
| dinutuximab | ganglioside | Various cancers, such as breast cancer, |
| | GD2 | myeloid cancers, and neuroblastoma |
| efalizumab | LFA-1 | autoimmune disorders, such as psoriasis |
| | (CD11a) | |
| ertumaxomab | HER2/neu | various cancers and tumors, such as breast |
| | | cancer and colorectal cancer |
| gemtuzumab | CD33 | myeloid cancer or immune disorder |
| ibritumomab | CD20 | B-cell cancers, such as lymphoma and |
| | | leukemia, and B-cell related immune |
| | | disorders, such as autoimmune disorders |
| ipilimumab | CD152 | T-cell related disorders and various cancers, |
| | | such as leukemia, melanoma |
| muromonab | CD3 | prevention of organ transplant rejections |
| natalizumab | integrin α4 | autoimmune disorders, such as multiple |
| | | sclerosis and Crohn's disease |
| obinutuzumab | CD20 | B-cell cancers, such as lymphoma and |
| | | leukemia, and B-cell related immune |
| | | disorders, such as autoimmune disorders |
| ocaratuzumab | CD20 | B-cell cancers, such as lymphoma and |
| | | leukemia, and B-cell related immune |
| | | disorders, such as autoimmune disorders |
| ocrelizumab | CD20 | B-cell cancers, such as lymphoma and |
| | | leukemia, and B-cell related immune |
| | | disorders, such as autoimmune disorders |
| ofatumumab | CD20 | B-cell cancers, such as lymphoma and |
| | | leukemia, and B-cell related immune |
| | | disorders, such as autoimmune disorders |
| palivizumab | F protein of | treat respiratory syncytial virus |
| | respiratory | |
| | syncytial virus | |
| panitumumab | EGFR | various cancers, such as colorectal cancer |
| | | and head and neck cancer |
| pertuzumab | HER2/neu | various cancers and tumors, such as breast |
| | | cancer and colorectal cancer |
| pro 140 | CCR5 | HIV infection and T-cell disorders |
| ramucirumab | VEGFR2 | various cancers and cancer related disorders, |
| | | such as solid tumors |
| rituximab | CD20 | B-cell cancers, such as lymphoma and |
| | | leukemia, and B-cell related immune |
| | | disorders, such as autoimmune disorders |
| tocilizumab | IL-6 receptor | autoimmune disorders, such as rheumatoid |
| or atlizumab | | arthritis |
| tositumomab | CD20 | B-cell cancers, such as lymphoma and |
| | | leukemia, and B-cell related immune |
| | | disorders, such as autoimmune disorders |
| trastuzumab | HER2/neu | various cancers and tumors, such as breast |
| | | cancer and colorectal cancer |
| ublituximab | CD20 | B-cell cancers, such as lymphoma and |
| | | leukemia, and B-cell related immune |
| | | disorders, such as autoimmune disorders |
| vedolizumab | integrin α4β7 | autoimmune disorders, such as Crohn's |
| | | disease and ulcerative colitis |
| CD20 binding | CD20 | B-cell cancers, such as lymphoma and |
| antibodies | | leukemia, and B-cell related immune |
| and scFv(s) | | disorders, such as autoimmune disorders (see |
| | | e.g. Geng S et al., Cell Mol Immunol 3: 439- |
| | | 43 (2006); Olafesn T et al., Protein Eng Des Sel |
| | | 23: 243-9 (2010)) |
| CD22 binding | CD22 | B-cell cancers or B-cell related immune |
| scFv(s) | | disorders (see e.g. Kawas S et al., MAbs 3: |
| | | 479-86 (2011)) |
| CD25 binding | CD25 | various cancers of the B-cell lineage and |
| scFv(s) | | immune disorders related to T-cells (see e.g. |
| | | Muramatsu H et al., Cancer Lett 225: 225-36 |
| | | (2005)) |
| CD30 binding | CD30 | B-cell cancers or B-cell/T-cell related |
| monoclonal | | immune disorders (see e.g. Klimka A et al., |
| antibody(s) | | Br J Cancer 83: 252-60 (2000)) |
| CD33 binding | CD33 | myeloid cancer or immune disorder (see e.g. |
| monoclonal | | Benedict C et al., J Immunol Methods 201: |
| antibody(s) | | 223-31 (1997)) |
| CD38 binding | CD38 | hematological cancers, B-cell related |
| immunoglobulin | | immune disorders, and T-cell related |
| domains | | immune disorders (see e.g. U.S. Pat. No. |
| | | 8,153,765) |
| CD40 binding | CD40 | various cancers and immune disorders (see |
| scFv(s) | | e.g. Ellmark P et al., Immunology 106: 456- |
| | | 63 (2002)) |
| CD52 binding | CD52 | B-cell cancers, such as lymphoma and |
| monoclonal | | leukemia, and B-cell related immune |
| antibody(s) | | disorders, such as autoimmune disorders (see |
| | | e.g. U.S. Pat. No. 7,910,104 B2) |
| CD56 binding | CD56 | immune disorders and various cancers, such |
| monoclonal | | as lung cancer, Merkel cell carcinoma, |
| antibody(s) | | myeloma (see e.g. Shin J et al., Hybridoma |
| | | 18: 521-7 (1999)) |
| CD79 binding | CD79 | B-cell cancers or B-cell related immune |
| monoclonal | | disorders (see e.g. Zhang L et al., |
| antibody(s) | | Ther Immunol 2: 191-202 (1995)) |
| CD133 binding | CD133 | various cancers, hematologic malignancies, |
| monoclonal | | and immune disorders (see e.g. Bidlingmaier |
| antibodies | | S et al., J Mol Med 86: 1025-32 (2008); |
| and scFv(s) | | Pavlon L et al., J Microsc 231: 374-83 |
| | | (2008); Rappa G et al., Stem Cells 26: 3008- |
| | | 17 (2008); Swaminathan S et al., J Immunol |
| | | Methods 361: 110-5 (2010); Wang J et al., |
| | | Hybridoma 29: 241-9 (2010); Zhu X et al., |
| | | Mol Cancer Ther 9: 2131-41 (2010); Xia J et |
| | | al., Sci Rep 3: 3320 (2013)) |
| CD248 binding | CD248 | various cancers, such as inhibiting |
| scFv(s) | | angiogenesis (see e.g. Zhao A et al., |
| | | J Immunol Methods 363: 221-32 (2011)) |
| EpCAM binding | EpCAM | various cancers, such as ovarian cancer, |
| monoclonal | | malignant ascites, gastric cancer (see e.g. |
| antibody(s) | | Schanzer J et al., J Immunother 29: 477-88 |
| | | (2006)) |
| PSMA binding | PSMA | prostate cancer (see e.g. Frigerio B et al., |
| monoclonal | | Eur J Cancer 49: 2223-32 (2013)) |
| antibody(s) | | |
| Eph-B2 binding | Eph-B2 | various cancers such as colorectal cancer and |
| monoclonal | | prostate cancer (see e.g. Abéngozar M et al., |
| antibody(s) | | Blood 119: 4565-76 (2012)) |
| Endoglin | Endoglin | various cancers, such as breast cancer and |
| binding | | colorectal cancers (see e.g. Völkel T et al., |
| monoclonal | | Biochim Biophys Res Acta 1663: 158-66 |
| antibody(s) | | (2004)) |
| FAP binding | FAP | various cancers, such as sarcomas and bone |
| monoclonal | | cancers (see e.g. Zhang J et al., FASEB J 27: |
| antibody(s) | | 581-9 (2013)) |
| CEA binding | CEA | various cancers, such as gastrointestinal |
| antibody(s) | | cancer, pancreatic cancer, lung cancer, and |
| and scFv(s) | | breast cancer (see e.g. Neumaier M et al., |
| | | Cancer Res 50: 2128-34 (1990); Pavoni E et |
| | | al., BMC Cancer 6: 4 (2006); Yazaki P et al., |
| | | Nucl Med Biol 35: 151-8 (2008); Zhao J et |
| | | al., Oncol Res 17: 217-22 (2008)) |
| CD24 binding | CD24 | various cancers, such as bladder cancer (see |
| monoclonal | | e.g. Kristiansen G et al., Lab Invest 90: 1102- |
| antibody(s) | | 16 (2010)) |
| LewisY | LewisY | various cancers, such as cervical cancer and |
| antigen | antigens | uterine cancer (see e.g. Power B et al., |
| binding | | Protein Sci 12: 734-47 (2003); monoclonal |
| scFv(s) | | antibody BR96 Feridani A et al., Cytometry |
| | | 71: 361-70 (2007)) |
| Broadly | Influenza | viral infections (see e.g. Prabakaran P et al., |
| neutralizing | surface | Front Microbiol 3: 277 (2012)) |
| antibodies | antigens (e.g. | |
| identified | hemaglutinins | |
| from patient | and matrix | |
| samples | protein 2) | |
| Broadly | Coronavirus | viral infections (see e.g. Prabakaran P et al., |
| neutralizing | surface | Front Microbiol 3: 277 (2012)) |
| antibodies | antigens | |
| identified | | |
| from patient | | |
| samples | | |
| Various | Filovirus | viral infections (see e.g. Olinger G et al., |
| antibodies | surface | Proc Natl Acad Sci USA 109: 18030-5 |
| | antigens (e.g. | (2012); Pettitt J et al., Sci Transl Med 5: |
| | VP35, VP40, | 199ra113 (2013); Stahelin R, Expert Opin |
| | and | Ther Targets 18: 115-20 (2014); Becquart P |
| | glycoprotein) | et al., PLoS One 9: e96360 (2014); Stahelin |
| | | R, Fron Microbiol 5: 300 (2014); Tran E et |
| | | al., J Virol 88: 10958-62 (2014); Murin C et |
| | | al., Proc Natl Acad Sci USA 111: 17182-7 |
| | | (2014)) |
| Broadly | Henipavirus | viral infections (see e.g. Prabakaran P et al., |
| neutralizing | surface | Front Microbiol 3: 277 (2012)) |
| antibodies | antigens | |
| identified | | |
| from patient | | |
| samples | | |
| Various | HIV surface | viral infections (see e.g. Kitidee K et al., |
| antibodies | antigens (e.g. | BMC Biotechnol 10: 80 (2010); Yu L, Guan |
| including | matrix | Y, Front Immunol 5: 250 (2014)) |
| broadly | protein | |
| neutralizing | Map17) | |
| antibodies | | |
| and scFvs | | |
| Broadly | Influenza | viral infections (see e.g. Prabakaran P et al., |
| neutralizing | surface | Front Microbiol 3: 277 (2012)) |
| antibodies | antigens (e.g. | |
| identified | hemaglutinins | |
| from patient | and matrix | |
| samples | protein 2) | |

While some embodiments of the invention have been described by way of illustration, it will be apparent that the invention may be put into practice with many modifications, variations and adaptations, and with the use of numerous equivalents or alternative solutions that are within the scope of persons skilled in the art, without departing from the spirit of the invention or exceeding the scope of the claims.

All publications, patents, and patent applications are herein incorporated by reference in their entirety to the same extent as if each individual publication, patent or patent application was specifically and individually indicated to be incorporated by reference in its entirety. The international patent application publications WO 2014/164680, WO 2014/164693, WO 2015/113005, WO 2015/113007, WO 2015/120058, WO 2015/138435, and WO 2015/138452 are each incorporated herein by reference in its entirety. The disclosure of international PCT patent applications PCT/US2015/019708 and PCT/US2015/035179 are each incorporated herein by reference in its entirety. The disclosures of U.S. provisional patent applications 61/951,110, 61/951,121, and 62/010,918 are each incorporated herein by reference in its entirety. The complete disclosures of all electronically available biological sequence information from GenBank (National Center for Biotechnology Information, U.S.) for amino acid and nucleotide sequences cited herein are each incorporated herein by reference in their entirety.

Sequence Listing

| ID Number | Text Description | Biological Sequence |
|---|---|---|
| SEQ ID NO: 1 | Shiga-like toxin 1 | KEFTLDFSTAKTYVDSLNVIRSA |
| | Subunit A (SLT-1A) | IGTPLQTISSGGTSLLMIDSGSGD |
| | | NLFAVDVRGIDPEEGRFNNLRLI |
| | | VERNNLYVTGFVNRTNNVFYRF |
| | | ADFSHVTFPGTTAVTLSGDSSYT |
| | | TLQRVAGISRTGMQINRHSLTTS |
| | | YLDLMSHSGTSLTQSVARAMLR |
| | | FVTVTAEALRFRQIQRGFRTTLD |
| | | DLSGRSYVMTAEDVDLTLNWG |
| | | RLSSVLPDYHGQDSVRVGRISFG |
| | | SINAILGSVALILNCHHHASRVA |
| | | RMASDEFPSMCPADGRVRGITH |
| | | NKILWDSSTLGAILMRRTISS |
| SEQ ID NO: 2 | Shiga toxin Subunit A | KEFTLDFSTAKTYVDSLNVIRSA |
| | (StxA) | IGTPLQTISSGGTSLLMIDSGTGD |
| | | NLFAVDVRGIDPEEGRFNNLRLI |
| | | VERNNLYVTGFVNRTNNVFYRF |
| | | ADFSHVTFPGTTAVTLSGDSSYT |
| | | TLQRVAGISRTGMQINRHSLTTS |
| | | YLDLMSHSGTSLTQSVARAMLR |
| | | FVTVTAEALRFRQIQRGFRTTLD |
| | | DLSGRSYVMTAEDVDLTLNWG |
| | | RLSSVLPDYHGQDSVRVGRISFG |
| | | SINAILGSVALILNCHHHASRVA |
| | | RMASDEFPSMCPADGRVRGITH |
| | | NKILWDSSTLGAILMRRTISS |
| SEQ ID NO: 3 | Shiga-like toxin 2 | DEFTVDFSSQKSYVDSLNSIRSAI |
| | Subunit A (SLT-2A) | STPLGNISQGGVSVSVINHVLGG |
| | | NYISLNVRGLDPYSERFNHLRLI |
| | | MERNNLYVAGFINTETNIFYRFS |
| | | DFSHISVPDVITVSMTTDSSYSSL |
| | | QRIADLERTGMQIGRHSLVGSY |
| | | LDLMEFRGRSMTRASSRAMLRF |
| | | VTVIAEALRFRQIQRGFRPALSE |
| | | ASPLYTMTAQDVDLTLNWGRIS |
| | | NVLPEYRGEEGVRIGRISFNSLS |
| | | AILGSVAVILNCHSTGSYSVRSV |
| | | SQKQKTECQIVGDRAAIKVNNV |
| | | LWEANTIAALLNRKPQDLTEPN |
| | | Q |
| SEQ ID NO: 4 | SLT-1A: : αCD38scFv | MKEFTLDFSTAKTYVDSLNVIRS |
| | variant 1 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPDIELT |
| | | QSPSSFSVSLGDRVTITCKASEDI |
| | | YNRLAWYQQKPGNAPRLLISGA |
| | | TSLETGVPSRFSGSGSGKDYTLSI |
| | | TSLQTEDVATYYCQQYWSTPTF |
| | | GGGTKLEIKGSTSGSGKPGSGEG |
| | | SKVQLQESGPSLVQPSQRLSITC |
| | | TVSGFSLISYGVHWVRQSPGKG |
| | | LEWLGVIWRGGSTDYNAAFMS |
| | | RLSITKDNSKSQVFFKMNSLQA |
| | | DDTAIYFCAKTLITTGYAMDYW |
| | | GQGTTVTVSS |
| SEQ ID NO: 5 | SLT-1A: : αCD38scFv | MKEFTLDFSTAKTYVDSLNVIRS |
| | variant 2 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPGILGF |
| | | VFTLDIELTQSPSSFSVSLGDRVT |
| | | ITCKASEDIYNRLAWYQQKPGN |
| | | APRLLISGATSLETGVPSRFSGSG |
| | | SGKDYTLSITSLQTEDVATYYCQ |
| | | QYWSTPTFGGGTKLEIKGSTSGS |
| | | GKPGSGEGSKVQLQESGPSLVQ |
| | | PSQRLSITCTVSGFSLISYGVHW |
| | | VRQSPGKGLEWLGVIWRGGSTD |
| | | YNAAFMSRLSITKDNSKSQVFF |
| | | KMNSLQADDTAIYFCAKTLITT |
| | | GYAMDYWGQGTTVTVSS |
| SEQ ID NO: 6 | SLT-1A: : αCD38scFv | MKEFTLDFSTAKTYVDSLNVIRS |
| | variant 3 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNSHHHASR |
| | | VAREFPKPSTPPGSSGGAPDIELT |
| | | QSPSSFSVSLGDRVTITCKASEDI |
| | | YNRLAWYQQKPGNAPRLLISGA |
| | | TSLETGVPSRFSGSGSGKDYTLSI |
| | | TSLQTEDVATYYCQQYWSTPTF |
| | | GGGTKLEIKGSTSGSGKPGSGEG |
| | | SKVQLQESGPSLVQPSQRLSITC |
| | | TVSGFSLISYGVHWVRQSPGKG |
| | | LEWLGVIWRGGSTDYNAAFMS |
| | | RLSITKDNSKSQVFFKMNSLQA |
| | | DDTAIYFCAKTLITTGYAMDYW |
| | | GQGTTVTVSS |
| SEQ ID NO: 7 | SLT-1A: : αCD38scFv | MKEFTLDFSTAKTYVDSLNVIRS |
| | variant 4 | AIGTPLQTISSGGTSLLMIDSGTG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNSHHHASR |
| | | VAREFPKPSTPPGSSGGAPGILGF |
| | | VFTLDIELTQSPSSFSVSLGDRVT |
| | | ITCKASEDIYNRLAWYQQKPGN |
| | | APRLLISGATSLETGVPSRFSGSG |
| | | SGKDYTLSITSLQTEDVATYYCQ |
| | | QYWSTPTFGGGTKLEIKGSTSGS |
| | | GKPGSGEGSKVQLQESGPSLVQ |
| | | PSQRLSITCTVSGFSLISYGVHW |
| | | VRQSPGKGLEWLGVIWRGGSTD |
| | | YNAAFMSRLSITKDNSKSQVFF |
| | | KMNSLQADDTAIYFCAKTLITT |
| | | GYAMDYWGQGTTVTVSS |
| SEQ ID NO: 8 | SLT-1A: : αHER2scFv | MKEFTLDFSTAKTYVDSLNVIRS |
| | variant 1 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPDIQM |
| | | TQSPSSLSASVGDRVTITCRASQ |
| | | DVNTAVAWYQQKPGKAPKLLI |
| | | YSASFLYSGVPSRFSGSRSGTDF |
| | | TLTISSLQPEDFATYYCQQHYTT |
| | | PPTFGQGTKVEIKRTGSTSGSGK |
| | | PGSGEGSEVQLVESGGGLVQPG |
| | | GSLRLSCAASGFNIKDTYIHWVR |
| | | QAPGKGLEWVARIYPTNGYTRY |
| | | ADSVKGRFTISADTSKNTAYLQ |
| | | MNSLRAEDTAVYYCSRWGGDG |
| | | FYAMDVWGQGTLVTVSS |
| SEQ ID NO: 9 | SLT-1A: : αHER2scFv | MKEFTLDFSTAKTYVDSLNVIRS |
| | variant 2 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPGILGF |
| | | VFTLDIQMTQSPSSLSASVGDRV |
| | | TITCRASQDVNTAVAWYQQKP |
| | | GKAPKLLIYSASFLYSGVPSRFS |
| | | GSRSGTDFTLTISSLQPEDFATY |
| | | YCQQHYTTPPTFGQGTKVEIKR |
| | | TGSTSGSGKPGSGEGSEVQLVES |
| | | GGGLVQPGGSLRLSCAASGFNI |
| | | KDTYIHWVRQAPGKGLEWVAR |
| | | IYPTNGYTRYADSVKGRFTISAD |
| | | TSKNTAYLQMNSLRAEDTAVY |
| | | YCSRWGGDGFYAMDVWGQGT |
| | | LVTVSS |
| SEQ ID NO: 10 | SLT-1A: : αHER2scFv | MKEFTLDFSTAKTYVDSLNVIRS |
| | variant 3 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNSHHHASR |
| | | VAREFPKPSTPPGSSGGAPDIQM |
| | | TQSPSSLSASVGDRVTITCRASQ |
| | | DVNTAVAWYQQKPGKAPKLLI |
| | | YSASFLYSGVPSRFSGSRSGTDF |
| | | TLTISSLQPEDFATYYCQQHYTT |
| | | PPTFGQGTKVEIKRTGSTSGSGK |
| | | PGSGEGSEVQLVESGGGLVQPG |
| | | GSLRLSCAASGFNIKDTYIHWVR |
| | | QAPGKGLEWVARIYPTNGYTRY |
| | | ADSVKGRFTISADTSKNTAYLQ |
| | | MNSLRAEDTAVYYCSRWGGDG |
| | | FYAMDVWGQGTLVTVSS |
| SEQ ID NO: 11 | SLT-1A: : αHER2scFv | MKEFTLDFSTAKTYVDSLNVIRS |
| | variant 4 | AIGTPLQTISSGGTSLLMIDSGTG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNSHHHASR |
| | | VAREFPKPSTPPGSSGGAPGILGF |
| | | VFTLDIQMTQSPSSLSASVGDRV |
| | | TITCRASQDVNTAVAWYQQKP |
| | | GKAPKLLIYSASFLYSGVPSRFS |
| | | GSRSGTDFTLTISSLQPEDFATY |
| | | YCQQHYTTPPTFGQGTKVEIKR |
| | | TGSTSGSGKPGSGEGSEVQLVES |
| | | GGGLVQPGGSLRLSCAASGFNI |
| | | KDTYIHWVRQAPGKGLEWVAR |
| | | IYPTNGYTRYADSVKGRFTISAD |
| | | TSKNTAYLQMNSLRAEDTAVY |
| | | YCSRWGGDGFYAMDVWGQGT |
| | | LVTVSS |
| SEQ ID NO: 12 | SLT-1A: : αCD19scFv | MKEFTLDFSTAKTYVDSLNVIRS |
| | variant 1 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPDIVM |
| | | TQAAPSIPVTPGESVSISCRSSKS |
| | | LLNSNGNTYLYWFLQRPGQSPQ |
| | | LLIYRMSNLASGVPDRFSGSGSG |
| | | TAFTLRISRVEAEDVGVYYCMQ |
| | | HLEYPFTFGAGTKLELKGSTSGS |
| | | GKPGSGEGSEVQLQQSGPELIKP |
| | | GASVKMSCKASGYTFTSYVMH |
| | | WVKQKPGQGLEWIGYINPYND |
| | | GTKYNEKFKGKATLTSDKSSST |
| | | AYMELSSLTSEDSAVYYCARGT |
| | | YYYGSRVFDYWGQGTTLTVSS |
| SEQ ID NO: 13 | SLT-1A: : αCD19scFv | MKEFTLDFSTAKTYVDSLNVIRS |
| | variant 2 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPGILGF |
| | | VFTLDIVMTQAAPSIPVTPGESV |
| | | SISCRSSKSLLNSNGNTYLYWFL |
| | | QRPGQSPQLLIYRMSNLASGVP |
| | | DRFSGSGSGTAFTLRISRVEAED |
| | | VGVYYCMQHLEYPFTFGAGTK |
| | | LELKGSTSGSGKPGSGEGSEVQL |
| | | QQSGPELIKPGASVKMSCKASG |
| | | YTFTSYVMHWVKQKPGQGLEW |
| | | IGYINPYNDGTKYNEKFKGKAT |
| | | LTSDKSSSTAYMELSSLTSEDSA |
| | | VYYCARGTYYYGSRVFDYWGQ |
| | | GTTLTVSS |
| SEQ ID NO: 14 | SLT-1A: : αCD19scFv | MKEFTLDFSTAKTYVDSLNVIRS |
| | variant 3 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNSHHHASR |
| | | VAREFPKPSTPPGSSGGAPDIVM |
| | | TQAAPSIPVTPGESVSISCRSSKS |
| | | LLNSNGNTYLYWFLQRPGQSPQ |
| | | LLIYRMSNLASGVPDRFSGSGSG |
| | | TAFTLRISRVEAEDVGVYYCMQ |
| | | HLEYPFTFGAGTKLELKGSTSGS |
| | | GKPGSGEGSEVQLQQSGPELIKP |
| | | GASVKMSCKASGYTFTSYVMH |
| | | WVKQKPGQGLEWIGYINPYND |
| | | GTKYNEKFKGKATLTSDKSSST |
| | | AYMELSSLTSEDSAVYYCARGT |
| | | YYYGSRVFDYWGQGTTLTVSS |
| SEQ ID NO: 15 | SLT-1A: : αCD19scFv | MKEFTLDFSTAKTYVDSLNVIRS |
| | variant 4 | AIGTPLQTISSGGTSLLMIDSGTG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNSHHHASR |
| | | VAREFPKPSTPPGSSGGAPGILGF |
| | | VFTLDIVMTQAAPSIPVTPGESV |
| | | SISCRSSKSLLNSNGNTYLYWFL |
| | | QRPGQSPQLLIYRMSNLASGVP |
| | | DRFSGSGSGTAFTLRISRVEAED |
| | | VGVYYCMQHLEYPFTFGAGTK |
| | | LELKGSTSGSGKPGSGEGSEVQL |
| | | QQSGPELIKPGASVKMSCKASG |
| | | YTFTSYVMHWVKQKPGQGLEW |
| | | IGYINPYNDGTKYNEKFKGKAT |
| | | LTSDKSSSTAYMELSSLTSEDSA |
| | | VYYCARGTYYYGSRVFDYWGQ |
| | | GTTLTVSS |
| SEQ ID NO: 16 | SLT-1A: : αCD74scFv | MKEFTLDFSTAKTYVDSLNVIRS |
| | variant 1 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPDIQL |
| | | TQSPLSLPVTLGQPASISCRSSQS |
| | | LVHRNGNTYLHWFQQRPGQSP |
| | | RLLIYTVSNRFSGVPDRFSGSGS |
| | | GTDFTLKISRVEAEDVGVYFCSQ |
| | | SSHVPPTFGAGTRLEIKGSTSGS |
| | | GKPGSGEGSTKGQVQLQQSGSE |
| | | LKKPGASVKVSCKASGYTFTNY |
| | | GVNWIKQAPGQGLQWMGWINP |
| | | NTGEPTFDDDFKGRFAFSLDTSV |
| | | STAYLQISSLKADDTAVYFCSRS |
| | | RGKNEAWFAYWGQGTLVTVSS |
| SEQ ID NO: 17 | SLT-1A: : αCD74scFv | MKEFTLDFSTAKTYVDSLNVIRS |
| | variant 2 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPGILGF |
| | | VFTLDIQLTQSPLSLPVTLGQPAS |
| | | ISCRSSQSLVHRNGNTYLHWFQ |
| | | QRPGQSPRLLIYTVSNRFSGVPD |
| | | RFSGSGSGTDFTLKISRVEAEDV |
| | | GVYFCSQSSHVPPTFGAGTRLEI |
| | | KGSTSGSGKPGSGEGSTKGQVQ |
| | | LQQSGSELKKPGASVKVSCKAS |
| | | GYTFTNYGVNWIKQAPGQGLQ |
| | | WMGWINPNTGEPTFDDDFKGRF |
| | | AFSLDTSVSTAYLQISSLKADDT |
| | | AVYFCSRSRGKNEAWFAYWGQ |
| | | GTLVTVSS |
| SEQ ID NO: 18 | SLT-1A: : αCD74scFv | MKEFTLDFSTAKTYVDSLNVIRS |
| | variant 3 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNSHHHASR |
| | | VAREFPKPSTPPGSSGGAPDIQL |
| | | TQSPLSLPVTLGQPASISCRSSQS |
| | | LVHRNGNTYLHWFQQRPGQSP |
| | | RLLIYTVSNRFSGVPDRFSGSGS |
| | | GTDFTLKISRVEAEDVGVYFCSQ |
| | | SSHVPPTFGAGTRLEIKGSTSGS |
| | | GKPGSGEGSTKGQVQLQQSGSE |
| | | LKKPGASVKVSCKASGYTFTNY |
| | | GVNWIKQAPGQGLQWMGWINP |
| | | NTGEPTFDDDFKGRFAFSLDTSV |
| | | STAYLQISSLKADDTAVYFCSRS |
| | | RGKNEAWFAYWGQGTLVTVSS |
| SEQ ID NO: 19 | SLT-1A: : αCD74scFv | MKEFTLDFSTAKTYVDSLNVIRS |
| | variant 4 | AIGTPLQTISSGGTSLLMIDSGTG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNSHHHASR |
| | | VAREFPKPSTPPGSSGGAPGILGF |
| | | VFTLDIQLTQSPLSLPVTLGQPAS |
| | | ISCRSSQSLVHRNGNTYLHWFQ |
| | | QRPGQSPRLLIYTVSNRFSGVPD |
| | | RFSGSGSGTDFTLKISRVEAEDV |
| | | GVYFCSQSSHVPPTFGAGTRLEI |
| | | KGSTSGSGKPGSGEGSTKGQVQ |
| | | LQQSGSELKKPGASVKVSCKAS |
| | | GYTFTNYGVNWIKQAPGQGLQ |
| | | WMGWINPNTGEPTFDDDFKGRF |
| | | AFSLDTSVSTAYLQISSLKADDT |
| | | AVYFCSRSRGKNEAWFAYWGQ |
| | | GTLVTVSS |
| SEQ ID NO: 20 | SLT-1A: : αHER2-V_{H}H | MKEFTLDFSTAKTYVDSLNVIRS |
| | variant 1 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VARAHHSEDPSSKAPKAPEVQL |
| | | VESGGGLVQAGGSLRLSCAASG |
| | | ITFSINTMGWYRQAPGKQRELV |
| | | ALISSIGDTYYADSVKGRFTISRD |
| | | NAKNTVYLQMNSLKPEDTAVY |
| | | YCKRFRTAAQGTDYWGQGTQV |
| | | TVSS |
| SEQ ID NO: 21 | SLT-1A: : αHER2-V_{H}H | MKEFTLDFSTAKTYVDSLNVIRS |
| | variant 2 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPEVQL |
| | | VESGGGLVQAGGSLRLSCAASG |
| | | ITFSINTMGWYRQAPGKQRELV |
| | | ALISSIGDTYYADSVKGRFTISRD |
| | | NAKNTVYLQMNSLKPEDTAVY |
| | | YCKRFRTAAQGTDYWGQGTQV |
| | | TVSS |
| SEQ ID NO: 22 | SLT-1A: : αHER2-V_{H}H | MKEFTLDFSTAKTYVDSLNVIRS |
| | variant 3 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VARAHHSEDPSSKAPKAPGILGF |
| | | VFTLEVQLVESGGGLVQAGGSL |
| | | RLSCAASGITFSINTMGWYRQAP |
| | | GKQRELVALISSIGDTYYADSVK |
| | | GRFTISRDNAKNTVYLQMNSLK |
| | | PEDTAVYYCKRFRTAAQGTDY |
| | | WGQGTQVTVSS |
| SEQ ID NO: 23 | SLT-1A: : αHER2-V_{H}H | MKEFTLDFSTAKTYVDSLNVIRS |
| | variant 4 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPGILGF |
| | | VFTLEVQLVESGGGLVQAGGSL |
| | | RLSCAASGITFSINTMGWYRQAP |
| | | GKQRELVALISSIGDTYYADSVK |
| | | GRFTISRDNAKNTVYLQMNSLK |
| | | PEDTAVYYCKRFRTAAQGTDY |
| | | WGQGTQVTVSS |
| SEQ ID NO: 24 | StxA: : αHER2-V_{H}H | MKEFTLDFSTAKTYVDSLNVIRS |
| | variant 1 | AIGTPLQTISSGGTSLLMIDSGTG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VARAHHSEDPSSKAPKAPEVQL |
| | | VESGGGLVQAGGSLRLSCAASG |
| | | ITFSINTMGWYRQAPGKQRELV |
| | | ALISSIGDTYYADSVKGRFTISRD |
| | | NAKNTVYLQMNSLKPEDTAVY |
| | | YCKRFRTAAQGTDYWGQGTQV |
| | | TVSS |
| SEQ ID NO: 25 | StxA: : αHER2- V_{H}H | MKEFTLDFSTAKTYVDSLNVIRS |
| | variant 2 | AIGTPLQTISSGGTSLLMIDSGTG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPEVQL |
| | | VESGGGLVQAGGSLRLSCAASG |
| | | ITFSINTMGWYRQAPGKQRELV |
| | | ALISSIGDTYYADSVKGRFTISRD |
| | | NAKNTVYLQMNSLKPEDTAVY |
| | | YCKRFRTAAQGTDYWGQGTQV |
| | | TVSS |
| SEQ ID NO: 26 | StxA: : αHER2-V_{H}H | MKEFTLDFSTAKTYVDSLNVIRS |
| | variant 3 | AIGTPLQTISSGGTSLLMIDSGTG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNSHHHASR |
| | | VARAHHSEDPSSKAPKAPGILGF |
| | | VFTLEVQLVESGGGLVQAGGSL |
| | | RLSCAASGITFSINTMGWYRQAP |
| | | GKQRELVALISSIGDTYYADSVK |
| | | GRFTISRDNAKNTVYLQMNSLK |
| | | PEDTAVYYCKRFRTAAQGTDY |
| | | WGQGTQVTVSS |
| SEQ ID NO: 27 | StxA: : αHER2-V_{H}H | MKEFTLDFSTAKTYVDSLNVIRS |
| | variant 4 | AIGTPLQTISSGGTSLLMIDSGTG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNSHHHASR |
| | | VAREFPKPSTPPGSSGGAPGILGF |
| | | VFTLEVQLVESGGGLVQAGGSL |
| | | RLSCAASGITFSINTMGWYRQAP |
| | | GKQRELVALISSIGDTYYADSVK |
| | | GRFTISRDNAKNTVYLQMNSLK |
| | | PEDTAVYYCKRFRTAAQGTDY |
| | | WGQGTQVTVSS |
| SEQ ID NO: 28 | SLT-1A: : αCD20-FN3 | MKEFTLDFSTAKTYVDSLNVIRS |
| | variant 1 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPASVS |
| | | DVPRDLEVVAATPTSLLISWCR |
| | | QRCADSYRITYGETGGNSPVQE |
| | | FTVPGSWKTATISGLKPGVDYTI |
| | | TVYVVTHYYGWDRYSHPISINY |
| | | RTGS |
| SEQ ID NO: 29 | SLT-1A: : αCD20-FN3 | MKEFTLDFSTAKTYVDSLNVIRS |
| | variant 2 | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPGILGF |
| | | VFTLASVSDVPRDLEVVAATPTS |
| | | LLISWCRQRCADSYRITYGETGG |
| | | NSPVQEFTVPGSWKTATISGLKP |
| | | GVDYTITVYVVTHYYGWDRYS |
| | | HPISINYRTGS |
| SEQ ID NO: 30 | StxA: : αCD20-FN3 | MKEFTLDFSTAKTYVDSLNVIRS |
| | variant 1 | AIGTPLQTISSGGTSLLMIDSGTG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNSHHHASR |
| | | VAREFPKPSTPPGSSGGAPASVS |
| | | DVPRDLEVVAATPTSLLISWCR |
| | | QRCADSYRITYGETGGNSPVQE |
| | | FTVPGSWKTATISGLKPGVDYTI |
| | | TVYVVTHYYGWDRYSHPISINY |
| | | RTGS |
| SEQ ID NO: 31 | StxA: : αCD20-FN3 | MKEFTLDFSTAKTYVDSLNVIRS |
| | variant 2 | AIGTPLQTISSGGTSLLMIDSGTG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHASR |
| | | VAREFPKPSTPPGSSGGAPGILGF |
| | | VFTLASVSDVPRDLEVVAATPTS |
| | | LLISWCRQRCADSYRITYGETGG |
| | | NSPVQEFTVPGSWKTATISGLKP |
| | | GVDYTITVYVVTHYYGWDRYS |
| | | HPISINYRTGS |
| SEQ ID NO: 32 | SLT-1A- | MKEFTLDFSTAKTYVDSLNVIRS |
| | FR: : aHER2scFv | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHAS |
| | | AVAAEFPKPSTPPGSSGGAPDIQ |
| | | MTQSPSSLSASVGDRVTITCRAS |
| | | QDVNTAVAWYQQKPGKAPKLL |
| | | IYSASFLYSGVPSRFSGSRSGTDF |
| | | TLTISSLQPEDFATYYCQQHYTT |
| | | PPTFGQGTKVEIKRTGSTSGSGK |
| | | PGSGEGSEVQLVESGGGLVQPG |
| | | GSLRLSCAASGFNIKDTYIHWVR |
| | | QAPGKGLEWVARIYPTNGYTRY |
| | | ADSVKGRFTISADTSKNTAYLQ |
| | | MNSLRAEDTAVYYCSRWGGDG |
| | | FYAMDVWGQGTLVTVSS |
| SEQ ID NO: 33 | SLT-1A- | MKEFTLDFSTAKTYVDSLNVIRS |
| | FR: : αCD19scFv | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHAS |
| | | AVAAEFPKPSTPPGSSGGAPDIV |
| | | MTQAAPSIPVTPGESVSISCRSSK |
| | | SLLNSNGNTYLYWFLQRPGQSP |
| | | QLLIYRMSNLASGVPDRFSGSGS |
| | | GTAFTLRISRVEAEDVGVYYCM |
| | | QHLEYPFTFGAGTKLELKGSTSG |
| | | SGKPGSGEGSEVQLQQSGPELIK |
| | | PGASVKMSCKASGYTFTSYVM |
| | | HWVKQKPGQGLEWIGYINPYN |
| | | DGTKYNEKFKGKATLTSDKSSS |
| | | TAYMELSSLTSEDSAVYYCARG |
| | | TYYYGSRVFDYWGQGTTLTVSS |
| SEQ ID NO: 34 | SLT-1A- | MKEFTLDFSTAKTYVDSLNVIRS |
| | FR: : αCD74scFv | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAM |
| | | LRFVTVTAEALRFRQIQRGFRTT |
| | | LDDLSGRSYVMTAEDVDLTLN |
| | | WGRLSSVLPDYHGQDSVRVGRI |
| | | SFGSINAILGSVALILNCHHHAS |
| | | AVAAEFPKPSTPPGSSGGAPDIQ |
| | | LTQSPLSLPVTLGQPASISCRSSQ |
| | | SLVHRNGNTYLHWFQQRPGQSP |
| | | RLLIYTVSNRFSGVPDRFSGSGS |
| | | GTDFTLKISRVEAEDVGVYFCSQ |
| | | SSHVPPTFGAGTRLEIKGSTSGS |
| | | GKPGSGEGSTKGQVQLQQSGSE |
| | | LKKPGASVKVSCKASGYTFTNY |
| | | GVNWIKQAPGQGLQWMGWINP |
| | | NTGEPTFDDDFKGRFAFSLDTSV |
| | | STAYLQISSLKADDTAVYFCSRS |
| | | RGKNEAWFAYWGQGTLVTVSS |
| SEQ ID NO: 35 | SLT-1A-FR: : HER2 | MKEFTLDFSTAKTYVDSLNVIRS |
| | binding region | AIGTPLQTISSGGTSLLMIDSGSG |
| | | DNLFAVDVRGIDPEEGRFNNLR |
| | | LIVERNNLYVTGFVNRTNNVFY |
| | | RFADFSHVTFPGTTAVTLSGDSS |
| | | YTTLQRVAGISRTGMQINRHSLT |
| | | TSYLDLMSHSGTSLTQSVARAE |
| | | FPKPSTPPGSSGGAPRGSHHMLR |
| | | FVTVTAEALRFRQIQRGFRTTLD |
| | | DLSGRSYVMTAEDVDLTLNWG |
| | | RLSSVLPDYHGQDSVRVGRISFG |
| | | SINAILGSVALILNCHHHASAVA |
| | | AHHHHGSDLGKKLLEAARAGQ |
| | | DDEVRILMANGADVNAKDEYG |
| | | LTPLYLATAHGHLEIVEVLLKN |
| | | GADVNAVDAIGFTPLHLAAFIG |
| | | HLEIAEVLLKHGADVNAQDKFG |
| | | KTAFDISIGNGNEDLAEILQKLN |

The invention is claimed as follows:

1. A cell-targeted molecule having an amino-terminus and comprising
a) a binding region comprising a peptide or polypeptide and capable of specifically binding at least one extracellular target biomolecule and
b) a Shiga toxin effector polypeptide region;
wherein the Shiga toxin effector polypeptide region is positioned at or proximal to the amino-terminus of the cell-targeted molecule.

2. The cell-targeted molecule of claim 1, wherein the cell-targeted molecule is capable when introduced to cells of exhibiting cytotoxicity that is greater than that of a second cell-targeted molecule having an amino-terminus and comprising the binding region and the Shiga toxin effector polypeptide region which is not positioned at or proximal to the amino-terminus of the second cell-targeted molecule.

3. The cell-targeted molecule of claim 2, wherein the binding region is linked to the cell-targeted molecule at a position carboxy-terminal to the Shiga toxin effector polypeptide region.

4. The cell-targeted molecule of claim 3, wherein the binding region comprises an immunoglobulin-type binding region comprising a polypeptide selected from the group consisting of:
single-domain antibody fragment, single-chain variable fragment, antibody variable fragment, complementary determining region 3 fragment, constrained FR3-CDR3-FR4 polypeptide, Fd fragment, antigen-binding fragment, fibronectin-derived 10^{th} fibronectin type III domain, tenascin type III domain, ankyrin repeat motif domain, low-density-lipoprotein-receptor-derived A-domain, lipocalin, Kunitz domain, Protein-A-derived Z domain, gamma-B crystalline-derived domain, ubiquitin-derived domain, Sac7d-derived polypeptide, Fyn-derived SH2 domain, miniprotein, C-type lectin-like domain scaffold, engineered antibody mimic, and any genetically manipulated counterparts of any of the foregoing which retain binding functionality.

5. The cell-targeted molecule of claim 4, whereby administration of the cell-targeted molecule to a first population of cells whose members are physically coupled to extracellular target biomolecules of the immunoglobulin-type binding region of the cell-targeted molecule, and a second population of cells whose members are not physically coupled to any extracellular target biomolecule of the immunoglobulin-type binding region, the cytotoxic effect of the cell-targeted molecule to members of said first population of cells relative to members of said second population of cells is at least 3-fold greater.

6. The cell-targeted molecule of claim 5, wherein the Shiga toxin effector polypeptide region comprises or consists essentially of a polypeptide represented by the amino acid sequence selected from the group consisting of:
i) amino acids 75 to 251 of SEQ ID NO:1, SEQ ID NO:2, or SEQ ID NO:3;
ii) amino acids 1 to 241 of SEQ ID NO:1, SEQ ID NO:2, or SEQ ID NO:3; and
iii) amino acids 1 to 251 of SEQ ID NO:1, SEQ ID NO:2, or SEQ ID NO:3.

7. The cell-targeted molecule of claim 5, wherein the immunoglobulin-type binding region is capable of binding to an extracellular target biomolecule selected from the group consisting of:
CD20, CD22, CD40, CD74, CD79, CD25, CD30, HER2/neu/ErbB2, EGFR, EpCAM, EphB2, prostate-specific membrane antigen, Cripto, CDCP1, endoglin, fibroblast activated protein, Lewis-Y, CD19, CD21, CS1/SLAMF7, CD33, CD52, CD133, CEA, gpA33, mucin, TAG-72, tyrosine-protein kinase transmembrane receptor, carbonic anhydrase IX, folate binding protein, ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANK, FAP, tenascin, CD64, mesothelin, BRCA1, MART-1/MelanA, gp100, tyrosinase, TRP-1, TRP-2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen, prostate specific antigen, prostate stem cell antigen, human aspartyl (asparaginyl) beta-hydroxylase, EphA2, HER3/ErbB-3, MUC1, MART-1/MelanA, gp100, tyrosinase associated antigen, HPV-E7, Epstein-Barr virus antigen, Bcr-Abl, alpha-fetoprotein antigen, 17-A1, bladder tumor antigen, CD38, CD15, CD23, CD53, CD88, CD129, CD183, CD191, CD193, CD244, CD294, CD305, C3AR, FceRIa, galectin-9, mrp-14, PD-L1, Siglec-8, Siglec-10, CD49d, CD13, CD44, CD54, CD63, CD69, CD123, TLR4, FceRIa, IgE, CD107a, CD203c, CD14, CD68, CD80, CD86, CD105, CD115, F4/80, ILT-3, galectin-3, CD11a-c, GITRL, MHC class I molecule, MHC class II molecule, CD284, CD107-Mac3, CD195, HLA-DR, CD16/32, CD282, and any immunogenic fragment of any of the foregoing.

8. The cell-targeted molecule of claim 7, wherein the immunoglobulin-type binding region comprises or consists essentially of the polypeptide sequence of amino acids 269-508 of any one of SEQ ID NOs: 4, 8, 12, or 16.

9. The cell-targeted molecule of claim 8, which comprises or consists essentially of the polypeptide shown in any one of SEQ ID NOs: 4-31.

10. The cell-targeted molecule of any one of claims 3-8, wherein the Shiga toxin effector polypeptide region comprises
a) a Shiga toxin A1 fragment region having a carboxy-terminus and
b) a disrupted furin-cleavage motif at the carboxy-terminus of the Shiga toxin A1 fragment region.

11. The cell-targeted molecule of claim 10, wherein the cell-targeted molecule is capable of exhibiting improved, in vivo tolerability compared to in vivo tolerability of a second cell-targeted molecule comprising the binding region and a second, Shiga toxin effector polypeptide region consisting of a wild-type, Shiga toxin A1 fragment, wherein the binding region is linked to the second cell targeted molecule carboxy-terminal to the wild-type, Shiga toxin A1 fragment.

12. The cell-targeted molecule of claim 10 or claim 11, wherein the cell-targeted molecule is capable of exhibiting cytotoxicity equivalent to a second cell-targeted molecule comprising the binding region and a second, Shiga toxin effector polypeptide region consisting of a wild-type, Shiga toxin A1 fragment, wherein the binding region is linked to the second cell-targeted molecule carboxy-terminal to the wild-type, Shiga toxin A1 fragment.

13. A cell-targeted molecule comprising
a) a binding region comprising one or more polypeptides and capable of specifically binding at least one extracellular target biomolecule,
b) a Shiga toxin effector polypeptide region having an amino-terminus and comprising a Shiga toxin A1 fragment region having a carboxy-terminus, and
c) a disrupted furin-cleavage motif at the carboxy-terminus of the A1 fragment region;
wherein the binding region is positioned within the cell-targeted molecule carboxy-terminal to the Shiga toxin A1 fragment region; and
wherein the cell-targeted molecule is capable when introduced to cells of exhibiting cytotoxicity that is in a range of from 0.1-fold to 1.5-fold of cytotoxicity exhibited by a second cell-targeted molecule comprising the binding region and a second, Shiga toxin effector polypeptide region consisting of a wild-type, Shiga toxin A1 fragment, and wherein the binding region is positioned within the second cell targeted molecule carboxy-terminal to the wild-type, Shiga toxin A1 fragment.

14. The cell-targeted molecule of claim 13, wherein the binding region comprises an immunoglobulin-type binding region comprising a polypeptide selected from the group consisting of:
single-domain antibody fragment, single-chain variable fragment, antibody variable fragment, complementary determining region 3 fragment, constrained FR3-CDR3-FR4 polypeptide, Fd fragment, antigen-binding fragment, fibronectin-derived 10^{th} fibronectin type III domain, tenascin type III domain, ankyrin repeat motif domain, low-density-lipoprotein-receptor-derived A-domain, lipocalin, Kunitz domain, Protein-A-derived Z domain, gamma-B crystalline-derived domain, ubiquitin-derived domain, Sac7d-derived polypeptide, Fyn-derived SH2 domain, miniprotein, C-type lectin-like domain scaffold, engineered antibody mimic, and any genetically manipulated counterparts of any of the foregoing which retain binding functionality.

15. The cell-targeted molecule of claim 14, whereby administration of the cell-targeted molecule to a first population of cells whose members are physically coupled to extracellular target biomolecules of the immunoglobulin-type binding region of the cell-targeted molecule, and a second population of cells whose members are not physically coupled to any extracellular target biomolecule of the immunoglobulin-type binding region, the cytotoxic effect of the cell-targeted molecule to members of said first population of cells relative to members of said second population of cells is at least 3-fold greater.

16. The cell-targeted molecule of claim 14, wherein the Shiga toxin effector polypeptide region comprises or consists essentially of a polypeptide represented by the amino acid sequence selected from the group consisting of:
1) amino acids 75 to 246 of SEQ ID NO:3;
2) amino acids 75 to 247 of SEQ ID NO:1 or SEQ ID NO:2;
3) amino acids 1 to 241 of SEQ ID NO:1, SEQ ID NO:2, or SEQ ID NO:3;
4) amino acids 1 to 246 of SEQ ID NO:3; and
5) amino acids 1 to 247 of SEQ ID NO:1 or SEQ ID NO:2.

17. The cell-targeted molecule of claim 16, wherein the immunoglobulin-type binding region is capable of binding to an extracellular target biomolecule selected from the group consisting of:
CD20, CD22, CD40, CD74, CD79, CD25, CD30, HER2/neu/ErbB2, EGFR, EpCAM, EphB2, prostate-specific membrane antigen, Cripto, CDCP1, endoglin, fibroblast activated protein, Lewis-Y, CD19, CD21, CS1/SLAMF7, CD33, CD52, CD133, CEA, gpA33, mucin, TAG-72, tyrosine-protein kinase transmembrane receptor, carbonic anhydrase IX, folate binding protein, ganglioside GD2, ganglioside GD3, ganglioside GM2, ganglioside Lewis-Y2, VEGFR, Alpha Vbeta3, Alpha5beta1, ErbB1/EGFR, Erb3, c-MET, IGF1R, EphA3, TRAIL-R1, TRAIL-R2, RANK, FAP, tenascin, CD64, mesothelin, BRCA1, MART-1/MelanA, gp100, tyrosinase, TRP-1, TRP-2, MAGE-1, MAGE-3, GAGE-1/2, BAGE, RAGE, NY-ESO-1, CDK-4, beta-catenin, MUM-1, caspase-8, KIAA0205, HPVE6, SART-1, PRAME, carcinoembryonic antigen, prostate specific antigen, prostate stem cell antigen, human aspartyl (asparaginyl) beta-hydroxylase, EphA2, HER3/ErbB-3, MUC1, MART-1/MelanA, gp100, tyrosinase associated antigen, HPV-E7, Epstein-Barr virus antigen, Bcr-Abl, alpha-fetoprotein antigen, 17-A1, bladder tumor antigen, CD38, CD15, CD23, CD53, CD88, CD129, CD183, CD191, CD193, CD244, CD294, CD305, C3AR, FceRIa, galectin-9, mrp-14, PD-L1, Siglec-8, Siglec-10, CD49d, CD13, CD44, CD54, CD63, CD69, CD123, TLR4, FceRIa, IgE, CD107a, CD203c, CD14, CD68, CD80, CD86, CD105, CD115, F4/80, ILT-3, galectin-3, CD11a-c, GITRL, MHC class I molecule, MHC class II molecule, CD284, CD107-Mac3, CD195, HLA-DR, CD16/32, CD282, and any immunogenic fragment of any of the foregoing.

18. The cell-targeted molecule of any one of claims 13-17, wherein the cell-targeted molecule is capable when introduced to cells of exhibiting a cytotoxicity that is greater than that of a third cell-targeted molecule having an amino-terminus, and comprising the binding region and the Shiga toxin effector polypeptide region which is not positioned at or proximal to the amino-terminus of the third cell-targeted molecule.

19. The cell-targeted molecule of claim 17, wherein the immunoglobulin-type binding region comprises or consists essentially of the polypeptide sequence represented by amino acids 269-508 of any one of SEQ ID NOs: 4, 8, 12, or 16.

20. The cell-targeted molecule of claim 17, which comprises or consists essentially of the polypeptide shown in any one of SEQ ID NOs: 32-35.

21. The cell-targeted molecule of any one of claims 13-17, wherein the cell-targeted molecule is capable of exhibiting improved, in vivo tolerability compared to in vivo tolerability of the second cell-targeted molecule.

22. A pharmaceutical composition comprising the cell-targeted molecule of any one of claims 1-21 and at least one pharmaceutically acceptable excipient or carrier.

23. A diagnostic composition comprising
the cell-targeted molecule of any one of claims 1-21 and
a detection promoting agent.

24. A polynucleotide capable of encoding the cell-targeted molecule of any one of claims 1-21 or a complement thereof, or a fragment of any of the foregoing.

25. An expression vector comprising the polynucleotide of claim 24.

26. A host cell comprising any one of the polynucleotides or expression vectors of claims 24-25.

27. A method of conferring greater cytotoxicity to a protein which comprises
a) an immunoglobulin-type binding region comprising one or more polypeptides and capable of specifically binding at least one extracellular target biomolecule, and
b) a Shiga toxin effector polypeptide with an amino-terminus;
the method comprising the step of arranging the amino-terminus of the Shiga toxin effector polypeptide more proximally to an amino-terminus of a polypeptide component of the protein.

28. A method of killing a cell, the method comprising the step of contacting the cell with the cell-targeted molecule of any one of claims 1-21 or the pharmaceutical composition of claim 22.

29. The method of claim 28, wherein the contacting occurs in vitro.

30. The method of claim 28, wherein the contacting occurs in vivo.

31. A method of treating a disease, disorder, or condition in a patient, the method comprising the step of administering to a patient in need thereof a therapeutically effective amount of the cell-targeted molecule of any one of claims 1-21 or the pharmaceutical composition of claim 22.

32. The method of claim 31, wherein the disease, disorder, or condition is selected from the group consisting of: cancer, tumor, immune disorder, and microbial infection.

33. The method of claim 32, wherein the cancer selected from the group consisting of:
bone cancer, breast cancer, central/peripheral nervous system cancer, gastrointestinal cancer, germ cell cancer, glandular cancer, head-neck cancer, hematological cancer, kidney-urinary tract cancer, liver cancer, lung/pleura cancer, prostate cancer, sarcoma, skin cancer, and uterine cancer; or
wherein the immune disorder is associated with a disease selected from the group consisting of:
amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriasis, psoriatic arthritis, rheumatoid arthritis, scleroderma, septic shock, Sjorgren's syndrome, ulcerative colitis, and vasculitis.

34. A method of detecting a cell, the method comprising the steps of:
contacting the cell with the diagnostic composition of claim 23 and
detecting the presence of the diagnostic composition.

35. A kit comprising the composition of matter of any one of claims 1-26 and an additional reagent and/or pharmaceutical delivery device.


SEQUENCE LISTING (35 sequences)

SEQ ID NO: 1 - protein, 293 aa, Escherichia coli
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 2 - protein, 293 aa, Shigella dysenteriae
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 3 - protein, 297 aa, Escherichia coli
DEFTVDFSSQKSYVDSLNSIRSAISTPLGNISQGGVSVSVINHVLGGNYISLNVRGLDPYSERFNHLRLIMERNNLYVAGFINTETNIFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQDLTEPNQ

SEQ ID NO: 4 - protein, 508 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPDIELTQSPSSFSVSLGDRVTITCKASEDIYNRLAWYQQKPGNAPRLLISGATSLETGVPSRFSGSGSGKDYTLSITSLQTEDVATYYCQQYWSTPTFGGGTKLEIKGSTSGSGKPGSGEGSKVQLQESGPSLVQPSQRLSITCTVSGFSLISYGVHWVRQSPGKGLEWLGVIWRGGSTDYNAAFMSRLSITKDNSKSQVFFKMNSLQADDTAIYFCAKTLITTGYAMDYWGQGTTVTVSS

SEQ ID NO: 5 - protein, 517 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLDIELTQSPSSFSVSLGDRVTITCKASEDIYNRLAWYQQKPGNAPRLLISGATSLETGVPSRFSGSGSGKDYTLSITSLQTEDVATYYCQQYWSTPTFGGGTKLEIKGSTSGSGKPGSGEGSKVQLQESGPSLVQPSQRLSITCTVSGFSLISYGVHWVRQSPGKGLEWLGVIWRGGSTDYNAAFMSRLSITKDNSKSQVFFKMNSLQADDTAIYFCAKTLITTGYAMDYWGQGTTVTVSS

SEQ ID NO: 6 - protein, 508 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPDIELTQSPSSFSVSLGDRVTITCKASEDIYNRLAWYQQKPGNAPRLLISGATSLETGVPSRFSGSGSGKDYTLSITSLQTEDVATYYCQQYWSTPTFGGGTKLEIKGSTSGSGKPGSGEGSKVQLQESGPSLVQPSQRLSITCTVSGFSLISYGVHWVRQSPGKGLEWLGVIWRGGSTDYNAAFMSRLSITKDNSKSQVFFKMNSLQADDTAIYFCAKTLITTGYAMDYWGQGTTVTVSS

SEQ ID NO: 7 - protein, 517 aa, Artificial Sequence
  Description of Artificial Sequence Synthetic polypeptide
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPGILGFVFTLDIELTQSPSSFSVSLGDRVTITCKASEDIYNRLAWYQQKPGNAPRLLISGATSLETGVPSRFSGSGSGKDYTLSITSLQTEDVATYYCQQYWSTPTFGGGTKLEIKGSTSGSGKPGSGEGSKVQLQESGPSLVQPSQRLSITCTVSGFSLISYGVHWVRQSPGKGLEWLGVIWRGGSTDYNAAFMSRLSITKDNSKSQVFFKMNSLQADDTAIYFCAKTLITTGYAMDYWGQGTTVTVSS

SEQ ID NO: 8 - protein, 512 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 9 - protein, 521 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 10 - protein, 512 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 11 - protein, 521 aa, Artificial Sequence
  Description of Artificial Sequence Synthetic polypeptide
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPGILGFVFTLDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 12 - protein, 516 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKGSTSGSGKPGSGEGSEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSS

SEQ ID NO: 13 - protein, 525 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKGSTSGSGKPGSGEGSEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSS

SEQ ID NO: 14 - protein, 516 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKGSTSGSGKPGSGEGSEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSS

SEQ ID NO: 15 - protein, 525 aa, Artificial Sequence
  Description of Artificial Sequence Synthetic polypeptide
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPGILGFVFTLDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKGSTSGSGKPGSGEGSEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSS

SEQ ID NO: 16 - protein, 518 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPDIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKGSTSGSGKPGSGEGSTKGQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 17 - protein, 527 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLDIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKGSTSGSGKPGSGEGSTKGQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 18 - protein, 518 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPDIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKGSTSGSGKPGSGEGSTKGQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 19 - protein, 527 aa, Artificial Sequence
  Description of Artificial Sequence Synthetic polypeptide
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPGILGFVFTLDIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKGSTSGSGKPGSGEGSTKGQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 20 - protein, 385 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARAHHSEDPSSKAPKAPEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 21 - protein, 386 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 22 - protein, 394 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARAHHSEDPSSKAPKAPGILGFVFTLEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 23 - protein, 395 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 24 - protein, 385 aa, Artificial Sequence
  Description of Artificial Sequence Synthetic polypeptide
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARAHHSEDPSSKAPKAPEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 25 - protein, 386 aa, Artificial Sequence
  Description of Artificial Sequence Synthetic polypeptide
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 26 - protein, 394 aa, Artificial Sequence
  Description of Artificial Sequence Synthetic polypeptide
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVARAHHSEDPSSKAPKAPGILGFVFTLEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 27 - protein, 395 aa, Artificial Sequence
  Description of Artificial Sequence Synthetic polypeptide
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPGILGFVFTLEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 28 - protein, 365 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPASVSDVPRDLEVVAATPTSLLISWCRQRCADSYRITYGETGGNSPVQEFTVPGSWKTATISGLKPGVDYTITVYVVTHYYGWDRYSHPISINYRTGS

SEQ ID NO: 29 - protein, 374 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLASVSDVPRDLEVVAATPTSLLISWCRQRCADSYRITYGETGGNSPVQEFTVPGSWKTATISGLKPGVDYTITVYVVTHYYGWDRYSHPISINYRTGS

SEQ ID NO: 30 - protein, 365 aa, Artificial Sequence
  Description of Artificial Sequence Synthetic polypeptide
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASRVAREFPKPSTPPGSSGGAPASVSDVPRDLEVVAATPTSLLISWCRQRCADSYRITYGETGGNSPVQEFTVPGSWKTATISGLKPGVDYTITVYVVTHYYGWDRYSHPISINYRTGS

SEQ ID NO: 31 - protein, 374 aa, Artificial Sequence
  Description of Artificial Sequence Synthetic polypeptide
MKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPGILGFVFTLASVSDVPRDLEVVAATPTSLLISWCRQRCADSYRITYGETGGNSPVQEFTVPGSWKTATISGLKPGVDYTITVYVVTHYYGWDRYSHPISINYRTGS

SEQ ID NO: 32 - protein, 512 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 33 - protein, 516 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKGSTSGSGKPGSGEGSEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSS

SEQ ID NO: 34 - protein, 518 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAAEFPKPSTPPGSSGGAPDIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKGSTSGSGKPGSGEGSTKGQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 35 - protein, 403 aa, Artificial Sequence
  Description of Artificial Sequence Synthetic polypeptide
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAEFPKPSTPPGSSGGAPRGSHHMLRFVTVTAEALRFRQIQRGFRTTLDDLSGRSYVMTAEDVDLTLNWGRLSSVLPDYHGQDSVRVGRISFGSINAILGSVALILNCHHHASAVAAHHHHGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN